SEQUENCE LISTING (377 sequences)

SEQ ID NO: 1 - protein, 19 aa, Homo sapiens
MDWTWRILFLVAAATGTHA

SEQ ID NO: 2 - protein, 107 aa, Homo sapiens
ETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPT

SEQ ID NO: 3 - protein, 116 aa, Homo sapiens
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPT

SEQ ID NO: 4 - protein, 107 aa, synthetic construct
ETRECIYYNANWELERTNQSGLERCEEEQDKRLHCYASWRNSSGTIELVKKGCWLDDFNCYDRQECVATEENPQVYFCCCEGNFCNERFTHLPEAGGPEVTYEPPPT

SEQ ID NO: 5 - protein, 107 aa, synthetic construct
ETRECIYYNANWELERTNQSGLERCENEQDKRLHCYASWRNSSGTIELVKKGCWLDDFNCYDRQECVATEENPQVYFCCCEGNFCNERFTHLPEAGGPEVTYEPPPT

SEQ ID NO: 6 - protein, 107 aa, synthetic construct
ETRECIYYNANWELERTNQSGLERCEQEQDKRLHCYASWRNSSGTIELVKKGCWLDDFNCYDRQECVATEENPQVYFCCCEGNFCNERFTHLPEAGGPEVTYEPPPT

SEQ ID NO: 7 - protein, 107 aa, synthetic construct
ETRECIYYNANWELERTNQSGLERCEKEQDKRLHCYASWRNSSGTIELVKKGCWLDDFNCYDRQECVATEENPQVYFCCCEGNFCNERFTHLPEAGGPEVTYEPPPT

SEQ ID NO: 8 - protein, 107 aa, synthetic construct
ETRECIYYNANWELERTNQSGLERCETEQDKRLHCYASWRNSSGTIELVKKGCWLDDFNCYDRQECVATEENPQVYFCCCEGNFCNERFTHLPEAGGPEVTYEPPPT

SEQ ID NO: 9 - protein, 107 aa, synthetic construct
ETRECIYYNANWELERTNQSGLERCEMEQDKRLHCYASWRNSSGTIELVKKGCWLDDFNCYDRQECVATEENPQVYFCCCEGNFCNERFTHLPEAGGPEVTYEPPPT

SEQ ID NO: 10 - protein, 107 aa, synthetic construct
ETRECIYYNANWELERTNQSGLERCEGEQDKRFHCYASWRNSSGTIELVKKGCWLDDFNCYDRQECVATEENPQVYFCCCEGNFCNERFTHLPEAGGPEVTYEPPPT

SEQ ID NO: 11 - protein, 107 aa, synthetic construct
ETRECIYYNANWELERTNQSGLERCEGEQDKRQHCYASWRNSSGTIELVKKGCWLDDFNCYDRQECVATEENPQVYFCCCEGNFCNERFTHLPEAGGPEVTYEPPPT

SEQ ID NO: 12 - protein, 107 aa, synthetic construct
ETRECIYYNANWELERTNQSGLERCEGEQDKRYHCYASWRNSSGTIELVKKGCWLDDFNCYDRQECVATEENPQVYFCCCEGNFCNERFTHLPEAGGPEVTYEPPPT

SEQ ID NO: 13 - protein, 107 aa, synthetic construct
ETRECIYYNANWELERTNQSGLERCEGEQDKRWHCYASWRNSSGTIELVKKGCWLDDFNCYDRQECVATEENPQVYFCCCEGNFCNERFTHLPEAGGPEVTYEPPPT

SEQ ID NO: 14 - protein, 107 aa, synthetic construct
ETRECIYYNANWELERTNQSGLERCEGEQDKRHHCYASWRNSSGTIELVKKGCWLDDFNCYDRQECVATEENPQVYFCCCEGNFCNERFTHLPEAGGPEVTYEPPPT

SEQ ID NO: 15 - protein, 107 aa, synthetic construct
ETRECIYYNANWELERTNQSGLERCEGEQDKRRHCYASWRNSSGTIELVKKGCWLDDFNCYDRQECVATEENPQVYFCCCEGNFCNERFTHLPEAGGPEVTYEPPPT

SEQ ID NO: 16 - protein, 107 aa, synthetic construct
ETRECIYYNANWELERTNQSGLERCEGEQDKREHCYASWRNSSGTIELVKKGCWLDDFNCYDRQECVATEENPQVYFCCCEGNFCNERFTHLPEAGGPEVTYEPPPT

SEQ ID NO: 17 - protein, 107 aa, synthetic construct
ETRECIYYNANWELERTNQSGLERCEGEQDKRKHCYASWRNSSGTIELVKKGCWLDDFNCYDRQECVATEENPQVYFCCCEGNFCNERFTHLPEAGGPEVTYEPPPT

SEQ ID NO: 18 - protein, 107 aa, synthetic construct
ETRECIYYNANWELERTNQSGLERCEGEQDKRMHCYASWRNSSGTIELVKKGCWLDDFNCYDRQECVATEENPQVYFCCCEGNFCNERFTHLPEAGGPEVTYEPPPT

SEQ ID NO: 19 - protein, 107 aa, synthetic construct
ETRECIYYNANWELERTNQSGLERCEGEQDKRLHCYASWRNSSGTIELVKKGCWLDDFNCYDRQECVAHEENPQVYFCCCEGNFCNERFTHLPEAGGPEVTYEPPPT

SEQ ID NO: 20 - protein, 107 aa, synthetic construct
ETRECIYYNANWELERTNQSGLERCEGEQDKRLHCYASWRNSSGTIELVKKGCWLDDFNCYDRQECVAQEENPQVYFCCCEGNFCNERFTHLPEAGGPEVTYEPPPT

SEQ ID NO: 21 - protein, 107 aa, synthetic construct
ETRECIYYNANWELERTNQSGLERCEGEQDKRLHCYASWRNSSGTIELVKKGCWLDDFNCYDRQECVAEEENPQVYFCCCEGNFCNERFTHLPEAGGPEVTYEPPPT

SEQ ID NO: 22 - protein, 107 aa, synthetic construct
ETRECIYYNANWELERTNQSGLERCEGEQDKRLHCYASWRNSSGTIELVKKGCWLDDFNCYDRQECVAREENPQVYFCCCEGNFCNERFTHLPEAGGPEVTYEPPPT

SEQ ID NO: 23 - protein, 107 aa, synthetic construct
ETRECIYYNANWELERTNQSGLERCEGEQDKRLHCYASWRNSSGTIELVKKGCWLDDFNCYDRQECVAYEENPQVYFCCCEGNFCNERFTHLPEAGGPEVTYEPPPT

SEQ ID NO: 24 - protein, 107 aa, synthetic construct
ETRECIYYNANWELERTNQSGLERCEGEQDKRLHCYASWRNSSGTIELVKKGCWLDDFNCYDRQECVAWEENPQVYFCCCEGNFCNERFTHLPEAGGPEVTYEPPPT

SEQ ID NO: 25 - protein, 107 aa, synthetic construct
ETRECIYYNANWELERTNQSGLERCEGEQDKRYHCYASWRNSSGTIELVKKGCWLDDFNCYDRQECVAREENPQVYFCCCEGNFCNERFTHLPEAGGPEVTYEPPPT

SEQ ID NO: 26 - protein, 107 aa, synthetic construct
ETRECIYYNANWELERTNQSGLERCEGEQDKRYHCYASWRNSSGTIELVKKGCWLDDFNCYDRQECVAYEENPQVYFCCCEGNFCNERFTHLPEAGGPEVTYEPPPT

SEQ ID NO: 27 - protein, 107 aa, synthetic construct
ETRECIYYNANWELERTNQSGLERCEGEQDKRYHCYASWRNSSGTIELVKKGCWLDDFNCYDRQECVAWEENPQVYFCCCEGNFCNERFTHLPEAGGPEVTYEPPPT

SEQ ID NO: 28 - protein, 107 aa, synthetic construct
ETRECIYYNANWELERTNQSGLERCEGEQDKRYHCYASWRNSSGTIELVKKGCWLDDFNCYDRQECVAHEENPQVYFCCCEGNFCNERFTHLPEAGGPEVTYEPPPT

SEQ ID NO: 29 - protein, 107 aa, synthetic construct
ETRECIYYNANWELERTNQSGLERCEGEQDKRYHCYASWRNSSGTIELVKKGCWLDDFNCYDRQECVAQEENPQVYFCCCEGNFCNERFTHLPEAGGPEVTYEPPPT

SEQ ID NO: 30 - protein, 107 aa, synthetic construct
ETRECIYYNANWELERTNQSGLERCEGEQDKRYHCYASWRNSSGTIELVKKGCWLDDFNCYDRQECVAEEENPQVYFCCCEGNFCNERFTHLPEAGGPEVTYEPPPT

SEQ ID NO: 31 - protein, 107 aa, synthetic construct
ETRECIYYNANWELERTNQSGLERCEGEQDKRFHCYASWRNSSGTIELVKKGCWLDDFNCYDRQECVAQEENPQVYFCCCEGNFCNERFTHLPEAGGPEVTYEPPPT

SEQ ID NO: 32 - protein, 107 aa, synthetic construct
ETRECIYYNANWELERTNQSGLERCEEEQDKRYHCYASWRNSSGTIELVKKGCWLDDFNCYDRQECVATEENPQVYFCCCEGNFCNERFTHLPEAGGPEVTYEPPPT

SEQ ID NO: 33 - protein, 107 aa, synthetic construct
ETRECIYYNANWELERTNQSGLERCEDEQDKRYHCYASWRNSSGTIELVKKGCWLDDFNCYDRQECVATEENPQVYFCCCEGNFCNERFTHLPEAGGPEVTYEPPPT

SEQ ID NO: 34 - protein, 107 aa, synthetic construct
ETRECIYYNANWELERTNQSGLERCENEQDKRYHCYASWRNSSGTIELVKKGCWLDDFNCYDRQECVATEENPQVYFCCCEGNFCNERFTHLPEAGGPEVTYEPPPT

SEQ ID NO: 35 - protein, 107 aa, synthetic construct
ETRECIYYNANWELERTNQSGLERCEQEQDKRYHCYASWRNSSGTIELVKKGCWLDDFNCYDRQECVATEENPQVYFCCCEGNFCNERFTHLPEAGGPEVTYEPPPT

SEQ ID NO: 36 - protein, 107 aa, synthetic construct
ETRECIYYNANWELERTNQSGLERCEKEQDKRYHCYASWRNSSGTIELVKKGCWLDDFNCYDRQECVATEENPQVYFCCCEGNFCNERFTHLPEAGGPEVTYEPPPT

SEQ ID NO: 37 - protein, 107 aa, synthetic construct
ETRECIYYNANWELERTNQSGLERCETEQDKRYHCYASWRNSSGTIELVKKGCWLDDFNCYDRQECVATEENPQVYFCCCEGNFCNERFTHLPEAGGPEVTYEPPPT

SEQ ID NO: 38 - protein, 107 aa, synthetic construct
ETRECIYYNANWELERTNQSGLERCEMEQDKRYHCYASWRNSSGTIELVKKGCWLDDFNCYDRQECVATEENPQVYFCCCEGNFCNERFTHLPEAGGPEVTYEPPPT

SEQ ID NO: 39 - protein, 107 aa, synthetic construct; identical to SEQ ID NO: 33

SEQ ID NO: 40 - protein, 107 aa, synthetic construct; identical to SEQ ID NO: 32

SEQ ID NO: 41 - protein, 107 aa, synthetic construct; identical to SEQ ID NO: 34

SEQ ID NO: 42 - protein, 107 aa, synthetic construct; identical to SEQ ID NO: 35

SEQ ID NO: 43 - protein, 107 aa, synthetic construct
ETRECIYYNANWELERTNQSGLERCEDEQDKRWHCYASWRNSSGTIELVKKGCWLDDFNCYDRQECVATEENPQVYFCCCEGNFCNERFTHLPEAGGPEVTYEPPPT

SEQ ID NO: 44 - protein, 107 aa, synthetic construct
ETRECIYYNANWELERTNQSGLERCEEEQDKRWHCYASWRNSSGTIELVKKGCWLDDFNCYDRQECVATEENPQVYFCCCEGNFCNERFTHLPEAGGPEVTYEPPPT

SEQ ID NO: 45 - protein, 107 aa, synthetic construct
ETRECIYYNANWELERTNQSGLERCENEQDKRWHCYASWRNSSGTIELVKKGCWLDDFNCYDRQECVATEENPQVYFCCCEGNFCNERFTHLPEAGGPEVTYEPPPT

SEQ ID NO: 46 - protein, 126 aa, synthetic construct
ETRECIYYNANWELERTNQSGLERCEQEQDKRWHCYASWRNSSGTIELVKKGCWLDDFNCYDRQECVATEENPQVYFCCCEGNFCNERFTHLPEAGGPEVTYEPPPTGGGGSGGGGSGGGGSGGGG

SEQ ID NO: 47 - protein, 107 aa, synthetic construct
ETRECIYYNANWELERTNQSGLERCETEQDKRWHCYASWRNSSGTIELVKKGCWLDDFNCYDRQECVATEENPQVYFCCCEGNFCNERFTHLPEAGGPEVTYEPPPT

SEQ ID NO: 48 - protein, 107 aa, synthetic construct
ETRECIYYNANWELERTNQSGLERCEMEQDKRWHCYASWRNSSGTIELVKKGCWLDDFNCYDRQECVATEENPQVYFCCCEGNFCNERFTHLPEAGGPEVTYEPPPT

SEQ ID NO: 49 - protein, 107 aa, synthetic construct
ETRECIYYNANWEQERTNQSGLERCEGEQDKRYHCYASWRNSSGTIELVKKGCWLDDFNCYDRQECVATEENPQVYFCCCEGNFCNERFTHLPEAGGPEVTYEPPPT

SEQ ID NO: 50 - protein, 107 aa, synthetic construct
ETRECIYYNANWEDERTNQSGLERCEGEQDKRYHCYASWRNSSGTIELVKKGCWLDDFNCYDRQECVATEENPQVYFCCCEGNFCNERFTHLPEAGGPEVTYEPPPT

SEQ ID NO: 51 - protein, 107 aa, synthetic construct
ETRECIYYNANWENERTNQSGLERCEGEQDKRYHCYASWRNSSGTIELVKKGCWLDDFNCYDRQECVATEENPQVYFCCCEGNFCNERFTHLPEAGGPEVTYEPPPT

SEQ ID NO: 52 - protein, 107 aa, synthetic construct
ETRECIYYNANWEEERTNQSGLERCEGEQDKRYHCYASWRNSSGTIELVKKGCWLDDFNCYDRQECVATEENPQVYFCCCEGNFCNERFTHLPEAGGPEVTYEPPPT

SEQ ID NO: 53 - protein, 107 aa, synthetic construct
ETRECIYYNANWEHERTNQSGLERCEGEQDKRYHCYASWRNSSGTIELVKKGCWLDDFNCYDRQECVATEENPQVYFCCCEGNFCNERFTHLPEAGGPEVTYEPPPT

SEQ ID NO: 54 - protein, 107 aa, synthetic construct
ETRECIYYNANWESERTNQSGLERCEGEQDKRYHCYASWRNSSGTIELVKKGCWLDDFNCYDRQECVATEENPQVYFCCCEGNFCNERFTHLPEAGGPEVTYEPPPT

SEQ ID NO: 55 - protein, 107 aa, synthetic construct
ETRECIYYNANWEEERTNQSGLERCEGEQDKRWHCYASWRNSSGTIELVKKGCWLDDFNCYDRQECVATEENPQVYFCCCEGNFCNERFTHLPEAGGPEVTYEPPPT

SEQ ID NO: 56 - protein, 107 aa, synthetic construct
ETRECIYYNANWEDERTNQSGLERCEGEQDKRWHCYASWRNSSGTIELVKKGCWLDDFNCYDRQECVATEENPQVYFCCCEGNFCNERFTHLPEAGGPEVTYEPPPT

SEQ ID NO: 57 - protein, 107 aa, synthetic construct
ETRECIYYNANWENERTNQSGLERCEGEQDKRWHCYASWRNSSGTIELVKKGCWLDDFNCYDRQECVATEENPQVYFCCCEGNFCNERFTHLPEAGGPEVTYEPPPT

SEQ ID NO: 58 - protein, 107 aa, synthetic construct
ETRECIYYNANWEQERTNQSGLERCEGEQDKRWHCYASWRNSSGTIELVKKGCWLDDFNCYDRQECVATEENPQVYFCCCEGNFCNERFTHLPEAGGPEVTYEPPPT

SEQ ID NO: 59 - protein, 107 aa, synthetic construct
ETRECIYYNANWELERTNQSGLERCEGEQDKRYHCYASWRNSSGTIELVKKGCWYDDFNCYDRQECVATEENPQVYFCCCEGNFCNERFTHLPEAGGPEVTYEPPPT

SEQ ID NO: 60 - protein, 107 aa, synthetic construct
ETRECIYYNANWELERTNQSGLERCEGEQDKRYHCYASWRNSSGTIELVKKGCWQDDFNCYDRQECVATEENPQVYFCCCEGNFCNERFTHLPEAGGPEVTYEPPPT

SEQ ID NO: 61 - protein, 107 aa, synthetic construct
ETRECIYYNANWELERTNQSGLERCEGEQDKRYHCYASWRNSSGTIELVKKGCWMDDFNCYDRQECVATEENPQVYFCCCEGNFCNERFTHLPEAGGPEVTYEPPPT

SEQ ID NO: 62 - protein, 107 aa, synthetic construct
ETRECIYYNANWELERTNQSGLERCEGEQDKRYHCYASWRNSSGTIELVKKGCWIDDFNCYDRQECVATEENPQVYFCCCEGNFCNERFTHLPEAGGPEVTYEPPPT

SEQ ID NO: 63 - protein, 5 aa, synthetic construct
GRGEA

SEQ ID NO: 64 - skipped

SEQ ID NO: 65 - protein, 115 aa, synthetic construct
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 66 - protein, 112 aa, synthetic construct
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPT

SEQ ID NO: 67 - protein, 110 aa, synthetic construct
ETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 68 - protein, 40 aa, synthetic construct
GGGGSGGGGSGGGGSGGGGSGGGGSGGGGSGGGGSGGGGS

SEQ ID NO: 69 - protein, 39 aa, synthetic construct
GGGGSGGGGSGGGGSGGGGSGGGGSGGGGSGGGGSGGGG

SEQ ID NO: 70 - protein, 38 aa, synthetic construct
GGGGSGGGGSGGGGSGGGGSGGGGSGGGGSGGGGSGGG

SEQ ID NO: 71 - protein, 37 aa, synthetic construct
GGGGSGGGGSGGGGSGGGGSGGGGSGGGGSGGGGSGG

SEQ ID NO: 72 - protein, 36 aa, synthetic construct
GGGGSGGGGSGGGGSGGGGSGGGGSGGGGSGGGGSG

SEQ ID NO: 73 - protein, 35 aa, synthetic construct
GGGGSGGGGSGGGGSGGGGSGGGGSGGGGSGGGGS

SEQ ID NO: 74 - protein, 34 aa, synthetic construct
GGGGSGGGGSGGGGSGGGGSGGGGSGGGGSGGGG

SEQ ID NO: 75 - protein, 33 aa, synthetic construct
GGGGSGGGGSGGGGSGGGGSGGGGSGGGGSGGG

SEQ ID NO: 76 - protein, 32 aa, synthetic construct
GGGGSGGGGSGGGGSGGGGSGGGGSGGGGSGG

SEQ ID NO: 77 - protein, 31 aa, synthetic construct
GGGGSGGGGSGGGGSGGGGSGGGGSGGGGSG

SEQ ID NO: 78 - protein, 30 aa, synthetic construct
GGGGSGGGGSGGGGSGGGGSGGGGSGGGGS

SEQ ID NO: 79 - protein, 29 aa, synthetic construct
GGGGSGGGGSGGGGSGGGGSGGGGSGGGG

SEQ ID NO: 80 - protein, 28 aa, synthetic construct
GGGGSGGGGSGGGGSGGGGSGGGGSGGG

SEQ ID NO: 81 - protein, 27 aa, synthetic construct
GGGGSGGGGSGGGGSGGGGSGGGGSGG

SEQ ID NO: 82 - protein, 26 aa, synthetic construct
GGGGSGGGGSGGGGSGGGGSGGGGSG

SEQ ID NO: 83 - protein, 25 aa, synthetic construct
GGGGSGGGGSGGGGSGGGGSGGGGS

SEQ ID NO: 84 - protein, 24 aa, synthetic construct
GGGGSGGGGSGGGGSGGGGSGGGG

SEQ ID NO: 85 - protein, 23 aa, synthetic construct
GGGGSGGGGSGGGGSGGGGSGGG

SEQ ID NO: 86 - protein, 22 aa, synthetic construct
GGGGSGGGGSGGGGSGGGGSGG

SEQ ID NO: 87 - protein, 21 aa, synthetic construct
GGGGSGGGGSGGGGSGGGGSG

SEQ ID NO: 88 - protein, 20 aa, synthetic construct
GGGGSGGGGSGGGGSGGGGS

SEQ ID NO: 89 - protein, 19 aa, synthetic construct
GGGGSGGGGSGGGGSGGGG

SEQ ID NO: 90 - protein, 18 aa, synthetic construct
GGGGSGGGGSGGGGSGGG

SEQ ID NO: 91 - protein, 17 aa, synthetic construct
GGGGSGGGGSGGGGSGG

SEQ ID NO: 92 - protein, 16 aa, synthetic construct
GGGGSGGGGSGGGGSG

SEQ ID NO: 93 - protein, 15 aa, synthetic construct
GGGGSGGGGSGGGGS

SEQ ID NO: 94 - protein, 14 aa, synthetic construct
GGGGSGGGGSGGGG

SEQ ID NO: 95 - protein, 13 aa, synthetic construct
GGGGSGGGGSGGG

SEQ ID NO: 96 - protein, 12 aa, synthetic construct
GGGGSGGGGSGG

SEQ ID NO: 97 - protein, 11 aa, synthetic construct
GGGGSGGGGSG

SEQ ID NO: 98 - protein, 10 aa, synthetic construct
GGGGSGGGGS

SEQ ID NO: 99 - protein, 9 aa, synthetic construct
GGGGSGGGG

SEQ ID NO: 100 - protein, 8 aa, synthetic construct
GGGGSGGG

SEQ ID NO: 101 - protein, 7 aa, synthetic construct
GGGGSGG

SEQ ID NO: 102 - protein, 6 aa, synthetic construct
GGGGSG

SEQ ID NO: 103 - protein, 5 aa, synthetic construct
GGGGS

SEQ ID NO: 104 - protein, 4 aa, synthetic construct
GGGG

SEQ ID NO: 105-106 - skipped (2 entries)

SEQ ID NO: 107 - protein, 4 aa, synthetic construct
TGGG

SEQ ID NO: 108 - protein, 5 aa, synthetic construct
TGGGG

SEQ ID NO: 109 - protein, 5 aa, synthetic construct
SGGGG

SEQ ID NO: 110 - protein, 4 aa, synthetic construct
SGGG

SEQ ID NO: 111 - protein, 11 aa, synthetic construct
TGGGGSGGGGS

SEQ ID NO: 112 - protein, 16 aa, synthetic construct
TGGGGSGGGGSGGGGS

SEQ ID NO: 113 - protein, 21 aa, synthetic construct
TGGGGSGGGGSGGGGSGGGGS

SEQ ID NO: 114 - protein, 26 aa, synthetic construct
TGGGGSGGGGSGGGGSGGGGSGGGGS

SEQ ID NO: 115 - protein, 31 aa, synthetic construct
TGGGGSGGGGSGGGGSGGGGSGGGGSGGGGS

SEQ ID NO: 116 - protein, 36 aa, synthetic construct
TGGGGSGGGGSGGGGSGGGGSGGGGSGGGGSGGGGS

SEQ ID NO: 117 - protein, 10 aa, synthetic construct
TGGGPKSCDK

SEQ ID NO: 118-119 - skipped (2 entries)

SEQ ID NO: 120 - protein, 4 aa, synthetic construct
AGGG

SEQ ID NO: 121 - protein, 4 aa, synthetic construct
GGSG

SEQ ID NO: 122 - protein, 4 aa, synthetic construct
GGAG

SEQ ID NO: 123 - protein, 5 aa, synthetic construct
GGGGG

SEQ ID NO: 124 - protein, 5 aa, synthetic construct
GGGGA

SEQ ID NO: 125 - protein, 4 aa, synthetic construct
GGGS

SEQ ID NO: 126 - protein, 4 aa, synthetic construct
GGGA

SEQ ID NO: 127-128 - skipped (2 entries)

SEQ ID NO: 129 - protein, 4 aa, synthetic construct
GSGS

SEQ ID NO: 130 - protein, 6 aa, synthetic construct
GAGAGA

SEQ ID NO: 131 - protein, 6 aa, synthetic construct
GSGSGS

SEQ ID NO: 132 - protein, 8 aa, synthetic construct
GAGAGAGA

SEQ ID NO: 133 - protein, 8 aa, synthetic construct
GSGSGSGS

SEQ ID NO: 134 - protein, 10 aa, synthetic construct
GAGAGAGAGA

SEQ ID NO: 135 - protein, 10 aa, synthetic construct
GSGSGSGSGS

SEQ ID NO: 136 - protein, 12 aa, synthetic construct
GAGAGAGAGAGA

SEQ ID NO: 137 - protein, 4 aa, synthetic construct
GAGA

SEQ ID NO: 138 - protein, 12 aa, synthetic construct
GSGSGSGSGSGS

SEQ ID NO: 139 - protein, 6 aa, synthetic construct
GGAGGA

SEQ ID NO: 140 - protein, 6 aa, synthetic construct
GGSGGS

SEQ ID NO: 141 - protein, 9 aa, synthetic construct
GGAGGAGGA

SEQ ID NO: 142 - protein, 9 aa, synthetic construct
GGSGGSGGS

SEQ ID NO: 143 - protein, 12 aa, synthetic construct
GGAGGAGGAGGA

SEQ ID NO: 144 - protein, 12 aa, synthetic construct
GGSGGSGGSGGS

SEQ ID NO: 145 - protein, 4 aa, synthetic construct
GGAG

SEQ ID NO: 146 - protein, 4 aa, synthetic construct
GGSG

SEQ ID NO: 147 - protein, 8 aa, synthetic construct
GGAGGGAG

SEQ ID NO: 148 - protein, 8 aa, synthetic construct
GGSGGGSG

SEQ ID NO: 149 - protein, 12 aa, synthetic construct
GGAGGGAGGGAG

SEQ ID NO: 150 - protein, 12 aa, synthetic construct
GGSGGGSGGGSG

SEQ ID NO: 151 - protein, 15 aa, synthetic construct
GGGGAGGGGAGGGGA

SEQ ID NO: 152 - protein, 4 aa, synthetic construct
AAAL

SEQ ID NO: 153 - protein, 4 aa, synthetic construct
AAAK

SEQ ID NO: 154 - protein, 4 aa, synthetic construct
AAAR

SEQ ID NO: 155 - protein, 14 aa, synthetic construct
EGKSSGSGSESKST

SEQ ID NO: 156 - protein, 12 aa, synthetic construct
GSAGSAAGSGEF

SEQ ID NO: 157 - protein, 12 aa, synthetic construct
AEAAAKEAAAKA

SEQ ID NO: 158 - protein, 18 aa, synthetic construct
KESGSVSSEQLAQFRSLD

SEQ ID NO: 159 - protein, 10 aa, synthetic construct
GENLYFQSGG

SEQ ID NO: 160 - protein, 8 aa, synthetic construct
SACYCELS

SEQ ID NO: 161 - protein, 5 aa, synthetic construct
RSIAT

SEQ ID NO: 162 - protein, 17 aa, synthetic construct
RPACKIPNDLKQKVMNH

SEQ ID NO: 163 - protein, 36 aa, synthetic construct
GGSAGGSGSGSSGGSSGASGTGTAGGTGSGSGTGSG

SEQ ID NO: 164 - protein, 17 aa, synthetic construct
AAANSSIDLISVPVDSR

SEQ ID NO: 165 - protein, 36 aa, synthetic construct
GGSGGGSEGGGSEGGGSEGGGSEGGGSEGGGSGGGS

SEQ ID NO: 166 - protein, 5 aa, synthetic construct
EAAAK

SEQ ID NO: 167 - protein, 5 aa, synthetic construct
PAPAP

SEQ ID NO: 168 - protein, 5 aa, synthetic construct
GGGAG

SEQ ID NO: 169 - protein, 6 aa, synthetic construct
GGGAGG

SEQ ID NO: 170 - protein, 7 aa, synthetic construct
GGGAGGG

SEQ ID NO: 171 - protein, 334 aa, synthetic construct
ETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 172 - protein, 345 aa, synthetic construct
ETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 173 - protein, 343 aa, synthetic construct
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 174 - protein, 345 aa, synthetic construct
ETRECIYYNANWELERTNQSGLERCEEEQDKRLHCYASWRNSSGTIELVKKGCWLDDFNCYDRQECVATEENPQVYFCCCEGNFCNERFTHLPEAGGPEVTYEPPPTGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 175 - protein, 345 aa, synthetic construct
ETRECIYYNANWELERTNQSGLERCEGEQDKRRHCYASWRNSSGTIELVKKGCWLDDFNCYDRQECVATEENPQVYFCCCEGNFCNERFTHLPEAGGPEVTYEPPPTGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 176 - protein, 345 aa, synthetic construct
ETRECIYYNANWELERTNQSGLERCEGEQDKRYHCYASWRNSSGTIELVKKGCWLDDFNCYDRQECVATEENPQVYFCCCEGNFCNERFTHLPEAGGPEVTYEPPPTGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 177 - protein, 345 aa, synthetic construct
ETRECIYYNANWELERTNQSGLERCEGEQDKRLHCYASWRNSSGTIELVKKGCWLDDFNCYDRQECVAHEENPQVYFCCCEGNFCNERFTHLPEAGGPEVTYEPPPTGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 178 - protein, 345 aa, synthetic construct
ETRECIYYNANWELERTNQSGLERCEGEQDKRLHCYASWRNSSGTIELVKKGCWLDDFNCYDRQECVAQEENPQVYFCCCEGNFCNERFTHLPEAGGPEVTYEPPPTGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 179 - protein, 334 aa, synthetic construct
ETRECIYYNANWELERTNQSGLERCEEEQDKRLHCYASWRNSSGTIELVKKGCWLDDFNCYDRQECVATEENPQVYFCCCEGNFCNERFTHLPEAGGPEVTYEPPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 180 - protein, 334 aa, synthetic construct
ETRECIYYNANWELERTNQSGLERCEGEQDKRYHCYASWRNSSGTIELVKKGCWLDDFNCYDRQECVATEENPQVYFCCCEGNFCNERFTHLPEAGGPEVTYEPPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 181 - protein, 334 aa, synthetic construct
ETRECIYYNANWELERTNQSGLERCEGEQDKRLHCYASWRNSSGTIELVKKGCWLDDFNCYDRQECVAHEENPQVYFCCCEGNFCNERFTHLPEAGGPEVTYEPPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 182 - protein, 334 aa, synthetic construct
ETRECIYYNANWELERTNQSGLERCEGEQDKRLHCYASWRNSSGTIELVKKGCWLDDFNCYDRQECVAQEENPQVYFCCCEGNFCNERFTHLPEAGGPEVTYEPPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 183 - protein, 334 aa, synthetic construct
ETRECIYYNANWELERTNQSGLERCEGEQDKRYHCYASWRNSSGTIELVKKGCWLDDFNCYDRQECVAHEENPQVYFCCCEGNFCNERFTHLPEAGGPEVTYEPPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 184 - protein, 341 aa, synthetic construct
ETRECIYYNANWELERTNQSGLERCEGEQDKRYHCYASWRNSSGTIELVKKGCWLDDFNCYDRQECVAHEENPQVYFCCCEGNFCNERFTHLPEAGGPEVTYEPPPTGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 185 - protein, 345 aa, synthetic construct
ETRECIYYNANWELERTNQSGLERCEGEQDKRYHCYASWRNSSGTIELVKKGCWLDDFNCYDRQECVAHEENPQVYFCCCEGNFCNERFTHLPEAGGPEVTYEPPPTGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 186 - protein, 334 aa, synthetic construct
ETRECIYYNANWELERTNQSGLERCEGEQDKRYHCYASWRNSSGTIELVKKGCWLDDFNCYDRQECVAQEENPQVYFCCCEGNFCNERFTHLPEAGGPEVTYEPPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 187 - protein, 341 aa, synthetic construct
ETRECIYYNANWELERTNQSGLERCEGEQDKRYHCYASWRNSSGTIELVKKGCWLDDFNCYDRQECVAQEENPQVYFCCCEGNFCNERFTHLPEAGGPEVTYEPPPTGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 188 - protein, 345 aa, synthetic construct
ETRECIYYNANWELERTNQSGLERCEGEQDKRYHCYASWRNSSGTIELVKKGCWLDDFNCYDRQECVAQEENPQVYFCCCEGNFCNERFTHLPEAGGPEVTYEPPPTGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 189 - protein, 334 aa, synthetic construct
ETRECIYYNANWELERTNQSGLERCEGEQDKRYHCYASWRNSSGTIELVKKGCWLDDFNCYDRQECVAEEENPQVYFCCCEGNFCNERFTHLPEAGGPEVTYEPPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 190 - protein, 341 aa, synthetic construct
ETRECIYYNANWELERTNQSGLERCEGEQDKRYHCYASWRNSSGTIELVKKGCWLDDFNCYDRQECVAEEENPQVYFCCCEGNFCNERFTHLPEAGGPEVTYEPPPTGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 191 - protein, 345 aa, synthetic construct
ETRECIYYNANWELERTNQSGLERCEGEQDKRYHCYASWRNSSGTIELVKKGCWLDDFNCYDRQECVAEEENPQVYFCCCEGNFCNERFTHLPEAGGPEVTYEPPPTGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 192 - protein, 334 aa, synthetic construct
ETRECIYYNANWELERTNQSGLERCEDEQDKRYHCYASWRNSSGTIELVKKGCWLDDFNCYDRQECVATEENPQVYFCCCEGNFCNERFTHLPEAGGPEVTYEPPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 193 - protein, 341 aa, synthetic construct
ETRECIYYNANWELERTNQSGLERCEDEQDKRYHCYASWRNSSGTIELVKKGCWLDDFNCYDRQECVATEENPQVYFCCCEGNFCNERFTHLPEAGGPEVTYEPPPTGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 194 - protein, 345 aa, synthetic construct
ETRECIYYNANWELERTNQSGLERCEDEQDKRYHCYASWRNSSGTIELVKKGCWLDDFNCYDRQECVATEENPQVYFCCCEGNFCNERFTHLPEAGGPEVTYEPPPTGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 195 - protein, 334 aa, synthetic construct
ETRECIYYNANWELERTNQSGLERCEEEQDKRYHCYASWRNSSGTIELVKKGCWLDDFNCYDRQECVATEENPQVYFCCCEGNFCNERFTHLPEAGGPEVTYEPPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 196 - protein, 341 aa, synthetic construct
ETRECIYYNANWELERTNQSGLERCEEEQDKRYHCYASWRNSSGTIELVKKGCWLDDFNCYDRQECVATEENPQVYFCCCEGNFCNERFTHLPEAGGPEVTYEPPPTGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 197 - protein, 345 aa, synthetic construct
ETRECIYYNANWELERTNQSGLERCEEEQDKRYHCYASWRNSSGTIELVKKGCWLDDFNCYDRQECVATEENPQVYFCCCEGNFCNERFTHLPEAGGPEVTYEPPPTGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 198 - protein, 334 aa, synthetic construct
ETRECIYYNANWELERTNQSGLERCEGEQDKRFHCYASWRNSSGTIELVKKGCWLDDFNCYDRQECVAQEENPQVYFCCCEGNFCNERFTHLPEAGGPEVTYEPPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 199 - protein, 341 aa, synthetic construct
ETRECIYYNANWELERTNQSGLERCEGEQDKRFHCYASWRNSSGTIELVKKGCWLDDFNCYDRQECVAQEENPQVYFCCCEGNFCNERFTHLPEAGGPEVTYEPPPTGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 200 - protein, 345 aa, synthetic construct
ETRECIYYNANWELERTNQSGLERCEGEQDKRFHCYASWRNSSGTIELVKKGCWLDDFNCYDRQECVAQEENPQVYFCCCEGNFCNERFTHLPEAGGPEVTYEPPPTGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 201 - protein, 334 aa, synthetic construct
ETRECIYYNANWELERTNQSGLERCEGEQDKRLHCYASWRNSSGTIELVKKGCWLDDFNCYDRQECVAEEENPQVYFCCCEGNFCNERFTHLPEAGGPEVTYEPPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 202 - protein, 341 aa, synthetic construct
ETRECIYYNANWELERTNQSGLERCEGEQDKRLHCYASWRNSSGTIELVKKGCWLDDFNCYDRQECVAEEENPQVYFCCCEGNFCNERFTHLPEAGGPEVTYEPPPTGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 203 - protein, 345 aa, synthetic construct
ETRECIYYNANWELERTNQSGLERCEGEQDKRLHCYASWRNSSGTIELVKKGCWLDDFNCYDRQECVAEEENPQVYFCCCEGNFCNERFTHLPEAGGPEVTYEPPPTGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 204 - protein, 334 aa, synthetic construct
ETRECIYYNANWELERTNQSGLERCEGEQDKRFHCYASWRNSSGTIELVKKGCWLDDFNCYDRQECVATEENPQVYFCCCEGNFCNERFTHLPEAGGPEVTYEPPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 205 - protein, 341 aa, synthetic construct
ETRECIYYNANWELERTNQSGLERCEGEQDKRFHCYASWRNSSGTIELVKKGCWLDDFNCYDRQECVATEENPQVYFCCCEGNFCNERFTHLPEAGGPEVTYEPPPTGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 206 - protein, 345 aa, synthetic construct
ETRECIYYNANWELERTNQSGLERCEGEQDKRFHCYASWRNSSGTIELVKKGCWLDDFNCYDRQECVATEENPQVYFCCCEGNFCNERFTHLPEAGGPEVTYEPPPTGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 207 - protein, 334 aa, synthetic construct
ETRECIYYNANWELERTNQSGLERCEGEQDKRQHCYASWRNSSGTIELVKKGCWLDDFNCYDRQECVATEENPQVYFCCCEGNFCNERFTHLPEAGGPEVTYEPPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 208 - protein, 341 aa, synthetic construct
ETRECIYYNANWELERTNQSGLERCEGEQDKRQHCYASWRNSSGTIELVKKGCWLDDFNCYDRQECVATEENPQVYFCCCEGNFCNERFTHLPEAGGPEVTYEPPPTGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 209 - protein, 345 aa, synthetic construct
ETRECIYYNANWELERTNQSGLERCEGEQDKRQHCYASWRNSSGTIELVKKGCWLDDFNCYDRQECVATEENPQVYFCCCEGNFCNERFTHLPEAGGPEVTYEPPPTGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 210 - protein, 341 aa, synthetic construct
ETRECIYYNANWELERTNQSGLERCEGEQDKRYHCYASWRNSSGTIELVKKGCWLDDFNCYDRQECVATEENPQVYFCCCEGNFCNERFTHLPEAGGPEVTYEPPPTGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 211 - protein, 345 aa, synthetic construct
ETRECIYYNANWELERTNQSGLERCEGEQDKRWHCYASWRNSSGTIELVKKGCWLDDFNCYDRQECVATEENPQVYFCCCEGNFCNERFTHLPEAGGPEVTYEPPPTGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 212 - protein, 345 aa, synthetic construct
ETRECIYYNANWELERTNQSGLERCEGEQDKREHCYASWRNSSGTIELVKKGCWLDDFNCYDRQECVATEENPQVYFCCCEGNFCNERFTHLPEAGGPEVTYEPPPTGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 213 - protein, 345 aa, synthetic construct
ETRECIYYNANWELERTNQSGLERCEGEQDKRKHCYASWRNSSGTIELVKKGCWLDDFNCYDRQECVATEENPQVYFCCCEGNFCNERFTHLPEAGGPEVTYEPPPTGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 214 - protein, 345 aa, synthetic construct
ETRECIYYNANWELERTNQSGLERCEGEQDKRMHCYASWRNSSGTIELVKKGCWLDDFNCYDRQECVATEENPQVYFCCCEGNFCNERFTHLPEAGGPEVTYEPPPTGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 215 - protein, 345 aa, synthetic construct
ETRECIYYNANWELERTNQSGLERCENEQDKRYHCYASWRNSSGTIELVKKGCWLDDFNCYDRQECVATEENPQVYFCCCEGNFCNERFTHLPEAGGPEVTYEPPPTGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 216 - protein, 345 aa, synthetic construct
ETRECIYYNANWELERTNQSGLERCEQEQDKRYHCYASWRNSSGTIELVKKGCWLDDFNCYDRQECVATEENPQVYFCCCEGNFCNERFTHLPEAGGPEVTYEPPPTGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 217 - protein, 345 aa, synthetic construct
ETRECIYYNANWELERTNQSGLERCEKEQDKRYHCYASWRNSSGTIELVKKGCWLDDFNCYDRQECVATEENPQVYFCCCEGNFCNERFTHLPEAGGPEVTYEPPPTGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 218 - protein, 345 aa, synthetic construct
ETRECIYYNANWELERTNQSGLERCETEQDKRYHCYASWRNSSGTIELVKKGCWLDDFNCYDRQECVATEENPQVYFCCCEGNFCNERFTHLPEAGGPEVTYEPPPTGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 219 - protein, 345 aa, synthetic construct
ETRECIYYNANWELERTNQSGLERCEMEQDKRYHCYASWRNSSGTIELVKKGCWLDDFNCYDRQECVATEENPQVYFCCCEGNFCNERFTHLPEAGGPEVTYEPPPTGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 220 - protein, 345 aa, synthetic construct
ETRECIYYNANWELERTNQSGLERCEGEQDKRYHCYASWRNSSGTIELVKKGCWLDDFNCYDRQECVAREENPQVYFCCCEGNFCNERFTHLPEAGGPEVTYEPPPTGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 221 - protein, 345 aa, synthetic construct
ETRECIYYNANWELERTNQSGLERCEGEQDKRYHCYASWRNSSGTIELVKKGCWLDDFNCYDRQECVAYEENPQVYFCCCEGNFCNERFTHLPEAGGPEVTYEPPPTGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 222 - protein, 345 aa, synthetic construct
ETRECIYYNANWELERTNQSGLERCEGEQDKRYHCYASWRNSSGTIELVKKGCWLDDFNCYDRQECVAWEENPQVYFCCCEGNFCNERFTHLPEAGGPEVTYEPPPTGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 223 - protein, 345 aa, synthetic construct
ETRECIYYNANWEEERTNQSGLERCEGEQDKRYHCYASWRNSSGTIELVKKGCWLDDFNCYDRQECVATEENPQVYFCCCEGNFCNERFTHLPEAGGPEVTYEPPPTGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 224 - protein, 345 aa, synthetic construct
ETRECIYYNANWEHERTNQSGLERCEGEQDKRYHCYASWRNSSGTIELVKKGCWLDDFNCYDRQECVATEENPQVYFCCCEGNFCNERFTHLPEAGGPEVTYEPPPTGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 225 - protein, 345 aa, synthetic construct
ETRECIYYNANWESERTNQSGLERCEGEQDKRYHCYASWRNSSGTIELVKKGCWLDDFNCYDRQECVATEENPQVYFCCCEGNFCNERFTHLPEAGGPEVTYEPPPTGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 226 - protein, 345 aa, synthetic construct
ETRECIYYNANWELERTNQSGLERCEGEQDKRYHCYASWRNSSGTIELVKKGCWYDDFNCYDRQECVATEENPQVYFCCCEGNFCNERFTHLPEAGGPEVTYEPPPTGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 227 - protein, 345 aa, synthetic construct
ETRECIYYNANWELERTNQSGLERCEGEQDKRYHCYASWRNSSGTIELVKKGCWQDDFNCYDRQECVATEENPQVYFCCCEGNFCNERFTHLPEAGGPEVTYEPPPTGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 228 - protein, 345 aa, synthetic construct
ETRECIYYNANWELERTNQSGLERCEGEQDKRYHCYASWRNSSGTIELVKKGCWMDDFNCYDRQECVATEENPQVYFCCCEGNFCNERFTHLPEAGGPEVTYEPPPTGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 229 - protein, 345 aa, synthetic construct
ETRECIYYNANWELERTNQSGLERCEGEQDKRYHCYASWRNSSGTIELVKKGCWIDDFNCYDRQECVATEENPQVYFCCCEGNFCNERFTHLPEAGGPEVTYEPPPTGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 230 - protein, 345 aa, synthetic construct
ETRECIYYNANWELERTNQSGLERCEGEQDKRWHCYASWRNSSGTIELVKKGCWLDDFNCYDRQECVATEENPQVYFCCCEGNFCNERFTHLPEAGGPEVTYEPPPTGGGGSGGGGSGGGGTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 231 - protein, 345 aa, synthetic construct
ETRECIYYNANWELERTNQSGLERCEGEQDKRWHCYASWRNSSGTIELVKKGCWLDDFNCYDRQECVATEENPQVYFCCCEGNFCNERFTHLPEAGGPEVTYEPPPTGGGGSGGGGSGGGGTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 232 - protein, 350 aa, synthetic construct
ETRECIYYNANWELERTNQSGLERCEGEQDKRWHCYASWRNSSGTIELVKKGCWLDDFNCYDRQECVATEENPQVYFCCCEGNFCNERFTHLPEAGGPEVTYEPPPTGGGGSGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 233 - protein, 350 aa, synthetic construct
ETRECIYYNANWELERTNQSGLERCEGEQDKRWHCYASWRNSSGTIELVKKGCWLDDFNCYDRQECVATEENPQVYFCCCEGNFCNERFTHLPEAGGPEVTYEPPPTGGGGSGGGGSGGGGSGGGGTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 234 - protein, 350 aa, synthetic construct
ETRECIYYNANWELERTNQSGLERCEGEQDKRWHCYASWRNSSGTIELVKKGCWLDDFNCYDRQECVATEENPQVYFCCCEGNFCNERFTHLPEAGGPEVTYEPPPTGGGGSGGGGSGGGGSGGGGTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 235 - protein, 350 aa, synthetic construct
ETRECIYYNANWELERTNQSGLERCEDEQDKRWHCYASWRNSSGTIELVKKGCWLDDFNCYDRQECVATEENPQVYFCCCEGNFCNERFTHLPEAGGPEVTYEPPPTGGGGSGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 236 - protein, 350 aa, synthetic construct
ETRECIYYNANWELERTNQSGLERCEEEQDKRWHCYASWRNSSGTIELVKKGCWLDDFNCYDRQECVATEENPQVYFCCCEGNFCNERFTHLPEAGGPEVTYEPPPTGGGGSGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 237 - protein, 350 aa, synthetic construct
ETRECIYYNANWELERTNQSGLERCENEQDKRWHCYASWRNSSGTIELVKKGCWLDDFNCYDRQECVATEENPQVYFCCCEGNFCNERFTHLPEAGGPEVTYEPPPTGGGGSGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 238 - protein, 350 aa, synthetic construct
ETRECIYYNANWELERTNQSGLERCEQEQDKRWHCYASWRNSSGTIELVKKGCWLDDFNCYDRQECVATEENPQVYFCCCEGNFCNERFTHLPEAGGPEVTYEPPPTGGGGSGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 239 - protein, 350 aa, synthetic construct
ETRECIYYNANWELERTNQSGLERCETEQDKRWHCYASWRNSSGTIELVKKGCWLDDFNCYDRQECVATEENPQVYFCCCEGNFCNERFTHLPEAGGPEVTYEPPPTGGGGSGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 240 - protein, 350 aa, synthetic construct
ETRECIYYNANWELERTNQSGLERCEMEQDKRWHCYASWRNSSGTIELVKKGCWLDDFNCYDRQECVATEENPQVYFCCCEGNFCNERFTHLPEAGGPEVTYEPPPTGGGGSGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 241 - protein, 350 aa, synthetic construct
ETRECIYYNANWELERTNQSGLERCEDEQDKRYHCYASWRNSSGTIELVKKGCWLDDFNCYDRQECVATEENPQVYFCCCEGNFCNERFTHLPEAGGPEVTYEPPPTGGGGSGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 242 - protein, 350 aa, synthetic construct
ETRECIYYNANWELERTNQSGLERCEEEQDKRYHCYASWRNSSGTIELVKKGCWLDDFNCYDRQECVATEENPQVYFCCCEGNFCNERFTHLPEAGGPEVTYEPPPTGGGGSGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 243 - protein, 350 aa, synthetic construct
ETRECIYYNANWELERTNQSGLERCENEQDKRYHCYASWRNSSGTIELVKKGCWLDDFNCYDRQECVATEENPQVYFCCCEGNFCNERFTHLPEAGGPEVTYEPPPTGGGGSGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 244 - protein, 350 aa, synthetic construct
ETRECIYYNANWELERTNQSGLERCEQEQDKRYHCYASWRNSSGTIELVKKGCWLDDFNCYDRQECVATEENPQVYFCCCEGNFCNERFTHLPEAGGPEVTYEPPPTGGGGSGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 245 - protein, 350 aa, synthetic construct
ETRECIYYNANWEEERTNQSGLERCEGEQDKRWHCYASWRNSSGTIELVKKGCWLDDFNCYDRQECVATEENPQVYFCCCEGNFCNERFTHLPEAGGPEVTYEPPPTGGGGSGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 246 - protein, 350 aa, synthetic construct
ETRECIYYNANWEDERTNQSGLERCEGEQDKRWHCYASWRNSSGTIELVKKGCWLDDFNCYDRQECVATEENPQVYFCCCEGNFCNERFTHLPEAGGPEVTYEPPPTGGGGSGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 247 - protein, 350 aa, synthetic construct
ETRECIYYNANWENERTNQSGLERCEGEQDKRWHCYASWRNSSGTIELVKKGCWLDDFNCYDRQECVATEENPQVYFCCCEGNFCNERFTHLPEAGGPEVTYEPPPTGGGGSGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 248 - protein, 350 aa, synthetic construct
ETRECIYYNANWEQERTNQSGLERCEGEQDKRWHCYASWRNSSGTIELVKKGCWLDDFNCYDRQECVATEENPQVYFCCCEGNFCNERFTHLPEAGGPEVTYEPPPTGGGGSGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 249 - protein, 350 aa, synthetic construct
ETRECIYYNANWEQERTNQSGLERCEGEQDKRYHCYASWRNSSGTIELVKKGCWLDDFNCYDRQECVATEENPQVYFCCCEGNFCNERFTHLPEAGGPEVTYEPPPTGGGGSGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 250 - protein, 350 aa, synthetic construct
ETRECIYYNANWEDERTNQSGLERCEGEQDKRYHCYASWRNSSGTIELVKKGCWLDDFNCYDRQECVATEENPQVYFCCCEGNFCNERFTHLPEAGGPEVTYEPPPTGGGGSGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 251 - protein, 350 aa, synthetic construct
ETRECIYYNANWENERTNQSGLERCEGEQDKRYHCYASWRNSSGTIELVKKGCWLDDFNCYDRQECVATEENPQVYFCCCEGNFCNERFTHLPEAGGPEVTYEPPPTGGGGSGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 252 - protein, 224 aa, Homo sapiens
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 253 - protein, 224 aa, Homo sapiens
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 254 - protein, 224 aa, Homo sapiens
THTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 255 - protein, 224 aa, Homo sapiens
THTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 256 - protein, 224 aa, Homo sapiens
THTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 257 - protein, 224 aa, Homo sapiens
THTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 258 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 259 - protein, 216 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 260 - protein, 220 aa, Homo sapiens
PPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 261 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 262 - protein, 231 aa, Homo sapiens
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 263 - protein, 231 aa, Homo sapiens
EPKSSDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 264 - protein, 224 aa, Homo sapiens; identical to SEQ ID NO: 257

SEQ ID NO: 265 - protein, 224 aa, Homo sapiens
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 266 - protein, 224 aa, Homo sapiens
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 267 - protein, 216 aa, Homo sapiens
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 268 - protein, 223 aa, Homo sapiens
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 269 - protein, 228 aa, Homo sapiens
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 270 - protein, 227 aa, Homo sapiens
ERKSSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPG

SEQ ID NO: 271 - protein, 227 aa, Homo sapiens
ERKSSVESPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPG

SEQ ID NO: 272 - protein, 227 aa, Homo sapiens
ERKSSVESPPSPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPG

SEQ ID NO: 273 - protein, 215 aa, Homo sapiens
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPG

SEQ ID NO: 274 - protein, 219 aa, Homo sapiens
PPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPG

SEQ ID NO: 275 - protein, 222 aa, Homo sapiens
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPG

SEQ ID NO: 276 - protein, 224 aa, Homo sapiens
PCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 277 - protein, 217 aa, Homo sapiens
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 278 - protein, 216 aa, Homo sapiens
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPG

SEQ ID NO: 279 - protein, 220 aa, Homo sapiens
PRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPG

SEQ ID NO: 280 - protein, 226 aa, Homo sapiens
DTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPG

SEQ ID NO: 281 - protein, 231 aa, Homo sapiens
EPKSSDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPG

SEQ ID NO: 282 - protein, 231 aa, Homo sapiens
EPKSSDTPPPSPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPG

SEQ ID NO: 283 - protein, 231 aa, Homo sapiens
EPKSSDTPPPSPRSPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPG

SEQ ID NO: 284 - protein, 217 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 285 - protein, 224 aa, Homo sapiens
GPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 286 - protein, 216 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 287 - protein, 220 aa, Homo sapiens
PSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 288 - protein, 228 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 289 - protein, 228 aa, Homo sapiens
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 290 - protein, 228 aa, Homo sapiens
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 291 - protein, 228 aa, Homo sapiens
ESKYGPPSPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 292 - protein, 228 aa, Homo sapiens
ESKYGPPSPSSPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 293 - DNA, 1035 bp, synthetic construct
  source 1..1035: mol_type = other DNA
gagaccagggagtgtatctactataacgccaattgggagctggagaggacaaaccagtccggactggagcggtgtgagggcgagcaggacaagagataccattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 294 - DNA, 1035 bp, synthetic construct
  source 1..1035: mol_type = other DNA
gagaccagggagtgtatctactataacgccaattgggagctggagaggacaaaccagtccggactggagcggtgtgagggcgagcaggacaagagataccattgctacgctagctggcgcaattccagcggcaccatcgagctggtgaagaagggctgttggctggacgatttcaactgttacgatagacaggagtgcgtggcccacgaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 295 - DNA, 1035 bp, synthetic construct; identical to SEQ ID NO: 294
  source 1..1035: mol_type = other DNA

SEQ ID NO: 296 - DNA, 1035 bp, synthetic construct
  source 1..1035: mol_type = other DNA
gagaccagggagtgtatctactataacgccaattgggagctggagaggacaaaccagtccggactggagcggtgtgagggcgagcaggacaagagatggcattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccggaggaggaggaacccatacatgtccaccttgcccagctccagagctgctgggaggcccttccgtgttcctgtttccacccaagccaaaggacaccctgtacatcaccagggagccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 297 - DNA, 63 bp, synthetic construct
  source 1..63: mol_type = other DNA
gccaccatggactggacctggagaatcctgttcctggtggctgctgctaccggaacacacgct

SEQ ID NO: 298 - DNA, 1002 bp, synthetic construct
  source 1..1002: mol_type = other DNA
gagacaagagagtgcatctactacaacgccaactgggaactcgagcggaccaaccagtctggcctggaaagatgtgaaggcgagcaggacaagcggctgcactgttacgcctcttggagaaactcctccggcaccatcgagctggtcaagaaaggctgctgggacgacgacttcaactgctacgaccggcaagagtgcgtggccaccgaagagaatccccaggtgtacttctgctgctgcgagggcaacttctgcaacgagcggttcacccatctgcctgaagctggcggacctgaagtgacctatgagcctcctccaaccggcggaggcacacatacttgtcctccatgtccagctccagagctgctcggagggcccagtgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagaatacaagtgcaaggtgtccaacaaggccctgcctgctcctatcgaaaagaccatctccaaggccaagggccagcctagggaaccccaggtgtacacattgcctccatctcgggacgagctgaccaagaaccaggtgtccctgacctgtctggtcaagggcttctacccctccgatatcgccgtggaatgggagtctaatggccagcctgagaacaactacaagaccacacctcctgtgctggactccgacggctcattcttcctgtactccaagctgacagtggacaagtccagatggcagcagggcaacgtgttctcctgctccgtgatgcacgaggccctgcacaatcactacacacagaagtccttaagcttatcccctggc

SEQ ID NO: 299 - DNA, 1002 bp, synthetic construct
  source 1..1002: mol_type = other DNA
gagaccagggagtgtatctactataacgccaattgggagctggagaggacaaaccagtccggactggagcggtgtgagggagagcaggacaagagactgcattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 300 - DNA, 1029 bp, synthetic construct
  source 1..1029: mol_type = other DNA
atcctgggccgcagcgagacacaggagtgtctgttctttaacgccaattgggagaaggataggaccaaccagacaggcgtggagccatgttatggcgacaaggataagaggcggcattgcttcgctacctggaagaacatctccggcagcatcgagatcgtgaagcagggctgttggctggacgatatcaattgttacgaccggacagattgcgtggagaagaaggactctcccgaggtgtatttttgctgttgcgagggcaacatgtgcaatgagaagttctcttactttcccgagatggaggtgacccagcctacatccaatccagtgaccccaaagccacctacaggaggaggaacccacacatgtccaccatgccctgctccagagctgctgggaggaccatccgtgttcctgtttcctccaaagcccaaggacaccctgatgatcagcagaacccccgaggtgacatgcgtggtggtggacgtgtctcacgaggaccccgaggtgaagtttaactggtacgtggatggcgtggaggtgcataatgccaagaccaagcctagagaggagcagtacaactccacctatcgcgtggtgagcgtgctgacagtgctgcatcaggattggctgaacggcaaggagtacaagtgtaaggtgtccaataaggctctgcccgtgcctatcgagaagaccatcagcaaggccaagggccagcctagggagccacaggtgtatacactgcccccttcccgggaggagatgaccaagaaccaggtgagcctgacatgcctggtgaagggcttctaccctagcgacatcgctgtggagtgggagtctaatggccagccagagaacaattataagaccacaccacccgtgctggacagcgatggctctttctttctgtactctaagctgaccgtggataagtcccgctggcagcagggcaacgtgttttcttgttccgtgatgcacgaggccctgcacaatcattacacacagaagagcctgtctctgtccccaggc

SEQ ID NO: 301 - DNA, 1035 bp, synthetic construct
  source 1..1035: mol_type = other DNA
gagaccagggagtgtatctactataacgccaattgggagctggagaggacaaaccagtccggactggagcggtgtgaggaggagcaggacaagagactgcattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 302 - DNA, 1035 bp, synthetic construct
  source 1..1035: mol_type = other DNA
gagaccagggagtgtatctactataacgccaattgggagctggagaggacaaaccagtccggactggagcggtgtgagggcgagcaggacaagagaaggcattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 303 - DNA, 1035 bp, synthetic construct
  source 1..1035: mol_type = other DNA
gagaccagggagtgtatctactataacgccaattgggagctggagaggacaaaccagtccggactggagcggtgtgagggcgagcaggacaagagactgcattgctacgctagctggcgcaattccagcggcaccatcgagctggtgaagaagggctgttggctggacgatttcaactgttacgatagacaggagtgcgtggcccacgaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 304 - DNA, 1035 bp, synthetic construct
  source 1..1035: mol_type = other DNA
gagaccagggagtgtatctactataacgccaattgggagctggagaggacaaaccagtccggactggagcggtgtgagggcgagcaggacaagagactgcattgctacgctagctggcgcaattccagcggcaccatcgagctggtgaagaagggctgttggctggacgatttcaactgttacgatagacaggagtgcgtggcccaggaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 305 - DNA, 1002 bp, synthetic construct
  source 1..1002: mol_type = other DNA
gagaccagggagtgtatctactataacgccaattgggagctggagaggacaaaccagtccggactggagcggtgtgaggaggagcaggacaagagactgcattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 306 - DNA, 1002 bp, synthetic construct
  source 1..1002: mol_type = other DNA
gagaccagggagtgtatctactataacgccaattgggagctggagaggacaaaccagtccggactggagcggtgtgagggagagcaggacaagagataccattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 307 - DNA, 1002 bp, synthetic construct
  source 1..1002: mol_type = other DNA
gagaccagggagtgtatctactataacgccaattgggagctggagaggacaaaccagtccggactggagcggtgtgagggagagcaggacaagagactgcattgctacgctagctggcgcaattccagcggcaccatcgagctggtgaagaagggctgttggctggacgatttcaactgttacgatagacaggagtgcgtggcccacgaggagaatcctcaggtgtatttttgctgttgcgagggcaacttctgcaatgagaggttcacccacctgccagaggctggaggaccagaggtgacctatgagccacctccaacaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 308 - DNA, 1002 bp, synthetic construct
  source 1..1002: mol_type = other DNA
gagaccagggagtgtatctactataacgccaattgggagctggagaggacaaaccagtccggactggagcggtgtgagggagagcaggacaagagactgcattgctacgctagctggcgcaattccagcggcaccatcgagctggtgaagaagggctgttggctggacgatttcaactgttacgatagacaggagtgcgtggcccaggaggagaatcctcaggtgtatttttgctgttgcgagggcaacttctgcaatgagaggttcacccacctgccagaggctggaggaccagaggtgacctatgagccacctccaacaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 309 - DNA, 1002 bp, synthetic construct
  source 1..1002: mol_type = other DNA
gagaccagggagtgtatctactataacgccaattgggagctggagaggacaaaccagtccggactggagcggtgtgagggagagcaggacaagagataccattgctacgctagctggcgcaattccagcggcaccatcgagctggtgaagaagggctgttggctggacgatttcaactgttacgatagacaggagtgcgtggcccacgaggagaatcctcaggtgtatttttgctgttgcgagggcaacttctgcaatgagaggttcacccacctgccagaggctggaggaccagaggtgacctatgagccacctccaacaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 310 - DNA, 1023 bp, synthetic construct
  source 1..1023: mol_type = other DNA
gagaccagggagtgtatctactataacgccaattgggagctggagaggacaaaccagtccggactggagcggtgtgagggcgagcaggacaagagataccattgctacgctagctggcgcaattccagcggcaccatcgagctggtgaagaagggctgttggctggacgatttcaactgttacgatagacaggagtgcgtggcccacgaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 311 - DNA, 1002 bp, synthetic construct
  source 1..1002: mol_type = other DNA
gagaccagggagtgtatctactataacgccaattgggagctggagaggacaaaccagtccggactggagcggtgtgagggagagcaggacaagagataccattgctacgctagctggcgcaattccagcggcaccatcgagctggtgaagaagggctgttggctggacgatttcaactgttacgatagacaggagtgcgtggcccaggaggagaatcctcaggtgtatttttgctgttgcgagggcaacttctgcaatgagaggttcacccacctgccagaggctggaggaccagaggtgacctatgagccacctccaacaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 312 - DNA, 1023 bp, synthetic construct
  source 1..1023: mol_type = other DNA
gagaccagggagtgtatctactataacgccaattgggagctggagaggacaaaccagtccggactggagcggtgtgagggcgagcaggacaagagataccattgctacgctagctggcgcaattccagcggcaccatcgagctggtgaagaagggctgttggctggacgatttcaactgttacgatagacaggagtgcgtggcccaggaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 313 - DNA, 1035 bp, synthetic construct
  source 1..1035: mol_type = other DNA
gagaccagggagtgtatctactataacgccaattgggagctggagaggacaaaccagtccggactggagcggtgtgagggcgagcaggacaagagataccattgctacgctagctggcgcaattccagcggcaccatcgagctggtgaagaagggctgttggctggacgatttcaactgttacgatagacaggagtgcgtggcccaggaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 314 - DNA, 1002 bp, synthetic construct
  source 1..1002: mol_type = other DNA
gagaccagggagtgtatctactataacgccaattgggagctggagaggacaaaccagtccggactggagcggtgtgagggagagcaggacaagagataccattgctacgctagctggcgcaattccagcggcaccatcgagctggtgaagaagggctgttggctggacgatttcaactgttacgatagacaggagtgcgtggccgaggaggagaatcctcaggtgtatttttgctgttgcgagggcaacttctgcaatgagaggttcacccacctgccagaggctggaggaccagaggtgacctatgagccacctccaacaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 315 - DNA, 1023 bp, synthetic construct
  source 1..1023: mol_type = other DNA
gagaccagggagtgtatctactataacgccaattgggagctggagaggacaaaccagtccggactggagcggtgtgagggcgagcaggacaagagataccattgctacgctagctggcgcaattccagcggcaccatcgagctggtgaagaagggctgttggctggacgatttcaactgttacgatagacaggagtgcgtggccgaggaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 316 - DNA, 1035 bp, synthetic construct
  source 1..1035: mol_type = other DNA
gagaccagggagtgtatctactataacgccaattgggagctggagaggacaaaccagtccggactggagcggtgtgagggcgagcaggacaagagataccattgctacgctagctggcgcaattccagcggcaccatcgagctggtgaagaagggctgttggctggacgatttcaactgttacgatagacaggagtgcgtggccgaggaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 317 - DNA, 1002 bp, synthetic construct
  source 1..1002: mol_type = other DNA
gagaccagggagtgtatctactataacgccaattgggagctggagaggacaaaccagtccggactggagcggtgtgaggacgagcaggacaagagataccattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 318 - DNA, 1023 bp, synthetic construct
  source 1..1023: mol_type = other DNA
gagaccagggagtgtatctactataacgccaattgggagctggagaggacaaaccagtccggactggagcggtgtgaggacgagcaggacaagagataccattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 319 - DNA, 1035 bp, synthetic construct
  source 1..1035: mol_type = other DNA
gagaccagggagtgtatctactataacgccaattgggagctggagaggacaaaccagtccggactggagcggtgtgaggacgagcaggacaagagataccattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 320 - DNA, 1002 bp, synthetic construct
  source 1..1002: mol_type = other DNA
gagaccagggagtgtatctactataacgccaattgggagctggagaggacaaaccagtccggactggagcggtgtgaggaggagcaggacaagagataccattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 321 - DNA, 1023 bp, synthetic construct
  source 1..1023: mol_type = other DNA
gagaccagggagtgtatctactataacgccaattgggagctggagaggacaaaccagtccggactggagcggtgtgaggaggagcaggacaagagataccattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 322 - DNA, 1035 bp, synthetic construct
  source 1..1035: mol_type = other DNA
gagaccagggagtgtatctactataacgccaattgggagctggagaggacaaaccagtccggactggagcggtgtgaggaggagcaggacaagagataccattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 323 - DNA, 1002 bp, synthetic construct
  source 1..1002: mol_type = other DNA
gagaccagggagtgtatctactataacgccaattgggagctggagaggacaaaccagtccggactggagcggtgtgagggagagcaggacaagagattccattgctacgctagctggcgcaattccagcggcaccatcgagctggtgaagaagggctgttggctggacgatttcaactgttacgatagacaggagtgcgtggcccaggaggagaatcctcaggtgtatttttgctgttgcgagggcaacttctgcaatgagaggttcacccacctgccagaggctggaggaccagaggtgacctatgagccacctccaacaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 324 - DNA, 1023 bp, synthetic construct
  source 1..1023: mol_type = other DNA
gagaccagggagtgtatctactataacgccaattgggagctggagaggacaaaccagtccggactggagcggtgtgagggcgagcaggacaagagattccattgctacgctagctggcgcaattccagcggcaccatcgagctggtgaagaagggctgttggctggacgatttcaactgttacgatagacaggagtgcgtggcccaggaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 325 - DNA, 1035 bp, synthetic construct
  source 1..1035: mol_type = other DNA
gagaccagggagtgtatctactataacgccaattgggagctggagaggacaaaccagtccggactggagcggtgtgagggcgagcaggacaagagattccattgctacgctagctggcgcaattccagcggcaccatcgagctggtgaagaagggctgttggctggacgatttcaactgttacgatagacaggagtgcgtggcccaggaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 326 - DNA, 1002 bp, synthetic construct
  source 1..1002: mol_type = other DNA
gagaccagggagtgtatctactataacgccaattgggagctggagaggacaaaccagtccggactggagcggtgtgagggagagcaggacaagagactgcattgctacgctagctggcgcaattccagcggcaccatcgagctggtgaagaagggctgttggctggacgatttcaactgttacgatagacaggagtgcgtggccgaggaggagaatcctcaggtgtatttttgctgttgcgagggcaacttctgcaatgagaggttcacccacctgccagaggctggaggaccagaggtgacctatgagccacctccaacaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 327 - DNA, 1023 bp, synthetic construct
  source 1..1023: mol_type = other DNA
gagaccagggagtgtatctactataacgccaattgggagctggagaggacaaaccagtccggactggagcggtgtgagggcgagcaggacaagagactgcattgctacgctagctggcgcaattccagcggcaccatcgagctggtgaagaagggctgttggctggacgatttcaactgttacgatagacaggagtgcgtggccgaggaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 328 - DNA, 1035 bp, synthetic construct
  source 1..1035: mol_type = other DNA
gagaccagggagtgtatctactataacgccaattgggagctggagaggacaaaccagtccggactggagcggtgtgagggcgagcaggacaagagactgcattgctacgctagctggcgcaattccagcggcaccatcgagctggtgaagaagggctgttggctggacgatttcaactgttacgatagacaggagtgcgtggccgaggaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 329 - DNA, 1002 bp, synthetic construct
  source 1..1002: mol_type = other DNA
gagaccagggagtgtatctactataacgccaattgggagctggagaggacaaaccagtccggactggagcggtgtgagggagagcaggacaagagattccattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 330 - DNA, 1023 bp, synthetic construct
  source 1..1023: mol_type = other DNA
gagaccagggagtgtatctactataacgccaattgggagctggagaggacaaaccagtccggactggagcggtgtgagggcgagcaggacaagagattccattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 331 - DNA, 1035 bp, synthetic construct
  source 1..1035: mol_type = other DNA
gagaccagggagtgtatctactataacgccaattgggagctggagaggacaaaccagtccggactggagcggtgtgagggcgagcaggacaagagattccattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 332 - DNA, 1002 bp, synthetic construct
  source 1..1002: mol_type = other DNA
gagaccagggagtgtatctactataacgccaattgggagctggagaggacaaaccagtccggactggagcggtgtgagggagagcaggacaagagacagcattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 333 - DNA, 1023 bp, synthetic construct
  source 1..1023: mol_type = other DNA
gagaccagggagtgtatctactataacgccaattgggagctggagaggacaaaccagtccggactggagcggtgtgagggcgagcaggacaagagacagcattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 334 - DNA, 1023 bp, synthetic construct
  source 1..1023: mol_type = other DNA
gagaccagggagtgtatctactataacgccaattgggagctggagaggacaaaccagtccggactggagcggtgtgagggcgagcaggacaagagataccattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 335 - DNA, 1035 bp, synthetic construct
  source 1..1035: mol_type = other DNA
gagaccagggagtgtatctactataacgccaattgggagctggagaggacaaaccagtccggactggagcggtgtgagggcgagcaggacaagagatggcattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 336 - DNA, 1035 bp, synthetic construct
  source 1..1035: mol_type = other DNA
gagaccagggagtgtatctactataacgccaattgggagctggagaggacaaaccagtccggactggagcggtgtgagggcgagcaggacaagagagagcattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 337 - DNA, 1035 bp, synthetic construct
  source 1..1035: mol_type = other DNA
gagaccagggagtgtatctactataacgccaattgggagctggagaggacaaaccagtccggactggagcggtgtgagggcgagcaggacaagagaaagcattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 338 - DNA, 1035 bp, synthetic construct
  source 1..1035: mol_type = other DNA
gagaccagggagtgtatctactataacgccaattgggagctggagaggacaaaccagtccggactggagcggtgtgagggcgagcaggacaagagaatgcattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 339 - DNA, 1035 bp, synthetic construct; identical to SEQ ID NO: 319
  source 1..1035: mol_type = other DNA

SEQ ID NO: 340 - DNA, 1035 bp, synthetic construct
  source 1..1035: mol_type = other DNA
gagaccagggagtgtatctactataacgccaattgggagctggagaggacaaaccagtccggactggagcggtgtgagaacgagcaggacaagagataccattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 341 - DNA, 1035 bp, synthetic construct
  source 1..1035: mol_type = other DNA
gagaccagggagtgtatctactataacgccaattgggagctggagaggacaaaccagtccggactggagcggtgtgagcaggagcaggacaagagataccattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 342 - DNA, 1035 bp, synthetic construct
  source 1..1035: mol_type = other DNA
gagaccagggagtgtatctactataacgccaattgggagctggagaggacaaaccagtccggactggagcggtgtgagaaggagcaggacaagagataccattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 343 - DNA, 1035 bp, synthetic construct
  source 1..1035: mol_type = other DNA
gagaccagggagtgtatctactataacgccaattgggagctggagaggacaaaccagtccggactggagcggtgtgagaccgagcaggacaagagataccattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 344 - DNA, 1035 bp, synthetic construct
  source 1..1035: mol_type = other DNA
gagaccagggagtgtatctactataacgccaattgggagctggagaggacaaaccagtccggactggagcggtgtgagatggagcaggacaagagataccattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 345 - DNA, 1035 bp, synthetic construct
  source 1..1035: mol_type = other DNA
gagaccagggagtgtatctactataacgccaattgggagctggagaggacaaaccagtccggactggagcggtgtgagggcgagcaggacaagagataccattgctacgctagctggcgcaattccagcggcaccatcgagctggtgaagaagggctgttggctggacgatttcaactgttacgatagacaggagtgcgtggccagggaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 346 - DNA, 1035 bp, synthetic construct
  source 1..1035: mol_type = other DNA
gagaccagggagtgtatctactataacgccaattgggagctggagaggacaaaccagtccggactggagcggtgtgagggcgagcaggacaagagataccattgctacgctagctggcgcaattccagcggcaccatcgagctggtgaagaagggctgttggctggacgatttcaactgttacgatagacaggagtgcgtggcctacgaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 347 - DNA, 1035 bp, synthetic construct
  source 1..1035: mol_type = other DNA
gagaccagggagtgtatctactataacgccaattgggagctggagaggacaaaccagtccggactggagcggtgtgagggcgagcaggacaagagataccattgctacgctagctggcgcaattccagcggcaccatcgagctggtgaagaagggctgttggctggacgatttcaactgttacgatagacaggagtgcgtggcctgggaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 348 - DNA, 1035 bp, synthetic construct
  source 1..1035: mol_type = other DNA
gagaccagggagtgtatctactataacgccaattgggaggaggagaggacaaaccagtccggactggagcggtgtgagggcgagcaggacaagagataccattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 349 - DNA, 1035 bp, synthetic construct
  source 1..1035: mol_type = other DNA
gagaccagggagtgtatctactataacgccaattgggagcacgagaggacaaaccagtccggactggagcggtgtgagggcgagcaggacaagagataccattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 350 - DNA, 1035 bp, synthetic construct
  source 1..1035: mol_type = other DNA
gagaccagggagtgtatctactataacgccaattgggagtccgagaggacaaaccagtccggactggagcggtgtgagggcgagcaggacaagagataccattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 351 - DNA, 1035 bp, synthetic construct
  source 1..1035: mol_type = other DNA
gagaccagggagtgtatctactataacgccaattgggagctggagaggacaaaccagtccggactggagcggtgtgagggcgagcaggacaagagataccattgctacgctagctggcgcaattccagcggcaccatcgagctggtgaagaagggctgttggtacgacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 352 - DNA, 1035 bp, synthetic construct
  source 1..1035: mol_type = other DNA
gagaccagggagtgtatctactataacgccaattgggagctggagaggacaaaccagtccggactggagcggtgtgagggcgagcaggacaagagataccattgctacgctagctggcgcaattccagcggcaccatcgagctggtgaagaagggctgttggcaggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 353 - DNA, 1035 bp, synthetic construct
  source 1..1035: mol_type = other DNA
gagaccagggagtgtatctactataacgccaattgggagctggagaggacaaaccagtccggactggagcggtgtgagggcgagcaggacaagagataccattgctacgctagctggcgcaattccagcggcaccatcgagctggtgaagaagggctgttggatggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 354 - DNA, 1035 bp, synthetic construct
  source 1..1035: mol_type = other DNA
gagaccagggagtgtatctactataacgccaattgggagctggagaggacaaaccagtccggactggagcggtgtgagggcgagcaggacaagagataccattgctacgctagctggcgcaattccagcggcaccatcgagctggtgaagaagggctgttggatcgacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 355 - DNA, 1035 bp, synthetic construct
  source 1..1035: mol_type = other DNA
gagaccagggagtgtatctactataacgccaattgggagctggagaggacaaaccagtccggactggagcggtgtgagggcgagcaggacaagagatggcattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggaggctctggaggaggaggctccggaggaggaggaacccatacatgtccaccttgcccagctccagagctgctgggaggcccttccgtgttcctgtttccacccaagccaaaggacaccctgtacatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 356 - DNA, 1050 bp, synthetic construct
  source 1..1050: mol_type = other DNA
gagaccagggagtgtatctactataacgccaattgggagctggagaggacaaaccagtccggactggagcggtgtgagggcgagcaggacaagagatggcattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggcggcggctccggcggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 357 - DNA, 1050 bp, synthetic construct
  source 1..1050: mol_type = other DNA
gagaccagggagtgtatctactataacgccaattgggagctggagaggacaaaccagtccggactggagcggtgtgagggcgagcaggacaagagatggcattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggcggcggctccggcggaggaggctctggaggaggaggctccggaggaggaggaacccatacatgtccaccttgcccagctccagagctgctgggaggcccttccgtgttcctgtttccacccaagccaaaggacaccctgtacatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 358 - DNA, 1050 bp, synthetic construct
  source 1..1050: mol_type = other DNA
gagaccagggagtgtatctactataacgccaattgggagctggagaggacaaaccagtccggactggagcggtgtgagggcgagcaggacaagagatggcattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggcggcggctccggcggaggaggctctggaggaggaggctccggaggaggaggaacccatacatgtccaccttgcccagctccagagctgctgggaggcccttccgtgttcctgtttccacccaagccaaaggacaccctgtacatcaccagggagccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 359 - DNA, 1050 bp, synthetic construct
  source 1..1050: mol_type = other DNA
gagaccagggagtgtatctactataacgccaattgggagctggagaggacaaaccagtccggactggagcggtgtgaggacgagcaggacaagagatggcattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggcggcggctccggcggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 360 - DNA, 1050 bp, synthetic construct
  source 1..1050: mol_type = other DNA
gagaccagggagtgtatctactataacgccaattgggagctggagaggacaaaccagtccggactggagcggtgtgaggaggagcaggacaagagatggcattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggcggcggctccggcggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 361 - DNA, 1050 bp, synthetic construct
  source 1..1050: mol_type = other DNA
gagaccagggagtgtatctactataacgccaattgggagctggagaggacaaaccagtccggactggagcggtgtgagaacgagcaggacaagagatggcattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggcggcggctccggcggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 362 - DNA, 1050 bp, synthetic construct
  source 1..1050: mol_type = other DNA
gagaccagggagtgtatctactataacgccaattgggagctggagaggacaaaccagtccggactggagcggtgtgagcaggagcaggacaagagatggcattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggcggcggctccggcggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 363 - DNA, 1050 bp, synthetic construct
  source 1..1050: mol_type = other DNA
gagaccagggagtgtatctactataacgccaattgggagctggagaggacaaaccagtccggactggagcggtgtgagaccgagcaggacaagagatggcattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggcggcggctccggcggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 364 - DNA, 1050 bp, synthetic construct
  source 1..1050: mol_type = other DNA
gagaccagggagtgtatctactataacgccaattgggagctggagaggacaaaccagtccggactggagcggtgtgagatggagcaggacaagagatggcattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggcggcggctccggcggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 365 - DNA, 1050 bp, synthetic construct
  source 1..1050: mol_type = other DNA
gagaccagggagtgtatctactataacgccaattgggagctggagaggacaaaccagtccggactggagcggtgtgaggacgagcaggacaagagataccattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggcggcggcggctccggaggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 366 - DNA, 1050 bp, synthetic construct
  source 1..1050: mol_type = other DNA
gagaccagggagtgtatctactataacgccaattgggagctggagaggacaaaccagtccggactggagcggtgtgaggaggagcaggacaagagataccattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggcggctccggcggcggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 367 - DNA, 1050 bp, synthetic construct
  source 1..1050: mol_type = other DNA
gagaccagggagtgtatctactataacgccaattgggagctggagaggacaaaccagtccggactggagcggtgtgagaacgagcaggacaagagataccattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggcggcggcggctccggaggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 368 - DNA, 1050 bp, synthetic construct
  source 1..1050: mol_type = other DNA
gagaccagggagtgtatctactataacgccaattgggagctggagaggacaaaccagtccggactggagcggtgtgagcaggagcaggacaagagataccattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggcggctccggcggcggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 369 - DNA, 1050 bp, synthetic construct
  source 1..1050: mol_type = other DNA
gagaccagggagtgtatctactataacgccaattgggaggaggagaggacaaaccagtccggactggagcggtgtgagggcgagcaggacaagagatggcattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggcggcggcggctccggaggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 370 - DNA, 1050 bp, synthetic construct
  source 1..1050: mol_type = other DNA
gagaccagggagtgtatctactataacgccaattgggaggacgagaggacaaaccagtccggactggagcggtgtgagggcgagcaggacaagagatggcattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggcggcggcggctccggaggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 371 - DNA, 1050 bp, synthetic construct
  source 1..1050: mol_type = other DNA
gagaccagggagtgtatctactataacgccaattgggagaacgagaggacaaaccagtccggactggagcggtgtgagggcgagcaggacaagagatggcattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggcggcggcggctccggaggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 372 - DNA, 1050 bp, synthetic construct
  source 1..1050: mol_type = other DNA
gagaccagggagtgtatctactataacgccaattgggagcaggagaggacaaaccagtccggactggagcggtgtgagggcgagcaggacaagagatggcattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggcggcggcggctccggaggaggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 373 - DNA, 1050 bp, synthetic construct
  source 1..1050: mol_type = other DNA
gagaccagggagtgtatctactataacgccaattgggagcaggagaggacaaaccagtccggactggagcggtgtgagggcgagcaggacaagagataccattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggcggctccggcggcggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 374 - DNA, 1050 bp, synthetic construct
  source 1..1050: mol_type = other DNA
gagaccagggagtgtatctactataacgccaattgggaggacgagaggacaaaccagtccggactggagcggtgtgagggcgagcaggacaagagataccattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggcggctccggcggcggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 375 - DNA, 1050 bp, synthetic construct
  source 1..1050: mol_type = other DNA
gagaccagggagtgtatctactataacgccaattgggagaacgagaggacaaaccagtccggactggagcggtgtgagggcgagcaggacaagagataccattgctacgctagctggcgcaattccagcggcaccatcgagctggtgaagaagggctgttggctggacgatttcaactgttacgatagacaggagtgcgtggccaccgaggagaatcctcaggtgtatttttgctgttgcgagggcaacttctgcaatgagaggttcacccacctgccagaggctggaggaccagaggtgacctatgagccacctccaacaggaggaggcggctccggcggcggaggctctggaggaggaggctccggaggaggaggaacccatacatgtccaccttgcccagctccagagctgctgggaggcccttccgtgttcctgtttccacccaagccaaaggacaccctgatgatctctaggaccccagaggtgacatgcgtggtggtggacgtgtcccacgaggaccccgaggtgaagtttaactggtacgtggatggcgtggaggtgcataatgctaagacaaagccaagggaggagcagtacaactccacctatcgggtggtgagcgtgctgacagtgctgcaccaggattggctgaacggcaaggagtataagtgtaaggtgagcaataaggctctgcctgccccaatcgagaagaccatctctaaggctaagggccagcccagagagcctcaggtgtacacactgcctccatcccgcgacgagctgaccaagaaccaggtgagcctgacatgcctggtgaagggcttctatcccagcgacatcgccgtggagtgggagtctaatggccagcctgagaacaattacaagaccacaccccctgtgctggacagcgatggctctttctttctgtattctaagctgaccgtggataagtcccggtggcagcagggcaacgtgttttcttgttccgtgatgcatgaggccctgcacaatcattacacacagaagagcctgtctctgtcccctggc

SEQ ID NO: 376 - protein, 464 aa, synthetic construct
MDWTWRILFLVAAATGTHAQVQLVQSGAEVKKPGASVKVSCKASGYTFTSSYINWVRQAPGQGLEWMGTINPVSGSTSYAQKFQGRVTMTRDTSISTAYMELSSLRSEDTAVYYCARGGWFDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 377 - protein, 236 aa, synthetic construct
MVLQTQVFISLLLWISGAYGDIALTQPASVSGSPGQSITISCTGTSSDVGSYNYVNWYQQHPGKAPKLMIYGVSKRPSGVSNRFSGSKSGNTASLTISGLQAEDEADYYCGTFAGGSYYGVFGGGTKLTVLGQPKSTPTLTVFPPSSEELKENKATLVCLISNFSPSGVTVAWKANGTPITQGVDTSNPTKEGNKFMASSFLHLTSDQWRSHNSFTCQVTHEGDTVEKSLSPAECL

(19) United States
(12) Patent Application Publication
(10) Pub. No.: US 2026/0193317 A1
(43) Pub. Date: Jul. 9, 2026

(54) COMBINATION THERAPY WITH ACTRIIB RECEPTOR VARIANTS AND GLP-1 AGONISTS

(71) Applicant: 35PHARMA Inc., Montreal (CA)

(72) Inventors: Maureen O'CONNOR-MCCOURT, Montreal (CA); Vannakambadi K. GANESH, Montreal (CA); Gilles TREMBLAY, Montreal (CA); Gauthier SCHANG, Montreal (CA); Thomas P. LOISEL, Montreal (CA); Michael J. LITTLE, Montreal (CA); Julia SCHOELERMANN, Montreal (CA); Ilia A. TIKHOMIROV, Montreal (CA)

(21) Appl. No.: 19/558,973

(22) Filed: Mar. 6, 2026

Related U.S. Application Data
(63) Continuation of application No. PCT/CA2024/051163, filed on Sep. 6, 2024.
(60) Provisional application No. 63/581,428, filed on Sep. 8, 2023.
(60) Provisional application No. 63/647,506, filed on May 14, 2024.
(60) Provisional application No. 63/678,479, filed on Aug. 1, 2024.

(51) Int. Cl. C07K 14/71; A61K 9/00; A61K 47/64; A61P 3/04; C07K 14/605

(52) U.S. Cl. CPC C07K 14/71; A61K 9/0019; A61K 47/6425; A61P 3/04; C07K 14/605; C07K 2319/02; C07K 2319/30; C07K 2319/31

(57) ABSTRACT

The present disclosure provides combinations of TGFβ superfamily ligand binding agents and GLP-1 agonists, compositions thereof, and methods of use in the treatment of metabolic disorders.

### CROSS-REFERENCE TO RELATED APPLICATIONS

This application a US continuation application claiming priority to International Application No. PCT/CA2024/051163, filed Sep. 6, 2024 and claiming priority to U.S. Provisional Application No. 63/581,428, filed on Sep. 8, 2023; U.S. Provisional Application No. 63/647,506, filed on May 14, 2024; and U.S. Provisional Application No. 63/678,479, filed on Aug. 1, 2024, each of which are incorporated herein by reference in their entireties.

### REFERENCE TO AN ELECTRONIC SEQUENCE LISTING

The contents of the electronic sequence listing (35PH_005_03US_SeqList_ST26.xml; Size: 451,022 bytes; and Date of Creation: Mar. 5, 2026) are herein incorporated by reference in their entirety.

### FIELD

The present disclosure relates to combinations of TGFβ superfamily ligand binding agents and GLP-1 agonists and uses thereof for the treatment of metabolic disorders.

### BACKGROUND

Treatment of metabolic disorders such as obesity remains elusive as the regulation of metabolism and fat storage involves complex biological feedback systems. To date, drugs effective in regulating metabolic dysfunction often exhibit several side effects, which limit their usefulness in patient populations, and in particular, in obese patient populations, who may require long term treatment. Thus, there is a need for additional compositions and methods to treat metabolic disorders including obesity.

### SUMMARY

In some embodiments, the present disclosure provides a combination comprising a TGFβ superfamily ligand binding agent comprising (a) a first polypeptide and a second polypeptide, wherein the first and second polypeptides each comprise: (i) a polypeptide comprising an amino acid sequence that is at least 90% identical to an Activin receptor type IIB (ActRIIB) ectodomain (ECD) variant, the ActRIIB ECD variant comprising an amino acid substitution at the position corresponding to position 33 of SEQ ID NO: 2; (ii) an Fc domain monomer; and (iii) a peptide linker joining the polypeptide to the Fc domain monomer; and (b) a Glucagon-like peptide 1 (GLP-1) agonist. The combination of claim 1, wherein the ActRIIB ECD variant demonstrates reduced inhibition of BMP-9 and/or BMP-10 compared to a wild-type ActRIIB ectodomain. In some embodiments, the amino acid substitution is selected from L33F, L33Q, L33Y, L33W, L33H, L33R, L33E, L33K, and L33M.

In some embodiments, the ActRIIB ECD variant comprises the amino acid substitution L33F. In some embodiments, the ActRIIB ECD variant comprises an amino acid sequence that is at least 90%, at least 95%, at least 96%, at least 97%, at least 98%, or at least 99% identical to SEQ ID NO: 10; or comprises or consists of the amino acid sequence of SEQ ID NO: 10.

In some embodiments, the ActRIIB ECD variant comprises the amino acid substitution L33Q. In some embodiments, the ActRIIB ECD variant comprises an amino acid sequence that is at least 90%, at least 95%, at least 96%, at least 97%, at least 98%, or at least 99% identical to SEQ ID NO: 11; or comprises or consists of the amino acid sequence of SEQ ID NO: 11.

In some embodiments, the ActRIIB ECD variant comprises the amino acid substitution L33Y. In some embodiments, the ActRIIB ECD variant comprises an amino acid sequence that is at least 90%, at least 95%, at least 96%, at least 97%, at least 98%, or at least 99% identical to SEQ ID NO: 12; or comprises or consists of the amino acid sequence of SEQ ID NO: 12.

In some embodiments, the ActRIIB ECD variant comprises the amino acid substitution L33W. In some embodiments, the ActRIIB ECD variant comprises an amino acid sequence that is at least 90%, at least 95%, at least 96%, at least 97%, at least 98%, or at least 99% identical to SEQ ID NO: 13; or comprises or consists of the amino acid sequence of SEQ ID NO: 13.

In some embodiments, the ActRIIB ECD variant comprises the amino acid substitution L33H. In some embodiments, the ActRIIB ECD variant comprises an amino acid sequence that is at least 90%, at least 95%, at least 96%, at least 97%, at least 98%, or at least 99% identical to SEQ ID NO: 14; or comprises or consists of the amino acid sequence of SEQ ID NO: 14.

In some embodiments, the ActRIIB ECD variant comprises the amino acid substitution L33R. In some embodiments, the ActRIIB ECD variant comprises an amino acid sequence that is at least 90%, at least 95%, at least 96%, at least 97%, at least 98%, or at least 99% identical to SEQ ID NO: 15; or comprises or consists of the amino acid sequence of SEQ ID NO: 15.

In some embodiments, the ActRIIB ECD variant comprises the amino acid substitution L33E. In some embodiments, the ActRIIB ECD variant comprises an amino acid sequence that is at least 90%, at least 95%, at least 96%, at least 97%, at least 98%, or at least 99% identical to SEQ ID NO: 16; or comprises or consists of the amino acid sequence of SEQ ID NO: 16.

In some embodiments, the ActRIIB ECD variant comprises the amino acid substitution L33K. In some embodiments, the ActRIIB ECD variant comprises an amino acid sequence that is at least 90%, at least 95%, at least 96%, at least 97%, at least 98%, or at least 99% identical to SEQ ID NO: 17; or comprises or consists of the amino acid sequence of SEQ ID NO: 17.

In some embodiments, the ActRIIB ECD variant comprises the amino acid substitution L33M. In some embodiments, the ActRIIB ECD variant comprises an amino acid sequence that is at least 90%, at least 95%, at least 96%, at least 97%, at least 98%, or at least 99% identical to SEQ ID NO: 18; or comprises or consists of the amino acid sequence of SEQ ID NO: 18.

In some embodiments, the ActRIIB ECD variant further comprises an amino acid substitution at position 27 of SEQ ID NO: 2. In some embodiments, the ActRIIB ECD variant further comprises an amino acid substitution at position 69 of SEQ ID NO: 2. In some embodiments, the ActRIIB ECD variant further comprises one or more amino acid substitutions which is G27D, G27E, T69E, T69Q, or T69H.

In some embodiments, the ActRIIB ECD variant further comprises one or more additional amino acids at the N or C terminus. In some embodiments, the ActRIIB ECD variant further comprises the following amino acids at the N terminus: GRGEA (SEQ ID NO: 63) and/or the following amino acids at the C-terminus: APT.

In some embodiments, the first and the second polypeptides comprise the following structure, from N- to C-terminus: ActRIIB-ECD-peptide linker-Fc domain monomer.

In some embodiments, the Fc domain monomer is an IgG1, IgG2, IgG3 or IgG4 isotype. In some embodiments, the Fc domain monomer is a human Fc domain monomer or a murine Fc domain monomer. In some embodiments, the Fc domain monomer is engineered to reduce aggregation or to modulate stability of a dimer of the polypeptide. In some embodiments, the Fc domain monomer comprises the amino acid substitutions of M252Y, S254T, and T256E (YTE). In some embodiments, the Fc domain monomer comprises the M252Y amino acid substitution. In some embodiments, the Fc domain monomer includes a D at position 356 and an L at position 358 (DL). In some embodiments, the Fc domain monomer includes an E at position 356 and an M at position 358 (EM). In some embodiments, the Fc domain monomer further comprises a Lysine residue (K) at the C terminus. In some embodiments, the Fc domain monomer comprises an amino acid sequence that is at least 85%, at least 90%, at least 95%, at least 96%, at least 97%, at least 98%, or at least 99% identical to any one of SEQ ID NOs: 252-292; or comprises or consists of an amino acid sequence selected from any one of SEQ ID NOs: 252-292. In some embodiments, the Fc domain monomer is an IgG1 isotype. In some embodiments, the Fc domain monomer comprises or consists of the amino acid sequence set forth in SEQ ID NO: 253, SEQ ID NO: 255, or SEQ ID NO: 256. In some embodiments, the Fc domain monomer in the first polypeptide forms a dimer with the Fc domain monomer in the second polypeptide.

In some embodiments, the peptide linker is Glycine-rich. In some embodiments, the peptide linker is between 10 and 40 amino acids long. In some embodiments, the peptide linker is at least 10 amino acids long, at least 14 amino acids long, at least 19 amino acids long, or at least 39 amino acids long. In some embodiments, the peptide linker is 10 amino acids long, 14 amino acids long, 19 amino acids long, or 39 amino acids long. In some embodiments, the peptide linker comprises the amino acid sequence set forth in any one of SEQ ID NOs: 89, 94, or 98.

In some embodiments, the ActRIIB-ECD comprises an amino acid sequence that is at least 95%, 96%, 97%, 98%, or 99% identical to any one of SEQ ID NOs:4-62; or comprises or consists of an amino acid sequence selected from any one of SEQ ID NOs: 4-62. In some embodiments, the ActRIIB-ECD comprises or consists of the amino acid sequence of SEQ ID NO: 10-18, or a sequence at least 95%, at least 96%, at least 97%, at least 98%, or at least 99% identical thereto. In some embodiments, the ActRIIB-ECD comprises or consists of the amino acid sequence of SEQ ID NO: 13 or a sequence at least 95%, at least 96%, at least 97%, at least 98%, or at least 99% identical thereto.

In some embodiments, the first and/or second polypeptide: comprise an amino acid sequence that is at least 95%, 96%, 97%, 98%, or 99% identical to an amino acid sequence selected from SEQ ID NOs: 174-251; or comprise or consist of an amino acid sequence selected from SEQ ID NOs: 174-251.

In some embodiments, the first and/or second polypeptide: comprises an amino acid sequence that is at least 95%, 96%, 97%, 98%, or 99% identical to an amino acid sequence selected from SEQ ID NOs: 175, 176, 180, 204-214, and 230-234; or comprises or consists of an amino acid sequence selected from SEQ ID NOs: 175, 176, 180, 204-214, and 230-234.

In some embodiments, the first and/or second polypeptide: comprises an amino acid sequence that is at least 95%, 96%, 97%, 98%, or 99% identical to an amino acid sequence selected from SEQ ID NOs: 211 and 230-234; or comprises or consists of an amino acid sequence selected from SEQ ID NOs: 211 and 230-234.

In some embodiments, the first and/or second polypeptide: comprises or consists of the amino acid sequence of SEQ ID NO: 231, or an amino acid sequence that is at least 95%, 96%, 97%, 98%, or 99% identical thereto; or comprises or consists of the amino acid sequence of SEQ ID NO: 231.

In some embodiments, the first and/or second polypeptide comprises or consists of the amino acid sequence of SEQ ID NO: 234, or an amino acid sequence that is at least 95%, 96%, 97%, 98%, or 99% identical thereto; or comprises or consists of the amino acid sequence of SEQ ID NO: 234.

In some embodiments, the first and/or second polypeptide further comprise an albumin-binding domain, a fibronectin domain, or a human serum albumin domain fused to the N- or C-terminus of the ActRIIB-ECD via a linker. In some embodiments, the first and/or second polypeptide further comprises a signal peptide of SEQ ID NO: 1 at the N-terminus of the ActRIIB-ECD. In some embodiments, the signal peptide is cleaved from the mature protein.

In some embodiments, the binding agent is conjugated with a targeting agent, a therapeutic moiety, a detectable moiety, or a diagnostic moiety. In some embodiments, the targeting agent, the therapeutic moiety, the detectable moiety, or the diagnostic moiety comprises an antibody or antigen binding fragment thereof, a binding agent having affinity for another member of the TGFβ superfamily or for another therapeutic target, a radiotherapy agent, an imaging agent, a fluorescent moiety, a cytotoxic agent, an anti-mitotic drug, a nanoparticle-based carrier, a polymer-conjugated drug, a nanocarrier, an imaging agent, a stabilizing agent, a drug, a nanocarrier, or a dendrimer.

In some embodiments, the first and second polypeptides form a dimer linked by at least one disulfide bond between the Fc domain monomer of the first polypeptide and the Fc domain monomer of the second polypeptide. In some embodiments, the binding agent: demonstrates similar or increased binding to human activin A, activin B, GDF-8, and/or GDF-11 and demonstrates reduced binding to human BMP-9 and/or BMP-10 compared to a polypeptide comprising a WT ActRIIB-ECD; does not substantially bind to human BMP-9; demonstrates reduced binding to human BMP-10 compared to a polypeptide comprising a WT ActRIIB-ECD; inhibits signaling of one or more of human activin A, activin B, GDF-8, and GDF-11; and/or does not substantially inhibit human BMP-9 and/or BMP-10 signaling.

In some embodiments, the GLP-1 agonist is an antibody, small molecule, peptide, or aptamer. In some embodiments, the GLP-1 agonist is selected from the group consisting of exenatide, exenatide extended-release, efpeglenatide, dulaglutide, liraglutide, lixisenatide, lotiglipron, semaglutide, tirzepatide, cotadutide, noiiglutide, oxyntomodulin, orforglipron, retatrutide, albiglutide, beinaglutide and PEG-loxenatide, pemvidutide, and danuglipron. In some embodiments, the GLP-1 agonist is selected from semaglutide and tirzepatide. In some embodiments, the GLP-1 agonist is a dual GLP-1 agonist and GIP agonist, a dual GLP-1 agonist and GCG agonist, or a triagonist of GIP/GLP-1/glucagon receptors.

In some embodiments, the binding agent and GLP-1 agonist are formulated in separate pharmaceutical compositions. In some embodiments, the binding agent and GLP-1 agonist are formulated in the same pharmaceutical composition. In some embodiments, the pharmaceutical composition is formulated for administration by injection or infusion. In some embodiments, the composition is formulated for intravenous, subcutaneous, intraperitoneal, or intramuscular administration.

In some embodiments, the present disclosure provides a kit comprising a combination described herein, and optionally, directions for use.

In some embodiments, the present disclosure provides a method of treating or preventing a metabolic disease or condition in a subject in need thereof, the method comprising administering a combination described herein to the subject. In some embodiments, the subject is a human. In some embodiments, the metabolic disorder is selected from the group consisting of obesity, diabetes, metabolic syndrome, anti-psychotic drug-associated obesity, glucocorticoid-induced obesity, hypothalamic obesity associated with craniopharyngioma, Prader-Willi syndrome, and a monogenetic disorder associated with obesity such as Bardet-Biedl syndrome. In some embodiments, the metabolic disorder is selected from obesity, Type 2 diabetes, and pre-diabetes. In some embodiments, the metabolic disorder is obesity.

In some embodiments, the method does not: cause a vascular complication in a subject; increase vascular permeability or leakage in a subject; increase red blood cell mass; increase hemoglobin; cause thrombocytopenia; cause a hematological complication in a subject; and/or does not cause a reduction in lean muscle mass in the subject. In some embodiments, the treatment increases lean mass in the subject. In some embodiments, the treatment reduces fat mass in the subject.

Further scope, applicability and advantages of the present technology will become apparent from the non-restrictive detailed description given hereinafter. It should be understood, however, that this detailed description, while indicating exemplary embodiments of the technology, is given by way of example only, with reference to the accompanying drawings.

### BRIEF DESCRIPTION OF THE DRAWINGS

For a better understanding of the technology and to show more clearly how it may be carried into effect, reference will now be made by way of example to the accompanying drawings, which illustrate aspects and features according to non-limiting embodiments of the present technology.

FIGS. 1A and 1B show polyacrylamide gel electrophoresis analysis under non-reducing and reducing conditions of representative ActRIIB-ECD polypeptide constructs. After expression and purification, 1 μg of each protein was loaded on the gel, as indicated: P75: Protein 75; P739: Protein 739; P750: Protein 750; P751: Protein 751; P753: Protein 753; P754: Protein 754; P1182: Protein 1182; P1185: Protein 1185; P1229: Protein 1229; P1371: Protein 1371; P1372: Protein 1372; P1373: Protein 1373; P1374: Protein 1374; P1375: Protein 1375; P1389: Protein 1389; P1406: Protein 1406; P1409: Protein 1409. “NR”: non-reducing conditions; “R”: reducing conditions.

FIG. 2A-FIG. 2Q show comparative charts (radar plots) in which IC50 values for neutralization of TGFβ superfamily ligands (activin A, activin B, GDF-8, GDF-11, BMP-9, and BMP-10) are displayed for exemplary test proteins, as indicated. Points at the center of the chart indicate low neutralization potency for a given cytokine (high IC50 value), whereas points at the edge of the chart indicate high neutralization potency for a given cytokine (low IC50 value). Exemplary test proteins displayed are (FIG. 2A) P739, P750, and P751, (FIG. 2B) P753, P754, and P1229, (FIG. 2C) P1373, P1371, and P1375, (FIG. 2D) P1182 and P1185, and (FIG. 2E) P1389, P1406, and P1409. All agents are compared to wild type ActRIIB-ECD (P75). FIG. 2F-FIG. 21 show representative results in the HEK-Blue cell-based assay for inhibition of activin A, activin B, GDF-8, and GDF-11, respectively, for exemplary proteins P75, P1229, P1371, P1372, P1373, P1374, and P1375. FIG. 2J-FIG. 2K show representative results in the HepG2 cell-based assay for inhibition of BMP-9 and BMP-10, respectively, for exemplary proteins P75, P1229, P1372, P1373, P1374, and P1375. FIG. 2L-FIG. 2O show representative results in the HEK-Blue cell-based assay for inhibition of activin A, activin B, GDF-8, and GDF-11, respectively, for exemplary proteins P75, P1229, P1373, P1483, P1484, P1485, and P1486. FIG. 2P-FIG. 2Q show representative results in the HepG2 cell-based assay for inhibition of BMP-9 and BMP-10, respectively, for exemplary proteins P75, P1229, P1373, P1483, P1484, P1485, and P1486. Error bars indicate standard error of the mean (SEM).

FIG. 3 shows results from the activin A ELISA using supernatants from the small-scale production of ActRIIB-ECD fusion proteins. Results are expressed as a % of the signal obtained with wild-type ActRIIB-ECD-Fc (P75). In this assay, a loss of signal suggests increased binding of the exemplary test agent to activin A. Error bars indicate standard error of the mean (SEM). This graph was generated using GraphPad Prism 9.0.

FIG. 4 shows results from the BMP-9 ELISA using supernatants from the small-scale production of ActRIIB-ECD fusion proteins. Results are expressed as a % of the signal obtained with wild-type ActRIIB-ECD-Fc (P75). In this assay, a loss of signal suggests decreased binding of the exemplary test agent to BMP-9. Error bars indicate standard error of the mean (SEM). This graph was generated using GraphPad Prism 9.0.

FIG. 5A-FIG. 5B show results from the BMP-10 assays using supernatants from the small-scale production of ActRIIB-ECD fusion proteins. (FIG. 5A) Results from the cell-based assays are expressed as a % of the signal obtained with BMP-10 alone. In this assay, a loss of signal suggests increased binding of the exemplary test agent to BMP-10. Error bars indicate standard error of the mean (SEM). (FIG. 5B) Bio-layer interferometry was used to derive KD values for exemplary test agents. These graphs were generated using GraphPad Prism 9.0.

FIG. 6 shows results from single-injection experiments in wild-type mice. Male mice were injected with a single dose of test agent as indicated (25 mg/kg, subcutaneous), and body weight gain was evaluated after 4 days. Results were normalized to the vehicle control, and error bars indicate standard error of the mean (SEM). Results were analyzed by one-way ANOVA followed by post-hoc Bonferroni-corrected multiple comparison test; *p<0.05, **p<0.01, ***p<0.001, ***p<0.0001 relative to the vehicle group.

FIG. 7A-FIG. 7B show body weight gain in male mice injected (subcutaneous) with test proteins. FIG. 7A shows results for P750 (1, 5, or 25 mg/kg) and FIG. 7B shows results for P1229 (5 or 25 mg/kg) and P75 (25 mg/kg). Injections were performed twice weekly for 11 days (P750) or 7 days (P1229 and P75). Results were normalized to the vehicle control, and error bars indicate standard error of the mean (SEM). Results were analyzed by two-way ANOVA followed by post-hoc Bonferroni-corrected multiple comparison test; *p<0.05, ***p<0.001, ***p<0.0001 relative to the vehicle group.

FIG. 8 provides an exemplary schematic of binding agents described herein.

FIG. 9A-FIG. 9D illustrate changes in skeletal muscle mass after a week of treatment with vehicle, P1229 (5 or 25 mg/kg), and P75 (25 mg/kg). Injections were done subcutaneously, twice weekly. FIG. 9A shows average tibialis anterior weight. FIG. 9B shows tibialis weight as normalized to vehicle control. FIG. 9C shows average gastrocnemius weight.

FIG. 9D shows gastrocnemius weight as normalized to vehicle control. Results were analyzed by one-way ANOVA followed by post-hoc Bonferroni-corrected multiple comparison test; *p<0.05, ***p<0.001 relative to the vehicle group.

FIG. 10 illustrates changes in Mss51 expression in tibialis anterior after a week of treatment with vehicle, P1229 (5 or 25 mg/kg), and P75 (25 mg/kg). Injections were done subcutaneously, twice weekly. Results were analyzed by one-way ANOVA followed by post-hoc Bonferroni-corrected multiple comparison test; ****p<0.0001 relative to the vehicle group.

FIG. 11A-FIG. 11F illustrate changes in body composition of diet-induced obese (DIO) mice injected subcutaneously with vehicle, P1229 (25 mg/kg, twice weekly), P75 (25 mg/kg, twice weekly), P1373 (5, 20, or 50 mg/kg twice weekly), or P1307 (CDD866, murine bimagrumab (an antibody targeting ActRIIB and ActRIIA), 20 mg/kg once weekly). FIGS. 11A and 11D show average lean mass, FIGS. 11B and 11E show average fat mass, and FIGS. 11C and 11F show the ratio of lean mass over fat mass. Parameters were assessed by echoMRI. Results were analyzed by one-way ANOVA followed by post-hoc Bonferroni-corrected multiple comparison test; *p<0.05 relative to the vehicle group.

FIG. 12A-FIG. 12B illustrate food consumption over time of DIO mice injected subcutaneously with vehicle, P1229 (25 mg/kg, twice weekly), P75 (25 mg/kg, twice weekly), P1373 (5, 20, or 50 mg/kg twice weekly), or P1307 (CDD866, or murine bimagrumab, 20 mg/kg once weekly). FIGS. 12C-H show skeletal muscle weights at the end of the study; FIG. 12C-FIG. 12D, FIG. 12E-FIG. 12F, and FIG. 12G-FIG. 12H show tibialis anterior, gastrocnemius, and soleus weights, respectively. Results were analyzed by one-way ANOVA followed by post-hoc Bonferroni-corrected multiple comparison test; *p<0.05, **p<0.01 relative to the vehicle group.

FIG. 13A-FIG. 13C illustrate changes in skeletal muscle gene expression in DIO mice after 3 weeks of subcutaneous injections with vehicle, P1229 (25 mg/kg, twice weekly), P75 (25 mg/kg, twice weekly), P1373 (5, 20, or 50 mg/kg twice weekly), or P1307 (CDD866, or murine bimagrumab, 20 mg/kg once weekly). FIG. 13A-FIG. 13B illustrate Mss51 expression in gastrocnemius muscle. FIG. 13C illustrates changes in the ratio of Serpine1 to Id1 expression ratio in soleus muscle. Results were analyzed by one-way ANOVA followed by post-hoc Bonferroni-corrected multiple comparison test; *p<0.05, **p<0.01, ***p<0.001 relative to the vehicle group.

FIG. 14A-FIG. 14F illustrate changes in hepatic gene expression in DIO mice after subcutaneous injections with vehicle, P1229 (25 mg/kg, twice weekly), P75 (25 mg/kg, twice weekly), P1373 (5, 20, or 50 mg/kg twice weekly), or P1307 (CDD866, or murinebimagrumab, 20 mg/kg once weekly). FIG. 14A-FIG. 14B show Ahsg gene expression, encoding for fetuin-A. FIG. 14C-FIG. 14D show Fgf21 gene expression, encoding for FGF21. FIG. 14E-FIG. 14F Show Inhbe gene expression, encoding for activin E. Results were analyzed by one-way ANOVA followed by post-hoc Bonferroni-corrected multiple comparison test; *p<0.05, **p<0.01, ***p<0.001 relative to the vehicle group.

FIG. 15A-FIG. 15B illustrate changes in follicle-stimulating hormone (FSH) production in DIO mice after subcutaneous injections with vehicle, P1229 (25 mg/kg, twice weekly), P75 (25 mg/kg, twice weekly), P1373 (5, 20, or 50 mg/kg twice weekly), or P1307 (CDD866, or murine bimagrumab, 20 mg/kg once weekly). Results were analyzed by one-way ANOVA followed by post-hoc Bonferroni-corrected multiple comparison test; ***p<0.001, ****p<0.0001 relative to the vehicle group.

FIG. 16A-FIG. 16E illustrate changes in body weight and body composition (assessed by NMR) in DIO mice after subcutaneous injections with vehicle, P1229 (10 or 40 mg/kg, twice weekly), P1307 (CDD866, 20 mg/kg weekly), semaglutide (40 μg/kg, daily), tirzepatide (45 μg/kg, daily), P1229 (40 mg/kg)+semaglutide, P1229 (40 mg/kg)+tirzepatide, or P1229 (40 mg/kg)+caloric restriction. Results were analyzed by two-way ANOVA followed by post-hoc Tukey-corrected multiple comparison test; *p<0.05, **p<0.01, ***p<0.001, ****p<0.0001. Statistical significance is only shown for D21 data.

FIG. 17A-FIG. 17G illustrate organ weights at the end of the study (D21) in DIO mice after subcutaneous injections with vehicle, P1229 (10 or 40 mg/kg, twice weekly), P1307 (CDD866, 20 mg/kg weekly), semaglutide (40 μg/kg, daily), tirzepatide (45 μg/kg, daily), P1229 (40 mg/kg)+semaglutide, P1229 (40 mg/kg)+tirzepatide, or P1229 (40 mg/kg)+caloric restriction. Results were analyzed by one-way ANOVA followed by post-hoc Tukey-corrected multiple comparison test; *p<0.05, **p<0.01, ***p<0.001, ****p<0.0001. All groups were compared to the vehicle group, and incretin mimetics alone were compared to their respective combination group with P1229.

FIG. 18A-FIG. 18B illustrate weight and body composition changes in mice after subcutaneous injections with the indicated compounds. FIG. 18A shows total weight loss.

FIG. 18B shows changes and fat mass and lean muscle mass.

FIG. 19 shows activity of ligand trap and GLP-1 RA in comparison to the coformulation of the ligand trap and GLP-1 RA.

FIG. 20A illustrates body weight changes in mice following a single subcutaneous injection of P1229 (50 mg/kg), semaglutide (2 mg/kg), or a co-formulation of P1229 (50 mg/kg) and semglutide (2 mg/kg). FIG. 20B-FIG. 20C illustrate pharmacokinetic profiles of semaglutide and P1229 in these animals. FIG. 20D illustrates the changes in circulating follicle-stimulating hormone (FSH) over the course of the study. FSH values are expressed as % baseline. Arrow indicates the onset of treatment.

### DETAILED DESCRIPTION

### Overview

The present disclosure provides combinations of TGFβ superfamily ligand binding agents and GLP-1 agonists, compositions thereof, and methods of use in the treatment of metabolic disorders. In some embodiments, TGFβ superfamily ligand binding agent and GLP-1 agonist are formulated in the same composition. In some embodiments, administration of a binding agent described herein and a GLP-1 agonist exhibits a beneficial effect, wherein administration of the binding agent decreases the dosage of a GLP-1 agonist required to see an effect, and/or increases the therapeutic effect of the GLP-1 agonist, relative to administration of the GLP-1 agonist alone. In some embodiments, a beneficial effect is demonstrated wherein a GLP-1 agonist decreases the dosage of the binding agent required to see an effect, and/or increases the therapeutic effect of the binding agent, relative to administration of the binding agent alone. In some embodiments, the beneficial effect is increased safety of the combination relative to a binding agent or a GLP-1 agonist alone. In some embodiments, the beneficial effect is increased efficacy of the combination relative to a binding agent or a GLP-1 agonist alone. In some embodiments, the beneficial effect is increased tolerability relative to a binding agent or a GLP-1 agonist alone. In some embodiments, the beneficial effect is reduced undesirable side effects relative to a binding agent or a GLP-1 agonist alone.

The present technology is explained in greater detail below. This description is not intended to be a detailed catalog of all the different ways in which the technology may be implemented, or all the features that may be added to the instant technology. For examples, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure, which variations and additions do not depart from the present technology. Hence, the following description is intended to illustrate some particular embodiments of the technology, and not to exhaustively specify all permutations, combinations and variations thereof.

### Definitions

In order to provide a clear and consistent understanding of the terms used in the present specification, a number of definitions are provided below. Moreover, unless defined otherwise, all technical and scientific terms as used herein have the same meaning as commonly understood to one of ordinary skill in the art to which this invention pertains.

The use of the terms “a” and “an” and “the” when used in conjunction with the term “comprising” in the claims and/or the specification may mean “one”, but it is also consistent with the meaning of “one or more”, “at least one”, and “one or more than one”. Similarly, the term “another” may mean at least a second or more. These terms are to be construed to cover both the singular and the plural, unless otherwise indicated herein or clearly contradicted by context.

As used herein, the terms “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps. The term “consisting of” is to be construed as close-ended.

The term “about” is used to indicate that a value or quantity refers to the actual given value and also the approximation of such given value that would reasonably be inferred based on the ordinary skill in the art, including equivalents and approximations due to the experimental and/or measurement conditions for such given value. For example, the term “about” in the context of a given value or range refers to a value or range that is within 20%, preferably within 15%, more preferably within 10%, more preferably within 9%, more preferably within 8%, more preferably within 7%, more preferably within 6%, and more preferably within 5% of the given value or range.

The expression “and/or” where used herein is to be taken as specific disclosure of each of the specified features or components with or without the other. For example, “A and/or B” is to be taken as specific disclosure of each of (i) A, (ii) B, and (iii) A and B, just as if each is set out individually herein. Unless specifically stated or obvious from context, as used herein the term “or” is understood to be inclusive and covers both “or” and “and”. For example, an embodiment of “a composition comprising A or B” would typically present an aspect with a composition comprising both A and B. “Or” should, however, be construed to exclude those aspects presented that cannot be combined without contradiction (e.g., a composition pH that is between 9 and 10 or between 7 and 8).

It is to be understood herein that terms such as “from 1 to 20” include any individual values comprised within and including 1 and 20. Therefore, the term “from 1 to 20” includes 1, 2, 3, 4, 5, 6, 7, 8, 9, 10, 11, 12, 13, 14, 15, 16, 17, 18, 19 and/or 20. Terms such as “from 1 to 20” also include any individual sub-ranges comprised within and including from 1 to 20. The term “from 1 to 20” therefore also includes sub-ranges such as “from 1 to 9”, “from 2 to 9”, “from 3 to 5”, from 5 to 9”, “from 5 to 20”, “from 8 to 20” etc. The same applies for similar expressions such as and not limited to “from 1 to 19”, “from 1 to 18”, “from 1 to 10”, “from 1 to 9”, “from 5 to 15”, etc.

It is to be understood herein that terms such as “from about 15 to about 35” include any individual values comprised within and including 15 and 35. Therefore, terms such as “from about 15 to about 35” include any number between and including 15 and 35 such as 15, 16, 17, 18, 19, 20, 21, 22, 23, 24, 25, 26, 27, 28, 29, 30, 31, 32, 33, 34 and/or 35. Terms such as “from about 15 to about 35” also include any individual sub-ranges comprised within and including from 15 to 35, “from about 16 to about 34”, “from about 16 to about 24”, from about 24 to about 34” and the like. The term “about” in the context of the number of amino acids means that the specified number of amino acids is specifically encompassed and allows a variation of +/−2 in the number of amino acid residues. As such, the terms such as “from about 15 to about 35” also includes “from 13 to 37”, “from 13 to 35”, “from 17 to 37”, from 17 to 35”, etc. The same applies for similar expressions such as and not limited to “from about 16 to about 34”, “from about 16 to about 24”, from about 24 to about 34” and the like.

It is to be understood herein that terms such as “at least 80% identical” include any individual values comprised within and including from 80% to 100% and including 80%, 81%, 82%, 83%, 84%, 85%, 86%, 87%, 88%, 89%, 90%, 91%, 92%, 93%, 94%, 95%, 96%, 97%, 98%, 99% and 100%. The term “at least 80% identical” also includes any individual sub-ranges comprised within and including from 80% to 100%, such as for example, “from 85% to 99%”, “from 97% to 100%”, “from 90% to 100%”, etc. The same applies for similar expressions such as, and not limited to, expressions such as “at least 70% identical”, “at least 90% identical”, and the like.

As used herein, the term “IC50” refers to the half maximal inhibitory concentration (i.e., the concentration of a substance that is required for 50% inhibition in vitro). It is a measure of the potency or effectiveness of a substance in inhibiting a specific biological or biochemical function. IC50 values are typically expressed as molar concentration. The IC50 of an inhibitor can be determined by constructing a dose-response curve and examining the effect of different concentrations of inhibitor on the specific biological or biochemical function in question.

As used herein, the term “inhibition potency” refers to effectiveness of a substance in inhibiting a specific biological or biochemical function such as, without limitation, binding between a protein receptor and its ligand, or activation of a cell receptor by its ligand. In some embodiments, potency of inhibition is determined by measuring the IC50 of an inhibitor for a particular ligand or substrate. In that case, relative inhibition potency for different inhibitors and/or ligands may be assessed by comparing IC50 values. For example, a relative inhibition potency of 3:1 means the ratio of IC50 values for two substances being compared is 3:1, wherein the first substance has a lower inhibition potency (i.e., a greater IC50) than the second substance. A relative inhibition potency of 1:3 means the ratio of IC50 values for two substances being compared is 1:3, wherein the first substance has a greater inhibition potency (i.e., a lower IC50) than the second substance. As the IC50 of an inhibitor can vary depending on the assay conditions, relative inhibition potency for different inhibitors and/or ligands is generally determined by comparing IC50 values obtained under the same assay conditions. The terms “inhibition potency”, “inhibitory potency”, “potency of inhibition” and “neutralization potency” are used interchangeably herein.

As used herein, the term “substantially the same” in reference to relative inhibition potency means that two proteins have a relative inhibition potency that is about the same, e.g., no more than about 2-fold different (+/−2-fold) under the same experimental conditions, e.g., the ratio of IC50 values for the two proteins is about 2:1, 1:2, or 1:1.

As used herein, the term “functionally equivalent” refers to variant sequences that have the same or substantially the same biological activity or function as the original sequence from which it is derived, e.g., no significant change in physiological, chemical, physiochemical or functional properties compared to the original sequence. The term “substantially identical” refers to sequences that are functionally equivalent to the original or reference sequence and have a high degree of sequence identity thereto. Generally, a substantially identical sequence is at least about 80%, at least about 85%, at least about 90%, at least about 95%, at least about 96%, at least about 97%, at least about 98%, or at least about 99% identical to the original or reference sequence and has the same function. In some cases when referring to nucleic acid sequences, a substantially identical sequence hybridizes to the original sequence under high stringency conditions, for example at salt and temperature conditions substantially equivalent to 0.5×SSC to about 5×SSC and 65° C. for both hybridization and wash.

The term “dimeric” refers to the presence of two polypeptides as described herein in a TGFβ superfamily ligand binding agent (also referred to herein as a “binding agent”). “Homodimeric” means the two polypeptides have the same amino acid sequence, whereas “heterodimeric” means the two polypeptides have different amino acid sequences.

The term “divalent” refers to the presence of two TGFβR superfamily ligand binding regions (e.g., the ectodomains) in a TGFβ superfamily ligand binding agent.

As used herein, a “recombinant polypeptide” is a polypeptide made through the use of recombinant DNA technology or genetic engineering. In the context of the present disclosure, recombinant polypeptides are often referred to as “polypeptide constructs” or simply as “polypeptides”.

Proteins (including fragments thereof, preferably biologically active fragments, and peptides, usually having less than 30 amino acids) comprise two or more amino acids coupled to each other via a covalent peptide bond (resulting in a chain of amino acids). The term “polypeptide” as used herein describes a group of molecules, which usually consist of more than 10 amino acids. The terms “polypeptide”, “polypeptide chain” and “chain” are used interchangeably herein. Polypeptides may further form multimers such as dimers, trimers, and higher oligomers, i.e., consisting of more than one polypeptide molecule. Polypeptide molecules forming such dimers, trimers etc. may be identical or non-identical. The corresponding higher order structures of such multimers are, consequently, termed homo- or heterodimers, homo- or heterotrimers etc. An example for a heteromultimer is an antibody molecule, which, in its naturally occurring form, consists of two identical light polypeptide chains and two identical heavy polypeptide chains. The terms “peptide”, “polypeptide” and “protein” also refer to naturally modified peptides/polypeptides/proteins wherein the modification is effected, e.g. by post-translational modifications like glycosylation, acetylation, phosphorylation and the like. A “peptide”, “polypeptide” or “protein” when referred to herein may also be chemically modified such as pegylated. Such modifications are well known in the art and described herein.

As used herein, the terms “(specifically) binds to”, (specifically) recognizes”, “specific for”, “is (specifically) directed to”, and “(specifically) reacts with” mean that a polypeptide interacts or specifically interacts with a given target(s), such as a specific member(s) of the TGFβ superfamily of ligands. Specific binding is believed to be effected by specific motifs in the amino acid sequence of a polypeptide. Thus, binding is achieved as a result of their primary, secondary and/or tertiary structure as well as the result of secondary modifications of said structures. The specific interaction of the target-interaction-site with its specific target may result in a simple binding of said site to the target. Moreover, the specific interaction of the target-interaction-site with its specific target may alternatively or additionally result in the initiation of a signal, e.g. due to the induction of a change of the conformation of the target, an oligomerization of the target, etc., or may block the target from performing another activity, such as binding to an endogenous receptor.

Generally, binding is considered specific when the binding affinity is about 10-12 to 10-9 M, 10-12 to 10-19 M, 10-11 to 10-9 M, or of about 10-11 to 10-9 M. Whether a polypeptide or binding agent specifically reacts with or binds to a target can be tested readily by, inter alia, comparing the reaction of the polypeptide or binding agent with a target with the reaction of the polypeptide or binding agent with other proteins. In some embodiments, a polypeptide or binding agent of the disclosure does not substantially bind to TGFβ superfamily ligands other than the desired ligands, e.g., does not substantially bind to BMP-9.

As used herein, the term “does not substantially bind” or “is not capable of binding” means that a polypeptide or binding agent of the present disclosure does not demonstrate detectable binding to a given target, e.g., does not show reactivity of more than 30%, not more than 20%, not more than 10%, or not more than 9%, 8%, 7%, 6%, 5% or 3% with the given target.

As used herein, the term “selectively binds” is used to mean that a polypeptide binds to a target site that is not shared with other proteins. In general, a selective binding agent will not cross-react with other proteins and exclusively binds to the designated target protein(s). In the context of the present disclosure, “selective for activin A and GDF-8” means that a polypeptide or binding agent binds or neutralizes the activin A and GDF-8 ligands exclusively, without substantially binding or neutralizing other TGFβ superfamily ligands such as, e.g., BMP-9.

“Half-life” means the time where 50% of an administered drug is eliminated through biological processes, e.g., metabolism, excretion, etc.

“Hepatic first-pass metabolism” refers to the propensity of a drug to be metabolized upon first contact with the liver, i.e., during its first pass through the liver.

“Volume of distribution” refers to the degree of retention of a drug throughout the various compartments of the body, such as, e.g., intracellular and extracellular spaces, tissues and organs, etc. and the distribution of the drug within these compartments.

“Degree of blood serum binding” refers to the propensity of a drug to interact with and bind to blood serum proteins, such as albumin, leading to a reduction or loss of biological activity of the drug.

The term “amino acid” or “amino acid residue” typically refers to an amino acid having its art recognized definition such as an amino acid selected from the group consisting of: alanine (Ala or A); arginine (Arg or R); asparagine (Asn or N); aspartic acid (Asp or D); cysteine (Cys or C); glutamine (Gln or Q); glutamic acid (Glu or E); glycine (Gly or G); histidine (His or H); isoleucine (He or I): leucine (Leu or L); lysine (Lys or K); methionine (Met or M); phenylalanine (Phe or F); pro line (Pro or P); serine (Ser or S); threonine (Thr or T); tryptophan (Trp or W); tyrosine (Tyr or Y); and valine (Val or V), although modified, synthetic, or rare amino acids may be used as desired. Generally, amino acids can be grouped as having a nonpolar side chain (e.g., Ala, Cys, He, Leu, Met, Phe, Pro, Val); a negatively charged side chain (e.g., Asp, Glu); a positively charged sidechain (e.g., Arg, His, Lys); or an uncharged polar side chain (e.g., Asn, Cys, Gin, Gly, His, Met, Phe, Ser, Thr, Trp, and Tyr).

“Percent (%) nucleic acid sequence identity” with respect to the nucleic acid sequence of the polypeptides or binding agents identified herein is defined as the percentage of nucleotide residues in a candidate sequence that are identical with the nucleotide residues in the coding sequence of the polypeptides or binding agents. A specific method utilizes the BLASTN module of WU-BLAST-2 set to the default parameters, with overlap span and overlap fraction set to 1 and 0.125, respectively.

### TGFβ Superfamily Ligand Binding Agents

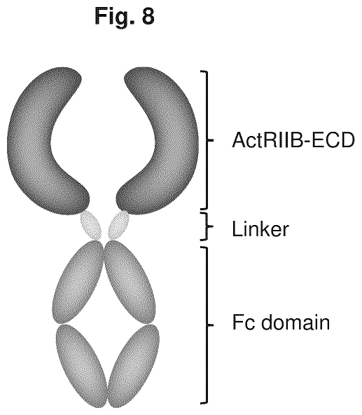

In some embodiments, the present disclosure provides combinations comprising a TGFβ superfamily ligand binding agent and a GLP-1 agonist, wherein the binding agent comprises an ActRIIB-ECD region, a linker region, and an Fc domain (also referred to herein as “binding agents” or “TGFβ ligand binding agents”). The individual components of the binding agents described herein are described in further detail in the following sections. In general, however, the binding agents described herein are dimeric proteins comprising two polypeptides each comprising an ActRIIB-ECD, a peptide linker, and an Fc domain monomer. The two polypeptides assemble via the Fc domain monomers to form the dimeric binding agents described herein. See schematic in FIG. 8. When assembled, the Fc domain monomers in each of the polypeptides form a dimeric Fc domain at one terminus and a divalent ActRIIB-ECD region at the other terminus. Binding agents of the present disclosure can bind to one or more ligand selected from activin A, activin B, GDF-8, and GDF-11 and inhibit signaling of the one or more ligand through their respective receptors, without substantially binding to BMP-9 and/or BMP-10, and/or inhibiting BMP-9 and/or BMP-10 signaling through its receptor. Binding agents may also have further biological activities or functions such as binding to other ligands or targets and the like, as further described herein.

In some embodiments, the binding agents of the present disclosure include two polypeptide chains that are associated via an Fc domain monomer of an antibody or via a constant CH2 domain, a constant CH3 domain and/or via a combination of CH2 and CH3. The constant region of the antibody may be from a human IgG1, IgG2, IgG3 or IgG4 antibody, or substantially identical thereto. The association of both polypeptide chains generally occurs during expression and secretion of the protein, e.g. in mammalian cells. The Fc domain monomer generally comprises a CH2, a CH3, or a CH2 and a CH3 from an antibody heavy chain that is of human origin and typically provides for disulfide crosslinking between single chain polypeptides. In an embodiment, the Fc domain monomer provides for at least one disulfide link between single chain polypeptides. In another embodiment, the Fc domain monomer provides for at least two disulfide links between single chain polypeptides. In some cases, the antibody heavy chain also provides for Protein A-based isolation of the dimeric polypeptide, e.g. after production in host cells.

Certain TGFβ superfamily ligand binding agents and point mutations in the ECD are described in the art. See e.g., WO 2021/158675; WO 2022/150590; WO 2021/158675; WO 2022/072882; WO 2021/189019; and WO 2021/189010. Although point mutations in the ActRIIB ECD have been described in the context of other TGFβ superfamily ligand binding agents, the effects of these mutations in the context of these previously described agents do not predict the effects of these same mutations in the context of the binding agents described herein. See e.g., PCT/CA2023/050116 describing the unpredictability of point mutations in the ActRIIB ECD when combined with linkers of varying lengths. As such, the efficacy of a particular binding agent described herein is determined not only by the mutations comprised in the extracellular ligand binding domain, but also by the length of the linker used.

In some embodiments, binding agents of the present disclosure comprise homodimers, i.e., dimers of a polypeptide having the sequence set forth in any one of SEQ ID NOs: 174-251, or a sequence at least 90%, at least 95%, at least 96%, at least 97%, at least 98%, or at least 99% identical thereto, or a functionally equivalent variant thereof. In other embodiments, binding agents comprise heterodimers, i.e., dimers of two different polypeptides, at least one of the polypeptides having the sequence set forth in any one of SEQ ID NOs: 174-251, or a sequence at least 90%, at least 95%, at least 96%, at least 97%, at least 98%, or at least 99% identical thereto, or a functionally equivalent variant thereof.

In some embodiments, the binding agents of the present disclosure comprise a dimer of a polypeptide comprising an ActRIIB-ECD comprising an L33W mutation and a long peptide linker. In some embodiments, the peptide linker is, or is at least, 10 amino acids in length. In some embodiments, the peptide linker is, or is at least, 14 amino acids in length. In some embodiments, the peptide linker is, or is at least, 19 amino acids in length. In some embodiments, the peptide linker is, or is at least, 39 amino acids in length. In some embodiments, the binding agents of the present disclosure comprise a dimer of a polypeptide comprising or consisting of an amino acid sequence selected from SEQ ID NOs: 211 and 230-234, or a sequence that is at least 90%, at least 95%, at least 96%, at least 97%, at least 98%, or at least 99% identical thereto, or a functionally equivalent variant thereof.

In some embodiments, the binding agents of the present disclosure comprise a dimer of a polypeptide comprising or consisting of SEQ ID NO: 211, or a sequence that is at least 90%, at least 95%, at least 96%, at least 97%, at least 98%, or at least 99% identical thereto, or a functionally equivalent variant thereof. In some embodiments, the binding agents of the present disclosure comprise a dimer of a polypeptide comprising or consisting of SEQ ID NO: 230, or a sequence that is at least 90%, at least 95%, at least 96%, at least 97%, at least 98%, or at least 99% identical thereto, or a functionally equivalent variant thereof. In some embodiments, the binding agents of the present disclosure comprise a dimer of a polypeptide comprising or consisting of SEQ ID NO: 231, or a sequence that is at least 90%, at least 95%, at least 96%, at least 97%, at least 98%, or at least 99% identical thereto, or a functionally equivalent variant thereof. In some embodiments, the binding agents of the present disclosure comprise a dimer of a polypeptide comprising or consisting of SEQ ID NO: 232, or a sequence that is at least 90%, at least 95%, at least 96%, at least 97%, at least 98%, or at least 99% identical thereto, or a functionally equivalent variant thereof. In some embodiments, the binding agents of the present disclosure comprise a dimer of a polypeptide comprising or consisting of SEQ ID NO: 233, or a sequence that is at least 90%, at least 95%, at least 96%, at least 97%, at least 98%, or at least 99% identical thereto, or a functionally equivalent variant thereof. In some embodiments, the binding agents of the present disclosure comprise a dimer of a polypeptide comprising or consisting of SEQ ID NO: 234, or a sequence that is at least 90%, at least 95%, at least 96%, at least 97%, at least 98%, or at least 99% identical thereto, or a functionally equivalent variant thereof.

### Activin Receptor Type IIB Ectodomain Variants

As used herein, the term “Activin receptor type IIB ectodomain variants” or “ActRIIB-ECD variants” refers to a polypeptide comprising the soluble, extracellular portion of the single transmembrane receptor, ActRIIB, that has at least one amino acid substitution relative to a wild type extracellular ActRIIB. The sequence of the wild type human ActRIIB-ECD is shown in SEQ ID NO: 2 (Table 1). Unless otherwise noted, indicated positions for amino acid substitutions are numbered according to the amino acid sequence of SEQ ID NO: 2. For the purposes of this disclosure, the “human wild type ActRIIB-ECD” refers to SEQ ID NO: 2.

In some embodiments, the ActRIIB-ECD variant comprises one or more amino acid substitutions at a position selected from L14, G27, L33, L55, and T69. In some embodiments, the ActRIIB-ECD variant polypeptide comprises one or more amino acid substitutions selected from L14E, L14H, L14S, L14N, L14Q, L14D, G27E, G27D, G27N, G27Q, G27K, G27T, G27M, L33R, L33Y, L33F, L33Q, L33W, L33E, L33K, L33M, L55Y, L55Q, L55M, L55I, L69H, L69Q, L69E, L69R, L69Y, and L69W. In some embodiments, the ActRIIB-ECD variant comprises one or more amino acid substitutions selected from L33R, L33Y, L33F, L33Q, L33W, L33E, L33K, and L33M. In some embodiments, the ActRIIB-ECD variant comprises an amino acid substitution of L33Y. In some embodiments, the ActRIIB-ECD variant comprises an amino acid substitution of L33W. Other amino acid substitutions in the ActRIIB-ECD are known in the art (e.g., WO 2021/158675; WO 2022/150590; WO 2021/158675; WO 2022/072882; WO 2021/189019; and WO 2021/189010, each of which are incorporated herein by reference). These additional mutations can be used in combination with the linkers described herein, and incorporated into the binding agents described herein, to alter ligand binding properties of the ActRIIB-ECD.

In some embodiments, the ActRIIB-ECD variant comprises the amino acid sequence set forth in any one of SEQ ID NOs: 4-62. In some embodiments, an ActRIIB-ECD variant comprises at least 85% (e.g., at least 85%, at least 87%, at least 90%, at least 92%, at least 95%, at least 96%, at least 97%, at least 98%, at least 99%, or greater) amino acid sequence identity to the sequence of a wild type human ActRIIB-ECD. In some embodiments, an ActRIIB-ECD variant may have at least 85% (e.g., at least 85%, at least 87%, at least 90%, at least 92%, at least 95%, at least 96%, at least 97%, at least 98%, at least 99%, or greater) amino acid sequence identity to the sequence set forth in SEQ ID NO: 2.

In some embodiments, the amino acid sequence of the ActRIIB-ECD variant comprises at least 95%, at least 96%, at least 97%, at least 98%, or at least 99% sequence identity to any one of SEQ ID NOs: 4-62.

In some embodiments, the ActRIIB-ECD variant comprises an amino acid substitution at position G27 and comprises an amino acid sequence that is at least 95%, at least 96%, at least 97%, at least 98%, or at least 99% sequence identity to any one of SEQ ID NOs: 4-9. In some embodiments, the ActRIIB-ECD variant comprises an amino acid substitution at position G27 and comprises or consists of an amino acid sequence selected from SEQ ID NOs: 4-9. In some embodiments, the ActRIIB-ECD variant comprises an amino acid substitution at position G27E and comprises or consists of the amino acid sequence of SEQ ID NO: 4. In some embodiments, the ActRIIB-ECD variant comprises an amino acid substitution at position G27N and comprises or consists of the amino acid sequence of SEQ ID NO: 5. In some embodiments, the ActRIIB-ECD variant comprises an amino acid substitution at position G27Q and comprises or consists of the amino acid sequence of SEQ ID NO: 6. In some embodiments, the ActRIIB-ECD variant comprises an amino acid substitution at position G27K and comprises or consists of the amino acid sequence of SEQ ID NO: 7. In some embodiments, the ActRIIB-ECD variant comprises an amino acid substitution at position G27T and comprises or consists of the amino acid sequence of SEQ ID NO: 8. In some embodiments, the ActRIIB-ECD variant comprises an amino acid substitution at position G27M and comprises or consists of the amino acid sequence of SEQ ID NO: 9.

In some embodiments, the ActRIIB-ECD variant comprises an amino acid substitution at position L33 and comprises an amino acid sequence that is at least 95%, at least 96%, at least 97%, at least 98%, or at least 99% sequence identity to any one of SEQ ID NOs: 10-18. In some embodiments, the ActRIIB-ECD variant comprises an amino acid substitution at position L33 and comprises or consists of an amino acid sequence selected from SEQ ID NOs: 10-18. In some embodiments, the ActRIIB-ECD variant comprises an amino acid substitution at position L33F and comprises or consists of the amino acid sequence of SEQ ID NO: 10. In some embodiments, the ActRIIB-ECD variant comprises an amino acid substitution at position L33Q and comprises or consists of the amino acid sequence of SEQ ID NO: 11. In some embodiments, the ActRIIB-ECD variant comprises an amino acid substitution at position L33Y and comprises or consists of the amino acid sequence of SEQ ID NO: 12. In some embodiments, the ActRIIB-ECD variant comprises an amino acid substitution at position L33W and comprises or consists of the amino acid sequence of SEQ ID NO: 13. In some embodiments, the ActRIIB-ECD variant comprises an amino acid substitution at position L33H and comprises or consists of the amino acid sequence of SEQ ID NO: 14. In some embodiments, the ActRIIB-ECD variant comprises an amino acid substitution at position L33R and comprises or consists of the amino acid sequence of SEQ ID NO: 15. In some embodiments, the ActRIIB-ECD variant comprises an amino acid substitution at position L33E and comprises or consists of the amino acid sequence of SEQ ID NO: 16. In some embodiments, the ActRIIB-ECD variant comprises an amino acid substitution at position L33K and comprises or consists of the amino acid sequence of SEQ ID NO: 17. In some embodiments, the ActRIIB-ECD variant comprises an amino acid substitution at position L33M and comprises or consists of the amino acid sequence of SEQ ID NO: 18.

In some embodiments, the ActRIIB-ECD variant comprises an amino acid substitution at position T69 and comprises an amino acid sequence that is at least 95%, at least 96%, at least 97%, at least 98%, or at least 99% sequence identity to SEQ ID NOs: 19-24. In some embodiments, the ActRIIB-ECD variant comprises an amino acid substitution at position G27 and comprises or consists of SEQ ID NO: 19-24. In some embodiments, the ActRIIB-ECD variant comprises an amino acid substitution at position T69H and comprises or consists of the amino acid sequence of SEQ ID NO: 19. In some embodiments, the ActRIIB-ECD variant comprises an amino acid substitution at position T69Q and comprises or consists of the amino acid sequence of SEQ ID NO: 20. In some embodiments, the ActRIIB-ECD variant comprises an amino acid substitution at position T69E and comprises or consists of the amino acid sequence of SEQ ID NO: 21. In some embodiments, the ActRIIB-ECD variant comprises an amino acid substitution at position T69R and comprises or consists of the amino acid sequence of SEQ ID NO: 22. In some embodiments, the ActRIIB-ECD variant comprises an amino acid substitution at position T69Y and comprises or consists of the amino acid sequence of SEQ ID NO: 23. In some embodiments, the ActRIIB-ECD variant comprises an amino acid substitution at position T69W and comprises or consists of the amino acid sequence of SEQ ID NO: 24.

In some embodiments, the ActRIIB-ECD variant comprises an amino acid substitution at position L33 and at position T69 and comprises an amino acid sequence that is at least 95%, at least 96%, at least 97%, at least 98%, or at least 99% sequence identity to SEQ ID NOs: 25-31. In some embodiments, the ActRIIB-ECD variant comprises an amino acid substitution at position L33 and at position T69 and comprises or consists of SEQ ID NO: 25-31. In some embodiments, the ActRIIB-ECD variant comprises an amino acid substitution at position L33Y and T69R and comprises or consists of the amino acid sequence of SEQ ID NO: 25. In some embodiments, the ActRIIB-ECD variant comprises an amino acid substitution at position L33Y and T69Y and comprises or consists of the amino acid sequence of SEQ ID NO: 26. In some embodiments, the ActRIIB-ECD variant comprises an amino acid substitution at position L33Y and T69W and comprises or consists of the amino acid sequence of SEQ ID NO: 27. In some embodiments, the ActRIIB-ECD variant comprises an amino acid substitution at position L33Y and T69H and comprises or consists of the amino acid sequence of SEQ ID NO: 28. In some embodiments, the ActRIIB-ECD variant comprises an amino acid substitution at position L33Y and T69Q and comprises or consists of the amino acid sequence of SEQ ID NO: 29. In some embodiments, the ActRIIB-ECD variant comprises an amino acid substitution at position L33Y and T69E and comprises or consists of the amino acid sequence of SEQ ID NO: 30. In some embodiments, the ActRIIB-ECD variant comprises an amino acid substitution at position L33F and T69Q and comprises or consists of the amino acid sequence of SEQ ID NO: 31.

In some embodiments, the ActRIIB-ECD variant comprises an amino acid substitution at position G27 and at position L33 and comprises an amino acid sequence that is at least 95%, at least 96%, at least 97%, at least 98%, or at least 99% sequence identity to SEQ ID NOs: 32-48. In some embodiments, the ActRIIB-ECD variant comprises an amino acid substitution at position G27 and at position L33 and comprises or consists of SEQ ID NO: 32-48. In some embodiments, the ActRIIB-ECD variant comprises an amino acid substitution at position G27E and L33Y and comprises or consists of the amino acid sequence of SEQ ID NO: 32. In some embodiments, the ActRIIB-ECD variant comprises an amino acid substitution at position G27D and L33Y and comprises or consists of the amino acid sequence of SEQ ID NO: 33. In some embodiments, the ActRIIB-ECD variant comprises an amino acid substitution at position G27N and L33Y and comprises or consists of the amino acid sequence of SEQ ID NO: 34. In some embodiments, the ActRIIB-ECD variant comprises an amino acid substitution at position G27Q and L33Y and comprises or consists of the amino acid sequence of SEQ ID NO: 35. In some embodiments, the ActRIIB-ECD variant comprises an amino acid substitution at position G27K and L33Y and comprises or consists of the amino acid sequence of SEQ ID NO: 36. In some embodiments, the ActRIIB-ECD variant comprises an amino acid substitution at position G27T and L33Y and comprises or consists of the amino acid sequence of SEQ ID NO: 37. In some embodiments, the ActRIIB-ECD variant comprises an amino acid substitution at position G27M and L33Y and comprises or consists of the amino acid sequence of SEQ ID NO: 38. In some embodiments, the ActRIIB-ECD variant comprises an amino acid substitution at position G27D and L33Y and comprises or consists of the amino acid sequence of SEQ ID NO: 39. In some embodiments, the ActRIIB-ECD variant comprises an amino acid substitution at position G27E and L33Y and comprises or consists of the amino acid sequence of SEQ ID NO: 40. In some embodiments, the ActRIIB-ECD variant comprises an amino acid substitution at position G27N and L33Y and comprises or consists of the amino acid sequence of SEQ ID NO: 41. In some embodiments, the ActRIIB-ECD variant comprises an amino acid substitution at position G27Q and L33Y and comprises or consists of the amino acid sequence of SEQ ID NO: 42. In some embodiments, the ActRIIB-ECD variant comprises an amino acid substitution at position G27D and L33W and comprises or consists of the amino acid sequence of SEQ ID NO: 43. In some embodiments, the ActRIIB-ECD variant comprises an amino acid substitution at position G27E and L33W and comprises or consists of the amino acid sequence of SEQ ID NO: 44. In some embodiments, the ActRIIB-ECD variant comprises an amino acid substitution at position G27N and L33W and comprises or consists of the amino acid sequence of SEQ ID NO: 45. In some embodiments, the ActRIIB-ECD variant comprises an amino acid substitution at position G27Q and L33W and comprises or consists of the amino acid sequence of SEQ ID NO: 46. In some embodiments, the ActRIIB-ECD variant comprises an amino acid substitution at position G27T and L33W and comprises or consists of the amino acid sequence of SEQ ID NO: 47. In some embodiments, the ActRIIB-ECD variant comprises an amino acid substitution at position G27M and L33W and comprises or consists of the amino acid sequence of SEQ ID NO: 48.

In some embodiments, the ActRIIB-ECD variant comprises an amino acid substitution at position L14 and at position L33 and comprises an amino acid sequence that is at least 95%, at least 96%, at least 97%, at least 98%, or at least 99% sequence identity to SEQ ID NOs: 49-58. In some embodiments, the ActRIIB-ECD variant comprises an amino acid substitution at position L14 and at position L33 and comprises or consists of SEQ ID NO: 49-58. In some embodiments, the ActRIIB-ECD variant comprises an amino acid substitution at position L14Q and L33Y and comprises or consists of the amino acid sequence of SEQ ID NO: 49. In some embodiments, the ActRIIB-ECD variant comprises an amino acid substitution at position L14D and L33Y and comprises or consists of the amino acid sequence of SEQ ID NO: 50. In some embodiments, the ActRIIB-ECD variant comprises an amino acid substitution at position L14N and L33Y and comprises or consists of the amino acid sequence of SEQ ID NO: 51. In some embodiments, the ActRIIB-ECD variant comprises an amino acid substitution at position L14E and L33Y and comprises or consists of the amino acid sequence of SEQ ID NO: 52. In some embodiments, the ActRIIB-ECD variant comprises an amino acid substitution at position L14H and L33Y and comprises or consists of the amino acid sequence of SEQ ID NO: 53. In some embodiments, the ActRIIB-ECD variant comprises an amino acid substitution at position L14S and L33Y and comprises or consists of the amino acid sequence of SEQ ID NO: 54. In some embodiments, the ActRIIB-ECD variant comprises an amino acid substitution at position L14E and L33W and comprises or consists of the amino acid sequence of SEQ ID NO: 55. In some embodiments, the ActRIIB-ECD variant comprises an amino acid substitution at position L14D and L33W and comprises or consists of the amino acid sequence of SEQ ID NO: 56. In some embodiments, the ActRIIB-ECD variant comprises an amino acid substitution at position L14N and L33W and comprises or consists of the amino acid sequence of SEQ ID NO: 57. In some embodiments, the ActRIIB-ECD variant comprises an amino acid substitution at position L14Q and L33W and comprises or consists of the amino acid sequence of SEQ ID NO: 58.

In some embodiments, the ActRIIB-ECD variant comprises an amino acid substitution at position L33 and at position L55 and comprises an amino acid sequence that is at least 95%, at least 96%, at least 97%, at least 98%, or at least 99% sequence identity to SEQ ID NOs: 59-62. In some embodiments, the ActRIIB-ECD variant comprises an amino acid substitution at position L33 and at position L55 and comprises or consists of SEQ ID NO: 59-62. In some embodiments, the ActRIIB-ECD variant comprises an amino acid substitution at position L33Y and L55Y and comprises or consists of the amino acid sequence of SEQ ID NO: 59. In some embodiments, the ActRIIB-ECD variant comprises an amino acid substitution at position L33Y and L55Q and comprises or consists of the amino acid sequence of SEQ ID NO: 60. In some embodiments, the ActRIIB-ECD variant comprises an amino acid substitution at position L33Y and L55M and comprises or consists of the amino acid sequence of SEQ ID NO: 61. In some embodiments, the ActRIIB-ECD variant comprises an amino acid substitution at position L33Y and L551 and comprises or consists of the amino acid sequence of SEQ ID NO: 62.

Exemplary ActRIIB ECDs are provided in Table 1. Amino acid substitutions are indicated by bold and enlarged text.

TABLE 1
Exemplary ActRIIB and ActRIIB variant ECDs

| ECD | AA Sequence | SEQ ID |
|---|---|---|
| wild type | ETRECIYYNANWELERTNQSGLERCEGEQDKRLHCYASWRNSSGTIELVKK | 2 |
| ActRIIB-ECD | GCWLDDENCYDRQECVATEENPQVYFCCCEGNFCNERFTHLPEAGGPEVTY | |
| | EPPPT | |
| wild type | ILGRSETQECLFFNANWEKDRINQTGVEPCYGDKDKRRHCFATWKNISGSI | 3 |
| ActRIIA-ECD | EIVKQGCWLDDINCYDRTDCVEKKDSPEVYFCCCEGNMCNEKFSYFPEMEV | |
| | TQPTSNPVTPKPPT | |
| ActRIIB-ECD | ETRECIYYNANWELERTNQSGLERCE [symbol] EQDKRLHCYASWRNSSGTIELVKK | 4 |
| G27E | GCWLDDENCYDRQECVATEENPQVYFCCCEGNFCNERFTHLPEAGGPEVTY | |
| | EPPPT | |
| ActRIIB-ECD | ETRECIYYNANWELERTNQSGLERCE [symbol] EQDKRLHCYASWRNSSGTIELVKK | 5 |
| G27N | GCWLDDENCYDRQECVATEENPQVYFCCCEGNFCNERFTHLPEAGGPEVTY | |
| | EPPPT | |
| ActRIIB-ECD | ETRECIYYNANWELERTNQSGLERCE [symbol] EQDKRLHCYASWRNSSGTIELVKK | 6 |
| G27Q | GCWLDDENCYDRQECVATEENPQVYFCCCEGNFCNERFTHLPEAGGPEVTY | |
| | EPPPT | |
| ActRIIB-ECD | ETRECIYYNANWELERTNQSGLERCE [symbol] EQDKRLHCYASWRNSSGTIELVKK | 7 |
| G27K | GCWLDDENCYDRQECVATEENPQVYFCCCEGNFCNERFTHLPEAGGPEVTY | |
| | EPPPT | |
| ActRIIB-ECD | ETRECIYYNANWELERTNQSGLERCE [symbol] EQDKRLHCYASWRNSSGTIELVKK | 8 |
| G27T | GCWLDDENCYDRQECVATEENPQVYFCCCEGNFCNERFTHLPEAGGPEVTY | |
| | EPPPT | |
| ActRIIB-ECD | ETRECIYYNANWELERTNQSGLERCE [symbol] EQDKRLHCYASWRNSSGTIELVKK | 9 |
| G27M | GCWLDDENCYDRQECVATEENPQVYFCCCEGNFCNERFTHLPEAGGPEVTY | |
| | EPPPT | |
| ActRIIB-ECD | ETRECIYYNANWELERTNQSGLERCEGEQDKR [symbol] HCYASWRNSSGTIELVKK | 10 |
| L33F | GCWLDDFNCYDRQECVATEENPQVYFCCCEGNFCNERFTHLPEAGGPEVTY | |
| | EPPPT | |
| ActRIIB-ECD | ETRECIYYNANWELERTNQSGLERCEGEQDKR [symbol] HCYASWRNSSGTIELVKK | 11 |
| L33Q | GCWLDDENCYDRQECVATEENPQVYFCCCEGNFCNERFTHLPEAGGPEVTY | |
| | EPPPT | |
| ActRIIB-ECD | ETRECIYYNANWELERTNQSGLERCEGEQDKR [symbol] HCYASWRNSSGTIELVKK | 12 |
| L33Y | GCWLDDENCYDRQECVATEENPQVYFCCCEGNFCNERFTHLPEAGGPEVTY | |
| | EPPPT | |
| ActRIIB-ECD | ETRECIYYNANWELERTNQSGLERCEGEQDKR [symbol] HCYASWRNSSGTIELVKK | 13 |
| L33W | GCWLDDENCYDRQECVATEENPQVYFCCCEGNFCNERFTHLPEAGGPEVTY | |
| | EPPPT | |
| ActRIIB-ECD | ETRECIYYNANWELERTNQSGLERCEGEQDKR [symbol] HCYASWRNSSGTIELVKK | 14 |
| L33H | GCWLDDENCYDRQECVATEENPQVYFCCCEGNFCNERFTHLPEAGGPEVTY | |
| | EPPPT | |
| ActRIIB-ECD | ETRECIYYNANWELERTNQSGLERCEGEQDKR [symbol] HCYASWRNSSGTIELVKK | 15 |
| L33R | GCWLDDENCYDRQECVATEENPQVYFCCCEGNFCNERFTHLPEAGGPEVTY | |
| | EPPPT | |
| ActRIIB-ECD | ETRECIYYNANWELERTNQSGLERCEGEQDKR [symbol] HCYASWRNSSGTIELVKK | 16 |
| L33E | GCWLDDENCYDRQECVATEENPQVYFCCCEGNFCNERFTHLPEAGGPEVTY | |
| | EPPPT | |
| ActRIIB-ECD | ETRECIYYNANWELERTNQSGLERCEGEQDKR [symbol] HCYASWRNSSGTIELVKK | 17 |
| L33K | GCWLDDENCYDRQECVATEENPQVYFCCCEGNFCNERFTHLPEAGGPEVTY | |
| | EPPPT | |
| ActRIIB-ECD | ETRECIYYNANWELERTNQSGLERCEGEQDKR [symbol] HCYASWRNSSGTIELVKK | 18 |
| L33M | GCWLDDENCYDRQECVATEENPQVYFCCCEGNFCNERFTHLPEAGGPEVTY | |
| | EPPPT | |
| ActRIIB-ECD | ETRECIYYNANWELERTNQSGLERCEGEQDKRLHCYASWRNSSGTIELVKK | 19 |
| T69H | GCWLDDENCYDRQECVA [symbol] EENPQVYFCCCEGNFCNERFTHLPEAGGPEVTY | |
| | EPPPT | |
| ActRIIB-ECD | ETRECIYYNANWELERTNQSGLERCEGEQDKRLHCYASWRNSSGTIELVKK | 20 |
| T69Q | GCWLDDFNCYDRQECVA [symbol] EENPQVYFCCCEGNFCNERFTHLPEAGGPEVTY | |
| | EPPPT | |
| ActRIIB-ECD | ETRECIYYNANWELERTNQSGLERCEGEQDKRLHCYASWRNSSGTIELVKK | 21 |
| T69E | GCWLDDENCYDRQECVA [symbol] EENPQVYFCCCEGNFCNERFTHLPEAGGPEVTY | |
| | EPPPT | |
| ActRIIB-ECD | ETRECIYYNANWELERTNQSGLERCEGEQDKRLHCYASWRNSSGTIELVKK | 22 |
| T69R | GCWLDDENCYDRQECVA [symbol] EENPQVYFCCCEGNFCNERFTHLPEAGGPEVTY | |
| | EPPPT | |
| ActRIIB-ECD | ETRECIYYNANWELERTNQSGLERCEGEQDKRLHCYASWRNSSGTIELVKK | 23 |
| T69Y | GCWLDDENCYDRQECVA [symbol] EENPQVYFCCCEGNFCNERFTHLPEAGGPEVTY | |
| | EPPPT | |
| ActRIIB-ECD | ETRECIYYNANWELERTNQSGLERCEGEQDKRLHCYASWRNSSGTIELVKK | 24 |
| T69W | GCWLDDENCYDRQECVA [symbol] EENPQVYFCCCEGNFCNERFTHLPEAGGPEVTY | |
| | EPPPT | |
| ActRIIB-ECD | ETRECIYYNANWELERTNQSGLERCEGEQDKR [symbol] HCYASWRNSSGTIELVKK | 25 |
| L33Y T69R | GCWLDDENCYDRQECVA [symbol] EENPQVYFCCCEGNFCNERFTHLPEAGGPEVTY | |
| | EPPPT | |
| ActRIIB-ECD | ETRECIYYNANWELERTNQSGLERCEGEQDKR [symbol] HCYASWRNSSGTIELVKK | 26 |
| L33Y T69Y | GCWLDDENCYDRQECVA [symbol] EENPQVYFCCCEGNFCNERFTHLPEAGGPEVTY | |
| | EPPPT | |
| ActRIIB-ECD | ETRECIYYNANWELERTNQSGLERCEGEQDKR [symbol] HCYASWRNSSGTIELVKK | 27 |
| L33Y T69W | GCWLDDENCYDRQECVA [symbol] EENPQVYFCCCEGNFCNERFTHLPEAGGPEVTY | |
| | EPPPT | |
| ActRIIB-ECD | ETRECIYYNANWELERTNQSGLERCEGEQDKR [symbol] HCYASWRNSSGTIELVKK | 28 |
| L33Y T69H | GCWLDDENCYDRQECVA [symbol] EENPQVYFCCCEGNFCNERFTHLPEAGGPEVTY | |
| | EPPPT | |
| ActRIIB-ECD | ETRECIYYNANWELERTNQSGLERCEGEQDKR [symbol] HCYASWRNSSGTIELVKK | 29 |
| L33Y T69Q | GCWLDDENCYDRQECVA [symbol] EENPQVYFCCCEGNFCNERFTHLPEAGGPEVTY | |
| | EPPPT | |
| ActRIIB-ECD | ETRECIYYNANWELERTNQSGLERCEGEQDKR [symbol] HCYASWRNSSGTIELVKK | 30 |
| L33Y T69E | GCWLDDENCYDRQECVA [symbol] EENPQVYFCCCEGNFCNERFTHLPEAGGPEVTY | |
| | EPPPT | |
| ActRIIB-ECD | ETRECIYYNANWELERTNQSGLERCEGEQDKRFHCYASWRNSSGTIELVKK | 31 |
| L33F T69Q | GCWLDDENCYDRQECVA [symbol] EENPQVYFCCCEGNFCNERFTHLPEAGGPEVTY | |
| | EPPPT | |
| ActRIIB-ECD | ETRECIYYNANWELERTNQSGLERCE [symbol] EQDKR [symbol] HCYASWRNSSGTIELVKK | 32 |
| G27E L33Y | GCWLDDENCYDRQECVATEENPQVYFCCCEGNFCNERFTHLPEAGGPEVTY | |
| | EPPPT | |
| ActRIIB-ECD | ETRECIYYNANWELERTNQSGLERCE [symbol] EQDKR [symbol] HCYASWRNSSGTIELVKK | 33 |
| G27D L33Y | GCWLDDENCYDRQECVATEENPQVYFCCCEGNFCNERFTHLPEAGGPEVTY | |
| | EPPPT | |
| ActRIIB-ECD | ETRECIYYNANWELERTNQSGLERCE [symbol] EQDKR [symbol] HCYASWRNSSGTIELVKK | 34 |
| G27N L33Y | GCWLDDENCYDRQECVATEENPQVYFCCCEGNFCNERFTHLPEAGGPEVTY | |
| | EPPPT | |
| ActRIIB-ECD | ETRECIYYNANWELERTNQSGLERCE [symbol] EQDKR [symbol] HCYASWRNSSGTIELVKK | 35 |
| G27Q L33Y | GCWLDDENCYDRQECVATEENPQVYFCCCEGNFCNERFTHLPEAGGPEVTY | |
| | EPPPT | |
| ActRIIB-ECD | ETRECIYYNANWELERTNQSGLERCE [symbol] EQDKR [symbol] HCYASWRNSSGTIELVKK | 36 |
| G27K L33Y | GCWLDDENCYDRQECVATEENPQVYFCCCEGNFCNERFTHLPEAGGPEVTY | |
| | EPPPT | |
| ActRIIB-ECD | ETRECIYYNANWELERTNQSGLERCE [symbol] EQDKR [symbol] HCYASWRNSSGTIELVKK | 37 |
| G27T L33Y | GCWLDDENCYDRQECVATEENPQVYFCCCEGNFCNERFTHLPEAGGPEVTY | |
| | EPPPT | |
| ActRIIB-ECD | ETRECIYYNANWELERTNQSGLERCE [symbol] EQDKR [symbol] HCYASWRNSSGTIELVKK | 38 |
| G27M L33Y | GCWLDDFNCYDRQECVATEENPQVYFCCCEGNFCNERFTHLPEAGGPEVTY | |
| | EPPPT | |
| ActRIIB-ECD | ETRECIYYNANWELERTNQSGLERCE [symbol] EQDKR [symbol] HCYASWRNSSGTIELVKK | 39 |
| G27D L33Y | GCWLDDENCYDRQECVATEENPQVYFCCCEGNFCNERFTHLPEAGGPEVTY | |
| | EPPPT | |
| ActRIIB-ECD | ETRECIYYNANWELERTNQSGLERCE [symbol] EQDKR [symbol] HCYASWRNSSGTIELVKK | 40 |
| G27E L33Y | GCWLDDENCYDRQECVATEENPQVYFCCCEGNFCNERFTHLPEAGGPEVTY | |
| | EPPPT | |
| ActRIIB-ECD | ETRECIYYNANWELERTNQSGLERCE [symbol] EQDKR [symbol] HCYASWRNSSGTIELVKK | 41 |
| G27N L33Y | GCWLDDENCYDRQECVATEENPQVYFCCCEGNFCNERFTHLPEAGGPEVTY | |
| | EPPPT | |
| ActRIIB-ECD | ETRECIYYNANWELERTNQSGLERCE [symbol] EQDKR [symbol] HCYASWRNSSGTIELVKK | 42 |
| G27Q L33Y | GCWLDDENCYDRQECVATEENPQVYFCCCEGNFCNERFTHLPEAGGPEVTY | |
| | EPPPT | |
| ActRIIB-ECD | ETRECIYYNANWELERTNQSGLERCE [symbol] EQDKR [symbol] HCYASWRNSSGTIELVKK | 43 |
| G27D L33W | GCWLDDENCYDRQECVATEENPQVYFCCCEGNFCNERFTHLPEAGGPEVTY | |
| | EPPPT | |
| ActRIIB-ECD | ETRECIYYNANWELERTNQSGLERCE [symbol] EQDKR [symbol] HCYASWRNSSGTIELVKK | 44 |
| G27E L33W | GCWLDDENCYDRQECVATEENPQVYFCCCEGNFCNERFTHLPEAGGPEVTY | |
| | EPPPT | |
| ActRIIB-ECD | ETRECIYYNANWELERTNQSGLERCE [symbol] EQDKR [symbol] HCYASWRNSSGTIELVKK | 45 |
| G27N L33W | GCWLDDENCYDRQECVATEENPQVYFCCCEGNFCNERFTHLPEAGGPEVTY | |
| | EPPPT | |
| ActRIIB-ECD | ETRECIYYNANWELERTNQSGLERCEWE [symbol] EQDKR [symbol] HCYASWRNSSGTIELVKK | 46 |
| G27Q L33W | GCWLDDENCYDRQECVATEENPQVYFCCCEGNFCNERFTHLPEAGGPEVTY | |
| | EPPPTGGGGSGGGGSGGGGSGGGG | |
| ActRIIB-ECD | ETRECIYYNANWELERTNQSGLERCE [symbol] EQDKR [symbol] HCYASWRNSSGTIELVKK | 47 |
| G27T L33W | GCWLDDENCYDRQECVATEENPQVYFCCCEGNFCNERFTHLPEAGGPEVTY | |
| | EPPPT | |
| ActRIIB-ECD | ETRECIYYNANWELERTNQSGLERCE [symbol] EQDKR [symbol] HCYASWRNSSGTIELVKK | 48 |
| G27M L33W | GCWLDDENCYDRQECVATEENPQVYFCCCEGNFCNERFTHLPEAGGPEVTY | |
| | EPPPT | |
| ActRIIB-ECD | ETRECIYYNANWE [symbol] ERTNQSGLERCEGEQDKR [symbol] HCYASWRNSSGTIELVKK | 49 |
| L14Q L33Y | GCWLDDENCYDRQECVATEENPQVYFCCCEGNFCNERFTHLPEAGGPEVTY | |
| | EPPPT | |
| ActRIIB-ECD | ETRECIYYNANWE [symbol] ERTNQSGLERCEGEQDKR [symbol] HCYASWRNSSGTIELVKK | 50 |
| L14D L33Y | GCWLDDENCYDRQECVATEENPQVYFCCCEGNFCNERFTHLPEAGGPEVTY | |
| | EPPPT | |
| ActRIIB-ECD | ETRECIYYNANWE [symbol] ERTNQSGLERCEGEQDKR [symbol] HCYASWRNSSGTIELVKK | 51 |
| L14N L33Y | GCWLDDENCYDRQECVATEENPQVYFCCCEGNFCNERFTHLPEAGGPEVTY | |
| | EPPPT | |
| ActRIIB-ECD | ETRECIYYNANWE [symbol] ERTNQSGLERCEGEQDKR [symbol] HCYASWRNSSGTIELVKK | 52 |
| L14E L33Y | GCWLDDENCYDRQECVATEENPQVYFCCCEGNFCNERFTHLPEAGGPEVTY | |
| | EPPPT | |
| ActRIIB-ECD | ETRECIYYNANWE [symbol] ERTNQSGLERCEGEQDKR [symbol] HCYASWRNSSGTIELVKK | 53 |
| L14H L33Y | GCWLDDENCYDRQECVATEENPQVYFCCCEGNFCNERFTHLPEAGGPEVTY | |
| | EPPPT | |
| ActRIIB-ECD | ETRECIYYNANWE [symbol] ERTNQSGLERCEGEQDKR [symbol] HCYASWRNSSGTIELVKK | 54 |
| L14S L33Y | GCWLDDENCYDRQECVATEENPQVYFCCCEGNFCNERFTHLPEAGGPEVTY | |
| | EPPPT | |
| ActRIIB-ECD | ETRECIYYNANWE [symbol] ERTNQSGLERCEGEQDKR [symbol] HCYASWRNSSGTIELVKK | 55 |
| L14E L33W | GCWLDDENCYDRQECVATEENPQVYFCCCEGNFCNERFTHLPEAGGPEVTY | |
| | EPPPT | |
| ActRIIB-ECD | ETRECIYYNANWE [symbol] ERTNQSGLERCEGEQDKR [symbol] HCYASWRNSSGTIELVKK | 56 |
| L14D L33W | GCWLDDENCYDRQECVATEENPQVYFCCCEGNFCNERFTHLPEAGGPEVTY | |
| | EPPPT | |
| ActRIIB-ECD | ETRECIYYNANWE [symbol] ERTNQSGLERCEGEQDKR [symbol] HCYASWRNSSGTIELVKK | 57 |
| L14N L33W | GCWLDDENCYDRQECVATEENPQVYFCCCEGNFCNERFTHLPEAGGPEVTY | |
| | EPPPT | |
| ActRIIB-ECD | ETRECIYYNANWE [symbol] ERTNQSGLERCEGEQDKR [symbol] HCYASWRNSSGTIELVKK | 58 |
| L14Q L33W | GCWLDDENCYDRQECVATEENPQVYFCCCEGNFCNERFTHLPEAGGPEVTY | |
| | EPPPT | |
| ActRIIB-ECD | ETRECIYYNANWELERTNQSGLERCEGEQDKR [symbol] HCYASWRNSSGTIELVKK | 59 |
| L33Y L55Y | GCW [symbol] DDENCYDRQECVATEENPQVYFCCCEGNFCNERFTHLPEAGGPEVTY | |
| | EPPPT | |
| ActRIIB-ECD | ETRECIYYNANWELERTNQSGLERCEGEQDKR [symbol] HCYASWRNSSGTIELVKK | 60 |
| L33Y L55Q | GCW [symbol] DDFNCYDRQECVATEENPQVYFCCCEGNFCNERFTHLPEAGGPEVTY | |
| | EPPPT | |
| ActRIIB-ECD | ETRECIYYNANWELERTNQSGLERCEGEQDKR [symbol] HCYASWRNSSGTIELVKK | 61 |
| L33Y L55M | GCW [symbol] DDENCYDRQECVATEENPQVYFCCCEGNFCNERFTHLPEAGGPEVTY | |
| | EPPPT | |
| ActRIIB-ECD | ETRECIYYNANWELERTNQSGLERCEGEQDKR [symbol] HCYASWRNSSGTIELVKK | 62 |
| L33Y L55I | GCW [symbol] DDENCYDRQECVATEENPQVYFCCCEGNFCNERFTHLPEAGGPEVTY | |
| | EPPPT | |

In some embodiments, an ActRIIB-ECD variant of the disclosure further includes an extension of up to 5 amino acids at the N-terminus. In some embodiments, the ActRIIB-ECD variant of the disclosure further includes an extension of 5 amino acids at the N-terminus, e.g., of GRGEA (SEQ ID NO: 63). In some embodiments, the ActRIIB-ECD variant of the disclosure further includes an extension at the N-terminus of 4 amino acids, 3 amino acids, 2 amino acids, or 1 amino acid, for example and without limitation, RGEA, GEA, EA, or A. In some embodiments, an ActRIIB-ECD variant of the disclosure further includes an extension of 3 amino acids at the C-terminus. In some embodiments, the ActRIIB-ECD variant of the disclosure further includes an extension of 3 amino acids at the C-terminus, e.g., of APT.

Exemplary ActRIIB ECDs with N-terminal and C-terminal extensions are provided in Table 2. The extension amino acids are indicated in bold and italicized text. In some embodiments, any one of SEQ ID NOs: 4-62 can further comprise an N- or C-terminal extension.

TABLE 2
ActRIIB-ECDs with N-terminal extensions

| ECD | AA Sequence | SEQ ID |
|---|---|---|
| WT ActRIIB-ECD | GRGEAETRECIYYNA | 65 |
| N and C terminal | NWELERTNQSGLERC | |
| extensions | EGEQDKRLHCYASWR | |
| | NSSGTIELVKKGCWL | |
| | DDENCYDRQECVATE | |
| | ENPQVYFCCCEGNFC | |
| | NERFTHLPEAGGPEV | |
| | TYEPPPTAPT | |
| WT ActRIIB-ECD | GRGEAETRECIYYNA | 66 |
| N terminal | NWELERTNQSGLERC | |
| extension | EGEQDKRLHCYASWR | |
| | NSSGTIELVKKGCWL | |
| | DDENCYDRQECVATE | |
| | ENPQVYFCCCEGNFC | |
| | NERFTHLPEAGGPEV | |
| | TYEPPPT | |
| WT ActRIIB-ECD | ETRECIYYNANWELE | 67 |
| C terminal | RTNQSGLERCEGEQD | |
| extension | KRLHCYASWRNSSGT | |
| | IELVKKGCWLDDENC | |
| | YDRQECVATEENPQV | |
| | YFCCCEGNFCNERFT | |
| | HLPEAGGPEVTYEPP | |
| | PTAPT | |

ActRIIB-ECD variants of the disclosure have been designed to maximize therapeutic efficacy in certain disease indications while minimizing adverse effects, specifically to prevent or reduce disruption of endogenous BMP-9 and/or BMP-10 signaling, while maintaining and/or increasing neutralization potency for other TGFβ superfamily ligands such as activin A, activin B, GDF-8, and/or GDF-11. ActRIIB-ECD variants of the disclosure exhibit: (1) similar or improved binding to activin A, activin B, GDF-8, and/or GDF-11 compared to wild type ActRIIB-ECD, which allows them to compete with endogenous receptors for ligand binding and reduce or inhibit endogenous receptor signaling; and (2) reduced binding to BMP-9 compared to wild type ActRIIB-ECD, which avoids toxicity associated with inhibition of BMP-9 signaling; and optionally (3) similar or reduced binding to or inhibition of BMP-10, which avoids toxicity associated with inhibition of BMP-10 signaling.

In some embodiments, ActRIIB-ECD variants of the disclosure bind to one or more ligand selected from activin A, activin B, GDF-8, and GDF-11 and inhibit signaling of the one or more ligand through their respective receptors, without substantially binding to BMP-9 or BMP-10 and/or inhibiting BMP-9 or BMP-10 signaling through their receptor(s).

In some embodiments, the inhibition potency of an ActRIIB-ECD variant of the disclosure for human BMP-9 signaling is reduced by about 5-fold compared to the inhibition potency of the human wild type ActRIIB-ECD for human BMP-9 signaling.

In some embodiments, the inhibition potency of an ActRIIB-ECD variant of the disclosure for human BMP-9 signaling is reduced by about 10-fold compared to the inhibition potency of the human wild type ActRIIB-ECD for human BMP-9 signaling.

In some embodiments, the inhibition potency of an ActRIIB-ECD variant of the disclosure for human BMP-9 signaling is reduced by about 100-fold compared to the inhibition potency of the human wild type ActRIIB-ECD for human BMP-9 signaling.

In some embodiments, the inhibition potency of an ActRIIB-ECD variant of the disclosure for human BMP-10 signaling is reduced by about 5-fold compared to the inhibition potency of the human wild type ActRIIB-ECD for human BMP-10 signaling.

In some embodiments, the inhibition potency of an ActRIIB-ECD variant of the disclosure for human BMP-10 signaling is reduced by about 10-fold compared to the inhibition potency of the human wild type ActRIIB-ECD for human BMP-10 signaling.

In some embodiments, the inhibition potency of an ActRIIB-ECD variant of the disclosure for human BMP-10 signaling is reduced by about 100-fold compared to the inhibition potency of the human wild type ActRIIB-ECD for human BMP-10 signaling.

In some embodiments, the inhibition potency of ActRIIB-ECD variant of the disclosure for the one or more ligand selected from activin A, activin B, GDF-8, and GDF-11 is increased or is substantially the same as the inhibition potency of the human wild type ActRIIB-ECD for the same one or more ligand.

In some embodiments, the ActRIIB-ECD variant of the disclosure has greater inhibition potency for activin A and lower inhibition potency for BMP-9 and/or BMP-10 compared to the human wild type ActRIIB-ECD.

In some embodiments, the ActRIIB-ECD variant of the disclosure has greater inhibition potency for activin B and lower inhibition potency for BMP-9 and/or BMP-10 compared to the human wild type ActRIIB-ECD.

In some embodiments, the ActRIIB-ECD variant of the disclosure has greater inhibition potency for both activin A and activin B and lower inhibition potency for BMP-9 and/or BMP-10 compared to the human wild type ActRIIB-ECD.

In some embodiments, the ActRIIB-ECD variant of the disclosure has greater inhibition potency for GDF-8 and lower inhibition potency for BMP-9 and/or BMP-10 compared to the human wild type ActRIIB-ECD.

In some embodiments, the ActRIIB-ECD variant of the disclosure has greater inhibition potency for GDF-11 and lower inhibition potency for BMP-9 and/or BMP-10 compared to the human wild type ActRIIB-ECD.

In some embodiments, the ActRIIB-ECD variant of the disclosure has reduced inhibition potency for BMP-10 compared to the human wild type ActRIIB-ECD.

In some embodiments, the ActRIIB-ECD variant of the disclosure has greater inhibition potency for both activin A and/or activin B; lower inhibition potency for BMP-9; and lower inhibition potency for BMP-10 compared to the human wild type ActRIIB-ECD.

In some embodiments, the ActRIIB-ECD variant of the disclosure does not cause a vascular complication in a subject. In some embodiments, the ActRIIB-ECD variant of the disclosure does not increase vascular permeability or leakage in a subject.

Consequently, in accordance with the disclosure there are provided herein novel polypeptides comprising an Activin receptor type IIB (ActRIIB) ectodomain (ECD) variant, the variant having one or more amino acid substitution relative to the sequence of the human wild type ActRIIB-ECD, having a tailored TGFβ superfamily ligand specificity in order to maximize therapeutic efficacy while minimizing adverse effects, specifically with the goal of preventing or reducing disruption of endogenous BMP-9 and/or BMP-10 signaling, while maintaining and/or increasing neutralization potency for other TGFβ superfamily ligands such as activin A, activin B, GDF-8, and/or GDF-11.

### Polypeptides Comprising ActRIIB ECD Variants

In some embodiments, the present disclosure provides polypeptides comprising an ActRIIB ECD variant fused, via a linker, to an Fc domain monomer. In some embodiments, the polypeptides comprise, from N-terminus to C-terminus, an ActRIIB ECD variant-peptide linker-Fc domain monomer. The polypeptides comprising ActRIIB ECDs can dimerize via cysteine bonds between Fc domain monomers to form the TGFβ superfamily ligand binding agents described herein.

### Linkers

In some embodiments, the ActRIIB ECD variants described herein are fused to a heterologous domain by way of a linker. In some embodiments, the heterologous domain increases stability of the polypeptide. In some embodiments, the heterologous domain is selected from the group consisting of an Fc domain monomer (e.g., a wild-type Fc domain monomer, an Fc domain monomer with one or more amino acid substitutions), an albumin-binding peptide, a fibronectin domain, or a human serum albumin domain.

As used herein, the terms “peptide linker” and “linker” are used interchangeably to refer to a short stretch of amino acids used to connect two functional domains together in a polypeptide chain. For example, in some embodiments of the polypeptides or binding agents of the disclosure, the ActRIIB-ECD variant and the Fc domain monomer are linked together on a polypeptide chain via one or more peptide linkers. Peptide linkers can also be used to attach other domains or modules or regions (such as half-life extending domains) to the polypeptides or binding agents of the disclosure. The term “long linker” as used herein refers to a linker that is at least 10 amino acids in length (i.e., 10, 11, 12, 13, 14, 15, 16, 17, 18, 19, 20 or more amino acids in length). The term “short linker” as used herein refers to a linker that is less than 10 amino acids in length (i.e., 9, 8, 7, 6, 5, 4, 3, 2, or 1 amino acids in length)

Suitable peptide linkers are known in the art, and include, for example, peptide linkers containing flexible amino acid residues such as glycine, alanine, and serine. In some embodiments, a linker can contain motifs, e.g., multiple or repeating motifs, of GA, GS, GG, GGA, GGS, GGG, GGGA (SEQ ID NO: 126), GGGS (SEQ ID NO: 125), GGGG (SEQ ID NO: 104), GGGGA (SEQ ID NO: 124), GGGGS (SEQ ID NO: 103), GGGGG (SEQ ID NO: 123), GGAG (SEQ ID NO: 122), GGSG (SEQ ID NO: 121), AGGG (SEQ ID NO: 120), or SGGG (SEQ ID NO: 110).

In some embodiments, a linker can contain 2 to 12 amino acids including motifs of GA or GS, e.g., GA, GS, GAGA (SEQ ID NO: 137), GSGS (SEQ ID NO: 129), GAGAGA (SEQ ID NO: 130), GSGSGS (SEQ ID NO: 131), GAGAGAGA (SEQ ID NO: 132), GSGSGSGS (SEQ ID NO: 133), GAGAGAGAGA (SEQ ID NO: 134), GSGSGSGSGS (SEQ ID NO: 135), GAGAGAGAGAGA (SEQ ID NO: 136), and GSGSGSGSGSGS (SEQ ID NO: 138). In some embodiments, a linker can contain 3 to 12 amino acids including motifs of GGA or GGS, e.g., GGA, GGS, GGAGGA (SEQ ID NO: 139), GGSGGS (SEQ ID NO: 140), GGAGGAGGA (SEQ ID NO: 141), GGSGGSGGS (SEQ ID NO: 142), GGAGGAGGAGGA (SEQ ID NO: 143), and GGSGGSGGSGGS (SEQ ID NO: 144). In some embodiments, a linker can contain 4 to 12 amino acids including motifs of GGAG (SEQ ID NO: 145), GGSG (SEQ ID NO: 146), GGAGGGAG (SEQ ID NO: 147), GGSGGGSG (SEQ ID NO: 148), GGAGGGAGGGAG (SEQ ID NO: 149), and GGSGGGSGGGSG (SEQ ID NO: 150). In some embodiments, a linker can contain motifs of GGGGA (SEQ ID NO: 124) or GGGGS (SEQ ID NO: 103), e.g, GGGGAGGGGAGGGGA (SEQ ID NO: 151) and GGGGSGGGGSGGGGS (SEQ ID NO: 93). In some embodiments, an amino acid linker between an ActRIIB-ECD variant and a heterologous domain (e.g., an Fc domain monomer (e.g., a wild-type Fc domain monomer, an Fc domain monomer with one or more amino acid substitutions), an albumin-binding peptide, a fibronectin domain, or a human serum albumin domain) may be GGG, GGGA (SEQ ID NO: 126), GGGG (SEQ ID NO: 104), GGGAG (SEQ ID NO: 168), GGGAGG (SEQ ID NO: 169), or GGGAGGG (SEQ ID NO: 170).

In the event that a linker is used, the linker is generally of a length and sequence sufficient to ensure that each of the domains can, independently from one another, retain their differential binding specificities and/or functions. In some embodiments, peptide linkers which furthermore do not promote any secondary structures are selected. The linkage of said domains to each other can be provided, e.g., by genetic engineering, as described herein. Methods for preparing fused and operatively linked polypeptide constructs and expressing them in mammalian cells or bacteria are well-known in the art (e.g., WO 99/54440 or Sambrook et al., Molecular Cloning: A Laboratory Manual, Cold Spring Harbor Laboratory Press, Cold Spring Harbor, N.Y., 2001).

In some embodiments, the linker comprises various permutations of amino acid sequences containing Gly and Ser. In some embodiments, the linkers are glycine and serine rich linkers. In some embodiments, the linker may be rich in glycine (e.g, 2-10, 2-5, 2-4, 2-3 glycine residues) or glycine and proline residues and may, for example, contain a single sequence of threonine/serine and glycines or repeating sequences of threonine/serine and/or glycines, e.g., GGG, GGGG (SEQ ID NO: 104), GGGS (SEQ ID NO: 125), TGGGG (SEQ ID NO: 108), SGGGG (SEQ ID NO: 109), TGGG (SEQ ID NO: 107), or SGGG (SEQ ID NO: 110) singlets, or repeats. Other near neutral amino acids, such as, but not limited to, Thr, Asn, Pro and Ala, may also be used in the linker sequence.

In some embodiments, the linker is 10 amino acids in length. In some embodiments, the linker is greater than 10 amino acids in length. In some embodiments, the linker has a length of at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids. In some embodiments, the linker is less than 40, 35, 30, 25, 22 or 20 amino acids. In some embodiments, the linker is 10-50, 10-40, 10-30, 10-25, 10-21, 10-15, 10-14, 12-14, 15-25, 17-22, 20, or 21 amino acids in length. In some embodiments, the linker is 14-40, 14-39, 14-35, 14-30, 14-25, or 14-20 amino acids in length. In some embodiments, the linker is at least 10 amino acids in length. In some embodiments, the linker is at least 14 amino acids in length. In some embodiments, the linker is at least 19 amino acids in length. In some embodiments, the linker is at least 39 amino acids in length. In some embodiments, the linker is 14 amino acids in length. In some embodiments, the linker is 19 amino acids in length. In some embodiments, the linker is 39 amino acids in length. In some embodiments, the linker is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length. In further embodiments, the linkers have a length of at least 12, 14, 15, 20, 21, 25, 30, 35, 40, 45 or 50 amino acids.

In other embodiments, the linker is less than 10 amino acids in length. In some embodiments, the linker is 3 amino acids in length. In some embodiments, the linker is 6 amino acids in length. In some embodiments, the linker is 9 amino acids in length.

In some embodiments, the linker comprises SEQ ID NO: 98. In some embodiments, the linker comprises SEQ ID NO: 94. In some embodiments, the linker comprises SEQ ID NO: 89.

In some embodiments, the linker consists of SEQ ID NO: 98. In some embodiments, the linker consists of SEQ ID NO: 94. In some embodiments, the linker consists of SEQ ID NO: 89.

In some embodiments, the linker comprises or consists of the sequence set forth in any one of SEQ ID NOs: 68-170.

In some embodiments, linkers are Glycine-rich, often Glycine/Serine-rich, peptides of up to 40 amino acids, or from 1 to 40 amino acids, from 2 to 39 amino acids, from 3 to 39 amino acids, from 3 to 14 amino acids, from 3 to 19 amino acids, from 5 to 25 amino acids, from 5 to 20 amino acids, from 5 to 15 amino acids, or from 15 to 25 amino acids. In some embodiments, peptide linkers comprise only a relatively small number of amino acid residues, e.g., 39 amino acids or less, 19 amino acids or less, 14 amino acids or less, 5 amino acids or less, or 3 amino acids of less. In certain embodiments, Gly-rich linkers are used. In one embodiment, a peptide linker may consist of the single amino acid Glycine (Gly). In another embodiment, a peptide linker comprises or consists of the amino acid sequence Gly-Gly-Gly-Gly-Ser, i.e. Gly4Ser, or polymers thereof, i.e. (Gly4Ser)n, where n is an integer of 1 or greater, or n is from 1 to 8 (e.g. 1, 2, 3, 4, 5, 6, 7, or 8).

In some embodiments, the linker comprises the amino acid sequence GlyGlyGlyGlySer (GGGGS) (SEQ ID NO: 103), or repetitions thereof (GGGGS)n, where n >2. In particular embodiments n >3, or n=3-10. In some embodiments, n >4, or n=4-10. In some embodiments, n is not greater than 4 in a (GGGGS)n linker. In some embodiments, n=4-10, 4-9, 4-8, 4-7, 4-6, 4-5, 5-8, 5-7, or 5-6. In some embodiments, n=3, 4, 5, 6, or 7. In some embodiments, n=4. In some embodiments, a linker comprising a (GGGGS)n sequence also comprises an N-terminal threonine.

In some embodiments, a linker can also contain amino acids other than glycine, alanine, and serine, e.g., AAAL (SEQ ID NO: 152), AAAK (SEQ ID NO: 153), AAAR (SEQ ID NO: 154), EGKSSGSGSESKST (SEQ ID NO: 155), GSAGSAAGSGEF (SEQ ID NO: 156), AEAAAKEAAAKA (SEQ ID NO: 157), KESGSVSSEQLAQFRSLD (SEQ ID NO: 158), GENLYFQSGG (SEQ ID NO: 159), SACYCELS (SEQ ID NO: 160), RSIAT (SEQ ID NO: 161), RPACKIPNDLKQKVMNH (SEQ ID NO: 162), GGSAGGSGSGSSGGSSGASGTGTAGGTGSGSGTGSG (SEQ ID NO: 163), AAANSSIDLISVPVDSR (SEQ ID NO: 164), or GGSGGGSEGGGSEGGGSEGGGSEGGGSEGGGSGGGS (SEQ ID NO: 165). In some embodiments, a linker can contain motifs, e.g., multiple or repeating motifs, of EAAAK (SEQ ID NO: 166). In some embodiments, a linker can contain motifs, e.g., multiple or repeating motifs, of praline-rich sequences such as (XP)n, in which X may be any amino acid (e.g., A, K, or E) and n is from 1-5, and PAPAP (SEQ ID NO: 167).

The length of the peptide linker and the amino acids used can be adjusted depending on the two proteins involved and the degree of flexibility desired in the final protein fusion polypeptide. The length of the linker can be adjusted to ensure proper protein folding and avoid aggregate formation.

Non-limiting examples of linkers are depicted in Table 3. It should be understood that the linkers are not meant to be particularly limited and any suitable linker may be used, as long as the desired functions (binding, neutralization, etc.) of the polypeptide or binding agent are provided.

TABLE 3
Exemplary linker sequences

| SEQ ID | Linker sequence |
|---|---|
| 68 | GGGGSGGGGSGGGGSGGGGSGGGGSGGGGSGGGGSGGGGS |
| 69 | GGGGSGGGGSGGGGSGGGGSGGGGSGGGGSGGGGSGGGG |
| 70 | GGGGSGGGGSGGGGSGGGGSGGGGSGGGGSGGGGSGGG |
| 71 | GGGGSGGGGSGGGGSGGGGSGGGGSGGGGSGGGGSGG |
| 72 | GGGGSGGGGSGGGGSGGGGSGGGGSGGGGSGGGGSG |
| 73 | GGGGSGGGGSGGGGSGGGGSGGGGSGGGGSGGGGS |
| 74 | GGGGSGGGGSGGGGSGGGGSGGGGSGGGGSGGGG |
| 75 | GGGGSGGGGSGGGGSGGGGSGGGGSGGGGSGGG |
| 76 | GGGGSGGGGSGGGGSGGGGSGGGGSGGGGSGG |
| 77 | GGGGSGGGGSGGGGSGGGGSGGGGSGGGGSG |
| 78 | GGGGSGGGGSGGGGSGGGGSGGGGSGGGGS |
| 79 | GGGGSGGGGSGGGGSGGGGSGGGGSGGGG |
| 80 | GGGGSGGGGSGGGGSGGGGSGGGGSGGG |
| 81 | GGGGSGGGGSGGGGSGGGGSGGGGSGG |
| 82 | GGGGSGGGGSGGGGSGGGGSGGGGSG |
| 83 | GGGGSGGGGSGGGGSGGGGSGGGGS |
| 84 | GGGGSGGGGSGGGGSGGGGSGGGG |
| 85 | GGGGSGGGGSGGGGSGGGGSGGG |
| 86 | GGGGSGGGGSGGGGSGGGGSGG |
| 87 | GGGGSGGGGSGGGGSGGGGSG |
| 88 | GGGGSGGGGSGGGGSGGGGS |
| 89 | GGGGSGGGGSGGGGSGGGG |
| 90 | GGGGSGGGGSGGGGSGGG |
| 91 | GGGGSGGGGSGGGGSGG |
| 92 | GGGGSGGGGSGGGGSG |
| 93 | GGGGSGGGGSGGGGS |
| 94 | GGGGSGGGGSGGGG |
| 95 | GGGGSGGGGSGGG |
| 96 | GGGGSGGGGSGG |
| 97 | GGGGSGGGGSG |
| 98 | GGGGSGGGGS |
| 99 | GGGGSGGGG |
| 100 | GGGGSGGG |
| 101 | GGGGSGG |
| 102 | GGGGSG |
| 103 | GGGGS |
| 104 | GGGG |
| 105 | GGG |
| 106 | GG |
| 107 | TGGG |
| 108 | TGGGG |
| 109 | SGGGG |
| 110 | SGGG |
| 111 | TGGGGSGGGGS |
| 112 | TGGGGSGGGGSGGGGS |
| 113 | TGGGGSGGGGSGGGGSGGGGS |
| 114 | TGGGGSGGGGSGGGGSGGGGSGGGGS |
| 115 | TGGGGSGGGGSGGGGSGGGGSGGGGSGGGGS |
| 116 | TGGGGSGGGGSGGGGSGGGGSGGGGSGGGGSGGGGS |
| 117 | TGGGPKSCDK |
| 118 | GA |
| 119 | GS |
| 120 | AGGG |
| 121 | GGSG |
| 122 | GGAG |
| 123 | GGGGG |
| 124 | GGGGA |
| 125 | GGGS |
| 126 | GGGA |
| 127 | GGS |
| 128 | GGA |
| 129 | GSGS |
| 130 | GAGAGA |
| 131 | GSGSGS |
| 132 | GAGAGAGA |
| 133 | GSGSGSGS |
| 134 | GAGAGAGAGA |
| 135 | GSGSGSGSGS |
| 136 | GAGAGAGAGAGA |
| 137 | GAGA |
| 138 | GSGSGSGSGSGS |
| 139 | GGAGGA |
| 140 | GGSGGS |
| 141 | GGAGGAGGA |
| 142 | GGSGGSGGS |
| 143 | GGAGGAGGAGGA |
| 144 | GGSGGSGGSGGS |
| 145 | GGAG |
| 146 | GGSG |
| 147 | GGAGGGAG |
| 148 | GGSGGGSG |
| 149 | GGAGGGAGGGAG |
| 150 | GGSGGGSGGGSG |
| 151 | GGGGAGGGGAGGGGA |
| 152 | AAAL |
| 153 | AAAK |
| 154 | AAAR |
| 155 | EGKSSGSGSESKST |
| 156 | GSAGSAAGSGEF |
| 157 | AEAAAKEAAAKA |
| 158 | KESGSVSSEQLAQFRSLD |
| 159 | GENLYFQSGG |
| 160 | SACYCELS |
| 161 | RSIAT |
| 162 | RPACKIPNDLKQKVMNH |
| 163 | GGSAGGSGSGSSGGSSGASGTGTAGGTGSGSGTGSG |
| 164 | AAANSSIDLISVPVDSR |
| 165 | GGSGGGSEGGGSEGGGSEGGGSEGGGSEGGGSGGGS |
| 166 | EAAAK |
| 167 | PAPAP |
| 168 | GGGAG |
| 169 | GGGAGG |
| 170 | GGGAGGG |

In some embodiments, the ActRIIB ECD variant polypeptide or binding agent of the disclosure comprises one or more linkers having the sequence set forth in any one of SEQ ID NOs: 89, 94, or 98. In some embodiments, the ActRIIB ECD variant polypeptide or binding agent comprises a Glycine-rich linker at the C-terminus of the ActRIIB ECD variant polypeptide that is 2, 3, 6, 10, 14, 19, or 39 amino acids long. In some embodiments, the ActRIIB ECD variant polypeptide or binding agent of the disclosure comprises a linker of SEQ ID NO: 89 at the C-terminus of the ActRIIB ECD variant polypeptide. In some embodiments, the ActRIIB ECD variant polypeptide or binding agent of the disclosure comprises a linker of SEQ ID NO: 94 at the C-terminus of the ActRIIB ECD variant polypeptide. In some embodiments, the ActRIIB ECD variant polypeptide or binding agent of the disclosure comprises a linker of SEQ ID NO: 98 at the C-terminus of the ActRIIB ECD variant polypeptide.

### Fc Domain Monomers and Fe Domains

In some embodiments, the present disclosure provides polypeptides comprising an ActRIIB-ECD variant described herein fused, via linker, to an Fc domain monomer. In some embodiments, the ActRIIB-ECD variant is fused at the C-terminus, via a linker, to the N-terminus of the Fc domain monomer.

As used herein, “Fc domain monomer” describes the single chain protein that, when associated with another Fc domain monomer, forms a functional Fc domain. The association of two Fc domain monomers creates one Fc domain. As used herein, “Fc domain” describes the minimum region (in the context of a larger polypeptide) or smallest protein folded structure (in the context of an isolated protein) that can bind to or be bound by an Fc receptor (FcR). When two Fc domain monomers associate, the resulting Fc domain has Fc receptor binding activity. Thus, an Fc domain is a dimeric structure that can bind an Fc receptor. Unless otherwise noted, all references herein to a “variant Fc domain” are to be understood as referring to a dimeric Fc domain, in which each Fc domain monomer comprises the referenced mutation.

It will be understood that Fc domain as used herein includes the polypeptides comprising the constant region of an antibody excluding the first constant region immunoglobulin domain. Thus, Fc refers to the last two constant region immunoglobulin domains (CH2, CH3) of IgG and optionally the flexible hinge N-terminal to these domains. Although the boundaries of the Fc domain monomer may vary, the human IgG heavy chain Fc domain monomer is usually defined to comprise residues C226 or P230 to its carboxyl-terminus. Unless otherwise noted, all references to amino acid positions in Fc domains and Fc domain monomers are according to the EU index as set forth in Kabat (1991, NIH Publication 91-3242, National Technical Information Service, Springfield, Va.). Fc may refer to this region in isolation, or this region in the context of a polypeptide construct. It is noted that polymorphisms have been observed at a number of Fc positions, including but not limited to Kabat 270, 272, 312, 315, 356, and 358, and thus slight differences between the sequences provided herein and sequences in the art may exist. The Fc domain monomer included in the polypeptides or binding agents of the present disclosure may be an IgG1, IgG2, IgG3, or IgG4 domain.

In exemplary embodiments, the polypeptides of the disclosure comprise one or more constant region of an antibody, e.g., the second constant domain (CH2) and/or the third constant domain (CH3) of an antibody heavy chain, or an Fc domain monomer of an antibody heavy chain. The antibody may be, for example and without limitation, an IgG antibody such as an IgG1, IgG2, IgG3 or IgG4 antibody. In particular embodiments, the antibody is a human antibody, e.g., the Fc domain monomer comprises a constant region of the heavy chain of a human IgG1, IgG2, IgG3 or IgG4. In some embodiments, the Fc domain monomer has at least 85%, at least 90%, at least 95%, at least 96%, at least 97%, at least 98%, or at least 99% sequence identity with a human IgG1, IgG2, IgG3 or IgG4 constant region. In a particular embodiment, the Fc domain monomer comprises or consists of an Fc domain monomer of a human IgG1 antibody. In another particular embodiment, the Fc domain monomer comprises or consists of an Fc domain monomer of a human IgG2 antibody. In another particular embodiment, the Fc domain monomer comprises or consists of an Fc domain monomer of a human IgG4 antibody. Exemplary Fc domain sequences (including both wild type sequences, polymorphisms thereof, and variant sequences) are provided in Table 4.

TABLE 4
Exemplary Fc domain sequences

| Isotype | AA Sequence | SEQ ID |
|---|---|---|
| IgG1-EEM | THTCPPCPAPELLGGPSVFLFPPKPKDTLMISRTPEVTCVVVDVSHEDPEVK | 252 |
| polymorph | FNWYVDGVEVHNAKTKPREEQYNSTYRVVSVLTVLHQDWLNGKEYKCKVSNK | |
| (“IgG1-EM”) | ALPAPIEKTISKAKGQPREPQVYTLPPSREEMTKNQVSLTCLVKGFYPSDIA | |
| | VEWESNGQPENNYKTTPPVLDSDGSFFLYSKLTVDKSRWQQGNVFSCSVMHE | |
| | ALHNHYTQKSLSLSPG | |
| IgG1-DEL | THTCPPCPAPELLGGPSVFLFPPKPKDTLMISRTPEVTCVVVDVSHEDPEVK | 253 |
| polymorph | FNWYVDGVEVHNAKTKPREEQYNSTYRVVSVLTVLHQDWLNGKEYKCKVSNK | |
| (“IgG1-DL”) | ALPAPIEKTISKAKGQPREPQVYTLPPSRDELTKNQVSLTCLVKGFYPSDIA | |
| | VEWESNGQPENNYKTTPPVLDSDGSFFLYSKLTVDKSRWQQGNVFSCSVMHE | |
| | ALHNHYTQKSLSLSPG | |
| IgG1-YTE- | THTCPPCPAPELLGGPSVFLFPPKPKDTLYITREPEVTCVVVDVSHEDPEVK | 254 |
| EM | FNWYVDGVEVHNAKTKPREEQYNSTYRVVSVLTVLHQDWLNGKEYKCKVSNK | |
| | ALPAPIEKTISKAKGQPREPQVYTLPPSREEMTKNQVSLTCLVKGFYPSDIA | |
| | VEWESNGQPENNYKTTPPVLDSDGSFFLYSKLTVDKSRWQQGNVFSCSVMHE | |
| | ALHNHYTQKSLSLSPG | |
| IgG1-YTE- | THTCPPCPAPELLGGPSVFLFPPKPKDTLYITREPEVTCVVVDVSHEDPEVK | 255 |
| DL | FNWYVDGVEVHNAKTKPREEQYNSTYRVVSVLTVLHQDWLNGKEYKCKVSNK | |
| | ALPAPIEKTISKAKGQPREPQVYTLPPSRDELTKNQVSLTCLVKGFYPSDIA | |
| | VEWESNGQPENNYKTTPPVLDSDGSFFLYSKLTVDKSRWQQGNVFSCSVMHE | |
| | ALHNHYTQKSLSLSPG | |
| IgG1-Y-DL | THTCPPCPAPELLGGPSVFLFPPKPKDTLYISRTPEVTCVVVDVSHEDPEVK | 256 |
| | FNWYVDGVEVHNAKTKPREEQYNSTYRVVSVLTVLHQDWLNGKEYKCKVSNK | |
| | ALPAPIEKTISKAKGQPREPQVYTLPPSRDELTKNQVSLTCLVKGFYPSDIA | |
| | VEWESNGQPENNYKTTPPVLDSDGSFFLYSKLTVDKSRWQQGNVFSCSVMHE | |
| | ALHNHYTQKSLSLSPG | |
| IgG1-Y-EM | THTCPPCPAPELLGGPSVFLFPPKPKDTLYISRTPEVTCVVVDVSHEDPEVK | 257 |
| | FNWYVDGVEVHNAKTKPREEQYNSTYRVVSVLTVLHQDWLNGKEYKCKVSNK | |
| | ALPAPIEKTISKAKGQPREPQVYTLPPSREEMTKNQVSLTCLVKGFYPSDIA | |
| | VEWESNGQPENNYKTTPPVLDSDGSFFLYSKLTVDKSRWQQGNVFSCSVMHE | |
| | ALHNHYTQKSLSLSPG | |
| IgG1-DL | APELLGGPSVFLFPPKPKDTLMISRTPEVTCVVVDVSHEDPEVKENWYVDGV | 258 |
| | EVHNAKTKPREEQYNSTYRVVSVLTVLHQDWLNGKEYKCKVSNKALPAPIEK | |
| | TISKAKGQPREPQVYTLPPSRDELTKNQVSLTCLVKGFYPSDIAVEWESNGQ | |
| | PENNYKTTPPVLDSDGSFFLYSKLTVDKSRWQQGNVFSCSVMHEALHNHYTQ | |
| | KSLSLSPGK | |
| IgG1-DL | APELLGGPSVFLFPPKPKDTLMISRTPEVTCVVVDVSHEDPEVKENWYVDGV | 259 |
| | EVHNAKTKPREEQYNSTYRVVSVLIVLHQDWLNGKEYKCKVSNKALPAPIEK | |
| | TISKAKGQPREPQVYTLPPSRDELTKNQVSLTCLVKGFYPSDIAVEWESNGQ | |
| | PENNYKTTPPVLDSDGSFFLYSKLTVDKSRWQQGNVFSCSVMHEALHNHYTQ | |
| | KSLSLSPG | |
| IgG1-DL | PPCPAPELLGGPSVFLFPPKPKDTLMISRTPEVTCVVVDVSHEDPEVKENWY | 260 |
| | VDGVEVHNAKTKPREEQYNSTYRVVSVLTVLHQDWLNGKEYKCKVSNKALPA | |
| | PIEKTISKAKGQPREPQVYTLPPSRDELTKNQVSLTCLVKGFYPSDIAVEWE | |
| | SNGQPENNYKTTPPVLDSDGSFFLYSKLTVDKSRWQQGNVFSCSVMHEALHN | |
| | HYTQKSLSLSPG | |
| IgG1-DL | DKTHTCPPCPAPELLGGPSVFLFPPKPKDTLMISRTPEVTCVVVDVSHEDPE | 261 |
| | VKFNWYVDGVEVHNAKTKPREEQYNSTYRVVSVLTVLHQDWLNGKEYKCKVS | |
| | NKALPAPIEKTISKAKGQPREPQVYTLPPSRDELTKNQVSLTCLVKGFYPSD | |
| | IAVEWESNGQPENNYKTTPPVLDSDGSFFLYSKLTVDKSRWQQGNVFSCSVM | |
| | HEALHNHYTQKSLSLSPG | |
| IgG1-DL | EPKSSDKTHTCPPCPAPELLGGPSVFLFPPKPKDTLMISRTPEVTCVVVDVS | 262 |
| | HEDPEVKFNWYVDGVEVHNAKTKPREEQYNSTYRVVSVLTVLHQDWLNGKEY | |
| | KCKVSNKALPAPIEKTISKAKGQPREPQVYTLPPSRDELTKNQVSLTCLVKG | |
| | FYPSDIAVEWESNGQPENNYKTTPPVLDSDGSFFLYSKLTVDKSRWQQGNVF | |
| | SCSVMHEALHNHYTQKSLSLSPG | |
| IgG1-DL | EPKSSDKTHTSPPSPAPELLGGPSVFLFPPKPKDTLMISRTPEVTCVVVDVS | 263 |
| | HEDPEVKFNWYVDGVEVHNAKTKPREEQYNSTYRVVSVLTVLHQDWLNGKEY | |
| | KCKVSNKALPAPIEKTISKAKGQPREPQVYTLPPSRDELTKNQVSLTCLVKG | |
| | FYPSDIAVEWESNGQPENNYKTTPPVLDSDGSFFLYSKLTVDKSRWQQGNVE | |
| | SCSVMHEALHNHYTQKSLSLSPG | |
| IgG1-EM | THTCPPCPAPELLGGPSVFLFPPKPKDTLYISRTPEVTCVVVDVSHEDPEVK | 264 |
| | FNWYVDGVEVHNAKTKPREEQYNSTYRVVSVLTVLHQDWLNGKEYKCKVSNK | |
| | ALPAPIEKTISKAKGQPREPQVYTLPPSREEMTKNQVSLTCLVKGFYPSDIA | |
| | VEWESNGQPENNYKTTPPVLDSDGSFFLYSKLTVDKSRWQQGNVFSCSVMHE | |
| | ALHNHYTQKSLSLSPG | |
| IgG1-DL | THTCPPCPAPELLGGPSVFLFPPKPKDTLMISRTPEVTCVVVDVSHEDPEVK | 265 |
| | FNWYVDGVEVHNAKTKPREEQYNSTYRVVSVLTVLHQDWLNGKEYKCKVSNK | |
| | ALPVPIEKTISKAKGQPREPQVYTLPPSRDELTKNQVSLTCLVKGFYPSDIA | |
| | VEWESNGQPENNYKTTPPVLDSDGSFFLYSKLTVDKSRWQQGNVFSCSVMHE | |
| | ALHNHYTQKSLSLSPG | |
| IgG1-EM | THTCPPCPAPELLGGPSVFLFPPKPKDTLMISRTPEVTCVVVDVSHEDPEVK | 266 |
| | FNWYVDGVEVHNAKTKPREEQYNSTYRVVSVLTVLHQDWLNGKEYKCKVSNK | |
| | ALPVPIEKTISKAKGQPREPQVYTLPPSREEMTKNQVSLTCLVKGFYPSDIA | |
| | VEWESNGQPENNYKTTPPVLDSDGSFFLYSKLTVDKSRWQQGNVFSCSVMHE | |
| | ALHNHYTQKSLSLSPG | |
| IgG2 | APPVAGPSVFLFPPKPKDTLMISRTPEVTCVVVDVSHEDPEVQFNWYVDGVE | 267 |
| | VHNAKTKPREEQFNSTFRVVSVLTVVHQDWLNGKEYKCKVSNKGLPAPIEKT | |
| | ISKTKGQPREPQVYTLPPSREEMTKNQVSLTCLVKGFYPSDISVEWESNGQP | |
| | ENNYKTTPPMLDSDGSFFLYSKLTVDKSRWQQGNVFSCSVMHEALHNHYTQK | |
| | SLSLSPGK | |
| IgG2 | VECPPCPAPPVAGPSVFLFPPKPKDTLMISRTPEVTCVVVDVSHEDPEVQEN | 268 |
| | WYVDGVEVHNAKTKPREEQFNSTFRVVSVLTVVHQDWLNGKEYKCKVSNKGL | |
| | PAPIEKTISKTKGQPREPQVYTLPPSREEMTKNQVSLTCLVKGFYPSDIAVE | |
| | WESNGQPENNYKTTPPMLDSDGSFFLYSKLTVDKSRWQQGNVFSCSVMHEAL | |
| | HNHYTQKSLSLSPGK | |
| IgG2 | ERKCCVECPPCPAPPVAGPSVFLFPPKPKDTLMISRTPEVTCVVVDVSHEDP | 269 |
| | EVQFNWYVDGVEVHNAKTKPREEQFNSTFRVVSVLTVVHQDWLNGKEYKCKV | |
| | SNKGLPAPIEKTISKTKGQPREPQVYTLPPSREEMTKNQVSLTCLVKGFYPS | |
| | DISVEWESNGQPENNYKTTPPMLDSDGSFFLYSKLTVDKSRWQQGNVFSCSV | |
| | MHEALHNHYTQKSLSLSPGK | |
| IgG2 | ERKSSVECPPCPAPPVAGPSVFLFPPKPKDTLMISRTPEVTCVVVDVSHEDP | 270 |
| | EVQFNWYVDGVEVHNAKTKPREEQFNSTFRVVSVLTVVHQDWLNGKEYKCKV | |
| | SNKGLPAPIEKTISKTKGQPREPQVYTLPPSREEMTKNQVSLTCLVKGFYPS | |
| | DISVEWESNGQPENNYKTTPPMLDSDGSFFLYSKLTVDKSRWQQGNVFSCSV | |
| | MHEALHNHYTQKSLSLSPG | |
| IgG2 | ERKSSVESPPCPAPPVAGPSVFLFPPKPKDTLMISRTPEVTCVVVDVSHEDP | 27 |
| | EVQFNWYVDGVEVHNAKTKPREEQFNSTFRVVSVLIVVHQDWLNGKEYKCKV | |
| | SNKGLPAPIEKTISKTKGQPREPQVYTLPPSREEMTKNQVSLTCLVKGFYPS | |
| | DISVEWESNGQPENNYKTTPPMLDSDGSFFLYSKLTVDKSRWQQGNVFSCSV | |
| | MHEALHNHYTQKSLSLSPG | |
| IgG2 | ERKSSVESPPSPAPPVAGPSVFLFPPKPKDTLMISRTPEVTCVVVDVSHEDP | 272 |
| | EVQFNWYVDGVEVHNAKTKPREEQFNSTFRVVSVLTVVHQDWLNGKEYKCKV | |
| | SNKGLPAPIEKTISKTKGQPREPQVYTLPPSREEMTKNQVSLTCLVKGFYPS | |
| | DISVEWESNGQPENNYKTTPPMLDSDGSFFLYSKLTVDKSRWQQGNVFSCSV | |
| | MHEALHNHYTQKSLSLSPG | |
| IgG2 | APPVAGPSVFLFPPKPKDTLMISRTPEVTCVVVDVSHEDPEVQFNWYVDGVE | 273 |
| | VHNAKTKPREEQFNSTFRVVSVLTVVHQDWLNGKEYKCKVSNKGLPAPIEKT | |
| | ISKTKGQPREPQVYTLPPSREEMTKNQVSLTCLVKGFYPSDISVEWESNGQP | |
| | ENNYKTTPPMLDSDGSFFLYSKLTVDKSRWQQGNVFSCSVMHEALHNHYTQK | |
| | SLSLSPG | |
| IgG2 | PPCPAPPVAGPSVFLFPPKPKDTLMISRTPEVTCVVVDVSHEDPEVQFNWYV | 274 |
| | DGVEVHNAKTKPREEQFNSTFRVVSVLTVVHQDWLNGKEYKCKVSNKGLPAP | |
| | IEKTISKTKGQPREPQVYTLPPSREEMTKNQVSLTCLVKGFYPSDISVEWES | |
| | NGQPENNYKTTPPMLDSDGSFFLYSKLTVDKSRWQQGNVFSCSVMHEALHNH | |
| | YTQKSLSLSPG | |
| IgG2 | VECPPCPAPPVAGPSVFLFPPKPKDTLMISRTPEVTCVVVDVSHEDPEVQEN | 275 |
| | WYVDGVEVHNAKTKPREEQFNSTFRVVSVLTVVHQDWLNGKEYKCKVSNKGL | |
| | PAPIEKTISKTKGQPREPQVYTLPPSREEMTKNQVSLTCLVKGFYPSDISVE | |
| | WESNGQPENNYKTTPPMLDSDGSFFLYSKLTVDKSRWQQGNVFSCSVMHEAL | |
| | HNHYTQKSLSLSPG | |
| IgG2 | PCPPCKCPAPNLLGGPSVFIFPPKIKDVLMISLSPIVTCVVVDVSEDDPDVQ | 276 |
| | ISWFVNNVEVHTAQTQTHREDYNSTLRVVSALPIQHQDWMSGKEFKCKVNNK | |
| | DLPAPIERTISKPKGSVRAPQVYVLPPPEEEMTKKQVTLTCMVTDFMPEDIY | |
| | VEWTNNGKTELNYKNTEPVLDSDGSYFMYSKLRVEKKNWVERNSYSCSVVHE | |
| | GLHNHHTTKSFSRTPG | |
| IgG3 | APELLGGPSVFLFPPKPKDTLMISRTPEVTCVVVDVSHEDPEVQFKWYVDGV | 277 |
| | EVHNAKTKPREEQYNSTFRVVSVLTVLHQDWLNGKEYKCKVSNKALPAPIEK | |
| | TISKTKGQPREPQVYTLPPSREEMTKNQVSLTCLVKGFYPSDIAVEWESSGQ | |
| | PENNYNTTPPMLDSDGSFFLYSKLTVDKSRWQQGNIFSCSVMHEALHNRFTQ | |
| | KSLSLSPGK | |
| IgG3 | APELLGGPSVFLFPPKPKDTLMISRTPEVTCVVVDVSHEDPEVQFKWYVDGV | 278 |
| | EVHNAKTKPREEQYNSTFRVVSVLTVLHQDWLNGKEYKCKVSNKALPAPIEK | |
| | TISKTKGQPREPQVYTLPPSREEMTKNQVSLTCLVKGFYPSDIAVEWESSGQ | |
| | PENNYNTTPPMLDSDGSFFLYSKLTVDKSRWQQGNIFSCSVMHEALHNRFTQ | |
| | KSLSLSPG | |
| IgG3 | PRCPAPELLGGPSVFLFPPKPKDTLMISRTPEVTCVVVDVSHEDPEVQFKWY | 279 |
| | VDGVEVHNAKTKPREEQYNSTFRVVSVLTVLHQDWLNGKEYKCKVSNKALPA | |
| | PIEKTISKTKGQPREPQVYTLPPSREEMTKNQVSLTCLVKGFYPSDIAVEWE | |
| | SSGQPENNYNTTPPMLDSDGSFFLYSKLTVDKSRWQQGNIFSCSVMHEALHN | |
| | RFTQKSLSLSPG | |
| IgG3 | DTPPPCPRCPAPELLGGPSVFLFPPKPKDTLMISRTPEVTCVVVDVSHEDPE | 280 |
| | VQFKWYVDGVEVHNAKTKPREEQYNSTFRVVSVLTVLHQDWLNGKEYKCKVS | |
| | NKALPAPIEKTISKTKGQPREPQVYTLPPSREEMTKNQVSLTCLVKGFYPSD | |
| | IAVEWESSGQPENNYNTTPPMLDSDGSFFLYSKLTVDKSRWQQGNIFSCSVM | |
| | HEALHNRFTQKSLSLSPG | |
| IgG3 | EPKSSDTPPPCPRCPAPELLGGPSVFLFPPKPKDTLMISRTPEVTCVVVDVS | 281 |
| | HEDPEVQFKWYVDGVEVHNAKTKPREEQYNSTFRVVSVLTVLHQDWLNGKEY | |
| | KCKVSNKALPAPIEKTISKTKGQPREPQVYTLPPSREEMTKNQVSLTCLVKG | |
| | FYPSDIAVEWESSGQPENNYNTTPPMLDSDGSFFLYSKLTVDKSRWQQGNIF | |
| | SCSVMHEALHNRFTQKSLSLSPG | |
| IgG3 | EPKSSDTPPPSPRCPAPELLGGPSVFLFPPKPKDTLMISRTPEVTCVVVDVS | 282 |
| | HEDPEVQFKWYVDGVEVHNAKTKPREEQYNSTFRVVSVLTVLHQDWLNGKEY | |
| | KCKVSNKALPAPIEKTISKTKGQPREPQVYTLPPSREEMTKNQVSLTCLVKG | |
| | FYPSDIAVEWESSGQPENNYNTTPPMLDSDGSFFLYSKLTVDKSRWQQGNIF | |
| | SCSVMHEALHNRFTQKSLSLSPG | |
| IgG3 | EPKSSDTPPPSPRSPAPELLGGPSVFLFPPKPKDTLMISRTPEVTCVVVDVS | 283 |
| | HEDPEVQFKWYVDGVEVHNAKTKPREEQYNSTFRVVSVLTVLHQDWLNGKEY | |
| | KCKVSNKALPAPIEKTISKTKGQPREPQVYTLPPSREEMTKNQVSLTCLVKG | |
| | FYPSDIAVEWESSGQPENNYNTTPPMLDSDGSFFLYSKLTVDKSRWQQGNIF | |
| | SCSVMHEALHNRFTQKSLSLSPG | |
| IgG4 | APEFLGGPSVFLFPPKPKDTLMISRTPEVTCVVVDVSQEDPEVQFNWYVDGV | 284 |
| | EVHNAKTKPREEQFNSTYRVVSVLTVLHQDWLNGKEYKCKVSNKGLPSSIEK | |
| | TISKAKGQPREPQVYTLPPSQEEMTKNQVSLTCLVKGFYPSDIAVEWESNGQ | |
| | PENNYKTTPPVLDSDGSFFLYSRLTVDKSRWQEGNVFSCSVMHEALHNHYTQ | |
| | KSLSLSLGK | |
| IgG4 | GPPCPPCPAPEFLGGPSVFLFPPKPKDTLMISRTPEVTCVVVDVSQEDPEVQ | 285 |
| | FNWYVDGVEVHNAKTKPREEQFNSTYRVVSVLTVLHQDWLNGKEYKCKVSNK | |
| | GLPSSIEKTISKAKGQPREPQVYTLPPSQEEMTKNQVSLTCLVKGFYPSDIA | |
| | VEWESNGQPENNYKTTPPVLDSDGSFFLYSRLTVDKSRWQEGNVFSCSVMHE | |
| | ALHNHYTQKSLSLSLG | |
| IgG4 | APEFLGGPSVFLFPPKPKDTLMISRTPEVTCVVVDVSQEDPEVQFNWYVDGV | 286 |
| | EVHNAKTKPREEQFNSTYRVVSVLTVLHQDWLNGKEYKCKVSNKGLPSSIEK | |
| | TISKAKGQPREPQVYTLPPSQEEMTKNQVSLTCLVKGFYPSDIAVEWESNGQ | |
| | PENNYKTTPPVLDSDGSFFLYSRLTVDKSRWQEGNVFSCSVMHEALHNHYTQ | |
| | KSLSLSLG | |
| IgG4 | PSCPAPEFLGGPSVFLFPPKPKDTLMISRTPEVTCVVVDVSQEDPEVQFNWY | 287 |
| | VDGVEVHNAKTKPREEQFNSTYRVVSVLTVLHQDWLNGKEYKCKVSNKGLPS | |
| | SIEKTISKAKGQPREPQVYTLPPSQEEMTKNQVSLTCLVKGFYPSDIAVEWE | |
| | SNGQPENNYKTTPPVLDSDGSFFLYSRLTVDKSRWQEGNVFSCSVMHEALHN | |
| | HYTQKSLSLSLG | |
| IgG4 | ESKYGPPCPSCPAPEFLGGPSVFLFPPKPKDTLMISRTPEVTCVVVDVSQED | 288 |
| | PEVQFNWYVDGVEVHNAKTKPREEQFNSTYRVVSVLTVLHQDWLNGKEYKCK | |
| | VSNKGLPSSIEKTISKAKGQPREPQVYTLPPSQEEMTKNQVSLTCLVKGFYP | |
| | SDIAVEWESNGQPENNYKTTPPVLDSDGSFFLYSRLTVDKSRWQEGNVFSCS | |
| | VMHEALHNHYTQKSLSLSLG | |
| IgG4 | ESKYGPPCPPCPAPEFLGGPSVFLFPPKPKDTLMISRTPEVTCVVVDVSQED | 289 |
| | PEVQFNWYVDGVEVHNAKTKPREEQFNSTYRVVSVLTVLHQDWLNGKEYKCK | |
| | VSNKGLPSSIEKTISKAKGQPREPQVYTLPPSQEEMTKNQVSLTCLVKGFYP | |
| | SDIAVEWESNGQPENNYKTTPPVLDSDGSFFLYSRLTVDKSRWQEGNVFSCS | |
| | VMHEALHNHYTQKSLSLSLG | |
| IgG4 | ESKYGPPCPPCPAPEFLGGPSVFLFPPKPKDTLMISRTPEVTCVVVDVSQED | 290 |
| | PEVQFNWYVDGVEVHNAKTKPREEQFNSTYRVVSVLTVLHQDWLNGKEYKCK | |
| | VSNKGLPSSIEKTISKAKGQPREPQVYTLPPSQEEMTKNQVSLTCLVKGFYP | |
| | SDIAVEWESNGQPENNYKTTPPVLDSDGSFFLYSKLTVDKSRWQEGNVFSCS | |
| | VMHEALHNHYTQKSLSLSLG | |
| IgG4 | ESKYGPPSPSCPAPEFLGGPSVFLFPPKPKDTLMISRTPEVTCVVVDVSQED | 291 |
| | PEVQFNWYVDGVEVHNAKTKPREEQFNSTYRVVSVLTVLHQDWLNGKEYKCK | |
| | VSNKGLPSSIEKTISKAKGQPREPQVYTLPPSQEEMTKNQVSLTCLVKGFYP | |
| | SDIAVEWESNGQPENNYKTTPPVLDSDGSFFLYSRLTVDKSRWQEGNVESCS | |
| | VMHEALHNHYTQKSLSLSLG | |
| IgG4 | ESKYGPPSPSSPAPEFLGGPSVFLFPPKPKDTLMISRTPEVTCVVVDVSQED | 292 |
| | PEVQFNWYVDGVEVHNAKTKPREEQFNSTYRVVSVLTVLHQDWLNGKEYKCK | |
| | VSNKGLPSSIEKTISKAKGQPREPQVYTLPPSQEEMTKNQVSLTCLVKGFYP | |
| | SDIAVEWESNGQPENNYKTTPPVLDSDGSFFLYSRLTVDKSRWQEGNVFSCS | |
| | VMHEALHNHYTQKSLSLSLG | |

In general, ActRIIB-ECD polypeptides are organized such that the Fc domain monomer is linked at its N-terminus to the C-terminus of the ActRIIB-ECD variant, so that for each ActRIIB-ECD polypeptide, the orientation of the construct is, from N-terminus to C-terminus, a single chain of (ActRIIB-ECD variant)-(linker)-(Fc domain monomer). However, the orientation of constructs is not particularly limited, and other orientations are contemplated. For example, in some embodiments the Fc domain monomer may be linked at its C-terminus to the N-terminus of the ActRIIB-ECD variant.

In an exemplary embodiment, the Fc domain monomer allows assembly of two or more polypeptide chains in a covalent manner, for example by disulfide linking between cysteine residues. In this way the Fc domain monomer acts as a dimerization domain, allowing assembly of two ActRIIB-ECD polypeptide chains to form a dimer. In accordance with the present disclosure, such dimers generally comprise two polypeptides, each polypeptide including an ActIIRB-ECD variant linked to the Fc domain monomer as described herein, thereby forming a divalent TGFβ superfamily ligand binding agent. The binding agents described herein therefore comprise two ActIIRB-ECD variants, a linker domain, and an Fc domain.

The Fc domain monomer generally comprises one or more cysteine residue for crosslinking of a first polypeptide with a second polypeptide in a homodimeric construct. For example, the Fc domain monomer may include at least two cysteine residues for forming a disulfide bridge between two polypeptides, thereby forming a dimer. In some embodiments of the present technology, the Fc domain monomer comprises or consists of the sequence set forth in any one of SEQ ID NOs: 252-292, or a sequence at least 90%, at least 95%, at least 96%, at least 97%, at least 98%, or at least 99% identical thereto. In a particular embodiment, the Fc domain monomer comprises or consists of the amino acid sequence set forth in SEQ ID NO: 253. In a particular embodiment, the Fc domain monomer comprises or consists of the amino acid sequence set forth in SEQ ID NO: 266. In a particular embodiment, the Fc domain monomer comprises or consists of the amino acid sequence set forth in SEQ ID NO: 256. In a particular embodiment, the Fc domain monomer comprises or consists of the amino acid sequence set forth in SEQ ID NO: 255.

In some embodiments, the present disclosure provides binding agents comprising a variant Fc domain, i.e., a non-naturally occurring Fc domain, for example an Fc domain comprising one or more non-naturally occurring amino acid residue, substitution, addition, deletion, etc.

In some embodiments of the technology, the Fc domain is a variant Fc domain that forms a variant Fc domain with a desirable property, such as increased half-life, on the polypeptide or binding agent compared to naturally occurring (wild-type) Fc sequences. As used herein, a “variant Fc domain” refers to a non-naturally occurring Fc domain, for example an Fc domain comprising one or more non-naturally occurring amino acid residues, one or more amino acid substitutions relative to a wild-type human constant domain, or one or more amino acid deletion, addition and/or modification.

There are many known polymorphs for the IgG1 Fc domain, including the “DEL” polymorph and the “EEM” polymorph. The DEL polymorph comprises the amino acids D-E-L at positions 356, 357, and 358, respectively (also referred to herein as “Fc-DL”, e.g., SEQ ID NO: 253). The EEM polymorph comprises the amino acids E-E-M at positions 356, 357, and 358, respectively (also referred to herein as “Fc-EM”, e.g., SEQ ID NO: 252). Two binding agents that are otherwise identical except for the presence of a DEL Fc domain or an EEM Fc domain are expected to demonstrate similar properties in terms of ligand binding and therapeutic efficacy. In some embodiments of the technology, the Fc domain is a DEL Fc domain (“DL”). In some embodiments of the technology, the Fc domain is an EEM Fc domain (“EM”). Other polymorphs may also be used, e.g., IgG1 polymorphs of SEQ ID NOs: 252-253 and 258-266, IgG2 polymorphs of SEQ ID NOs: 267-276, IgG3 polymorphs of SEQ ID NOs: 277-283, and IgG4 polymorphs of SEQ ID NOs: 284-292.

In some embodiments, a variant Fc domain formed by two variant Fc domain monomers has altered binding properties for an Fc receptor such as FcRn, relative to a comparable molecule (e.g., a protein having the same amino acid sequence except having a wild type Fc domain monomer). The serum half-life of proteins comprising Fc domains may be increased by increasing the binding affinity of the Fc domain for FcRn. In one embodiment, the Fc domain variant has enhanced serum half-life relative to a comparable molecule. In a particular embodiment, the Fc domain variant comprises at least one amino acid substitution at one or more positions selected from the group consisting of M252Y, S254T and T256 (referred to herein as “YTE”; e.g., SEQ ID NO: 254 and 255). In another embodiment, the Fc domain variant comprises a Y at position 252 (e.g., SEQ ID NO: 256 and 257, referred to herein as “Fc-Y”). In another embodiment, the Fc domain variant comprises a T at position 254. In another embodiment, the Fc domain variant comprises an E at position 256.

Consequently, in some embodiments of the present technology, the ActRIIB-ECD polypeptide comprises a variant Fc domain monomer that forms an Fc domain with increased in vivo half-life relative to a comparable molecule. In some such embodiments, the Fc domain monomer of the ActRIIB-ECD polypeptide comprises at least one substitution of an amino acid residue selected from the group consisting of: residue 252, 254, and 256.

In some embodiments, an ActRIIB-ECD polypeptide comprises a variant Fc domain monomer comprising at least one amino acid substitution selected from the group consisting of M252Y, S254T, and T256E. In such embodiments, the variant Fc domain monomer may further comprise one or more additional amino acid substitution(s) such as, without limitation, E356D and M358L.

In some embodiments, an ActRIIB-ECD polypeptide comprises a variant Fc domain monomer comprising the following amino acid substitutions: M252Y, S254T, and T256E, referred to herein as “FcYTE” or “YTE”. In some embodiments, the FcYTE domain monomer is a DEL polymorph (referred to herein as YTE-DL, e.g., SEQ ID NO: 255). In some embodiments, the FcYTE domain monomer is an EEM polymorph (referred to herein as YTE-EM, e.g., SEQ ID NO: 254).

In some embodiments, an ActRIIB-ECD polypeptide comprises a variant Fc domain monomer comprising the following amino acid substitutions: M252Y, referred to herein as “FcY”. In some embodiments, the FcY domain monomer is a DEL polymorph (referred to herein as Y-DL, e.g., SEQ ID NO: 256). In some embodiments, the FcY domain monomer is an EEM polymorph (referred to herein as Y-EM, e.g., SEQ ID NO: 257).

In some embodiments, an ActRIIB-ECD polypeptide comprises an Fc domain monomer comprising a Lysine residue (K) at the C-terminus.

In some embodiments, a variant Fc domain (e.g., an Fc domain formed by two variant Fc domain monomers) for use in the ActRIIB-ECD polypeptides of the disclosure comprises one or more amino acid substitution that reduces aggregation and/or increases stability and/or increases half-life of the ActRIIB-ECD polypeptide compared to naturally occurring Fc sequences. In some embodiments, the Fc domain is selected to provide one or more effector function such as antibody dependent cellular cytotoxicity (ADCC), complement activation (complement dependent cytotoxicity or CDC), opsonization, and the like. In an embodiment, a variant Fc domain has enhanced binding to an Fc receptor relative to a comparable molecule. In a specific embodiment, a variant Fc domain has enhanced binding to the neonatal Fc receptor FcRn. In another embodiment, the variant Fc domain and/or the polypeptide or binding agent containing the variant Fc domain has a binding affinity for FcRn that is at least 2 fold, or at least 3 fold, or at least 5 fold, or at least 7 fold, or at least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold greater than that of a comparable molecule. The serum half-life of proteins comprising Fc domain may be increased by increasing the binding affinity of the Fc domain monomer for FcRn. Consequently, in one embodiment the polypeptide or binding agent comprising the variant Fc domain has an enhanced serum half-life relative to a comparable molecule.

Examples for means to extend serum half-life of the binding agents of the disclosure include peptides, proteins or domains of proteins, which are fused or otherwise attached to the binding agents. The group of peptides, proteins or protein domains includes peptides binding to other proteins with preferred pharmacokinetic profile in the human body such as serum albumin (see WO 2009/127691). An alternative concept of such half-life extending peptides includes peptides binding to the neonatal Fc receptor (FcRn, see WO 2007/098420), which can also be used in the binding agents of the present disclosure. The concept of attaching larger domains of proteins or complete proteins includes e.g. the fusion of human serum albumin, variants or mutants of human serum albumin (see WO 2011/051489, WO 2012/059486, WO 2012/150319, WO 2013/135896, WO 2014/072481, WO 2013/075066) or domains thereof as well as the fusion of constant region of immunoglobulins (Fc domains) and variants thereof, as described herein. Such variants of Fc domains may be optimized/modified in order to allow the desired pairing of dimers or multimers, to abolish Fc receptor binding (e.g., the Fcg receptor), to enhance binding to FcRn, or for other reasons. A further concept known in the art to extend the half-life of small protein compounds in the human body is the pegylation of those compounds such as the polypeptide or binding agent of the present disclosure.

In one embodiment, the present disclosure provides binding agents, wherein the Fc domain comprises a non-naturally occurring amino acid residue at one or more positions selected from the group consisting of 234, 235, 236, 237, 238, 239, 240, 241, 243, 244, 245, 247, 251, 252, 254, 255, 256, 262, 263, 264, 265, 266, 267, 268, 269, 279, 280, 284, 292, 296, 297, 298, 299, 305, 313, 316, 325, 326, 327, 328, 329, 330, 332, 333, 334, 339, 341, 343, 370, 373, 378, 392, 416, 419, 421, 440 and 443 as numbered by the EU index as set forth in Kabat. Optionally, the Fc domain may comprise a non-naturally occurring amino acid residue at additional and/or alternative positions known to one skilled in the art (see, e.g., U.S. Pat. Nos. 5,624,821; 6,277,375; 6,737,056; PCT Patent Publications WO 01/58957; WO 02/06919; WO 04/016750; WO 04/029207; WO 04/035752; WO 04/074455; WO 04/099249; WO 04/063351; WO 05/070963; WO 05/040217, WO 05/092925 and WO 06/020114). In a specific embodiment, the present disclosure provides an Fc variant protein composition, wherein the Fc domain comprises at least one amino acid substitution selected from the group consisting of 234D, 234E, 234N, 234Q, 234T, 234H, 234Y, 2341, 234V, 234F, 235A, 235D, 235R. 235W, 235P, 235S, 235N, 235Q, 235T, 235H, 235Y, 2351, 235V, 235F, 236E, 239D, 239E, 239N, 239Q, 239F, 239T, 239H, 239Y, 2401, 240A, 240T, 240M, 241W, 241 L, 241Y, 241E, 241R. 243W, 243L 243Y, 243R, 243Q, 244H, 245A, 247L, 247V, 247G, 251F, 252Y, 254T, 255L, 256E, 256M, 2621, 262A, 262T, 262E, 2631, 263A, 263T, 263M, 264L, 2641, 264W, 264T, 264R, 264F, 264M, 264Y, 264E, 265G, 265N, 265Q, 265Y, 265F, 265V, 2651, 265L, 265H, 265T, 2661, 266A, 266T, 266M, 267Q, 267L, 268E, 269H, 269Y, 269F, 269R, 270E, 280A, 284M, 292P, 292L, 296E, 296Q, 296D, 296N, 296S, 296T, 296L, 2961, 296H, 269G, 297S, 297D, 297E, 298H, 298I, 298T, 298F, 2991, 299L, 299A, 299S, 299V, 299H, 299F, 299E, 3051, 313F, 316D, 325Q, 325L, 3251, 325D, 325E, 325A, 325T, 325V, 325H, 327G, 327W, 327N, 327L, 328S, 328M, 328D, 328E, 328N, 328Q, 328F, 328I, 328V, 328T, 328H, 328A, 329F, 329H, 329Q, 330K, 330G, 330T, 330C, 330L, 330Y, 330V, 3301, 330F, 330R, 330H, 332D, 332S, 332W, 332F, 332E, 332N, 332Q, 332T, 332H, 332Y, 332A, 339T, 370E, 370N, 378D, 392T, 396L, 416G, 419H, 421K, 440Y and 434W as numbered by the EU index as set forth in Kabat. Optionally, the Fc domain may comprise additional and/or alternative amino acid substitutions known to one skilled in the art (see, e.g., U.S. Pat. Nos. 5,624,821; 6,277,375; 6,737,056; PCT Patent Publications WO 01/58957; WO 02/06919; WO 04/016750; WO 04/029207; WO 04/035752 and WO 05/040217).

### Additional Domains

It is envisaged that the ActRIIB-ECD polypeptide and/or binding agent of the disclosure may have, in addition to its function to bind to the target TGFβ superfamily ligand(s) as specified, a further binding specificity or a further function. In some embodiments of the present technology, a ActRIIB-ECD polypeptide or binding agent may be conjugated with a targeting agent, a therapeutic moiety, a detectable moiety and/or a diagnostic moiety. In some embodiments, a polypeptide may possess a further function such as a fully functional Fc constant domain mediating antibody-dependent cellular cytotoxicity through recruitment of effector cells like NK cells, by providing a label (fluorescent etc.), by providing a therapeutic agent such as a toxin or radionuclide, and/or by providing means to enhance serum half-life, etc.

In some embodiments, the ActRIIB-ECD polypeptides described herein comprise an ActRIIB-ECD, a linker, an Fc domain monomer, and one or more additional domains. In some embodiments, the one or more additional domains is selected from a fibronectin domain, a human serum albumin domain, As used herein, the term “fibronectin domain” refers to a high molecular weight glycoprotein of the extracellular matrix, or a fragment thereof, that binds to, e.g., membrane-spanning receptor proteins such as integrins and extracellular matrix components such as collagens and fibrins. In some embodiments, a fibronectin domain is a fibronectin type III domain having amino acids 610-702 of the sequence of UniProt ID NO: P02751. In other embodiments, a fibronectin domain is an adnectin protein.

In some embodiments, a polypeptide or binding agent of the disclosure includes an ActRIIB-ECD variant fused to one or more fibronectin domain. Binding to fibronectin domains can improve the pharmacokinetics of protein pharmaceuticals. A fibronectin domain is a high molecular weight glycoprotein of the extracellular matrix, or a fragment thereof, that binds to, e.g., membrane-spanning receptor proteins such as integrins and extracellular matrix components such as collagens and fibrins. In some embodiments of the present invention, a fibronectin domain is joined to the N- or C-terminus (e.g., C-terminus) of an ActRIIB-ECD variant described herein (e.g., an ActRIIB-ECD variant having the amino acid sequence set forth in any one of SEQ ID NOs: 4-62) to increase the serum half-life of the ActRIIB-ECD variant. A fibronectin domain can be joined, either directly or through a linker, to the N- or C-terminus of an ActRIIB-ECD variant, or a polypeptide thereof, or a binding agent thereof. In some embodiments, a polypeptide or binding agent of the disclosure may be fused to the N- or C-terminus of a fibronectin domain, e.g., through conventional genetic or chemical means, e.g., chemical conjugation. If desired, a linker (e.g., a spacer) can be inserted between the ActRIIB-ECD variant and the fibronectin domain. Without being bound by theory, it is expected that in some embodiments inclusion of a fibronectin domain in an ActRIIB-ECD variant described herein may lead to prolonged retention of the therapeutic protein through its binding to integrins and extracellular matrix components such as collagens and fibrins.

As one example, fibronectin domains that can be used in the methods and compositions and polypeptides of the disclosure are generally known in the art. In one embodiment, the fibronectin domain is a fibronectin type III domain having amino acids 610-702 of the sequence of UniProt ID NO: P02751. In another embodiment, the fibronectin domain is an adnectin protein.

As used herein, the term “human serum albumin” refers to the albumin protein present in human blood plasma. Human serum albumin is the most abundant protein in the blood. It constitutes about half of the blood serum protein. In some embodiments, a human serum albumin has the sequence of UniProt ID NO: P02768.

In some embodiments, an ActRIIB variant or a polypeptide or a binding agent described herein may be fused to serum albumin. Binding to serum albumins can improve the pharmacokinetics of protein pharmaceuticals. Serum albumin is a globular protein that is the most abundant blood protein in mammals. Serum albumin is produced in the liver and constitutes about half of the blood serum proteins. It is monomeric and soluble in the blood. Some of the most crucial functions of serum albumin include transporting hormones, fatty acids, and other proteins in the body, buffering pH, and maintaining osmotic pressure needed for proper distribution of bodily fluids between blood vessels and body tissues. In some embodiments, serum albumin is human serum albumin. In some embodiments, a human serum albumin is joined to the N- or C-terminus (e.g., C-terminus) of an ActRIIB-ECD variant described herein (e.g., an ActRIIB-ECD variant having the amino acid sequence set forth in any one of SEQ ID NOs: 4-62) to increase the serum half-life of the ActRIIB-ECD variant. A human serum albumin can be joined, either directly or through a linker, to the N- or C-terminus of an ActRIIB-ECD variant.

As one example, serum albumins that can be used in the polypeptides and methods and compositions described herein are generally known in the art. In one embodiment, the serum albumin includes the sequence of UniProt ID NO: P02768. In some embodiments, a polypeptide or binding agent of the disclosure may be fused to the N- or C-terminus of a human serum albumin, e.g., through conventional genetic or chemical means, e.g., chemical conjugation. If desired, a linker (e.g., a spacer) can be inserted between the ActRIIB-ECD variant and the human serum albumin. Without being bound by theory, it is expected that in some embodiments inclusion of a human serum albumin in an ActRIIB-ECD variant described herein may lead to prolonged retention of the therapeutic protein.

In some embodiments, a polypeptide or binding agent of the disclosure further includes a moiety (e.g., Fc domain monomer, a wild-type Fc domain, an Fc domain with amino acid substitutions (e.g., one or more substitutions that reduce dimerization, an albumin-binding peptide, a fibronectin domain, or a human serum albumin), which may be fused to the N- or C-terminus (e.g., C-terminus) of the ActRIIB-ECD variant, the polypeptide, or the binding agent by way of a linker or other covalent bonds. A polypeptide including an ActRIIB-ECD variant fused to an Fc domain monomer may form a dimer (e.g., homodimer or heterodimer) through the interaction between two Fc domain monomers, which combine to form an Fc domain in the dimer. Furthermore, in some embodiments, a polypeptide or binding agent described herein has a serum half-life of at least 7 days in humans.

### Exemplary TGFβ Superfamily Binding Agents

The overall structures of exemplary binding agents described herein are provided in Table 5. The amino acid sequences of each binding agent are provided in Table 6.

TABLE 5
Structure of exemplary binding agents

| Binding | | | | |
|---|---|---|---|---|
| agent | ActRII ECD | Linker | Fc domain | SEQ ID |
| P75 | WT hActRIIB | 3 aa | IgG1-DL | 171 |
| | (SEQ ID: 2) | (GGG) | (SEQ ID: 253) | |
| P757 | WT hActRIIB | 14 aa | IgG1-DL | 172 |
| | (SEQ ID: 2) | (SEQ ID: 94) | (SEQ ID: 253) | |
| P444 | WT hActRIIA | 3 aa | IgG1 | 173 |
| | (SEQ ID:3) | (GGG) | (SEQ ID: 266) | |
| P739 | hActRIIB G27E | 14 aa | IgG1-DL | 174 |
| | (SEQ ID: 4) | (SEQ ID: 94) | (SEQ ID: 253) | |
| P750 | hActRIIB L33R | 14 aa | IgG1-DL | 175 |
| | (SEQ ID: 15) | (SEQ ID: 94) | (SEQ ID: 253) | |
| P751 | hActRIIB L33Y | 14 aa | IgG1-DL | 176 |
| | (SEQ ID: 12) | (SEQ ID: 94) | (SEQ ID: 253) | |
| P753 | hActRIIB T69H | 14 aa | IgG1-DL | 177 |
| | (SEQ ID: 19) | (SEQ ID: 94) | (SEQ ID: 253) | |
| P754 | hActRIIB T69Q | 14 aa | IgG1-DL | 178 |
| | (SEQ ID: 20) | (SEQ ID: 94) | (SEQ ID: 253) | |
| P765 | hActRIIB G27E | 3 aa | IgG1-DL | 179 |
| | (SEQ ID: 4) | (GGG) | (SEQ ID: 253) | |
| P777 | hActRIIB L33Y | 3 aa | IgG1-DL | 180 |
| | (SEQ ID: 12) | (GGG) | (SEQ ID: 253) | |
| P779 | hActRIIB T69H | 3 aa | IgG1-DL | 181 |
| | (SEQ ID: 19) | (GGG) | (SEQ ID: 253) | |
| P780 | hActRIIB T69Q | 3 aa | IgG1-DL | 182 |
| | (SEQ ID: 20) | (GGG) | (SEQ ID: 253) | |
| P1171 | hActRIIB L33Y T69H | 3 aa | IgG1-DL | 183 |
| | (SEQ ID: 28) | (GGG) | (SEQ ID: 253) | |
| P1172 | hActRIIB L33Y T69H | 10 aa | IgG1-DL | 184 |
| | (SEQ ID: 28) | (SEQ ID: 98) | (SEQ ID: 253) | |
| P1173 | hActRIIB L33Y T69H | 14 aa | IgG1-DL | 185 |
| | (SEQ ID: 28) | (SEQ ID: 94) | (SEQ ID: 253) | |
| P1174 | hActRIIB L33Y T69Q | 3 aa | IgG1-DL | 186 |
| | (SEQ ID: 29) | (GGG) | (SEQ ID: 253) | |
| P1175 | hActRIIB L33Y T69Q | 10 aa | IgG1-DL | 187 |
| | (SEQ ID: 29) | (SEQ ID: 98) | (SEQ ID: 253) | |
| P1176 | hActRIIB L33Y T69Q | 14 aa | IgG1-DL | 188 |
| | (SEQ ID: 29) | (SEQ ID: 94) | (SEQ ID: 253) | |
| P1177 | hActRIIB L33Y T69E | 3 aa | IgG1-DL | 189 |
| | (SEQ ID: 30) | (GGG) | (SEQ ID: 253) | |
| P1178 | hActRIIB L33Y T69E | 10 aa | IgG1-DL | 190 |
| | (SEQ ID: 30) | (SEQ ID: 98) | (SEQ ID: 253) | |
| P1179 | hActRIIB L33Y T69E | 14 aa | IgG1-DL | 191 |
| | (SEQ ID: 30) | (SEQ ID: 94) | (SEQ ID: 253) | |
| P1180 | hActRIIB L33Y G27D | 3 aa | IgG1-DL | 192 |
| | (SEQ ID: 33) | (GGG) | (SEQ ID: 253) | |
| P1181 | hActRIIB L33Y G27D | 10 aa | IgG1-DL | 193 |
| | (SEQ ID: 33) | (SEQ ID: 98) | (SEQ ID: 253) | |
| P1182 | hActRIIB L33Y G27D | 14 aa | IgG1-DL | 194 |
| | (SEQ ID: 33) | (SEQ ID: 94) | (SEQ ID: 253) | |
| P1183 | hActRIIB L33Y G27E | 3 aa | IgG1-DL | 195 |
| | (SEQ ID: 32) | (GGG) | (SEQ ID: 253) | |
| P1184 | hActRIIB L33Y G27E | 10 aa | IgG1-DL | 196 |
| | (SEQ ID: 32) | (SEQ ID: 98) | (SEQ ID: 253) | |
| P1185 | hActRIIB L33Y G27E | 14 aa | IgG1-DL | 197 |
| | (SEQ ID: 32) | (SEQ ID: 94) | (SEQ ID: 253) | |
| P1186 | hActRIIB L33F T69Q | 3 aa | IgG1-DL | 198 |
| | (SEQ ID: 31) | (GGG) | (SEQ ID: 253) | |
| P1187 | hActRIIB L33F T69Q | 10 aa | IgG1-DL | 199 |
| | (SEQ ID: 31) | (SEQ ID: 98) | (SEQ ID: 253) | |
| P1188 | hActRIIB L33F T69Q | 14 aa | IgG1-DL | 200 |
| | (SEQ ID: 31) | (SEQ ID: 94) | (SEQ ID: 253) | |
| P1201 | hActRIIB T69E | 3 aa | IgG1-DL | 20 |
| | (SEQ ID: 21) | (GGG) | (SEQ ID: 253) | |
| P1202 | hActRIIB T69E | 10 aa | IgG1-DL | 202 |
| | (SEQ ID: 21) | (SEQ ID: 98) | (SEQ ID: 253) | |
| P1203 | hActRIIB T69E | 14 aa | IgG1-DL | 203 |
| | (SEQ ID: 21) | (SEQ ID: 94) | (SEQ ID: 253) | |
| P1204 | hActRIIB L33F | 3 aa | IgG1-DL | 204 |
| | (SEQ ID: 10) | (GGG) | (SEQ ID: 253) | |
| P1205 | hActRIIB L33F | 10 aa | IgG1-DL | 205 |
| | (SEQ ID: 10) | (SEQ ID: 98) | (SEQ ID: 253) | |
| P1206 | hActRIIB L33F | 14 aa | IgG1-DL | 206 |
| | (SEQ ID: 10) | (SEQ ID: 94) | (SEQ ID: 253) | |
| P1207 | hActRIIB L33Q | 3 aa | IgG1-DL | 207 |
| | (SEQ ID: 11) | (GGG) | (SEQ ID: 253) | |
| P1208 | hActRIIB L33Q | 10 aa | IgG1-DL | 208 |
| | (SEQ ID: 11) | (SEQ ID: 98) | (SEQ ID: 253) | |
| P1209 | hActRIIB L33Q | 14 aa | IgG1-DL | 209 |
| | (SEQ ID: 11) | (SEQ ID: 94) | (SEQ ID: 253) | |
| P1210 | hActRIIB L33Y | 10 aa | IgG1-DL | 210 |
| | (SEQ ID: 12) | (SEQ ID: 98) | (SEQ ID: 253) | |
| P1229 | hActRIIB L33W | 14 aa | IgG1-DL | 21 |
| | (SEQ ID: 13) | (SEQ ID: 94) | (SEQ ID: 253) | |
| P1230 | hActRIIB L33E | 14 aa | IgG1-DL | 212 |
| | (SEQ ID: 16) | (SEQ ID:94) | (SEQ ID: 253) | |
| P1231 | hActRIIB L33K | 14 aa | IgG1-DL | 213 |
| | (SEQ ID: 17) | (SEQ ID: 94) | (SEQ ID: 253) | |
| P1232 | hActRIIB L33M | 14 aa | IgG1-DL | 214 |
| | (SEQ ID: 18) | (SEQ ID: 94) | (SEQ ID: 253) | |
| P1235 | hActRIIB L33Y G27N | 14 aa | IgG1-DL | 215 |
| | (SEQ ID: 34) | (SEQ ID: 94) | (SEQ ID: 253) | |
| P1236 | hActRIIB L33Y G27Q | 14 aa | IgG1-DL | 216 |
| | (SEQ ID: 35) | (SEQ ID: 94) | (SEQ ID: 253) | |
| P1237 | hActRIIB L33Y G27K | 14 aa | IgG1-DL | 217 |
| | (SEQ ID: 36) | (SEQ ID: 94) | (SEQ ID: 253) | |
| P1238 | hActRIIB L33Y G27T | 14 aa | IgG1-DL | 218 |
| | (SEQ ID: 37) | (SEQ ID: 94) | (SEQ ID: 253) | |
| P1239 | hActRIIB L33Y G27M | 14 aa | IgG1-DL | 219 |
| | (SEQ ID: 38) | (SEQ ID: 94) | (SEQ ID: 253) | |
| P1240 | hActRIIB L33Y T69R | 14 aa | IgG1-DL | 220 |
| | (SEQ ID: 25) | (SEQ ID: 94) | (SEQ ID: 253) | |
| P1241 | hActRIIB L33Y T69Y | 14 aa | IgG1-DL | 221 |
| | (SEQ ID: 26) | (SEQ ID: 94) | (SEQ ID: 253) | |
| P1242 | hActRIIB L33Y T69W | 14 aa | IgG1-DL | 222 |
| | (SEQ ID: 27) | (SEQ ID: 94) | (SEQ ID: 253) | |
| P1269 | hActRIIB L33Y, L14E | 14 aa | IgG1-DL | 223 |
| | (SEQ ID: 52) | (SEQ ID: 94) | (SEQ ID: 253) | |
| P1270 | hActRIIB L33Y, L14H | 14 aa | IgG1-DL | 224 |
| | (SEQ ID: 53) | (SEQ ID: 94) | (SEQ ID: 253) | |
| P1271 | hActRIIB L33Y, L14S | 14 aa | IgG1-DL | 225 |
| | (SEQ ID: 54) | (SEQ ID: 94) | (SEQ ID: 253) | |
| P1272 | hActRIIB L33Y, L55Y | 14 aa | IgG1-DL | 226 |
| | (SEQ ID: 59) | (SEQ ID: 94) | (SEQ ID: 253) | |
| P1273 | hActRIIB L33Y, L55Q | 14 aa | IgG1-DL | 227 |
| | (SEQ ID: 60) | (SEQ ID: 94) | (SEQ ID: 253) | |
| P1274 | hActRIIB L33Y, L55M | 14 aa | IgG1-DL | 228 |
| | (SEQ ID: 61) | (SEQ ID: 94) | (SEQ ID: 253) | |
| P1275 | hActRIIB L33Y, L55I | 14 aa | IgG1-DL | 229 |
| | (SEQ ID: 62) | (SEQ ID: 94) | (SEQ ID: 253) | |
| P1371 | hActRIIB L33W | 14 aa | IgG1 Y-DL | 230 |
| | (SEQ ID: 13) | (SEQ ID: 94) | (SEQ ID: 256) | |
| P1372 | hActRIIB L33W | 14 aa | IgG1 YTE-DL | 231 |
| | (SEQ ID: 13) | (SEQ ID: 94) | (SEQ ID: 255) | |
| P1373 | hActRIIB L33W | 19 aa | IgG1-DL | 232 |
| | (SEQ ID: 13) | (SEQ ID: 89) | (SEQ ID: 253) | |
| P1374 | hActRIIB L33W | 19 aa | IgG1 Y-DL | 233 |
| | (SEQ ID: 13) | (SEQ ID: 89) | (SEQ ID: 256) | |
| P1375 | hActRIIB L33W | 19 aa | IgG1 YTE-DL | 234 |
| | (SEQ ID: 13) | (SEQ ID: 89) | (SEQ ID: 255) | |
| P1385 | hActRIIB G27D, L33W | 19 aa | IgG1-DL | 235 |
| | (SEQ ID: 43) | (SEQ ID: 89) | (SEQ ID: 253) | |
| P1386 | hActRIIB G27E, L33W | 19 aa | IgG1-DL | 236 |
| | (SEQ ID: 44) | (SEQ ID: 89) | (SEQ ID: 253) | |
| P1387 | hActRIIB G27N, L33W | 19 aa | IgG1-DL | 237 |
| | (SEQ ID: 45) | (SEQ ID: 89) | (SEQ ID: 253) | |
| P1388 | hActRIIB G27Q, L33W | 19 aa | IgG1-DL | 238 |
| | (SEQ ID: 46) | (SEQ ID: 89) | (SEQ ID: 253) | |
| P1389 | hActRIIB G27T, L33W | 19 aa | IgG1-DL | 239 |
| | (SEQ ID: 47) | (SEQ ID: 89) | (SEQ ID: 253) | |
| P1390 | hActRIIB G27M, L33W | 19 aa | IgG1-DL | 240 |
| | (SEQ ID: 48) | (SEQ ID: 89) | (SEQ ID: 253) | |
| P1391 | hActRIIB G27D, L33Y | 19 aa | IgG1-DL | 241 |
| | (SEQ ID: 39) | (SEQ ID: 89) | (SEQ ID: 253) | |
| P1392 | hActRIIB G27E, L33Y | 19 aa | IgG1-DL | 242 |
| | (SEQ ID: 40) | (SEQ ID: 89) | (SEQ ID: 253) | |
| P1395 | hActRIIB G27N, L33Y | 19 aa | IgG1-DL | 243 |
| | (SEQ ID: 41) | (SEQ ID: 89) | (SEQ ID: 253) | |
| P1396 | hActRIIB G27Q, L33Y | 19 aa | IgG1-DL | 244 |
| | (SEQ ID: 42) | (SEQ ID: 89) | (SEQ ID: 253) | |
| P1406 | hActRIIB L14E, L33W | 19 aa | IgG1-DL | 245 |
| | (SEQ ID: 55) | (SEQ ID: 89) | (SEQ ID: 253) | |
| P1407 | hActRIIB L14D, L33W | 19 aa | IgG1-DL | 246 |
| | (SEQ ID: 56) | (SEQ ID: 89) | (SEQ ID: 253) | |
| P1408 | hActRIIB L14N, L33W | 19 aa | IgG1-DL | 247 |
| | (SEQ ID: 57) | (SEQ ID: 89) | (SEQ ID: 253) | |
| P1409 | hActRIIB L14Q, L33W | 19 aa | IgG1-DL | 248 |
| | (SEQ ID: 58) | (SEQ ID: 89) | (SEQ ID: 253) | |
| P1410 | hActRIIB L14Q, L33Y | 19 aa | IgG1-DL | 249 |
| | (SEQ ID: 49) | (SEQ ID: 89) | (SEQ ID: 253) | |
| P1411 | hActRIIB L14D, L33Y | 19 aa | IgG1-DL | 250 |
| | (SEQ ID: 50) | (SEQ ID: 89) | (SEQ ID: 253) | |
| P1412 | hActRIIB L14N, L33Y | 19 aa | IgG1-DL | 251 |
| | (SEQ ID: 51) | (SEQ ID: 89) | (SEQ ID: 253) | |

TABLE 6
Exemplary binding agent amino acid sequences

| | | SEQ |
|---|---|---|
| Agent | AA Sequence | ID |
| P75 | ETRECIYYNANWELERTNQSGLERCEGEQDKRLHCYASWRNSSGTIELVKKGCWLDDENC | 171 |
| | YDRQECVATEENPQVYFCCCEGNFCNERFTHLPEAGGPEVTYEPPPTGGGTHTCPPCPAP | |
| | ELLGGPSVFLFPPKPKDTLMISRTPEVTCVVVDVSHEDPEVKFNWYVDGVEVHNAKTKPR | |
| | EEQYNSTYRVVSVLTVLHQDWLNGKEYKCKVSNKALPAPIEKTISKAKGQPREPQVYTLP | |
| | PSRDELTKNQVSLTCLVKGFYPSDIAVEWESNGQPENNYKTTPPVLDSDGSFFLYSKLTV | |
| | DKSRWQQGNVFSCSVMHEALHNHYTQKSLSLSPG | |
| P757 | ETRECIYYNANWELERTNQSGLERCEGEQDKRLHCYASWRNSSGTIELVKKGCWLDDENC | 172 |
| | YDRQECVATEENPQVYFCCCEGNFCNERFTHLPEAGGPEVTYEPPPTGGGGSGGGGSGGG | |
| | GTHTCPPCPAPELLGGPSVFLFPPKPKDTLMISRTPEVTCVVVDVSHEDPEVKFNWYVDG | |
| | VEVHNAKTKPREEQYNSTYRVVSVLIVLHQDWLNGKEYKCKVSNKALPAPIEKTISKAKG | |
| | QPREPQVYTLPPSRDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| P444 | ILGRSETQECLFFNANWEKDRINQTGVEPCYGDKDKRRHCFATWKNISGSIEIVKQGCWL | 173 |
| | DDINCYDRTDCVEKKDSPEVYFCCCEGNMCNEKFSYFPEMEVTQPTSNPVTPKPPTGGGT | |
| | HTCPPCPAPELLGGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALPVPIEKTISKAKGQP | |
| | REPQVYTLPPSREEMTKNQVSLTCLVKGFYPSDIAVEWESNGQPENNYKTTPPVLDSDGS | |
| | FFLYSKLTVDKSRWQQGNVFSCSVMHEALHNHYTQKSLSLSPG | |
| P739 | ETRECIYYNANWELERTNQSGLERCEEEQDKRLHCYASWRNSSGTIELVKKGCWLDDENC | 174 |
| | YDRQECVATEENPQVYFCCCEGNFCNERFTHLPEAGGPEVTYEPPPTGGGGSGGGGSGGG | |
| | GTHTCPPCPAPELLGGPSVFLFPPKPKDTLMISRTPEVTCVVVDVSHEDPEVKENWYVDG | |
| | VEVHNAKTKPREEQYNSTYRVVSVLTVLHQDWLNGKEYKCKVSNKALPAPIEKTISKAKG | |
| | QPREPQVYTLPPSRDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| P750 | ETRECIYYNANWELERINQSGLERCEGEQDKRRHCYASWRNSSGTIELVKKGCWLDDENC | 175 |
| | YDRQECVATEENPQVYFCCCEGNFCNERFTHLPEAGGPEVTYEPPPTGGGGSGGGGSGGG | |
| | GTHTCPPCPAPELLGGPSVFLFPPKPKDTLMISRTPEVTCVVVDVSHEDPEVKENWYVDG | |
| | VEVHNAKTKPREEQYNSTYRVVSVLTVLHQDWLNGKEYKCKVSNKALPAPIEKTISKAKG | |
| | QPREPQVYTLPPSRDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| P751 | ETRECIYYNANWELERTNQSGLERCEGEQDKRYHCYASWRNSSGTIELVKKGCWLDDFNC | 176 |
| | YDRQECVATEENPQVYFCCCEGNFCNERFTHLPEAGGPEVTYEPPPTGGGGSGGGGSGGG | |
| | GTHTCPPCPAPELLGGPSVFLFPPKPKDTLMISRTPEVTCVVVDVSHEDPEVKENWYVDG | |
| | VEVHNAKTKPREEQYNSTYRVVSVLTVLHQDWLNGKEYKCKVSNKALPAPIEKTISKAKG | |
| | QPREPQVYTLPPSRDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| P753 | ETRECIYYNANWELERTNQSGLERCEGEQDKRLHCYASWRNSSGTIELVKKGCWLDDENC | 177 |
| | YDRQECVAHEENPQVYFCCCEGNFCNERFTHLPEAGGPEVTYEPPPTGGGGSGGGGSGGG | |
| | GTHTCPPCPAPELLGGPSVFLFPPKPKDTLMISRTPEVTCVVVDVSHEDPEVKENWYVDG | |
| | VEVHNAKTKPREEQYNSTYRVVSVLTVLHQDWLNGKEYKCKVSNKALPAPIEKTISKAKG | |
| | QPREPQVYTLPPSRDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| P754 | ETRECIYYNANWELERTNQSGLERCEGEQDKRLHCYASWRNSSGTIELVKKGCWLDDENC | 178 |
| | YDRQECVAQEENPQVYFCCCEGNFCNERFTHLPEAGGPEVTYEPPPTGGGGSGGGGSGGG | |
| | GTHTCPPCPAPELLGGPSVFLFPPKPKDTLMISRTPEVTCVVVDVSHEDPEVKENWYVDG | |
| | VEVHNAKTKPREEQYNSTYRVVSVLTVLHQDWLNGKEYKCKVSNKALPAPIEKTISKAKG | |
| | QPREPQVYTLPPSRDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| P765 | ETRECIYYNANWELERTNQSGLERCEEEQDKRLHCYASWRNSSGTIELVKKGCWLDDENC | 179 |
| | YDRQECVATEENPQVYFCCCEGNFCNERFTHLPEAGGPEVTYEPPPTGGGTHTCPPCPAP | |
| | ELLGGPSVFLFPPKPKDTLMISRTPEVTCVVVDVSHEDPEVKFNWYVDGVEVHNAKTKPR | |
| | EEQYNSTYRVVSVLTVLHQDWLNGKEYKCKVSNKALPAPIEKTISKAKGQPREPQVYTLP | |
| | PSRDELTKNQVSLTCLVKGFYPSDIAVEWESNGQPENNYKTTPPVLDSDGSFFLYSKLTV | |
| | DKSRWQQGNVFSCSVMHEALHNHYTQKSLSLSPG | |
| P777 | ETRECIYYNANWELERTNQSGLERCEGEQDKRYHCYASWRNSSGTIELVKKGCWLDDENC | 180 |
| | YDRQECVATEENPQVYFCCCEGNFCNERFTHLPEAGGPEVTYEPPPTGGGTHTCPPCPAP | |
| | ELLGGPSVFLFPPKPKDTLMISRTPEVTCVVVDVSHEDPEVKFNWYVDGVEVHNAKTKPR | |
| | EEQYNSTYRVVSVLTVLHQDWLNGKEYKCKVSNKALPAPIEKTISKAKGQPREPQVYTLP | |
| | PSRDELTKNQVSLTCLVKGFYPSDIAVEWESNGQPENNYKTTPPVLDSDGSFFLYSKLTV | |
| | DKSRWQQGNVFSCSVMHEALHNHYTQKSLSLSPG | |
| P779 | ETRECIYYNANWELERTNQSGLERCEGEQDKRLHCYASWRNSSGTIELVKKGCWLDDENC | 181 |
| | YDRQECVAHEENPQVYFCCCEGNFCNERFTHLPEAGGPEVTYEPPPTGGGTHTCPPCPAP | |
| | ELLGGPSVFLFPPKPKDTLMISRTPEVTCVVVDVSHEDPEVKFNWYVDGVEVHNAKTKPR | |
| | EEQYNSTYRVVSVLTVLHQDWLNGKEYKCKVSNKALPAPIEKTISKAKGQPREPQVYTLP | |
| | PSRDELTKNQVSLTCLVKGFYPSDIAVEWESNGQPENNYKTTPPVLDSDGSFFLYSKLTV | |
| | DKSRWQQGNVFSCSVMHEALHNHYTQKSLSLSPG | |
| P780 | ETRECIYYNANWELERTNQSGLERCEGEQDKRLHCYASWRNSSGTIELVKKGCWLDDENC | 182 |
| | YDRQECVAQEENPQVYFCCCEGNFCNERFTHLPEAGGPEVTYEPPPTGGGTHTCPPCPAP | |
| | ELLGGPSVFLFPPKPKDTLMISRTPEVTCVVVDVSHEDPEVKFNWYVDGVEVHNAKTKPR | |
| | EEQYNSTYRVVSVLTVLHQDWLNGKEYKCKVSNKALPAPIEKTISKAKGQPREPQVYTLP | |
| | PSRDELTKNQVSLTCLVKGFYPSDIAVEWESNGQPENNYKTTPPVLDSDGSFFLYSKLTV | |
| | DKSRWQQGNVFSCSVMHEALHNHYTQKSLSLSPG | |
| P1171 | ETRECIYYNANWELERTNQSGLERCEGEQDKRYHCYASWRNSSGTIELVKKGCWLDDENC | 183 |
| | YDRQECVAHEENPQVYFCCCEGNFCNERFTHLPEAGGPEVTYEPPPTGGGTHTCPPCPAP | |
| | ELLGGPSVFLFPPKPKDTLMISRTPEVTCVVVDVSHEDPEVKFNWYVDGVEVHNAKTKPR | |
| | EEQYNSTYRVVSVLTVLHQDWLNGKEYKCKVSNKALPAPIEKTISKAKGQPREPQVYTLP | |
| | PSRDELTKNQVSLTCLVKGFYPSDIAVEWESNGQPENNYKTTPPVLDSDGSFFLYSKLTV | |
| | DKSRWQQGNVFSCSVMHEALHNHYTQKSLSLSPG | |
| P1172 | ETRECIYYNANWELERTNQSGLERCEGEQDKRYHCYASWRNSSGTIELVKKGCWLDDENC | 184 |
| | YDRQECVAHEENPQVYFCCCEGNFCNERFTHLPEAGGPEVTYEPPPTGGGGSGGGGSTHT | |
| | CPPCPAPELLGGPSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPIEKTISKAKGQPRE | |
| | PQVYTLPPSRDELTKNQVSLTCLVKGFYPSDIAVEWESNGQPENNYKTTPPVLDSDGSFF | |
| | LYSKLTVDKSRWQQGNVFSCSVMHEALHNHYTQKSLSLSPG | |
| P1173 | ETRECIYYNANWELERTNQSGLERCEGEQDKRYHCYASWRNSSGTIELVKKGCWLDDENC | 185 |
| | YDRQECVAHEENPQVYFCCCEGNFCNERFTHLPEAGGPEVTYEPPPTGGGGSGGGGSGGG | |
| | GTHTCPPCPAPELLGGPSVFLFPPKPKDTLMISRTPEVTCVVVDVSHEDPEVKENWYVDG | |
| | VEVHNAKTKPREEQYNSTYRVVSVLTVLHQDWLNGKEYKCKVSNKALPAPIEKTISKAKG | |
| | QPREPQVYTLPPSRDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| P1174 | ETRECIYYNANWELERTNQSGLERCEGEQDKRYHCYASWRNSSGTIELVKKGCWLDDENC | 186 |
| | YDRQECVAQEENPQVYFCCCEGNFCNERFTHLPEAGGPEVTYEPPPTGGGTHTCPPCPAP | |
| | ELLGGPSVFLFPPKPKDTLMISRTPEVTCVVVDVSHEDPEVKFNWYVDGVEVHNAKTKPR | |
| | EEQYNSTYRVVSVLTVLHQDWLNGKEYKCKVSNKALPAPIEKTISKAKGQPREPQVYTLP | |
| | PSRDELTKNQVSLTCLVKGFYPSDIAVEWESNGQPENNYKTTPPVLDSDGSFFLYSKLTV | |
| | DKSRWQQGNVFSCSVMHEALHNHYTQKSLSLSPG | |
| P1175 | ETRECIYYNANWELERTNQSGLERCEGEQDKRYHCYASWRNSSGTIELVKKGCWLDDENC | 187 |
| | YDRQECVAQEENPQVYFCCCEGNFCNERFTHLPEAGGPEVTYEPPPTGGGGSGGGGSTHT | |
| | CPPCPAPELLGGPSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPIEKTISKAKGQPRE | |
| | PQVYTLPPSRDELTKNQVSLTCLVKGFYPSDIAVEWESNGQPENNYKTTPPVLDSDGSFF | |
| | LYSKLTVDKSRWQQGNVFSCSVMHEALHNHYTQKSLSLSPG | |
| P1176 | ETRECIYYNANWELERTNQSGLERCEGEQDKRYHCYASWRNSSGTIELVKKGCWLDDENC | 188 |
| | YDRQECVAQEENPQVYFCCCEGNFCNERFTHLPEAGGPEVTYEPPPTGGGGSGGGGSGGG | |
| | GTHTCPPCPAPELLGGPSVFLFPPKPKDTLMISRTPEVTCVVVDVSHEDPEVKENWYVDG | |
| | VEVHNAKTKPREEQYNSTYRVVSVLTVLHQDWLNGKEYKCKVSNKALPAPIEKTISKAKG | |
| | QPREPQVYTLPPSRDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| P1177 | ETRECIYYNANWELERTNQSGLERCEGEQDKRYHCYASWRNSSGTIELVKKGCWLDDENC | 189 |
| | YDRQECVAEEENPQVYFCCCEGNFCNERFTHLPEAGGPEVTYEPPPTGGGTHTCPPCPAP | |
| | ELLGGPSVFLFPPKPKDTLMISRTPEVTCVVVDVSHEDPEVKFNWYVDGVEVHNAKTKPR | |
| | EEQYNSTYRVVSVLTVLHQDWLNGKEYKCKVSNKALPAPIEKTISKAKGQPREPQVYTLP | |
| | PSRDELTKNQVSLTCLVKGFYPSDIAVEWESNGQPENNYKTTPPVLDSDGSFFLYSKLTV | |
| | DKSRWQQGNVFSCSVMHEALHNHYTQKSLSLSPG | |
| P1178 | ETRECIYYNANWELERTNQSGLERCEGEQDKRYHCYASWRNSSGTIELVKKGCWLDDENC | 190 |
| | YDRQECVAEEENPQVYFCCCEGNFCNERFTHLPEAGGPEVTYEPPPTGGGGSGGGGSTHT | |
| | CPPCPAPELLGGPSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPIEKTISKAKGQPRE | |
| | PQVYTLPPSRDELTKNQVSLTCLVKGFYPSDIAVEWESNGQPENNYKTTPPVLDSDGSFF | |
| | LYSKLTVDKSRWQQGNVFSCSVMHEALHNHYTQKSLSLSPG | |
| P1179 | ETRECIYYNANWELERTNQSGLERCEGEQDKRYHCYASWRNSSGTIELVKKGCWLDDENC | 191 |
| | YDRQECVAEEENPQVYFCCCEGNFCNERFTHLPEAGGPEVTYEPPPTGGGGSGGGGSGGG | |
| | GTHTCPPCPAPELLGGPSVFLFPPKPKDTLMISRTPEVTCVVVDVSHEDPEVKENWYVDG | |
| | VEVHNAKTKPREEQYNSTYRVVSVLTVLHQDWLNGKEYKCKVSNKALPAPIEKTISKAKG | |
| | QPREPQVYTLPPSRDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| P1180 | ETRECIYYNANWELERTNQSGLERCEDEQDKRYHCYASWRNSSGTIELVKKGCWLDDENC | 192 |
| | YDRQECVATEENPQVYFCCCEGNFCNERFTHLPEAGGPEVTYEPPPTGGGTHTCPPCPAP | |
| | ELLGGPSVFLFPPKPKDTLMISRTPEVTCVVVDVSHEDPEVKFNWYVDGVEVHNAKTKPR | |
| | EEQYNSTYRVVSVLTVLHQDWLNGKEYKCKVSNKALPAPIEKTISKAKGQPREPQVYTLP | |
| | PSRDELTKNQVSLTCLVKGFYPSDIAVEWESNGQPENNYKTTPPVLDSDGSFFLYSKLTV | |
| | DKSRWQQGNVFSCSVMHEALHNHYTQKSLSLSPG | |
| P1181 | ETRECIYYNANWELERTNQSGLERCEDEQDKRYHCYASWRNSSGTIELVKKGCWLDDENC | 193 |
| | YDRQECVATEENPQVYFCCCEGNFCNERFTHLPEAGGPEVTYEPPPTGGGGSGGGGSTHT | |
| | CPPCPAPELLGGPSVFLFPPKPKDTLMISRTPEVTCVVVDVSHEDPEVKENWYVDGVEVH | |
| | NAKTKPREEQYNSTYRVVSVLTVLHQDWLNGKEYKCKVSNKALPAPIEKTISKAKGQPRE | |
| | PQVYTLPPSRDELTKNQVSLTCLVKGFYPSDIAVEWESNGQPENNYKTTPPVLDSDGSFF | |
| | LYSKLTVDKSRWQQGNVFSCSVMHEALHNHYTQKSLSLSPG | |
| P1182 | ETRECIYYNANWELERTNQSGLERCEDEQDKRYHCYASWRNSSGTIELVKKGCWLDDENC | 194 |
| | YDRQECVATEENPQVYFCCCEGNFCNERFTHLPEAGGPEVTYEPPPTGGGGSGGGGSGGG | |
| | GTHTCPPCPAPELLGGPSVFLFPPKPKDTLMISRTPEVTCVVVDVSHEDPEVKENWYVDG | |
| | VEVHNAKTKPREEQYNSTYRVVSVLIVLHQDWLNGKEYKCKVSNKALPAPIEKTISKAKG | |
| | QPREPQVYTLPPSRDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| P1183 | ETRECIYYNANWELERTNQSGLERCEEEQDKRYHCYASWRNSSGTIELVKKGCWLDDENC | 195 |
| | YDRQECVATEENPQVYFCCCEGNFCNERFTHLPEAGGPEVTYEPPPTGGGTHTCPPCPAP | |
| | ELLGGPSVFLFPPKPKDTLMISRTPEVTCVVVDVSHEDPEVKENWYVDGVEVHNAKTKPR | |
| | EEQYNSTYRVVSVLTVLHQDWLNGKEYKCKVSNKALPAPIEKTISKAKGQPREPQVYTLP | |
| | PSRDELTKNQVSLTCLVKGFYPSDIAVEWESNGQPENNYKTTPPVLDSDGSFFLYSKLTV | |
| | DKSRWQQGNVFSCSVMHEALHNHYTQKSLSLSPG | |
| P1184 | ETRECIYYNANWELERTNQSGLERCEEEQDKRYHCYASWRNSSGTIELVKKGCWLDDENC | 196 |
| | YDRQECVATEENPQVYFCCCEGNFCNERFTHLPEAGGPEVTYEPPPTGGGGSGGGGSTHT | |
| | CPPCPAPELLGGPSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPIEKTISKAKGQPRE | |
| | PQVYTLPPSRDELTKNQVSLTCLVKGFYPSDIAVEWESNGQPENNYKTTPPVLDSDGSFF | |
| | LYSKLTVDKSRWQQGNVFSCSVMHEALHNHYTQKSLSLSPG | |
| P1185 | ETRECIYYNANWELERTNQSGLERCEEEQDKRYHCYASWRNSSGTIELVKKGCWLDDENC | 197 |
| | YDRQECVATEENPQVYFCCCEGNFCNERFTHLPEAGGPEVTYEPPPTGGGGSGGGGSGGG | |
| | GTHTCPPCPAPELLGGPSVFLFPPKPKDTLMISRTPEVTCVVVDVSHEDPEVKENWYVDG | |
| | VEVHNAKTKPREEQYNSTYRVVSVLTVLHQDWLNGKEYKCKVSNKALPAPIEKTISKAKG | |
| | QPREPQVYTLPPSRDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| P1186 | ETRECIYYNANWELERTNQSGLERCEGEQDKRFHCYASWRNSSGTIELVKKGCWLDDENC | 198 |
| | YDRQECVAQEENPQVYFCCCEGNFCNERFTHLPEAGGPEVTYEPPPTGGGTHTCPPCPAP | |
| | ELLGGPSVFLFPPKPKDTLMISRTPEVTCVVVDVSHEDPEVKFNWYVDGVEVHNAKTKPR | |
| | EEQYNSTYRVVSVLTVLHQDWLNGKEYKCKVSNKALPAPIEKTISKAKGQPREPQVYTLP | |
| | PSRDELTKNQVSLTCLVKGFYPSDIAVEWESNGQPENNYKTTPPVLDSDGSFFLYSKLTV | |
| | DKSRWQQGNVFSCSVMHEALHNHYTQKSLSLSPG | |
| P1187 | ETRECIYYNANWELERTNQSGLERCEGEQDKRFHCYASWRNSSGTIELVKKGCWLDDFNC | 199 |
| | YDRQECVAQEENPQVYFCCCEGNFCNERFTHLPEAGGPEVTYEPPPTGGGGSGGGGSTHT | |
| | CPPCPAPELLGGPSVFLFPPKPKDTLMISRTPEVTCVVVDVSHEDPEVKENWYVDGVEVH | |
| | NAKTKPREEQYNSTYRVVSVLTVLHQDWLNGKEYKCKVSNKALPAPIEKTISKAKGQPRE | |
| | PQVYTLPPSRDELTKNQVSLTCLVKGFYPSDIAVEWESNGQPENNYKTTPPVLDSDGSFF | |
| | LYSKLTVDKSRWQQGNVFSCSVMHEALHNHYTQKSLSLSPG | |
| P1188 | ETRECIYYNANWELERTNQSGLERCEGEQDKRFHCYASWRNSSGTIELVKKGCWLDDENC | 200 |
| | YDRQECVAQEENPQVYFCCCEGNFCNERFTHLPEAGGPEVTYEPPPTGGGGSGGGGSGGG | |
| | GTHTCPPCPAPELLGGPSVFLFPPKPKDTLMISRTPEVTCVVVDVSHEDPEVKENWYVDG | |
| | VEVHNAKTKPREEQYNSTYRVVSVLTVLHQDWLNGKEYKCKVSNKALPAPIEKTISKAKG | |
| | QPREPQVYTLPPSRDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| P1201 | ETRECIYYNANWELERTNQSGLERCEGEQDKRLHCYASWRNSSGTIELVKKGCWLDDFNC | 201 |
| | YDRQECVAEEENPQVYFCCCEGNFCNERFTHLPEAGGPEVTYEPPPTGGGTHTCPPCPAP | |
| | ELLGGPSVFLFPPKPKDTLMISRTPEVTCVVVDVSHEDPEVKENWYVDGVEVHNAKTKPR | |
| | EEQYNSTYRVVSVLTVLHQDWLNGKEYKCKVSNKALPAPIEKTISKAKGQPREPQVYTLP | |
| | PSRDELTKNQVSLTCLVKGFYPSDIAVEWESNGQPENNYKTTPPVLDSDGSFFLYSKLTV | |
| | DKSRWQQGNVFSCSVMHEALHNHYTQKSLSLSPG | |
| P1202 | ETRECIYYNANWELERTNQSGLERCEGEQDKRLHCYASWRNSSGTIELVKKGCWLDDENC | 202 |
| | YDRQECVAEEENPQVYFCCCEGNFCNERFTHLPEAGGPEVTYEPPPTGGGGSGGGGSTHT | |
| | CPPCPAPELLGGPSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPIEKTISKAKGQPRE | |
| | PQVYTLPPSRDELTKNQVSLTCLVKGFYPSDIAVEWESNGQPENNYKTTPPVLDSDGSFF | |
| | LYSKLTVDKSRWQQGNVFSCSVMHEALHNHYTQKSLSLSPG | |
| P1203 | ETRECIYYNANWELERTNQSGLERCEGEQDKRLHCYASWRNSSGTIELVKKGCWLDDFNC | 203 |
| | YDRQECVAEEENPQVYFCCCEGNFCNERFTHLPEAGGPEVTYEPPPTGGGGSGGGGSGGG | |
| | GTHTCPPCPAPELLGGPSVFLFPPKPKDTLMISRTPEVTCVVVDVSHEDPEVKENWYVDG | |
| | VEVHNAKTKPREEQYNSTYRVVSVLTVLHQDWLNGKEYKCKVSNKALPAPIEKTISKAKG | |
| | QPREPQVYTLPPSRDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| P1204 | ETRECIYYNANWELERTNQSGLERCEGEQDKRFHCYASWRNSSGTIELVKKGCWLDDENC | 204 |
| | YDRQECVATEENPQVYFCCCEGNFCNERFTHLPEAGGPEVTYEPPPTGGGTHTCPPCPAP | |
| | ELLGGPSVFLFPPKPKDTLMISRTPEVTCVVVDVSHEDPEVKFNWYVDGVEVHNAKTKPR | |
| | EEQYNSTYRVVSVLTVLHQDWLNGKEYKCKVSNKALPAPIEKTISKAKGQPREPQVYTLP | |
| | PSRDELTKNQVSLTCLVKGFYPSDIAVEWESNGQPENNYKTTPPVLDSDGSFFLYSKLTV | |
| | DKSRWQQGNVFSCSVMHEALHNHYTQKSLSLSPG | |
| P1205 | ETRECIYYNANWELERTNQSGLERCEGEQDKRFHCYASWRNSSGTIELVKKGCWLDDENC | 205 |
| | YDRQECVATEENPQVYFCCCEGNFCNERFTHLPEAGGPEVTYEPPPTGGGGSGGGGSTHT | |
| | CPPCPAPELLGGPSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPIEKTISKAKGQPRE | |
| | PQVYTLPPSRDELTKNQVSLTCLVKGFYPSDIAVEWESNGQPENNYKTTPPVLDSDGSFF | |
| | LYSKLTVDKSRWQQGNVFSCSVMHEALHNHYTQKSLSLSPG | |
| P1206 | ETRECIYYNANWELERTNQSGLERCEGEQDKRFHCYASWRNSSGTIELVKKGCWLDDENC | 206 |
| | YDRQECVATEENPQVYFCCCEGNFCNERFTHLPEAGGPEVTYEPPPTGGGGSGGGGSGGG | |
| | GTHTCPPCPAPELLGGPSVFLFPPKPKDTLMISRTPEVTCVVVDVSHEDPEVKENWYVDG | |
| | VEVHNAKTKPREEQYNSTYRVVSVLTVLHQDWLNGKEYKCKVSNKALPAPIEKTISKAKG | |
| | QPREPQVYTLPPSRDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| P1207 | ETRECIYYNANWELERTNQSGLERCEGEQDKRQHCYASWRNSSGTIELVKKGCWLDDENC | 207 |
| | YDRQECVATEENPQVYFCCCEGNFCNERFTHLPEAGGPEVTYEPPPTGGGTHTCPPCPAP | |
| | ELLGGPSVFLFPPKPKDTLMISRTPEVTCVVVDVSHEDPEVKENWYVDGVEVHNAKTKPR | |
| | EEQYNSTYRVVSVLIVLHQDWLNGKEYKCKVSNKALPAPIEKTISKAKGQPREPQVYTLP | |
| | PSRDELTKNQVSLTCLVKGFYPSDIAVEWESNGQPENNYKTTPPVLDSDGSFFLYSKLTV | |
| | DKSRWQQGNVFSCSVMHEALHNHYTQKSLSLSPG | |
| P1208 | ETRECIYYNANWELERTNQSGLERCEGEQDKRQHCYASWRNSSGTIELVKKGCWLDDENC | 208 |
| | YDRQECVATEENPQVYFCCCEGNFCNERFTHLPEAGGPEVTYEPPPTGGGGSGGGGSTHT | |
| | CPPCPAPELLGGPSVFLFPPKPKDTLMISRTPEVTCVVVDVSHEDPEVKENWYVDGVEVH | |
| | NAKTKPREEQYNSTYRVVSVLTVLHQDWLNGKEYKCKVSNKALPAPIEKTISKAKGQPRE | |
| | PQVYTLPPSRDELTKNQVSLTCLVKGFYPSDIAVEWESNGQPENNYKTTPPVLDSDGSFF | |
| | LYSKLTVDKSRWQQGNVFSCSVMHEALHNHYTQKSLSLSPG | |
| P1209 | ETRECIYYNANWELERTNQSGLERCEGEQDKRQHCYASWRNSSGTIELVKKGCWLDDENC | 209 |
| | YDRQECVATEENPQVYFCCCEGNFCNERFTHLPEAGGPEVTYEPPPTGGGGSGGGGSGGG | |
| | GTHTCPPCPAPELLGGPSVFLFPPKPKDTLMISRTPEVTCVVVDVSHEDPEVKFNWYVDG | |
| | VEVHNAKTKPREEQYNSTYRVVSVLTVLHQDWLNGKEYKCKVSNKALPAPIEKTISKAKG | |
| | QPREPQVYTLPPSRDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| P1210 | ETRECIYYNANWELERTNQSGLERCEGEQDKRYHCYASWRNSSGTIELVKKGCWLDDENC | 210 |
| | YDRQECVATEENPQVYFCCCEGNFCNERFTHLPEAGGPEVTYEPPPTGGGGSGGGGSTHT | |
| | CPPCPAPELLGGPSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPIEKTISKAKGQPRE | |
| | PQVYTLPPSRDELTKNQVSLTCLVKGFYPSDIAVEWESNGQPENNYKTTPPVLDSDGSFF | |
| | LYSKLTVDKSRWQQGNVFSCSVMHEALHNHYTQKSLSLSPG | |
| P1229 | ETRECIYYNANWELERTNQSGLERCEGEQDKRWHCYASWRNSSGTIELVKKGCWLDDFNC | 211 |
| | YDRQECVATEENPQVYFCCCEGNFCNERFTHLPEAGGPEVTYEPPPTGGGGSGGGGSGGG | |
| | GTHTCPPCPAPELLGGPSVFLFPPKPKDTLMISRTPEVTCVVVDVSHEDPEVKENWYVDG | |
| | VEVHNAKTKPREEQYNSTYRVVSVLTVLHQDWLNGKEYKCKVSNKALPAPIEKTISKAKG | |
| | QPREPQVYTLPPSRDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| P1230 | ETRECIYYNANWELERTNQSGLERCEGEQDKREHCYASWRNSSGTIELVKKGCWLDDENC | 212 |
| | YDRQECVATEENPQVYFCCCEGNFCNERFTHLPEAGGPEVTYEPPPTGGGGSGGGGSGGG | |
| | GTHTCPPCPAPELLGGPSVFLFPPKPKDTLMISRTPEVTCVVVDVSHEDPEVKENWYVDG | |
| | VEVHNAKTKPREEQYNSTYRVVSVLTVLHQDWLNGKEYKCKVSNKALPAPIEKTISKAKG | |
| | QPREPQVYTLPPSRDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| P1231 | ETRECIYYNANWELERTNQSGLERCEGEQDKRKHCYASWRNSSGTIELVKKGCWLDDENC | 213 |
| | YDRQECVATEENPQVYFCCCEGNFCNERFTHLPEAGGPEVTYEPPPTGGGGSGGGGSGGG | |
| | GTHTCPPCPAPELLGGPSVFLFPPKPKDTLMISRTPEVTCVVVDVSHEDPEVKFNWYVDG | |
| | VEVHNAKTKPREEQYNSTYRVVSVLTVLHQDWLNGKEYKCKVSNKALPAPIEKTISKAKG | |
| | QPREPQVYTLPPSRDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| P1232 | ETRECIYYNANWELERTNQSGLERCEGEQDKRMHCYASWRNSSGTIELVKKGCWLDDENC | 214 |
| | YDRQECVATEENPQVYFCCCEGNFCNERFTHLPEAGGPEVTYEPPPTGGGGSGGGGSGGG | |
| | GTHTCPPCPAPELLGGPSVFLFPPKPKDTLMISRTPEVTCVVVDVSHEDPEVKENWYVDG | |
| | VEVHNAKTKPREEQYNSTYRVVSVLTVLHQDWLNGKEYKCKVSNKALPAPIEKTISKAKG | |
| | QPREPQVYTLPPSRDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| P1235 | ETRECIYYNANWELERTNQSGLERCENEQDKRYHCYASWRNSSGTIELVKKGCWLDDENC | 215 |
| | YDRQECVATEENPQVYFCCCEGNFCNERFTHLPEAGGPEVTYEPPPTGGGGSGGGGSGGG | |
| | GTHTCPPCPAPELLGGPSVFLFPPKPKDTLMISRTPEVTCVVVDVSHEDPEVKFNWYVDG | |
| | VEVHNAKTKPREEQYNSTYRVVSVLTVLHQDWLNGKEYKCKVSNKALPAPIEKTISKAKG | |
| | QPREPQVYTLPPSRDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| P1236 | ETRECIYYNANWELERTNQSGLERCEQEQDKRYHCYASWRNSSGTIELVKKGCWLDDENC | 216 |
| | YDRQECVATEENPQVYFCCCEGNFCNERFTHLPEAGGPEVTYEPPPTGGGGSGGGGSGGG | |
| | GTHTCPPCPAPELLGGPSVFLFPPKPKDTLMISRTPEVTCVVVDVSHEDPEVKENWYVDG | |
| | VEVHNAKTKPREEQYNSTYRVVSVLTVLHQDWLNGKEYKCKVSNKALPAPIEKTISKAKG | |
| | QPREPQVYTLPPSRDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| P1237 | ETRECIYYNANWELERTNQSGLERCEKEQDKRYHCYASWRNSSGTIELVKKGCWLDDENC | 217 |
| | YDRQECVATEENPQVYFCCCEGNFCNERFTHLPEAGGPEVTYEPPPTGGGGSGGGGSGGG | |
| | GTHTCPPCPAPELLGGPSVFLFPPKPKDTLMISRTPEVTCVVVDVSHEDPEVKENWYVDG | |
| | VEVHNAKTKPREEQYNSTYRVVSVLTVLHQDWLNGKEYKCKVSNKALPAPIEKTISKAKG | |
| | QPREPQVYTLPPSRDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| P1238 | ETRECIYYNANWELERTNQSGLERCETEQDKRYHCYASWRNSSGTIELVKKGCWLDDENC | 218 |
| | YDRQECVATEENPQVYFCCCEGNFCNERFTHLPEAGGPEVTYEPPPTGGGGSGGGGSGGG | |
| | GTHTTCPPCPAPELLGGPSVFLFPPKPKDTLMISRTPEVTCVVVDVSHEDPEVKENWYVDG | |
| | VEVHNAKTKPREEQYNSTYRVVSVLTVLHQDWLNGKEYKCKVSNKALPAPIEKTISKAKG | |
| | QPREPQVYTLPPSRDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| P1239 | ETRECIYYNANWELERTNQSGLERCEMEQDKRYHCYASWRNSSGTIELVKKGCWLDDENC | 219 |
| | YDRQECVATEENPQVYFCCCEGNFCNERFTHLPEAGGPEVTYEPPPTGGGGSGGGGSGGG | |
| | GTHTTCPPCPAPELLGGPSVFLFPPKPKDTLMISRTPEVTCVVVDVSHEDPEVKENWYVDG | |
| | VEVHNAKTKPREEQYNSTYRVVSVLTVLHQDWLNGKEYKCKVSNKALPAPIEKTISKAKG | |
| | QPREPQVYTLPPSRDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| P1240 | ETRECIYYNANWELERTNQSGLERCEGEQDKRYHCYASWRNSSGTIELVKKGCWLDDENC | 220 |
| | YDRQECVAREENPQVYFCCCEGNFCNERFTHLPEAGGPEVTYEPPPTGGGGSGGGGSGGG | |
| | GTHTTCPPCPAPELLGGPSVFLFPPKPKDTLMISRTPEVTCVVVDVSHEDPEVKENWYVDG | |
| | VEVHNAKTKPREEQYNSTYRVVSVLTVLHQDWLNGKEYKCKVSNKALPAPIEKTISKAKG | |
| | QPREPQVYTLPPSRDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| P1241 | ETRECIYYNANWELERTNQSGLERCEGEQDKRYHCYASWRNSSGTIELVKKGCWLDDENC | 221 |
| | YDRQECVAYEENPQVYFCCCEGNFCNERFTHLPEAGGPEVTYEPPPTGGGGSGGGGSGGG | |
| | GTHTTCPPCPAPELLGGPSVFLFPPKPKDTLMISRTPEVTCVVVDVSHEDPEVKENWYVDG | |
| | VEVHNAKTKPREEQYNSTYRVVSVLTVLHQDWLNGKEYKCKVSNKALPAPIEKTISKAKG | |
| | QPREPQVYTLPPSRDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| P1242 | ETRECIYYNANWELERTNQSGLERCEGEQDKRYHCYASWRNSSGTIELVKKGCWLDDENC | 222 |
| | YDRQECVAWEENPQVYFCCCEGNFCNERFTHLPEAGGPEVTYEPPPTGGGGSGGGGSGGG | |
| | GTHTTCPPCPAPELLGGPSVFLFPPKPKDTLMISRTPEVTCVVVDVSHEDPEVKFNWYVDG | |
| | VEVHNAKTKPREEQYNSTYRVVSVLTVLHQDWLNGKEYKCKVSNKALPAPIEKTISKAKG | |
| | QPREPQVYTLPPSRDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| P1269 | ETRECIYYNANWEEERTNQSGLERCEGEQDKRYHCYASWRNSSGTIELVKKGCWLDDENC | 223 |
| | YDRQECVATEENPQVYFCCCEGNFCNERFTHLPEAGGPEVTYEPPPTGGGGSGGGGSGGG | |
| | GTHTTCPPCPAPELLGGPSVFLFPPKPKDTLMISRTPEVTCVVVDVSHEDPEVKENWYVDG | |
| | VEVHNAKTKPREEQYNSTYRVVSVLIVLHQDWLNGKEYKCKVSNKALPAPIEKTISKAKG | |
| | QPREPQVYTLPPSRDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| P1270 | ETRECIYYNANWEHERTNQSGLERCEGEQDKRYHCYASWRNSSGTIELVKKGCWLDDENC | 224 |
| | YDRQECVATEENPQVYFCCCEGNFCNERFTHLPEAGGPEVTYEPPPTGGGGSGGGGSGGG | |
| | GTHTTCPPCPAPELLGGPSVFLFPPKPKDTLMISRTPEVTCVVVDVSHEDPEVKENWYVDG | |
| | VEVHNAKTKPREEQYNSTYRVVSVLTVLHQDWLNGKEYKCKVSNKALPAPIEKTISKAKG | |
| | QPREPQVYTLPPSRDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| P1271 | ETRECIYYNANWESERTNQSGLERCEGEQDKRYHCYASWRNSSGTIELVKKGCWLDDENC | 225 |
| | YDRQECVATEENPQVYFCCCEGNFCNERFTHLPEAGGPEVTYEPPPTGGGGSGGGGSGGG | |
| | GTHTTCPPCPAPELLGGPSVFLFPPKPKDTLMISRTPEVTCVVVDVSHEDPEVKENWYVDG | |
| | VEVHNAKTKPREEQYNSTYRVVSVLTVLHQDWLNGKEYKCKVSNKALPAPIEKTISKAKG | |
| | QPREPQVYTLPPSRDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| P1272 | ETRECIYYNANWELERTNQSGLERCEGEQDKRYHCYASWRNSSGTIELVKKGCWYDDENC | 226 |
| | YDRQECVATEENPQVYFCCCEGNFCNERFTHLPEAGGPEVTYEPPPTGGGGSGGGGSGGG | |
| | GTHTTCPPCPAPELLGGPSVFLFPPKPKDTLMISRTPEVTCVVVDVSHEDPEVKENWYVDG | |
| | VEVHNAKTKPREEQYNSTYRVVSVLTVLHQDWLNGKEYKCKVSNKALPAPIEKTISKAKG | |
| | QPREPQVYTLPPSRDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| P1273 | ETRECIYYNANWELERTNQSGLERCEGEQDKRYHCYASWRNSSGTIELVKKGCWQDDENC | 227 |
| | YDRQECVATEENPQVYFCCCEGNFCNERFTHLPEAGGPEVTYEPPPTGGGGSGGGGSGGG | |
| | GTHTTCPPCPAPELLGGPSVFLFPPKPKDTLMISRTPEVTCVVVDVSHEDPEVKENWYVDG | |
| | VEVHNAKTKPREEQYNSTYRVVSVLTVLHQDWLNGKEYKCKVSNKALPAPIEKTISKAKG | |
| | QPREPQVYTLPPSRDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| P1274 | ETRECIYYNANWELERTNQSGLERCEGEQDKRYHCYASWRNSSGTIELVKKGCWMDDENC | 228 |
| | YDRQECVATEENPQVYFCCCEGNFCNERFTHLPEAGGPEVTYEPPPTGGGGSGGGGSGGG | |
| | GTHTTCPPCPAPELLGGPSVFLFPPKPKDTLMISRTPEVTCVVVDVSHEDPEVKFNWYVDG | |
| | VEVHNAKTKPREEQYNSTYRVVSVLTVLHQDWLNGKEYKCKVSNKALPAPIEKTISKAKG | |
| | QPREPQVYTLPPSRDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| P1275 | ETRECIYYNANWELERTNQSGLERCEGEQDKRYHCYASWRNSSGTIELVKKGCWIDDENC | 229 |
| | YDRQECVATEENPQVYFCCCEGNFCNERFTHLPEAGGPEVTYEPPPTGGGGSGGGGSGGG | |
| | GTHTTCPPCPAPELLGGPSVFLFPPKPKDTLMISRTPEVTCVVVDVSHEDPEVKENWYVDG | |
| | VEVHNAKTKPREEQYNSTYRVVSVLTVLHQDWLNGKEYKCKVSNKALPAPIEKTISKAKG | |
| | QPREPQVYTLPPSRDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| P1371 | ETRECIYYNANWELERTNQSGLERCEGEQDKRWHCYASWRNSSGTIELVKKGCWLDDENC | 230 |
| | YDRQECVATEENPQVYFCCCEGNFCNERFTHLPEAGGPEVTYEPPPTGGGGSGGGGSGGG | |
| | GTHTTCPPCPAPELLGGPSVFLFPPKPKDTLYISRTPEVTCVVVDVSHEDPEVKENWYVDG | |
| | VEVHNAKTKPREEQYNSTYRVVSVLTVLHQDWLNGKEYKCKVSNKALPAPIEKTISKAKG | |
| | QPREPQVYTLPPSRDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| P1372 | ETRECIYYNANWELERTNQSGLERCEGEQDKRWHCYASWRNSSGTIELVKKGCWLDDENC | 231 |
| | YDRQECVATEENPQVYFCCCEGNFCNERFTHLPEAGGPEVTYEPPPTGGGGSGGGGSGGG | |
| | GTHTTCPPCPAPELLGGPSVFLFPPKPKDTLYITREPEVTCVVVDVSHEDPEVKFNWYVDG | |
| | VEVHNAKTKPREEQYNSTYRVVSVLTVLHQDWLNGKEYKCKVSNKALPAPIEKTISKAKG | |
| | QPREPQVYTLPPSRDELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| P1373 | ETRECIYYNANWELERTNQSGLERCEGEQDKRWHCYASWRNSSGTIELVKKGCWLDDENC | 232 |
| | YDRQECVATEENPQVYFCCCEGNFCNERFTHLPEAGGPEVTYEPPPTGGGGSGGGGSGGG | |
| | GSGGGGTHTCPPCPAPELLGGPSVFLFPPKPKDTLMISRTPEVTCVVVDVSHEDPEVKEN | |
| | WYVDGVEVHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESNGQPENNYKTTPP | |
| | VLDSDGSFFLYSKLTVDKSRWQQGNVFSCSVMHEALHNHYTQKSLSLSPG | |
| P1374 | ETRECIYYNANWELERTNQSGLERCEGEQDKRWHCYASWRNSSGTIELVKKGCWLDDENC | 233 |
| | YDRQECVATEENPQVYFCCCEGNFCNERFTHLPEAGGPEVTYEPPPTGGGGSGGGGSGGG | |
| | GSGGGGTHTCPPCPAPELLGGPSVFLFPPKPKDTLYISRTPEVTCVVVDVSHEDPEVKEN | |
| | WYVDGVEVHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESNGQPENNYKTTPP | |
| | VLDSDGSFFLYSKLTVDKSRWQQGNVFSCSVMHEALHNHYTQKSLSLSPG | |
| P1375 | ETRECIYYNANWELERTNQSGLERCEGEQDKRWHCYASWRNSSGTIELVKKGCWLDDFNC | 234 |
| | YDRQECVATEENPQVYFCCCEGNFCNERFTHLPEAGGPEVTYEPPPTGGGGSGGGGSGGG | |
| | GSGGGGTHTCPPCPAPELLGGPSVFLFPPKPKDTLYITREPEVTCVVVDVSHEDPEVKFN | |
| | WYVDGVEVHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESNGQPENNYKTTPP | |
| | VLDSDGSFFLYSKLTVDKSRWQQGNVFSCSVMHEALHNHYTQKSLSLSPG | |
| P1385 | ETRECIYYNANWELERTNQSGLERCEDEQDKRWHCYASWRNSSGTIELVKKGCWLDDENC | 235 |
| | YDRQECVATEENPQVYFCCCEGNFCNERFTHLPEAGGPEVTYEPPPTGGGGSGGGGSGGG | |
| | GSGGGGTHTCPPCPAPELLGGPSVFLFPPKPKDTLMISRTPEVTCVVVDVSHEDPEVKEN | |
| | WYVDGVEVHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESNGQPENNYKTTPP | |
| | VLDSDGSFFLYSKLTVDKSRWQQGNVFSCSVMHEALHNHYTQKSLSLSPG | |
| P1386 | ETRECIYYNANWELERTNQSGLERCEEEQDKRWHCYASWRNSSGTIELVKKGCWLDDENC | 236 |
| | YDRQECVATEENPQVYFCCCEGNFCNERFTHLPEAGGPEVTYEPPPTGGGGSGGGGSGGG | |
| | GSGGGGTHTCPPCPAPELLGGPSVFLFPPKPKDTLMISRTPEVTCVVVDVSHEDPEVKEN | |
| | WYVDGVEVHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESNGQPENNYKTTPP | |
| | VLDSDGSFFLYSKLTVDKSRWQQGNVFSCSVMHEALHNHYTQKSLSLSPG | |
| P1387 | ETRECIYYNANWELERTNQSGLERCENEQDKRWHCYASWRNSSGTIELVKKGCWLDDENC | 237 |
| | YDRQECVATEENPQVYFCCCEGNFCNERFTHLPEAGGPEVTYEPPPTGGGGSGGGGSGGG | |
| | GSGGGGTHTCPPCPAPELLGGPSVFLFPPKPKDTLMISRTPEVTCVVVDVSHEDPEVKEN | |
| | WYVDGVEVHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESNGQPENNYKTTPP | |
| | VLDSDGSFFLYSKLTVDKSRWQQGNVFSCSVMHEALHNHYTQKSLSLSPG | |
| P1388 | ETRECIYYNANWELERTNQSGLERCEQEQDKRWHCYASWRNSSGTIELVKKGCWLDDENC | 238 |
| | YDRQECVATEENPQVYFCCCEGNFCNERFTHLPEAGGPEVTYEPPPTGGGGSGGGGSGGG | |
| | GSGGGGTHTCPPCPAPELLGGPSVFLFPPKPKDTLMISRTPEVTCVVVDVSHEDPEVKEN | |
| | WYVDGVEVHNAKTKPREEQYNSTYRVVSVLIVLHQDWLNGKEYKCKVSNKALPAPIEKTI | |
| | SKAKGQPREPQVYTLPPSRDELTKNQVSLTCLVKGFYPSDIAVEWESNGQPENNYKTTPP | |
| | VLDSDGSFFLYSKLTVDKSRWQQGNVFSCSVMHEALHNHYTQKSLSLSPG | |
| P1389 | ETRECIYYNANWELERTNQSGLERCETEQDKRWHCYASWRNSSGTIELVKKGCWLDDENC | 239 |
| | YDRQECVATEENPQVYFCCCEGNFCNERFTHLPEAGGPEVTYEPPPTGGGGSGGGGSGGG | |
| | GSGGGGTHTCPPCPAPELLGGPSVFLFPPKPKDTLMISRTPEVTCVVVDVSHEDPEVKEN | |
| | WYVDGVEVHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESNGQPENNYKTTPP | |
| | VLDSDGSFFLYSKLTVDKSRWQQGNVFSCSVMHEALHNHYTQKSLSLSPG | |
| P1390 | ETRECIYYNANWELERTNQSGLERCEMEQDKRWHCYASWRNSSGTIELVKKGCWLDDENC | 240 |
| | YDRQECVATEENPQVYFCCCEGNFCNERFTHLPEAGGPEVTYEPPPTGGGGSGGGGSGGG | |
| | GSGGGGTHTCPPCPAPELLGGPSVFLFPPKPKDTLMISRTPEVTCVVVDVSHEDPEVKEN | |
| | WYVDGVEVHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESNGQPENNYKTTPP | |
| | VLDSDGSFFLYSKLTVDKSRWQQGNVFSCSVMHEALHNHYTQKSLSLSPG | |
| P1391 | ETRECIYYNANWELERTNQSGLERCEDEQDKRYHCYASWRNSSGTIELVKKGCWLDDENC | 241 |
| | YDRQECVATEENPQVYFCCCEGNFCNERFTHLPEAGGPEVTYEPPPTGGGGSGGGGSGGG | |
| | GSGGGGTHTCPPCPAPELLGGPSVFLFPPKPKDTLMISRTPEVTCVVVDVSHEDPEVKEN | |
| | WYVDGVEVHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESNGQPENNYKTTPP | |
| | VLDSDGSFFLYSKLTVDKSRWQQGNVFSCSVMHEALHNHYTQKSLSLSPG | |
| P1392 | ETRECIYYNANWELERTNQSGLERCEEEQDKRYHCYASWRNSSGTIELVKKGCWLDDENC | 242 |
| | YDRQECVATEENPQVYFCCCEGNFCNERFTHLPEAGGPEVTYEPPPTGGGGSGGGGGGG | |
| | GSGGGGTHTCPPCPAPELLGGPSVFLFPPKPKDTLMISRTPEVTCVVVDVSHEDPEVKEN | |
| | WYVDGVEVHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESNGQPENNYKTTPP | |
| | VLDSDGSFFLYSKLTVDKSRWQQGNVFSCSVMHEALHNHYTQKSLSLSPG | |
| P1395 | ETRECIYYNANWELERTNQSGLERCENEQDKRYHCYASWRNSSGTIELVKKGCWLDDENC | 243 |
| | YDRQECVATEENPQVYFCCCEGNFCNERFTHLPEAGGPEVTYEPPPTGGGGSGGGGSGGG | |
| | GSGGGGTHTCPPCPAPELLGGPSVFLFPPKPKDTLMISRTPEVTCVVVDVSHEDPEVKEN | |
| | WYVDGVEVHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESNGQPENNYKTTPP | |
| | VLDSDGSFFLYSKLTVDKSRWQQGNVFSCSVMHEALHNHYTQKSLSLSPG | |
| P1396 | ETRECIYYNANWELERTNQSGLERCEQEQDKRYHCYASWRNSSGTIELVKKGCWLDDENC | 244 |
| | YDRQECVATEENPQVYFCCCEGNFCNERFTHLPEAGGPEVTYEPPPTGGGGSGGGGSGGG | |
| | GSGGGGTHTCPPCPAPELLGGPSVFLFPPKPKDTLMISRTPEVTCVVVDVSHEDPEVKEN | |
| | WYVDGVEVHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESNGQPENNYKTTPP | |
| | VLDSDGSFFLYSKLTVDKSRWQQGNVFSCSVMHEALHNHYTQKSLSLSPG | |
| P1406 | ETRECIYYNANWEEERTNQSGLERCEGEQDKRWHCYASWRNSSGTIELVKKGCWLDDENC | 245 |
| | YDRQECVATEENPQVYFCCCEGNFCNERFTHLPEAGGPEVTYEPPPTGGGGSGGGGSGGG | |
| | GSGGGGTHTCPPCPAPELLGGPSVFLFPPKPKDTLMISRTPEVTCVVVDVSHEDPEVKEN | |
| | WYVDGVEVHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESNGQPENNYKTTPP | |
| | VLDSDGSFFLYSKLTVDKSRWQQGNVFSCSVMHEALHNHYTQKSLSLSPG | |
| P1407 | ETRECIYYNANWEDERTNQSGLERCEGEQDKRWHCYASWRNSSGTIELVKKGCWLDDENC | 246 |
| | YDRQECVATEENPQVYFCCCEGNFCNERFTHLPEAGGPEVTYEPPPTGGGGSGGGGSGGG | |
| | GSGGGGTHTCPPCPAPELLGGPSVFLFPPKPKDTLMISRTPEVTCVVVDVSHEDPEVKEN | |
| | WYVDGVEVHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESNGQPENNYKTTPP | |
| | VLDSDGSFFLYSKLTVDKSRWQQGNVFSCSVMHEALHNHYTQKSLSLSPG | |
| P1408 | ETRECIYYNANWENERTNQSGLERCEGEQDKRWHCYASWRNSSGTIELVKKGCWLDDENC | 247 |
| | YDRQECVATEENPQVYFCCCEGNFCNERFTHLPEAGGPEVTYEPPPTGGGGSGGGGSGGG | |
| | GSGGGGTHTCPPCPAPELLGGPSVFLFPPKPKDTLMISRTPEVTCVVVDVSHEDPEVKEN | |
| | WYVDGVEVHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESNGQPENNYKTTPP | |
| | VLDSDGSFFLYSKLTVDKSRWQQGNVFSCSVMHEALHNHYTQKSLSLSPG | |
| P1409 | ETRECIYYNANWEQERTNQSGLERCEGEQDKRWHCYASWRNSSGTIELVKKGCWLDDENC | 248 |
| | YDRQECVATEENPQVYFCCCEGNFCNERFTHLPEAGGPEVTYEPPPTGGGGSGGGGSGGG | |
| | GSGGGGTHTCPPCPAPELLGGPSVFLFPPKPKDTLMISRTPEVTCVVVDVSHEDPEVKEN | |
| | WYVDGVEVHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESNGQPENNYKTTPP | |
| | VLDSDGSFFLYSKLTVDKSRWQQGNVFSCSVMHEALHNHYTQKSLSLSPG | |
| P1410 | ETRECIYYNANWEQERTNQSGLERCEGEQDKRYHCYASWRNSSGTIELVKKGCWLDDFNC | 249 |
| | YDRQECVATEENPQVYFCCCEGNFCNERFTHLPEAGGPEVTYEPPPTGGGGSGGGGSGGG | |
| | GSGGGGTHTCPPCPAPELLGGPSVFLFPPKPKDTLMISRTPEVTCVVVDVSHEDPEVKEN | |
| | WYVDGVEVHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESNGQPENNYKTTPP | |
| | VLDSDGSFFLYSKLTVDKSRWQQGNVFSCSVMHEALHNHYTQKSLSLSPG | |
| P1411 | ETRECIYYNANWEDERTNQSGLERCEGEQDKRYHCYASWRNSSGTIELVKKGCWLDDENC | 250 |
| | YDRQECVATEENPQVYFCCCEGNFCNERFTHLPEAGGPEVTYEPPPTGGGGSGGGGSGGG | |
| | GSGGGGTHTCPPCPAPELLGGPSVFLFPPKPKDTLMISRTPEVTCVVVDVSHEDPEVKEN | |
| | WYVDGVEVHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESNGQPENNYKTTPP | |
| | VLDSDGSFFLYSKLTVDKSRWQQGNVFSCSVMHEALHNHYTQKSLSLSPG | |
| P1412 | ETRECIYYNANWENERTNQSGLERCEGEQDKRYHCYASWRNSSGTIELVKKGCWLDDENC | 251 |
| | YDRQECVATEENPQVYFCCCEGNFCNERFTHLPEAGGPEVTYEPPPTGGGGSGGGGSGGG | |
| | GSGGGGTHTCPPCPAPELLGGPSVFLFPPKPKDTLMISRTPEVTCVVVDVSHEDPEVKEN | |
| | WYVDGVEVHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESNGQPENNYKTTPP | |
| | VLDSDGSFFLYSKLTVDKSRWQQGNVFSCSVMHEALHNHYTQKSLSLSPG | |

*Bold and italicized text indicates the linker sequence; Bold text indicates the N-terminal extension amino acids

In some embodiments, the binding agent comprises, from N-terminus to C-terminus, an ActRIIB ECD, a peptide linker, and an Fc domain. In some embodiments, the ActRIIB ECD comprises one or more amino acid substitutions. In some embodiments, the one or more amino acid substitutions are substitutions at a position selected from L14, G27, L33, L55, and L69, wherein the amino acid numbering is based on SEQ ID NO: 2. In some embodiments, the amino acid substitution at position L14 is selected from L14E, L14H, L14S, L14N, L14Q, and L14D. In some embodiments, the amino acid substitution at position G27 is selected from G27E, G27D, G27N, G27Q, G27Q, G27K, G27T, and G27M. In some embodiments, the amino acid substitution at position L33 is selected from L33R, L33Y, L33F, L33Q, L33W, L33E, L33K, and L33M. In some embodiments, the amino acid substitution at position L55 is selected from L55Y, L55Q, L55M, and L55I. In some embodiments, the amino acid substitution at position T69 is selected from T69H, T69Q, T69E, T69R, T69Y, and T69W.

In some embodiments the TGFβ superfamily ligand binding agent is a homodimeric polypeptide, wherein each polypeptide chain comprises from N-terminus to C-terminus, an ActRIIB ECD comprising the amino acid substitution of G27E, a peptide linker that is 14aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 4, a peptide linker that is 14aa in length, and an IgG1-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 4, a peptide linker that is 14aa in length, and an IgG1-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 174 (P739). In some embodiments the TGFβ superfamily ligand binding agent is a homodimeric polypeptide, wherein each polypeptide chain comprises or consists of the amino acid sequence of SEQ ID NO: 174 (P739).

In some embodiments the TGFβ superfamily ligand binding agent is a homodimeric polypeptide, wherein each polypeptide chain comprises from N-terminus to C-terminus, an ActRIIB ECD comprising the amino acid substitution of L33R, a peptide linker that is 14aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 15, a peptide linker that is 14aa in length, and an IgG1-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 15, a peptide linker that is 14aa in length, and an IgG1-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 175 (P750). In some embodiments the TGFβ superfamily ligand binding agent is a homodimeric polypeptide, wherein each polypeptide chain comprises or consists of the amino acid sequence of SEQ ID NO: 175 (P750).

In some embodiments the TGFβ superfamily ligand binding agent is a homodimeric polypeptide, wherein each polypeptide chain comprises from N-terminus to C-terminus, an ActRIIB ECD comprising the amino acid substitution of L33Y, a peptide linker that is 14aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 12, a peptide linker that is 14aa in length, and an IgG1-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 12, a peptide linker that is 14aa in length, and an IgG1-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 176 (P751). In some embodiments, the TGFβ superfamily ligand binding agent is a homodimeric polypeptide, wherein each polypeptide chain comprises or consists of the amino acid sequence of SEQ ID NO: 176 (P751).

In some embodiments the TGFβ superfamily ligand binding agent is a homodimeric polypeptide, wherein each polypeptide chain comprises from N-terminus to C-terminus, an ActRIIB ECD comprising the amino acid substitution of T69H, a peptide linker that is 14aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 19, a peptide linker that is 14aa in length, and an IgG1-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 19, a peptide linker that is 14aa in length, and an IgG1-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 177 (P753). In some embodiments, the TGFβ superfamily ligand binding agent is a homodimeric polypeptide, wherein each polypeptide chain comprises or consists of the amino acid sequence of SEQ ID NO: 177 (P753).

In some embodiments the TGFβ superfamily ligand binding agent is a homodimeric polypeptide, wherein each polypeptide chain comprises from N-terminus to C-terminus, an ActRIIB ECD comprising the amino acid substitution of T69Q, a peptide linker that is 14aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 20, a peptide linker that is 14aa in length, and an IgG1-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 20, a peptide linker that is 14aa in length, and an IgG1-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 178 (P754). In some embodiments, the TGFβ superfamily ligand binding agent is a homodimeric polypeptide, wherein each polypeptide chain comprises or consists of the amino acid sequence of SEQ ID NO: 178 (P754).

In some embodiments the TGFβ superfamily ligand binding agent is a homodimeric polypeptide, wherein each polypeptide chain comprises from N-terminus to C-terminus, an ActRIIB ECD comprising the amino acid substitution of G27E, a peptide linker that is 3aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 4, a peptide linker that is 3aa in length, and an IgG1-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 4, a peptide linker that is 3aa in length, and an IgG1-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 179 (P765). In some embodiments, the TGFβ superfamily ligand binding agent is a homodimeric polypeptide, wherein each polypeptide chain comprises or consists of the amino acid sequence of SEQ ID NO: 179 (P765).

In some embodiments the TGFβ superfamily ligand binding agent is a homodimeric polypeptide, wherein each polypeptide chain comprises from N-terminus to C-terminus, an ActRIIB ECD comprising the amino acid substitution of L33Y, a peptide linker that is 3aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 12, a peptide linker that is 3aa in length, and an IgG1-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 12, a peptide linker that is 3aa in length, and an IgG1-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 180 (P777). In some embodiments, the TGFβ superfamily ligand binding agent is a homodimeric polypeptide, wherein each polypeptide chain comprises or consists of the amino acid sequence of SEQ ID NO: 180 (P777).

In some embodiments the TGFβ superfamily ligand binding agent is a homodimeric polypeptide, wherein each polypeptide chain comprises from N-terminus to C-terminus, an ActRIIB ECD comprising the amino acid substitution of T69H, a peptide linker that is 3aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 19, a peptide linker that is 3aa in length, and an IgG1-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 19, a peptide linker that is 3aa in length, and an IgG1-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 181 (P779). In some embodiments, the TGFβ superfamily ligand binding agent is a homodimeric polypeptide, wherein each polypeptide chain comprises or consists of the amino acid sequence of SEQ ID NO: 181 (P779).

In some embodiments the TGFβ superfamily ligand binding agent is a homodimeric polypeptide, wherein each polypeptide chain comprises from N-terminus to C-terminus, an ActRIIB ECD comprising the amino acid substitution of T69Q, a peptide linker that is 3aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 20, a peptide linker that is 3aa in length, and an IgG1-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 20, a peptide linker that is 3aa in length, and an IgG1-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 182 (P780). In some embodiments, the TGFβ superfamily ligand binding agent is a homodimeric polypeptide, wherein each polypeptide chain comprises or consists of the amino acid sequence of SEQ ID NO: 182 (P780).

In some embodiments the TGFβ superfamily ligand binding agent is a homodimeric polypeptide, wherein each polypeptide chain comprises from N-terminus to C-terminus, an ActRIIB ECD comprising the amino acid substitutions of L33Y and T69H, a peptide linker that is 3aa, 10aa, or 14aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 28, a peptide linker that is 3aa, 10aa, or 14aa in length, and an IgG1-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 28, a peptide linker that is 3aa, 10aa, or 14aa in length, and an IgG1-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 183 (P1171). In some embodiments, the TGFβ superfamily ligand binding agent is a homodimeric polypeptide, wherein each polypeptide chain comprises or consists of the amino acid sequence of SEQ ID NO: 183 (P1171). In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 184 (P1172). In some embodiments, the TGFβ superfamily ligand binding agent is a homodimeric polypeptide, wherein each polypeptide chain comprises or consists of the amino acid sequence of SEQ ID NO: 184 (P1172). In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 185 (P1173). In some embodiments, the TGFβ superfamily ligand binding agent is a homodimeric polypeptide, wherein each polypeptide chain comprises or consists of the amino acid sequence of SEQ ID NO: 185 (P1173).

In some embodiments the TGFβ superfamily ligand binding agent is a homodimeric polypeptide, wherein each polypeptide chain comprises from N-terminus to C-terminus, an ActRIIB ECD comprising the amino acid substitutions of L33Y and T69Q, a peptide linker that is 3aa, 10aa, or 14aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 29, a peptide linker that is 3aa, 10aa, or 14aa in length, and an IgG1-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 29, a peptide linker that is 3aa, 10aa, or 14aa in length, and an IgG1-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 186 (P1174). In some embodiments, the TGFβ superfamily ligand binding agent is a homodimeric polypeptide, wherein each polypeptide chain comprises or consists of the amino acid sequence of SEQ ID NO: 186 (P1174). In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 187 (P1175). In some embodiments, the TGFβ superfamily ligand binding agent is a homodimeric polypeptide, wherein each polypeptide chain comprises or consists of the amino acid sequence of SEQ ID NO: 187 (P1175). In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 188 (P1176). In some embodiments, the TGFβ superfamily ligand binding agent is a homodimeric polypeptide, wherein each polypeptide chain comprises or consists of the amino acid sequence of SEQ ID NO: 188 (P1176).

In some embodiments the TGFβ superfamily ligand binding agent is a homodimeric polypeptide, wherein each polypeptide chain comprises from N-terminus to C-terminus, an ActRIIB ECD comprising the amino acid substitutions of L33Y and T69E, a peptide linker that is 3aa, 10aa, or 14aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 30, a peptide linker that is 3aa, 10aa, or 14aa in length, and an IgG1-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 30, a peptide linker that is 3aa, 10aa, or 14aa in length, and an IgG1-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 189 (P1177). In some embodiments, the TGFβ superfamily ligand binding agent is a homodimeric polypeptide, wherein each polypeptide chain comprises or consists of the amino acid sequence of SEQ ID NO: 189 (P1177). In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 190 (P1178). In some embodiments, the TGFβ superfamily ligand binding agent is a homodimeric polypeptide, wherein each polypeptide chain comprises or consists of the amino acid sequence of SEQ ID NO: 190 (P1178). In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 191 (P1179). In some embodiments, the TGFβ superfamily ligand binding agent is a homodimeric polypeptide, wherein each polypeptide chain comprises or consists of the amino acid sequence of SEQ ID NO: 191 (P1179).

In some embodiments the TGFβ superfamily ligand binding agent is a homodimeric polypeptide, wherein each polypeptide chain comprises from N-terminus to C-terminus, an ActRIIB ECD comprising the amino acid substitutions of L33Y and G27D, a peptide linker that is 3aa, 10aa, or 14aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 33, a peptide linker that is 3aa, 10aa, or 14aa in length, and an IgG1-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 33, a peptide linker that is 3aa, 10aa, or 14aa in length, and an IgG1-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 192 (P1180). In some embodiments, the TGFβ superfamily ligand binding agent is a homodimeric polypeptide, wherein each polypeptide chain comprises or consists of the amino acid sequence of SEQ ID NO: 192 (P1180). In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 193 (P1181). In some embodiments, the TGFβ superfamily ligand binding agent is a homodimeric polypeptide, wherein each polypeptide chain comprises or consists of the amino acid sequence of SEQ ID NO: 193 (P1181). In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 194 (P1182). In some embodiments, the TGFβ superfamily ligand binding agent is a homodimeric polypeptide, wherein each polypeptide chain comprises or consists of the amino acid sequence of SEQ ID NO: 194 (P1182).

In some embodiments the TGFβ superfamily ligand binding agent is a homodimeric polypeptide, wherein each polypeptide chain comprises from N-terminus to C-terminus, an ActRIIB ECD comprising the amino acid substitutions of L33Y and G27E, a peptide linker that is 3aa, 10aa, or 14aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 32, a peptide linker that is 3aa, 10aa, or 14aa in length, and an IgG1-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 32, a peptide linker that is 3aa, 10aa, or 14aa in length, and an IgG1-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 195 (P1183). In some embodiments, the TGFβ superfamily ligand binding agent is a homodimeric polypeptide, wherein each polypeptide chain comprises or consists of the amino acid sequence of SEQ ID NO: 195 (P1183). In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 196 (P1184). In some embodiments, the TGFβ superfamily ligand binding agent is a homodimeric polypeptide, wherein each polypeptide chain comprises or consists of the amino acid sequence of SEQ ID NO: 196 (P1184). In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 197 (P1185). In some embodiments, the TGFβ superfamily ligand binding agent is a homodimeric polypeptide, wherein each polypeptide chain comprises or consists of the amino acid sequence of SEQ ID NO: 197 (P1185).

In some embodiments the TGFβ superfamily ligand binding agent is a homodimeric polypeptide, wherein each polypeptide chain comprises from N-terminus to C-terminus, an ActRIIB ECD comprising the amino acid substitutions of L33F and T69Q, a peptide linker that is 3aa, 10aa, or 14aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 31, a peptide linker that is 3aa, 10aa, or 14aa in length, and an IgG1-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 31, a peptide linker that is 3aa, 10aa, or 14aa in length, and an IgG1-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 198 (P1186). In some embodiments, the TGFβ superfamily ligand binding agent is a homodimeric polypeptide, wherein each polypeptide chain comprises or consists of the amino acid sequence of SEQ ID NO: 198 (P1186). In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 199 (P1187). In some embodiments, the TGFβ superfamily ligand binding agent is a homodimeric polypeptide, wherein each polypeptide chain comprises or consists of the amino acid sequence of SEQ ID NO: 199 (P1187). In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 200 (P1188). In some embodiments, the TGFβ superfamily ligand binding agent is a homodimeric polypeptide, wherein each polypeptide chain comprises or consists of the amino acid sequence of SEQ ID NO: 200 (P1188).

In some embodiments the TGFβ superfamily ligand binding agent is a homodimeric polypeptide, wherein each polypeptide chain comprises from N-terminus to C-terminus, an ActRIIB ECD comprising the amino acid substitution of T69E, a peptide linker that is 3aa, 10aa, or 14aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 21, a peptide linker that is 3aa, 10aa, or 14aa in length, and an IgG1-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 21, a peptide linker that is 3aa, 10aa, or 14aa in length, and an IgG1-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 201 (P1201). In some embodiments, the TGFβ superfamily ligand binding agent is a homodimeric polypeptide, wherein each polypeptide chain comprises or consists of the amino acid sequence of SEQ ID NO: 201 (P1201). In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 202 (P1202). In some embodiments, the TGFβ superfamily ligand binding agent is a homodimeric polypeptide, wherein each polypeptide chain comprises or consists of the amino acid sequence of SEQ ID NO: 202 (P1202). In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 203 (P1203). In some embodiments, the TGFβ superfamily ligand binding agent is a homodimeric polypeptide, wherein each polypeptide chain comprises or consists of the amino acid sequence of SEQ ID NO: 203 (P1203).

In some embodiments the TGFβ superfamily ligand binding agent is a homodimeric polypeptide, wherein each polypeptide chain comprises from N-terminus to C-terminus, an ActRIIB ECD comprising the amino acid substitution of L33F, a peptide linker that is 3aa, 10aa, or 14aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 10, a peptide linker that is 3aa, 10aa, or 14aa in length, and an IgG1-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 10, a peptide linker that is 3aa, 10aa, or 14aa in length, and an IgG1-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 204 (P1204). In some embodiments, the TGFβ superfamily ligand binding agent is a homodimeric polypeptide, wherein each polypeptide chain comprises or consists of the amino acid sequence of SEQ ID NO: 204 (P1204). In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 205 (P1205). In some embodiments, the TGFβ superfamily ligand binding agent is a homodimeric polypeptide, wherein each polypeptide chain comprises or consists of the amino acid sequence of SEQ ID NO: 205 (P1205). In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 206 (P1206). In some embodiments, the TGFβ superfamily ligand binding agent is a homodimeric polypeptide, wherein each polypeptide chain comprises or consists of the amino acid sequence of SEQ ID NO: 206 (P1206).

In some embodiments the TGFβ superfamily ligand binding agent is a homodimeric polypeptide, wherein each polypeptide chain comprises from N-terminus to C-terminus, an ActRIIB ECD comprising the amino acid substitution of L33Q, a peptide linker that is 3aa, 10aa, or 14aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 11, a peptide linker that is 3aa, 10aa, or 14aa in length, and an IgG1-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 11, a peptide linker that is 3aa, 10aa, or 14aa in length, and an IgG1-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 207 (P1207). In some embodiments, the TGFβ superfamily ligand binding agent is a homodimeric polypeptide, wherein each polypeptide chain comprises or consists of the amino acid sequence of SEQ ID NO: 207 (P1207). In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 208 (P1208). In some embodiments, the TGFβ superfamily ligand binding agent is a homodimeric polypeptide, wherein each polypeptide chain comprises or consists of the amino acid sequence of SEQ ID NO: 208 (P1208). In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 209 (P1209). In some embodiments, the TGFβ superfamily ligand binding agent is a homodimeric polypeptide, wherein each polypeptide chain comprises or consists of the amino acid sequence of SEQ ID NO: 209 (P1209).

In some embodiments the TGFβ superfamily ligand binding agent is a homodimeric polypeptide, wherein each polypeptide chain comprises from N-terminus to C-terminus, an ActRIIB ECD comprising the amino acid substitution of L33Y, a peptide linker that is 10aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 12, a peptide linker that is 10aa in length, and an IgG1-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 12, a peptide linker that is 10aa in length, and an IgG1-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 210 (P1210). In some embodiments, the TGFβ superfamily ligand binding agent is a homodimeric polypeptide, wherein each polypeptide chain comprises or consists of the amino acid sequence of SEQ ID NO: 210 (P1210).

In some embodiments the TGFβ superfamily ligand binding agent is a homodimeric polypeptide, wherein each polypeptide chain comprises from N-terminus to C-terminus, an ActRIIB ECD comprising the amino acid substitution of L33W, a peptide linker that is 14aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 13, a peptide linker that is 14aa in length, and an IgG1-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 13, a peptide linker that is 14aa in length, and an IgG1-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 211 (P1229). In some embodiments, the TGFβ superfamily ligand binding agent is a homodimeric polypeptide, wherein each polypeptide chain comprises or consists of the amino acid sequence of SEQ ID NO: 211 (P1229).

In some embodiments the TGFβ superfamily ligand binding agent is a homodimeric polypeptide, wherein each polypeptide chain comprises from N-terminus to C-terminus, an ActRIIB ECD comprising the amino acid substitution of L33E, a peptide linker that is 14aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 16, a peptide linker that is 14aa in length, and an IgG1-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 16, a peptide linker that is 14aa in length, and an IgG1-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 212 (P1230). In some embodiments, the TGFβ superfamily ligand binding agent is a homodimeric polypeptide, wherein each polypeptide chain comprises or consists of the amino acid sequence of SEQ ID NO: 212 (P1230).

In some embodiments the TGFβ superfamily ligand binding agent is a homodimeric polypeptide, wherein each polypeptide chain comprises from N-terminus to C-terminus, an ActRIIB ECD comprising the amino acid substitution of L33K, a peptide linker that is 14aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 17, a peptide linker that is 14aa in length, and an IgG1-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 17, a peptide linker that is 14aa in length, and an IgG1-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 213 (P1231). In some embodiments, the TGFβ superfamily ligand binding agent is a homodimeric polypeptide, wherein each polypeptide chain comprises or consists of the amino acid sequence of SEQ ID NO: 213 (P1231).

In some embodiments the TGFβ superfamily ligand binding agent is a homodimeric polypeptide, wherein each polypeptide chain comprises from N-terminus to C-terminus, an ActRIIB ECD comprising the amino acid substitution of L33M, a peptide linker that is 14aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 18, a peptide linker that is 14aa in length, and an IgG1-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 18, a peptide linker that is 14aa in length, and an IgG1-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 214 (P1232). In some embodiments, the TGFβ superfamily ligand binding agent is a homodimeric polypeptide, wherein each polypeptide chain comprises or consists of the amino acid sequence of SEQ ID NO: 214 (P1232).

In some embodiments the TGFβ superfamily ligand binding agent is a homodimeric polypeptide, wherein each polypeptide chain comprises from N-terminus to C-terminus, an ActRIIB ECD comprising the amino acid substitutions of L33Y and G27N, a peptide linker that is 14aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 34, a peptide linker that is 14aa in length, and an IgG1-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 34, a peptide linker that is 14aa in length, and an IgG1-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 215 (P1235). In some embodiments, the TGFβ superfamily ligand binding agent is a homodimeric polypeptide, wherein each polypeptide chain comprises or consists of the amino acid sequence of SEQ ID NO: 215 (P1235).

In some embodiments the TGFβ superfamily ligand binding agent is a homodimeric polypeptide, wherein each polypeptide chain comprises from N-terminus to C-terminus, an ActRIIB ECD comprising the amino acid substitutions of L33Y and G27Q, a peptide linker that is 14aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 35, a peptide linker that is 14aa in length, and an IgG1-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 35, a peptide linker that is 14aa in length, and an IgG1-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 216 (P1236). In some embodiments, the TGFβ superfamily ligand binding agent is a homodimeric polypeptide, wherein each polypeptide chain comprises or consists of the amino acid sequence of SEQ ID NO: 216 (P1236).

In some embodiments the TGFβ superfamily ligand binding agent is a homodimeric polypeptide, wherein each polypeptide chain comprises from N-terminus to C-terminus, an ActRIIB ECD comprising the amino acid substitutions of L33Y and G27K, a peptide linker that is 14aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 36, a peptide linker that is 14aa in length, and an IgG1-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 36, a peptide linker that is 14aa in length, and an IgG1-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 217 (P1237). In some embodiments, the TGFβ superfamily ligand binding agent is a homodimeric polypeptide, wherein each polypeptide chain comprises or consists of the amino acid sequence of SEQ ID NO: 217 (P1237).

In some embodiments the TGFβ superfamily ligand binding agent is a homodimeric polypeptide, wherein each polypeptide chain comprises from N-terminus to C-terminus, an ActRIIB ECD comprising the amino acid substitutions of L33Y and G27Y, a peptide linker that is 14aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 37, a peptide linker that is 14aa in length, and an IgG1-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 37, a peptide linker that is 14aa in length, and an IgG1-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 218 (P1238). In some embodiments, the TGFβ superfamily ligand binding agent is a homodimeric polypeptide, wherein each polypeptide chain comprises or consists of the amino acid sequence of SEQ ID NO: 218 (P1238).

In some embodiments the TGFβ superfamily ligand binding agent is a homodimeric polypeptide, wherein each polypeptide chain comprises from N-terminus to C-terminus, an ActRIIB ECD comprising the amino acid substitutions of L33Y and G27M, a peptide linker that is 14aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 38, a peptide linker that is 14aa in length, and an IgG1-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 38, a peptide linker that is 14aa in length, and an IgG1-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 219 (P1239). In some embodiments, the TGFβ superfamily ligand binding agent is a homodimeric polypeptide, wherein each polypeptide chain comprises or consists of the amino acid sequence of SEQ ID NO: 219 (P1239).

In some embodiments the TGFβ superfamily ligand binding agent is a homodimeric polypeptide, wherein each polypeptide chain comprises from N-terminus to C-terminus, an ActRIIB ECD comprising the amino acid substitutions of L33Y and T69R, a peptide linker that is 14aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 25, a peptide linker that is 14aa in length, and an IgG1-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 25, a peptide linker that is 14aa in length, and an IgG1-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 220 (P1240). In some embodiments, the TGFβ superfamily ligand binding agent is a homodimeric polypeptide, wherein each polypeptide chain comprises or consists of the amino acid sequence of SEQ ID NO: 220 (P1240).

In some embodiments the TGFβ superfamily ligand binding agent is a homodimeric polypeptide, wherein each polypeptide chain comprises from N-terminus to C-terminus, an ActRIIB ECD comprising the amino acid substitutions of L33Y and T69Y, a peptide linker that is 14aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 26, a peptide linker that is 14aa in length, and an IgG1-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 26, a peptide linker that is 14aa in length, and an IgG1-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 221 (P1241). In some embodiments, the TGFβ superfamily ligand binding agent is a homodimeric polypeptide, wherein each polypeptide chain comprises or consists of the amino acid sequence of SEQ ID NO: 221 (P1241).

In some embodiments the TGFβ superfamily ligand binding agent is a homodimeric polypeptide, wherein each polypeptide chain comprises from N-terminus to C-terminus, an ActRIIB ECD comprising the amino acid substitutions of L33Y and T69W, a peptide linker that is 14aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 27, a peptide linker that is 14aa in length, and an IgG1-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 27, a peptide linker that is 14aa in length, and an IgG1-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 222 (P1242). In some embodiments, the TGFβ superfamily ligand binding agent is a homodimeric polypeptide, wherein each polypeptide chain comprises or consists of the amino acid sequence of SEQ ID NO: 222 (P1242).

In some embodiments the TGFβ superfamily ligand binding agent is a homodimeric polypeptide, wherein each polypeptide chain comprises from N-terminus to C-terminus, an ActRIIB ECD comprising the amino acid substitutions of L33Y and L14E, a peptide linker that is 14aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 52, a peptide linker that is 14aa in length, and an IgG1-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 52, a peptide linker that is 14aa in length, and an IgG1-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 223 (P1269). In some embodiments, the TGFβ superfamily ligand binding agent is a homodimeric polypeptide, wherein each polypeptide chain comprises or consists of the amino acid sequence of SEQ ID NO: 223 (P1269).

In some embodiments the TGFβ superfamily ligand binding agent is a homodimeric polypeptide, wherein each polypeptide chain comprises from N-terminus to C-terminus, an ActRIIB ECD comprising the amino acid substitutions of L33Y and L14H, a peptide linker that is 14aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 53, a peptide linker that is 14aa in length, and an IgG1-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 53, a peptide linker that is 14aa in length, and an IgG1-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 224 (P1270). In some embodiments, the TGFβ superfamily ligand binding agent is a homodimeric polypeptide, wherein each polypeptide chain comprises or consists of the amino acid sequence of SEQ ID NO: 224 (P1270).

In some embodiments the TGFβ superfamily ligand binding agent is a homodimeric polypeptide, wherein each polypeptide chain comprises from N-terminus to C-terminus, an ActRIIB ECD comprising the amino acid substitutions of L33Y and L14S, a peptide linker that is 14aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 54, a peptide linker that is 14aa in length, and an IgG1-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 54, a peptide linker that is 14aa in length, and an IgG1-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 225 (P1271). In some embodiments, the TGFβ superfamily ligand binding agent is a homodimeric polypeptide, wherein each polypeptide chain comprises or consists of the amino acid sequence of SEQ ID NO: 225 (P1271).

In some embodiments the TGFβ superfamily ligand binding agent is a homodimeric polypeptide, wherein each polypeptide chain comprises from N-terminus to C-terminus, an ActRIIB ECD comprising the amino acid substitutions of L33Y and L55Y, a peptide linker that is 14aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 59, a peptide linker that is 14aa in length, and an IgG1-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 59, a peptide linker that is 14aa in length, and an IgG1-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 226 (P1272). In some embodiments, the TGFβ superfamily ligand binding agent is a homodimeric polypeptide, wherein each polypeptide chain comprises or consists of the amino acid sequence of SEQ ID NO: 226 (P1272).

In some embodiments the TGFβ superfamily ligand binding agent is a homodimeric polypeptide, wherein each polypeptide chain comprises from N-terminus to C-terminus, an ActRIIB ECD comprising the amino acid substitutions of L33Y and L55Q, a peptide linker that is 14aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 60, a peptide linker that is 14aa in length, and an IgG1-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 60, a peptide linker that is 14aa in length, and an IgG1-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 227 (P1273). In some embodiments, the TGFβ superfamily ligand binding agent is a homodimeric polypeptide, wherein each polypeptide chain comprises or consists of the amino acid sequence of SEQ ID NO: 227 (P1273).

In some embodiments the TGFβ superfamily ligand binding agent is a homodimeric polypeptide, wherein each polypeptide chain comprises from N-terminus to C-terminus, an ActRIIB ECD comprising the amino acid substitutions of L33Y and L55M, a peptide linker that is 14aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 61, a peptide linker that is 14aa in length, and an IgG1-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 61, a peptide linker that is 14aa in length, and an IgG1-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 228 (P1274). In some embodiments, the TGFβ superfamily ligand binding agent is a homodimeric polypeptide, wherein each polypeptide chain comprises or consists of the amino acid sequence of SEQ ID NO: 228 (P1274).

In some embodiments the TGFβ superfamily ligand binding agent is a homodimeric polypeptide, wherein each polypeptide chain comprises from N-terminus to C-terminus, an ActRIIB ECD comprising the amino acid substitutions of L33Y and L55I, a peptide linker that is 14aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 62, a peptide linker that is 14aa in length, and an IgG1-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 62, a peptide linker that is 14aa in length, and an IgG1-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 229 (P1275). In some embodiments, the TGFβ superfamily ligand binding agent is a homodimeric polypeptide, wherein each polypeptide chain comprises or consists of the amino acid sequence of SEQ ID NO: 229 (P1275).

In some embodiments the TGFβ superfamily ligand binding agent is a homodimeric polypeptide, wherein each polypeptide chain comprises from N-terminus to C-terminus, an ActRIIB ECD comprising the amino acid substitution of L33W, a peptide linker that is 14aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 13, a peptide linker that is 14aa in length, and an IgG1 Y-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 13, a peptide linker that is 14aa in length, and an IgG1 Y-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 230 (P1371). In some embodiments, the TGFβ superfamily ligand binding agent is a homodimeric polypeptide, wherein each polypeptide chain comprises or consists of the amino acid sequence of SEQ ID NO: 230 (P1371).

In some embodiments the TGFβ superfamily ligand binding agent is a homodimeric polypeptide, wherein each polypeptide chain comprises from N-terminus to C-terminus, an ActRIIB ECD comprising the amino acid substitution of L33W, a peptide linker that is 14aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 13, a peptide linker that is 14aa in length, and an IgG1 YTE-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 13, a peptide linker that is 14aa in length, and an IgG1 YTE-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 231 (P1372). In some embodiments, the TGFβ superfamily ligand binding agent is a homodimeric polypeptide, wherein each polypeptide chain comprises or consists of the amino acid sequence of SEQ ID NO: 231 (P1372).

In some embodiments the TGFβ superfamily ligand binding agent is a homodimeric polypeptide, wherein each polypeptide chain comprises from N-terminus to C-terminus, an ActRIIB ECD comprising the amino acid substitution of L33W, a peptide linker that is 19aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 13, a peptide linker that is 19aa in length, and an IgG1 DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 13, a peptide linker that is 19aa in length, and an IgG1 DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 232 (P1373). In some embodiments, the TGFβ superfamily ligand binding agent is a homodimeric polypeptide, wherein each polypeptide chain comprises or consists of the amino acid sequence of SEQ ID NO: 232 (P1373).

In some embodiments the TGFβ superfamily ligand binding agent is a homodimeric polypeptide, wherein each polypeptide chain comprises from N-terminus to C-terminus, an ActRIIB ECD comprising the amino acid substitution of L33W, a peptide linker that is 19aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 13, a peptide linker that is 19aa in length, and an IgG1 Y-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 13, a peptide linker that is 19aa in length, and an IgG1 Y-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 233 (P1374). In some embodiments, the TGFβ superfamily ligand binding agent is a homodimeric polypeptide, wherein each polypeptide chain comprises or consists of the amino acid sequence of SEQ ID NO: 233 (P1374).

In some embodiments the TGFβ superfamily ligand binding agent is a homodimeric polypeptide, wherein each polypeptide chain comprises from N-terminus to C-terminus, an ActRIIB ECD comprising the amino acid substitution of L33W, a peptide linker that is 19aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 13, a peptide linker that is 19aa in length, and an IgG1 YTE-DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 13, a peptide linker that is 19aa in length, and an IgG1 YTE-DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 234 (P1375). In some embodiments, the TGFβ superfamily ligand binding agent is a homodimeric polypeptide, wherein each polypeptide chain comprises or consists of the amino acid sequence of SEQ ID NO: 234 (P1375).

In some embodiments the TGFβ superfamily ligand binding agent is a homodimeric polypeptide, wherein each polypeptide chain comprises from N-terminus to C-terminus, an ActRIIB ECD comprising the amino acid substitutions of G27D and L33W, a peptide linker that is 19aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 43, a peptide linker that is 19aa in length, and an IgG1 DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 43, a peptide linker that is 19aa in length, and an IgG1 DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 235 (P1385). In some embodiments, the TGFβ superfamily ligand binding agent is a homodimeric polypeptide, wherein each polypeptide chain comprises or consists of the amino acid sequence of SEQ ID NO: 235 (P1385).

In some embodiments the TGFβ superfamily ligand binding agent is a homodimeric polypeptide, wherein each polypeptide chain comprises from N-terminus to C-terminus, an ActRIIB ECD comprising the amino acid substitutions of G27E and L33W, a peptide linker that is 19aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 44, a peptide linker that is 19aa in length, and an IgG1 DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 44, a peptide linker that is 19aa in length, and an IgG1 DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 236 (P1386). In some embodiments, the TGFβ superfamily ligand binding agent is a homodimeric polypeptide, wherein each polypeptide chain comprises or consists of the amino acid sequence of SEQ ID NO: 236 (P1386).

In some embodiments the TGFβ superfamily ligand binding agent is a homodimeric polypeptide, wherein each polypeptide chain comprises from N-terminus to C-terminus, an ActRIIB ECD comprising the amino acid substitutions of G27N and L33W, a peptide linker that is 19aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 45, a peptide linker that is 19aa in length, and an IgG1 DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 45, a peptide linker that is 19aa in length, and an IgG1 DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 237 (P1387). In some embodiments, the TGFβ superfamily ligand binding agent is a homodimeric polypeptide, wherein each polypeptide chain comprises or consists of the amino acid sequence of SEQ ID NO: 237 (P1387).

In some embodiments the TGFβ superfamily ligand binding agent is a homodimeric polypeptide, wherein each polypeptide chain comprises from N-terminus to C-terminus, an ActRIIB ECD comprising the amino acid substitutions of G27Q and L33W, a peptide linker that is 19aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 46, a peptide linker that is 19aa in length, and an IgG1 DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 46, a peptide linker that is 19aa in length, and an IgG1 DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 238 (P1388). In some embodiments, the TGFβ superfamily ligand binding agent is a homodimeric polypeptide, wherein each polypeptide chain comprises or consists of the amino acid sequence of SEQ ID NO: 238 (P1388).

In some embodiments the TGFβ superfamily ligand binding agent is a homodimeric polypeptide, wherein each polypeptide chain comprises from N-terminus to C-terminus, an ActRIIB ECD comprising the amino acid substitutions of G27T and L33W, a peptide linker that is 19aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 47, a peptide linker that is 19aa in length, and an IgG1 DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 47, a peptide linker that is 19aa in length, and an IgG1 DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 239 (P1389). In some embodiments, the TGFβ superfamily ligand binding agent is a homodimeric polypeptide, wherein each polypeptide chain comprises or consists of the amino acid sequence of SEQ ID NO: 239 (P1389).

In some embodiments the TGFβ superfamily ligand binding agent is a homodimeric polypeptide, wherein each polypeptide chain comprises from N-terminus to C-terminus, an ActRIIB ECD comprising the amino acid substitutions of G27M and L33W, a peptide linker that is 19aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 48, a peptide linker that is 19aa in length, and an IgG1 DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 48, a peptide linker that is 19aa in length, and an IgG1 DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 240 (P1390). In some embodiments, the TGFβ superfamily ligand binding agent is a homodimeric polypeptide, wherein each polypeptide chain comprises or consists of the amino acid sequence of SEQ ID NO: 240 (P1390).

In some embodiments the TGFβ superfamily ligand binding agent is a homodimeric polypeptide, wherein each polypeptide chain comprises from N-terminus to C-terminus, an ActRIIB ECD comprising the amino acid substitutions of G27D and L33Y, a peptide linker that is 19aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 39, a peptide linker that is 19aa in length, and an IgG1 DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 39, a peptide linker that is 19aa in length, and an IgG1 DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 241 (P1391). In some embodiments, the TGFβ superfamily ligand binding agent is a homodimeric polypeptide, wherein each polypeptide chain comprises or consists of the amino acid sequence of SEQ ID NO: 241 (P1391).

In some embodiments the TGFβ superfamily ligand binding agent is a homodimeric polypeptide, wherein each polypeptide chain comprises from N-terminus to C-terminus, an ActRIIB ECD comprising the amino acid substitutions of G27E and L33Y, a peptide linker that is 19aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 40, a peptide linker that is 19aa in length, and an IgG1 DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 40, a peptide linker that is 19aa in length, and an IgG1 DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 242 (P1392). In some embodiments, the TGFβ superfamily ligand binding agent is a homodimeric polypeptide, wherein each polypeptide chain comprises or consists of the amino acid sequence of SEQ ID NO: 242 (P1392).

In some embodiments the TGFβ superfamily ligand binding agent is a homodimeric polypeptide, wherein each polypeptide chain comprises from N-terminus to C-terminus, an ActRIIB ECD comprising the amino acid substitutions of G27N and L33Y, a peptide linker that is 19aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 41, a peptide linker that is 19aa in length, and an IgG1 DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 41, a peptide linker that is 19aa in length, and an IgG1 DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 243 (P1395). In some embodiments, the TGFβ superfamily ligand binding agent is a homodimeric polypeptide, wherein each polypeptide chain comprises or consists of the amino acid sequence of SEQ ID NO: 243 (P1395).

In some embodiments the TGFβ superfamily ligand binding agent is a homodimeric polypeptide, wherein each polypeptide chain comprises from N-terminus to C-terminus, an ActRIIB ECD comprising the amino acid substitutions of G27Q and L33Y, a peptide linker that is 19aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 42, a peptide linker that is 19aa in length, and an IgG1 DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 42, a peptide linker that is 19aa in length, and an IgG1 DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 244 (P1396). In some embodiments, the TGFβ superfamily ligand binding agent is a homodimeric polypeptide, wherein each polypeptide chain comprises or consists of the amino acid sequence of SEQ ID NO: 244 (P1396).

In some embodiments the TGFβ superfamily ligand binding agent is a homodimeric polypeptide, wherein each polypeptide chain comprises from N-terminus to C-terminus, an ActRIIB ECD comprising the amino acid substitutions of L14E and L33W, a peptide linker that is 19aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 55, a peptide linker that is 19aa in length, and an IgG1 DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 55, a peptide linker that is 19aa in length, and an IgG1 DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 245 (P1406). In some embodiments, the TGFβ superfamily ligand binding agent is a homodimeric polypeptide, wherein each polypeptide chain comprises or consists of the amino acid sequence of SEQ ID NO: 245 (P1406).

In some embodiments the TGFβ superfamily ligand binding agent is a homodimeric polypeptide, wherein each polypeptide chain comprises from N-terminus to C-terminus, an ActRIIB ECD comprising the amino acid substitutions of L14D and L33W, a peptide linker that is 19aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 56, a peptide linker that is 19aa in length, and an IgG1 DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 56, a peptide linker that is 19aa in length, and an IgG1 DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 246 (P1407). In some embodiments, the TGFβ superfamily ligand binding agent is a homodimeric polypeptide, wherein each polypeptide chain comprises or consists of the amino acid sequence of SEQ ID NO: 246 (P1407).

In some embodiments the TGFβ superfamily ligand binding agent is a homodimeric polypeptide, wherein each polypeptide chain comprises from N-terminus to C-terminus, an ActRIIB ECD comprising the amino acid substitutions of L14N and L33W, a peptide linker that is 19aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 57, a peptide linker that is 19aa in length, and an IgG1 DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 57, a peptide linker that is 19aa in length, and an IgG1 DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 247 (P1408). In some embodiments, the TGFβ superfamily ligand binding agent is a homodimeric polypeptide, wherein each polypeptide chain comprises or consists of the amino acid sequence of SEQ ID NO: 247 (P1408).

In some embodiments the TGFβ superfamily ligand binding agent is a homodimeric polypeptide, wherein each polypeptide chain comprises from N-terminus to C-terminus, an ActRIIB ECD comprising the amino acid substitutions of L14Q and L33W, a peptide linker that is 19aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 58, a peptide linker that is 19aa in length, and an IgG1 DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 58, a peptide linker that is 19aa in length, and an IgG1 DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 248 (P1409). In some embodiments, the TGFβ superfamily ligand binding agent is a homodimeric polypeptide, wherein each polypeptide chain comprises or consists of the amino acid sequence of SEQ ID NO: 248 (P1409).

In some embodiments the TGFβ superfamily ligand binding agent is a homodimeric polypeptide, wherein each polypeptide chain comprises from N-terminus to C-terminus, an ActRIIB ECD comprising the amino acid substitutions of L14Q and L33Y, a peptide linker that is 19aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 49, a peptide linker that is 19aa in length, and an IgG1 DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 49, a peptide linker that is 19aa in length, and an IgG1 DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 249 (P1410). In some embodiments, the TGFβ superfamily ligand binding agent is a homodimeric polypeptide, wherein each polypeptide chain comprises or consists of the amino acid sequence of SEQ ID NO: 249 (P1410).

In some embodiments the TGFβ superfamily ligand binding agent is a homodimeric polypeptide, wherein each polypeptide chain comprises from N-terminus to C-terminus, an ActRIIB ECD comprising the amino acid substitutions of L14D and L33Y, a peptide linker that is 19aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 50, a peptide linker that is 19aa in length, and an IgG1 DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 50, a peptide linker that is 19aa in length, and an IgG1 DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 250 (P1411). In some embodiments, the TGFβ superfamily ligand binding agent is a homodimeric polypeptide, wherein each polypeptide chain comprises or consists of the amino acid sequence of SEQ ID NO: 250 (P1411).

In some embodiments the TGFβ superfamily ligand binding agent is a homodimeric polypeptide, wherein each polypeptide chain comprises from N-terminus to C-terminus, an ActRIIB ECD comprising the amino acid substitutions of L14N and L33Y, a peptide linker that is 19aa in length, and an IgG1 Fc domain monomer. In some embodiments, the TGFβ superfamily ligand binding agent is a homodimeric polypeptide, wherein each polypeptide chain comprises from N-terminus to C-terminus, an ActRIIB ECD comprising an amino acid sequence that is at least 95%, 96%, 97%, 98%, or 99% identical to SEQ ID NO: 51, a peptide linker that is 19aa in length, and an IgG1 DL Fc domain monomer. In some embodiments, the TGFβ superfamily ligand binding agent is a homodimeric polypeptide, wherein each polypeptide chain comprises from N-terminus to C-terminus, an ActRIIB ECD comprising or consisting of the amino acid sequence of SEQ ID NO: 51, a peptide linker that is 19aa in length, and an IgG1 DL Fc domain monomer. In some embodiments, the TGFβ superfamily ligand binding agent is a homodimeric polypeptide, wherein each polypeptide chain comprises an amino acid sequence that is at least 95%, 96%, 97%, 98%, or 99% identical to SEQ ID NO: 251 (P1412). In some embodiments, the TGFβ superfamily ligand binding agent is a homodimeric polypeptide, wherein each polypeptide chain comprises or consists of the amino acid sequence of SEQ ID NO: 251 (P1412).

In some embodiments, the polypeptides or binding agents of the disclosure are “isolated” or “substantially pure”. “Isolated” or “substantially pure”, when used to describe the polypeptides or binding agents disclosed herein, means a polypeptide or binding agent that has been identified, separated and/or recovered from a component of its production environment. Preferably, the polypeptide or binding agent is free or substantially free of association with all other components from its production environment. Contaminant components of its production environment, such as that resulting from recombinant transfected cells, are materials that would typically interfere with diagnostic or therapeutic uses for the polypeptide, and may include enzymes, hormones, and other proteinaceous or non-proteinaceous solutes. The polypeptides or binding agents may, e.g., constitute at least about 5%, or at least about 50% by weight of the total protein in a given sample. It is understood that the isolated protein may constitute from 5% to 99.9% by weight of the total protein content, depending on the circumstances. The polypeptide or binding agent may be made at a significantly higher concentration through the use of an inducible promoter or high expression promoter, such that it is made at increased concentration levels. The definition includes the production of a polypeptide or binding agent in a wide variety of organisms and/or host cells that are known in the art. In preferred embodiments, the polypeptide or binding agent will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Ordinarily, however, an isolated polypeptide or binding agent will be prepared by at least one purification step, such as, for example and without limitation, affinity and/or ion-exchange chromatography, e.g., binding to a Protein A column.

In some embodiments, binding agents of the present disclosure are characterized, for example, by one or more of the following: a particularly high affinity for one or more of activin A, activin B, GDF-8, and GDF-11; high neutralization potency (low IC50 values) for one or more of activin A, activin B, GDF-8, and GDF-11; a particularly low or undetectable affinity for BMP-9 and/or BMP-10; low or not detectable neutralization potency (high IC50 value) for BMP-9 and/or BMP-10; high thermostability; high plasma stability; long or extended half-life, low turbidity; high protein homogeneity; and/or high manufacturability.

The biological activity of a polypeptide or binding agent, or pharmaceutical composition thereof, of the disclosure can be determined for instance by cellular neutralization assays, binding assays, competition assays and the like. “Efficacy” or “in vivo efficacy” as used herein refers to the response to therapy using a combination described herein or composition thereof. The success or in vivo efficacy of the therapy using a combination described herein or composition thereof refers to the effectiveness of the combination or composition for its intended purpose, e.g., the ability of the combination described herein or composition thereof to cause its desired effect, i.e. treatment, amelioration, or prevention of a metabolic disorder. The in vivo efficacy may be monitored by established standard methods for the respective disease entities. In addition, various disease specific clinical chemistry parameters and other established standard methods may be used.

Another major challenge in the development of drugs such as a pharmaceutical composition of the disclosure is the predictable modulation of pharmacokinetic properties. To this end, a pharmacokinetic profile of the drug candidate, i.e., a profile of the pharmacokinetic parameters that affect the ability of a particular drug to treat a given condition, can be established. Pharmacokinetic parameters of the drug influencing the ability of a drug for treating a certain disease entity include, but are not limited to: half-life, volume of distribution, hepatic first-pass metabolism and the degree of blood serum binding. The efficacy of a given drug agent can be influenced by each of the parameters mentioned above.

Pharmacokinetic parameters also include bioavailability, lag time (Tlag), Tmax, absorption rates, more onset and/or Cmax for a given amount of drug administered. “Bioavailability” means the amount of a drug in the blood compartment. “Lag time” means the time delay between the administration of the drug and its detection and measurability in blood or plasma. “Tmax” is the time after which maximal blood concentration of the drug is reached, and “Cmax” is the blood concentration maximally obtained with a given drug. The time to reach a blood or tissue concentration of the drug which is required for its biological effect is influenced by all parameters.

In some embodiments, the binding agent of the present disclosure has a half-life of about 3 days or longer, about 5 days or longer, about 1 week or longer, about 2 weeks or longer, about 3 weeks or longer, about 4 weeks or longer, about 5 weeks or longer, about 6 weeks or longer, or about two months or longer.

In some embodiments, the binding agent of the present disclosure may show a favorable thermostability with aggregation temperatures of about 45° C. or higher, about 45 to about 50° C., about 52-about 54° C., about 56 to about 60° C., or about 60° C. or higher. The thermostability parameter can be determined in terms of polypeptide aggregation temperature as follows: Protein solution at a test concentration (e.g., 100 g/ml, 250 g/ml) is transferred into a single use cuvette and placed in a Dynamic Light Scattering (DLS) device. The sample is heated from 40° C. to 70° C. at a heating rate of 0.5° C./min with constant acquisition of the measured radius. Increase of radius indicating melting of the protein and aggregation is used to calculate the aggregation temperature of the polypeptide. Other methods known in the art may be used.

In an embodiment the binding agent according to the disclosure is stable at 2-8° C. for at least 1 month, 2 months, or 3 months. In an embodiment the polypeptide or binding agent according to the disclosure is stable at 25-40° C. for at least 4 weeks. In an embodiment the binding agent according to the disclosure is stable after undergoing 3 Freeze/Thaw cycles. In an embodiment the binding agent according to the disclosure is stable at −20° C. for 1 month, 2 month, 3 months or longer.

Alternatively, temperature melting curves can be determined by Differential Scanning Calorimetry (DSC) to determine intrinsic biophysical protein stabilities of the polypeptides or binding agents. These experiments may be performed using a MicroCal LLC (Northampton, Mass., U.S.A) VP-DSC device. The energy uptake of a sample containing a binding agent is recorded from 20° C. to 90° C. compared to a sample containing only the formulation buffer. For recording of the respective melting curve, the overall sample temperature is increased stepwise. At each temperature T energy uptake of the sample and the formulation buffer reference is recorded. The difference in energy uptake Cp (kcal/mole/° C.) of the sample minus the reference is plotted against the respective temperature. The melting temperature is defined as the temperature at the first maximum of energy uptake.

In a further embodiment, binding agents according to the disclosure are stable at acidic pH. The more tolerant the binding agent behaves at unphysiologic pH such as pH 5.5 (a pH which is required to run e.g. a cation exchange chromatography), the higher is the recovery of binding agent eluted from an ion exchange column relative to the total amount of loaded protein. Recovery of binding agents from an ion (e.g., cation) exchange column at pH 5.5 may be 50% or more, 60% or more, 65% or more, 70% or more, 72% or more, 74% or more, 76% or more, 78% or more, 80% or more, 90% or more, 95% or more, or 99% or more.

### Amino Acid Sequence Modifications

Amino acid sequence modifications of the binding agents described herein are contemplated. For example, it may be desirable to improve the binding affinity, effector functions, half-life and/or other biological properties of the binding agent. Amino acid sequence modification/variants of the binding agents are generally prepared by introducing appropriate nucleotide changes into the encoding nucleic acid, or by peptide synthesis. All of the below described amino acid sequence modifications should result in a binding agent which still retains the desired biological activity (e.g., binding to one or more of activin A, activin B, GDF-8, GDF-11, without substantial binding to BMP-9 and BMP-10) of the unmodified parental molecule.

As used herein, the term “functionally equivalent” refers to modified sequences that have the same or substantially the same biological activity or function as the original sequence from which it is derived, e.g., no significant change in physiological, chemical, physico-chemical or functional properties compared to the original sequence. The term “substantially identical” refers to sequences that are functionally equivalent to the original or reference sequence and have a high degree of sequence identity thereto. Generally, a substantially identical sequence is at least about 90%, at least about 95%, at least about 96%, at least about 97%, at least about 98%, or at least about 99% identical to the original or reference sequence and has the same function. In some cases when referring to nucleic acid sequences, a substantially identical sequence hybridizes to the original sequence under high stringency conditions, for example at salt and temperature conditions substantially equivalent to 0.5×SSC to about 5×SSC and 65° C. for both hybridization and wash. In general, modified sequences that are substantially identical or functionally equivalent to sequences provided in accordance with the present disclosure are meant to be encompassed.

Amino acid modifications include, for example, deletions from, and/or insertions into, and/or substitutions of, residues within the amino acid sequences of the binding agents. Any combination of deletion, insertion, and substitution is made to arrive at the final construct, provided that the final construct possesses the desired characteristics. The amino acid changes also may alter post-translational processes of the binding agents, such as changing the number or position of glycosylation sites. In a particular embodiment, one or more amino acid is changed to alter a glycosylation site.

For example, 1, 2, 3, 4, 5, or 6 amino acids may be inserted or deleted in a binding agent. Preferably, amino acid sequence insertions include amino- and/or carboxyl-terminal fusions ranging in length from 1, 2, 3, 4, 5, 6, 7, 8, 9 or 10 residues to polypeptides containing a hundred or more residues, as well as intra-sequence insertions of single or multiple amino acid residues. An insertional variant of the polypeptides or binding agents of the disclosure includes fusion to the N-terminus or to the C-terminus of the polypeptides or binding agents of an enzyme or fusion to a polypeptide which increases the serum half-life of the binding agents.

It is envisioned that modifications of the binding agents described herein are also encompassed. Modifications encompassed by the present disclosure include those having a variation in the amino acid sequence of the polypeptides or binding agents. Modifications of the binding agents include, for example, those having similar or improved binding affinity, avidity, ligand specificity, potency of inhibition, stability, manufacturability, half-life, and/or reduced aggregation in comparison with the binding agents disclosed herein.

One site of interest for substitutional mutagenesis includes the Fc domain monomer, as described hereinabove. Exemplary embodiments of modified polypeptides or binding agents of the present disclosure may comprise those having a modified IgG1, IgG2, IgG3, or IgG4 constant region or a portion thereof. In an embodiment, the polypeptides or binding agents comprise an IgG1 constant region (modified or unmodified). In an embodiment, the polypeptides or binding agents comprise an IgG2 constant region (modified or unmodified). In an embodiment, the polypeptides or binding agents comprise an IgG3 constant region (modified or unmodified). In an embodiment, the polypeptides or binding agents comprise an IgG4 constant region (modified or unmodified).

Modifications encompassed by the present disclosure include those which may comprise an insertion, a deletion or an amino acid substitution (conservative or non-conservative). These modifications may have at least one amino acid residue in its amino acid sequence removed and a different residue inserted in its place. It should be understood that variation may occur in multiple regions of the binding agents, as long as the desired binding or biological activity is maintained.

It is known in the art that modifications and variants may be generated by substitutional mutagenesis and retain the biological activity (i.e., functional equivalence) of the binding agents of the present disclosure. These modifications or variants have at least one amino acid residue in the amino acid sequence removed and a different residue inserted in its place, e.g., one or more conservative amino acid substitution. In general, a conservative amino acid substitution is the substitution of an amino acid residue for another amino acid residue with similar chemical properties (e.g., size, charge, or polarity).

Generally, the degree of similarity and identity between variant polypeptide chains is determined herein using the Blast2 sequence program (Tatusova, T. A. and Madden, T. L., 1999) using default settings, i.e., blastp program, BLOSUM62 matrix (open gap 11 and extension gap penalty 1; gapx dropoff 50, expect 10.0, word size 3) and activated filters.

However, the level of identity may also be determined over the entire length of a given sequence. Percent identity will therefore be indicative of amino acids which are identical in comparison with the original peptide and which may occupy the same or similar position. Percent similarity will be indicative of amino acids which are identical and those which are replaced with conservative amino acid substitution in comparison with the original peptide at the same or similar position.

In some embodiments, modifications of the binding agents of the present disclosure therefore comprise amino acid sequences which have at least 90%, at least 91%, at least 92%, at least 93%, at least 94%, at least 95%, at least 96%, at least 97%, at least 98%, or at least 99% sequence identity with an original sequence or a portion of an original sequence.

In some embodiments, substitutions are conservative substitutions. However, any substitution (including non-conservative substitution) is envisaged as long as the binding agents retain their capability to bind and/or inhibit the desired TGFβ superfamily ligands, without substantially binding or inhibiting BMP-9 and/or BMP-10.

Generally, the nucleic acid sequence homology, similarity, or identity between the nucleotide sequences encoding binding agents of the disclosure and the nucleotide sequences depicted herein are at least 60%, and more typically with preferably increasing homologies or identities of at least 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%, and almost 100%.

### GLP-1 Agonists

In some embodiments, the present disclosure provides combinations comprising a TGFβ superfamily ligand binding agent and a glucagon-like peptide 1 (GLP-1) agonist. Herein, the term “GLP-1 agonist” refers to a class of agents that binds to and activates the GLP-1 receptor. GLP-1 receptor agonists may also be referred to as “incretin mimetics” or “GLP-1 receptor agonists”.

The glucagon-like peptide-1 receptor is a pancreatic beta cell receptor that stimulates insulin secretion upon binding of the hormone glucagon-like peptide 1 (GLP-1). GLP-1 is a type of incretin hormone, all of which regulate insulin release in response to blood sugar. GLP-1 agonists mimic the action of GLP-1 peptide and activate the GLP-1 receptor upon binding, stimulating insulin secretion. However, GLP-1 agonists exhibit significant side effects and tolerability issues at effective doses. GLP-1 agonists have also been shown to decrease lean mass of a subject during treatment. Warnings and precautions for GLP-1 agonists (e.g., semaglutide) include but are not limited to: thyroid C-cell tumors, acute pancreatitis, acute gallbladder disease, hypoglycemia, acute kidney injury, severe adverse gastrointestinal reactions, hypersensitivity, e.g., anaphylactic reactions and angioedema, diabetic retinopathy complications in subjects with Type 2 Diabetes, heart rate increase, and suicidal behavior and ideation. Adverse reactions reported in at least 5% of subjects treated with a GLP-1 agonist (e.g., semaglutide), include nausea, diarrhea, vomiting, constipation, abdominal pain, headache, fatigue, dyspepsia, dizziness, abdominal distension, eructation, hypoglycemia in patients with type 2 diabetes, flatulence, gastroenteritis, and gastroesophageal reflux disease. Thus, there is a need for optimized dosage and treatments with GLP-1 agonists.

As provided herein, the GLP-1 agonist of the disclosure may be a peptide, antibody, small molecule, or an aptamer. In some embodiments, the GLP-1 agonist is a peptide or peptide analog, including but not limited to: exenatide, exenatide extended-release, efpeglenatide, dulaglutide, liraglutide, lixisenatide, lotiglipron, semaglutide, tirzepatide, cotadutide, noiiglutide, oxyntomodulin, orforglipron, retatrutide, albiglutide, beinaglutide and PEG-loxenatide, pemvidutide, and danuglipron. In some embodiments, the GLP-1 agonist is a small molecule non-peptide agonist, e.g, danuglipron. In some embodiments, the GLP-1 agonist is semaglutide. In some embodiments, the GLP-1 agonist is tirzepatide. In some embodiments, the GLP-1 agonist is retatrutide.

In some embodiments, the GLP-1 agonist is a dual agonist that binds and activates or deactivates a second receptor in addition to a GLP-1 receptor, wherein the second receptor is glucose-dependent insulinotropic (GIP) receptor or a glucagon (GCG) receptor. An exemplary dual acting GLP-1 agonist that binds both GLP-1 receptor and GIP receptor is tirzepatide. Exemplary dual acting GLP-1 agonists that binds both GLP-1 receptor and GCG receptor are cotadutide, noiiglutide, and oxyntomodulin, and are included in this disclosure as possible therapeutic agents to be used in combination with the TGFβ superfamily ligand binding agent. In some embodiments, the GLP-1 agonist is also a GIP antagonist. In some embodiments, the GLP-1 agonist is also a GCG antagonist. In some embodiments, the GLP-1 agonist is a triagonist of GIP/GLP-1/glucagon receptor, e.g., a GGG triagonist, e.g., LY343794.

In some embodiments, the GLP-1 agonist is GLP-1(7-37); GLP-1(7-36)-NH₂; liraglutide; albiglutide; taspoglutide; dulaglutide; semaglutide; LY2428757; exendin-4; exendin-3; Leu¹⁴-exendin-4; Leu¹⁴,Phe²⁵-exendin-4; Leu¹⁴,Ala¹⁹,Phe²⁵-exendin-4; exendin-4(1-30); Leu¹⁴-exendin-4(1-30); Leu¹⁴,Phe²⁵-exendin-4(1-30); Leu¹⁴,Ala¹⁹,Phe²⁵-exendin-4(1-30); exendin-4(1-28); Leu¹⁴-exendin-4(1-28); Leu¹⁴,Phe²⁵-exendin-4(1-28); Leu¹⁴,Ala¹⁹,Phe²⁵-exendin-4(1-28); Leu¹⁴,Lys^{17,20},Ala¹⁹,Glu²¹,Phe⁵,Gln²⁸-exendin-4; Leu¹⁴,Lys^{17,20},Ala¹⁹,Glu²¹,Gln²⁸-exendin-4; octylGly¹⁴,Gln²⁸-exendin-4; Leu¹⁴,Gln²⁸,octylGly³⁴-exendin-4; Phe⁴,Leu¹⁴,Gln²⁸,Lys³³,Glu³⁴, Ile^{35,36},Ser³⁷-exendin-4(1-37); Phe⁴,Leu¹⁴,Lys^{17,20,35}, Ala¹⁹,Glu²¹,Gln28-exendin-4; Val¹¹,IIe¹³,Leu¹⁴Ala¹⁶,Lys²¹,Phe²⁵-exendin-4; exendin-4-Lys⁴⁰; GLP-1(7-37); GLP-1(7-36)-NH₂; Aib^{8,35},Arg^{26,34},Phe³¹-GLP-1(7-36); Arg³⁴-GLP-1(7-37); Glu³⁰-GLP-1(7-37); Lys²²-GLP-1(7-37); Gly^{8,36},Glu²²-GLP-1(7-37); Val⁸,Glu²²,Gly³⁶-GLP-1(7-37), Gly^{8,36}, Glu²²,Lys³³,Asn³⁴-GLP-1(7-37); Val^{8,50}, Glu²²,Lys³³, Asn³⁴,Gy³⁶-GLP-1(7-37);, Gly^{8,36},GIu²²,Pro³⁷-GLP-1(7-37); Val⁸, GIu²², Gly³⁶,Pro³⁷-GLP-1(7-37); Gly^{8,36} Gu²²,Lys³³, Asn³⁴,Pro³⁷-GLP-1(7-37); Val⁸,Glu²²,Lys³³,Asn³⁴ Gly³⁶,Pro³⁷-GLP-1(7-37); Gly^{8,36},Glu²²-GLP-1(7-36); Val⁸,Glu²²,Gy³⁶-GLP-1(7-36); Val⁸,Glu²²,Asn³⁴,Gy³⁶-GLP-1(7-36), Gly^{8,36},GIu²²,Asn³⁴-GLP-1(7-36); [W-(17-carboxyheptadecanoic acid)Lys20]exendin-4-NH₂; [W-(17-carboxyheptadecanoyl)Lys³²]exendin-4-NH₂; [desamino-His^{l},N^{e}-(17-carboxyheptadecanoyl)Lys²⁰]exendin-4-NH₂; [Arg^{12,27},NLe¹⁴,W-(17-carboxy-heptadecanoyl)Lys³²]exendin-4-NH₂; [W-(19-carboxy-nonadecanoylamino)Lys²⁰]-exendin-4-NH₂); [W-(15-carboxypentadecanoylamino)Lys²⁰]-exendin-4-NH₂; [W-(13-carboxytridecanoylamino)Lys²⁰]exendin-4-NH₂; [W-(11-carboxy-undecanoyl-amino)Lys ²⁰]exendin-4-NH₂; exendin-4-Lys⁴⁰(e-MPA)-NH₂; exendin-4-Lys⁴⁰(e-AEEA-AEEA-MPA)-NH₂; exendin-4-Lys⁴⁰(e-AEEA-MPA)-NH₂; exendin-4-Lys⁴⁰(e-MPA)-albumin; exendin-4-Lys⁴⁰(e-AEEA-AEEA-MPA)-albumin; exendin-4-Lys⁴⁰(e-AEEA-MPA)-albumin; desamino-His⁷,Arg²⁶,Lys³⁴(W-(yGlu(N-a-hexadecanoyl)))-GLP-1(7-37); desamino-His ⁷,Arg²⁶,Lys³⁴(N^{e}-octanoyl)-GLP-1(7-37); Arg^{26,34},Lys³⁸(Ne-(ω-carboxypentadecoyl))-GLP-1 (7-38); Arg^{26,34},Lys³⁶(N-(y-Glu(N-a-hexadecanoyl)))-GLP-1(7-36); [Aib⁸;Lys³⁷]GLP-1_(7-37); [Aib⁸, Lys²⁶]GLP-1_(7-37); [Aib^{8,22};Lys³⁶]GLP-1(7-36)-Amide; [Aib^{8,22}, BLeu³², Lys³⁶]GLP-1(7-36)-Amide; Aib^{8,22}, Lys³⁷]GLP-1(7-37)-Amide; [Aib^{8,22},BLeu³²,Lys³⁷]GLP-(7-37)-Amide; or a peptide having at least 80% sequence identity thereto. In some embodiments, the GLP-1 agonist is a peptide or analog thereof described in U.S. Pat. No. 10,905,772, which is incorporated herein by reference in its entirety.

### Pharmaceutical Compositions

In some embodiments, the individual components (e.g., the binding agents and GLP-1 agonists) of the combinations provided herein are formulated in separate pharmaceutical compositions. In some embodiments, the individual components (e.g., the binding agents and GLP-1 agonists) of the combinations provided herein are formulated in the same pharmaceutical composition. The pharmaceutical composition generally comprises the binding agent disclosed herein and/or a GLP-1 agonist and a pharmaceutically acceptable carrier.

The preparation of pharmaceutical compositions can be carried out as known in the art (see, for example, Remington: The Science and Practice of Pharmacy, 20th Edition, 2000). For example, a therapeutic compound and/or composition, together with one or more solid or liquid pharmaceutical carrier substances and/or additives (or auxiliary substances) and, if desired, in combination with other pharmaceutically active compounds having therapeutic or prophylactic action, are brought into a suitable administration form or dosage form which can then be used as a pharmaceutical in human or veterinary medicine. Pharmaceutical preparations can also contain additives, of which many are known in the art, for example fillers, disintegrants, binders, lubricants, wetting agents, stabilizers, emulsifiers, dispersants, preservatives, sweeteners, colorants, flavorings, aromatizers, thickeners, diluents, buffer substances, solvents, solubilizers, agents for achieving a depot effect, salts for altering the osmotic pressure, coating agents or antioxidants.

The term “pharmaceutical composition” means a composition comprising a binding agent and/or a GLP-1 agonist as described herein and at least one component comprising pharmaceutically acceptable carriers, diluents, adjuvants, excipients, or vehicles, such as preserving agents, fillers, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms.

The term “pharmaceutically acceptable carrier” is used to mean any carrier, diluent, adjuvant, excipient, or vehicle, as described herein or as known in the art. Examples of suspending agents include ethoxylated isostearyl alcohols, polyoxyethylene sorbitol and sorbitan esters, microcrystalline cellulose, aluminum metahydroxide, bentonite, agar-agar and tragacanth, or mixtures of these substances. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monosterate and gelatin. Non-limiting examples of suitable carriers, diluents, solvents, or vehicles include water, salt solutions, phosphate buffered saline (PBS), gelatins, oils, alcohols, polyols, suitable mixtures thereof, vegetable oils (such as olive oil), and injectable organic esters such as ethyl oleate. Non-limiting examples of excipients include lactose, milk sugar, sodium citrate, calcium carbonate, and dicalcium phosphate. Non-limiting examples of disintegrating agents include starch, alginic acids, and certain complex silicates. Non-limiting examples of lubricants include magnesium stearate, sodium lauryl sulphate, talc, as well as high molecular weight polyethylene glycols.

The term “pharmaceutically acceptable” means it is, within the scope of sound medical judgment, suitable for use in contact with the cells of a subject, e.g., humans and animals, without undue toxicity, irritation, allergic response, and the like, and are commensurate with a reasonable benefit/risk ratio.

Supplementary active compounds can also be incorporated into the compositions. For example, a pharmaceutical composition provided herein may further comprise at least one additional therapeutic agent, as discussed further below.

The pharmaceutical compositions described herein may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

In some embodiments, a pharmaceutical composition provided herein can be administered orally, for example in the form of pills, tablets, lacquered tablets, sugar-coated tablets, granules, hard and soft gelatin capsules, aqueous, alcoholic or oily solutions, syrups, emulsions or suspensions, or rectally, for example in the form of suppositories.

In other embodiments, a pharmaceutical composition provided herein can be administered parenterally, for example subcutaneously, intramuscularly or intravenously in the form of solutions for injection or infusion. Other suitable administration forms are, for example, percutaneous or topical administration, for example in the form of ointments, creams, tinctures, sprays or transdermal therapeutic systems, or the inhalative administration in the form of nasal sprays or aerosol mixtures, or, for example, microcapsules, implants or wafers.

Pharmaceutical compositions typically must be sterile and stable under the conditions of manufacture and storage. A composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of injectable compositions can be brought about by including in the composition an agent which delays absorption, for example, monostearate salts and gelatin. Moreover, a compound can be administered in a time release formulation, for example in a composition which includes a slow release polymer. The compound can be prepared with carriers that will protect against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG).

Many methods for the preparation of such formulations are generally known to those skilled in the art. Sterile injectable solutions can be prepared by incorporating an active compound, such as a binding agent provided herein, in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, common methods of preparation are vacuum drying and freeze-drying which yields a powder of the active ingredient plus any additional desired ingredient from a previously sterile-filtered solution thereof. Compounds may also be formulated with one or more additional compounds that enhance their solubility.

In some embodiments, a pharmaceutical composition of the disclosure comprises a sterile injectable solution. In some embodiments, the sterile injectable solution comprises a sterile powder which is reconstituted with an acceptable solution, e.g., water.

In some embodiments, the pharmaceutical compositions of the present disclosure comprises a binding agent and a GLP-1 agonist, NaPO4, glycine, and histidine at a pH of about 7.4-7.6. In some embodiments, the formulation does not comprise a surfactant (e.g., polysorbate 80).

In some embodiments, glycine is present at a concentration of about 10 mM to about 150 mM (e.g., 10, 15, 20, 25, 30, 35, 40, 45, 50, 55, 60, 65, 70, 75, 80, 85, 90, 95, 100, 105, 110, 115, 120, 125, 130, 135, 140, 145, and 150 mM). In some embodiments, glycine is present at a concentration of about 10 mM to about 125 mM, about 10 mM to about 100 mM, about 10 mM to about 75 mM, about 10 mM to about 50 mM, about 10 mM to about 25 mM, about 25 mM to about 150 mM, about 25 mM to about 125 mM, about 25 mM to about 100 mM, about 25 mM to about 75 mM, about 25 mM to about 50 mM, about 50 mM to about 150 mM, about 50 mM to about 125 mM, about 50 mM to about 100 mM, about 50 mM to about 75 mM, about 75 mM to about 150 mM, about 75 mM to about 125 mM, or about 75 mM to about 100 mM. In some embodiments, glycine is present at a concentration of about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM, about 50 mM, about 51 mM, about 52 mM, about 53 mM, about 54 mM, about 55 mM, about 56 mM, about 57 mM, about 58 mM, about 59 mM, about 60 mM, about 61 mM, about 62 mM, about 63 mM, about 64 mM, about 65 mM, about 66 mM, about 67 mM, about 68 mM, about 69 mM, about 70 mM, about 71 mM, about 72 mM, about 73 mM, about 74 mM, about 75 mM, about 76 mM, about 77 mM, about 78 mM, about 79 mM, about 80 mM, about 81 mM, about 82 mM, about 83 mM, about 84 mM, about 85 mM, about 86 mM, about 87 mM, about 88 mM, about 89 mM, about 90 mM, about 91 mM, about 92 mM, about 93 mM, about 94 mM, about 95 mM, about 96 mM, about 97 mM, about 98 mM, about 99 mM, about 100 mM, about 101 mM, about 102 mM, about 103 mM, about 104 mM, about 105 mM, about 106 mM, about 107 mM, about 108 mM, about 109 mM, about 110 mM, about 111 mM, about 112 mM, about 113 mM, about 114 mM, about 115 mM, about 116 mM, about 117 mM, about 118 mM, about 119 mM, about 120 mM, about 121 mM, about 122 mM, about 123 mM, about 124 mM, about 125 mM, about 126 mM, about 127 mM, about 128 mM, about 129 mM, about 130 mM, about 131 mM, about 132 mM, about 133 mM, about 134 mM, about 135 mM, about 136 mM, about 137 mM, about 138 mM, about 139 mM, about 140 mM, about 141 mM, about 142 mM, about 143 mM, about 144 mM, about 145 mM, about 146 mM, about 147 mM, about 148 mM, about 149 mM or about 150 mM, including all ranges, subranges and values therebetween. In some embodiments, glycine is present at 100 mM. In some embodiments, glycine is present at about 90 mM to about 110 mM, about 95 mM to about 110 mM, about 100 mM to about 110 mM, about 105 mM to about 110 mM, about 90 mM to about 105 mM, about 95 mM to about 105 mM, about 100 mM to about 105 mM, about 90 mM to about 100 mM, or about 95 mM to about 100 mM.

In some embodiments, the pharmaceutical composition comprises histidine. In some embodiments, histidine is present at a concentration of about 10 mM to about 175 mM (e.g., 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, and 175 mM). In some embodiments, histidine is present at a concentration of about 10 mM to about 150 mM, about 10 mM to about 125 mM, about 10 mM to about 100 mM, about 10 mM to about 75 mM, about 10 mM to about 50 mM, about 10 mM to about 25 mM, about 25 mM to about 150 mM, about 25 mM to about 125 mM, about 25 mM to about 100 mM, about 25 mM to about 75 mM, about 25 mM to about 50 mM, about 50 mM to about 150 mM, about 50 mM to about 125 mM, about 50 mM to about 100 mM, about 50 mM to about 75 mM, about 75 mM to about 175 mM, about 75 mM to about 150 mM, about 75 mM to about 125 mM, about 75 mM to about 100 mM, about 100 mM to about 175 mM, about 100 mM to about 150 mM, or about 100 mM to about 125 mM. In some embodiments, histidine is present at a concentration of about 125 mM to about 175 mM, about 125 mM to about 150 mM, or about 150 mM to about 175 mM. In some embodiments, histidine is present at a concentration about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM, 50 mM, about 51 mM, about 52 mM, about 53 mM, about 54 mM, about 55 mM, about 56 mM, about 57 mM, about 58 mM, about 59 mM, about 60 mM, about 61 mM, about 62 mM, about 63 mM, about 64 mM, about 65 mM, about 66 mM, about 67 mM, about 68 mM, about 69 mM, about 70 mM, about 71 mM, about 72 mM, about 73 mM, about 74 mM, about 75 mM, about 76 mM, about 77 mM, about 78 mM, about 79 mM, about 80 mM, about 81 mM, about 82 mM, about 83 mM, about 84 mM, about 85 mM, about 86 mM, about 87 mM, about 88 mM, about 89 mM, about 90 mM, about 91 mM, about 92 mM, about 93 mM, about 94 mM, about 95 mM, about 96 mM, about 97 mM, about 98 mM, about 99 mM, about 100 mM, about 101 mM, about 102 mM, about 103 mM, about 104 mM, about 105 mM, about 106 mM, about 107 mM, about 108 mM, about 109 mM, about 110 mM, about 111 mM, about 112 mM, about 113 mM, about 114 mM, about 115 mM, about 116 mM, about 117 mM, about 118 mM, about 119 mM, about 120 mM, about 121 mM, about 122 mM, about 123 mM, about 124 mM, about or 125 mM, including all ranges, subranges and values therebetween. In some embodiments, histidine is present at 75 mM. In some embodiments, histidine is present at about 65 mM to about 85 mM, about 70 mM to about 85 mM, about 75 mM to about 85 mM, about 80 mM to about 85 mM, about 65 mM to about 80 mM, about 70 mM to about 80 mM, about 75 mM to about 80 mM, about 65 mM to about 75 mM, or about 70 mM to about 75 mM.

In some embodiments, the pharmaceutical composition comprises NaPO₄. In some embodiments, NaPO₄ is present at a concentration of about 5 mM to about 30 mM (e.g., 5, 10, 15, 20, 25, or 30 mM). In some embodiments, NaPO₄ is present at a concentration of about 5 mM to about 25 mM, about 5 mM to about 20 mM, about 5 mM to about 15 mM, about 5 mM to about 10 mM, about 10 mM to about 3 mM, about 10 mM to about 25 mM, about 10 mM to about 20 mM, about 10 mM to about 15 mM, about 15 mM to about 30 mM, about 15 mM to about 25 mM, about 15 mM to about 20 mM, about 20 mM to about 30 mM, about 20 mM to about 25 mM, or about 25 mM to about 20 mM. In some embodiments, NaPO₄ is present at a concentration of about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, or about 30 mM. In some embodiments, NaPO₄ is present at a concentration of about 20 nM.

In some embodiments, the pharmaceutical compositions of the present disclosure comprises a binding agent and a GLP-1 agonist, about 5-30 mM NaPO4, about 90-110 mM glycine, and about 65-85 mM histidine at a pH of about 7.4-7.6. In some embodiments, the pharmaceutical composition does not comprise a surfactant (e.g., polysorbate 80). In some embodiments, the pharmaceutical compositions of the present disclosure comprises a binding agent and a GLP-1 agonist, about 20 mM NaPO4, about 100 mM glycine, and about 75 mM histidine at a pH of about 7.5. In some embodiments, the pharmaceutical composition does not comprise a surfactant (e.g., polysorbate 80). In some embodiments, the pharmaceutical compositions of the present disclosure comprises a binding agent and a GLP-1 agonist, about 10 mM NaPO4, about 100 mM glycine, and about 75 mM histidine at a pH of about 7.5. In some embodiments, the pharmaceutical composition does not comprise a surfactant (e.g., polysorbate 80).

It is often advantageous to formulate compositions (such as parenteral compositions) in dosage unit form for ease of administration and uniformity of dosage. The term “unit dosage form” refers to a physically discrete unit suitable as unitary dosages for human subjects and other animals, each unit containing a predetermined quantity of active material calculated to produce the desired therapeutic effect, in association with a suitable pharmaceutical carrier. The specification for the dosage unit forms of the invention may vary and are dictated by and directly dependent on (a) the unique characteristics of the therapeutic compound and the particular therapeutic effect to be achieved, and (b) the limitations inherent in the art of compounding such a therapeutic compound for the prevention or treatment of a TGFβ-superfamily associated disease or condition. Dosages are discussed further below.

The dosage regimen will be determined by the attending physician and clinical factors. As is well known in the medical arts, dosages for any one patient depend upon many factors, including the patient's size, body surface area, age, the particular compound to be administered, sex, time and route of administration, general health, and other drugs being administered concurrently.

For any compound, the therapeutically effective dose may be estimated initially either in cell culture assays or in animal models such as mice, rats, rabbits, dogs, or pigs. An animal model may also be used to determine the concentration range and route of administration. Such information may then be used to determine useful doses and routes for administration in humans. These techniques are well known to one skilled in the art and a therapeutically effective dose refers to that amount of active ingredient that ameliorates the symptoms or condition. Therapeutic efficacy and toxicity may be determined by standard pharmaceutical procedures in cell cultures or with experimental animals, such as by calculating and contrasting the ED50 (the dose therapeutically effective in 50% of the population) and LD50 (the dose lethal to 50% of the population). Any of the pharmaceutical compositions described herein may be applied to any subject in need of therapy, including, but not limited to, mammals such as dogs, cats, cows, horses, rabbits, monkeys, and especially humans.

The terms “effective dose”, “effective dosage” and “effective amount” are used interchangeably to refer to an amount sufficient to achieve or at least partially achieve the desired effect. The term “therapeutically effective” dose or amount is defined as an amount sufficient to treat or to ameliorate or at least partially arrest the disease and its complications in a patient already suffering from the disease. A therapeutically effective amount of a combination described herein or composition thereof of the disclosure generally results in a decrease in severity of disease symptoms, an increase in frequency or duration of disease symptom-free periods or a prevention of impairment or disability due to the disease affliction. In some embodiments, a therapeutically effective amount is an amount or dose of a combination described herein or composition thereof that treats a metabolic disorder as described herein.

Amounts or doses effective for this use will depend on the disorder to be treated (the indication), the delivered binding agent, the therapeutic context and objectives, the severity of the disease, prior therapy, the patient's clinical history and response to the therapeutic agent, the route of administration, the size (body weight, body surface or organ size) and/or condition (the age and general health) of the patient, and the general state of the patient's own immune system. The proper dose can be adjusted according to the judgment of the attending physician such that it can be administered to the patient once or over a series of administrations, and in order to obtain the optimal therapeutic effect.

### Methods of Use

Compositions described herein and compositions thereof are useful in the treatment of metabolic disorders and cardiometabolic disease. Treatment of obesity or overweight condition and their related comorbidities, such as Type II Diabetes, dyslipidemia, hyperglycemia, cardiovascular disease, and other metabolic disorders, represent a substantial, unmet medical need. Obesity is a risk factor of overall mortality and is estimated to have caused 3.4 million deaths worldwide in 2010 (Lim et al., 2012, The Lancet, vol. 380, pp. 2224-2260). Obesity, diabetes, high blood sugar and related conditions are high risk factors for serious or life-threatening complications such as heart disease, stroke, kidney problems, nerve damage, and eye disorders. In particular, cardiometabolic diseases such as stroke, heart attack, and heart and blood vessel diseases can be ameliorated by lowering blood sugar levels or achieving weight loss in patients with type 2 diabetes or obesity. Pharmacotherapeutics that can improve body composition, by changing the ratio of lean mass versus fat mass and thereby achieve weight loss or decrease of central adiposity, and/or improve patient glycemic status, are highly sought.

In some embodiments, the present disclosure provides a method of treating a metabolic disorder in a subject in need thereof comprising administration of a combination described herein or a composition thereof. A combination therapy, or a “combination” contemplated herein includes the co-administration of a TGFβ superfamily ligand binding agent and a GLP-1 agonist, wherein the TGFβ superfamily ligand binding agent may be administered prior to, after, or at the same time as the GLP-1 agonist.

The term “treatment” refers to both therapeutic treatment and prophylactic or preventative measures. Treatment includes the administration of a combination described herein or composition thereof to the body, an isolated tissue, or cell from a subject who has a metabolic disorder, a symptom of a metabolic disorder, or a predisposition toward a metabolic disorder, with the purpose to cure, heal, alleviate, relieve, alter, remedy, ameliorate, improve, or affect the metabolic disorder, the symptom of the metabolic disorder, or the predisposition toward the metabolic disorder. The term “prevention” as used herein means the avoidance of the occurrence or re-occurrence of a metabolic disorder, by the administration of a combination described herein or composition thereof to a subject in need thereof.

A metabolic disorder as used herein refers to a disorder affecting dysregulation of mammalian metabolism. Non-limiting examples of metabolic disorders include Type 1 Diabetes, Type 2 Diabetes, diabetic complications (such as, for example, retinopathy, nephropathy or neuropathies, diabetic foot, ulcus, macroangiopathies), metabolic acidosis or ketosis, hyperglycemia, reactive hypoglycemia, hyperinsulinemia, glucose metabolism disorder, insulin resistance, metabolic syndrome, Dyslipidaemias, atherosclerosis and related diseases, obesity, antipsychotic drug-associated obesity, glucocorticoid-induced obesity, non-alcoholic fatty liver disease, hypothalamic obesity associated with craniopharyngioma, monogenetic disorder associated obesity, pre-diabetes, hypertension, chronic heart failure, acute renal failure, edema, and hyperuricemia. The monogenetic disorders associated with obesity in humans, may include but are not limited to Bardet-Biedl syndrome, and a disorder arising from a mutation in one or more of the following genes ADCY3, ALMS1, ARL6, BBS1, BBS2, BBS4, BBS5, BBS7, BBS9, BBS10, BBS12, BDNF, CCDC28B, CEP290, CREBBP, EP300, GNAS, IER3IP1, MC3R, MKKS, MKS1, MRAP2, NTRK2, PCSK1, PHF6, POMC, SH2B1, SIM1, TMEM67, TRIM32, TTC8 and VPS13B, or combinations thereof. A metabolic disorder may also be associated with a complex genetic disorder, e.g., Prader-Willi syndrome. In some embodiments, the metabolic disorder is obesity. In some embodiments, the metabolic disorder is Type 2 diabetes.

The term “inhibition” or “inhibiting” is used herein to refer generally to reducing, slowing, restricting, delaying, suppressing, blocking, neutralizing, hindering, or preventing a process, such as without limitation reducing or slowing progression, growth, or spread of a disease or condition.

In some embodiments, improvement is determined by comparing clinical variables to measurements made before treatment, or alternatively to typical values measured in healthy adults. In some embodiments, treatment or prevention are within the context of the present disclosure if there is a measurable difference between the performances of subjects treated using the combinations, compositions and methods provided herein as compared to members of a placebo group, historical control, or between subsequent tests given to the same subject.

The term “subject” includes living organisms with a metabolic disease or condition, or who are susceptible to or at risk thereof. Examples of subjects include mammals, e.g., humans, monkeys, cows, rabbits, sheep, goats, pigs, dogs, cats, rats, mice, and transgenic species thereof. The animal can also be an animal model for a disorder, e.g., a mouse model, a xenograft recipient, and the like. In certain embodiments, the subject is a human.

In some embodiments, the subject has a body mass index (BMI) of 30 or greater. In some embodiments, the subject has a BMI of 27 or greater and has one or more obesity-related co-morbidities. As used herein, Body Mass Index or “BMI” is calculated as weight in kilograms (kg) divided by height in meters squared (m²), rounded to one decimal place. As used herein, “obesity” in adult humans is defined as a BMI greater than or equal to 30 kg/m². “Obesity” in human youth is defined as a BMI greater than or equal to the age- and sex-specific 95th percentile of the 2000 CDC growth charts. The term “overweight” is defined as a BMI of greater than or equal to 25 and less than 30.

The terms “obesity related co-morbidity,” “obesity related condition” and “obesity related disorder” may be used interchangeably and refer to a health condition depending from the obesity of the subject. In some embodiments, the obesity related co-morbidity or condition increases the mortality risk of the subject. Obesity related co-morbidities include but are not limited to: high blood pressure (hypertension), high LDL cholesterol, low HDL cholesterol, high levels of triglycerides (dyslipidemia), Type 2 diabetes, coronary heart disease, stroke, gallbladder disease, osteoarthritis, sleep apnea, breathing problems, cancer, gastroesophageal reflux disease, severe COVID-19, overall mortality, lower quality of life, mental illness such as clinical depression, anxiety, and other mental disorders, and body pain and difficulty with physical functioning.

“Lean mass” is defined as total body mass of a subject minus the fat mass and minus the bone mass of the subject. Lean mass and fat mass may be measured by, for example, bioelectrical impedance analysis (BIA), magnetic resonance imaging (MRI) or dual X-ray absorptiometry (DXA).

Exemplary doses of a binding agent include milligram or microgram amounts of the binding agent per kilogram of subject or sample weight (e.g., about 50 micrograms per kilogram to about 500 milligrams per kilogram, about 1 milligram per kilogram to about 100 milligrams per kilogram, about 1 milligram per kilogram to about 50 milligram per kilogram, about 1 milligram per kilogram to about 10 milligrams per kilogram, or about 3 milligrams per kilogram to about 5 milligrams per kilogram). Additional exemplary doses include doses of about 5 to about 500 mg, about 25 to about 300 mg, about 25 to about 200 mg, about 50 to about 150 mg, or about 50, about 100, about 150 mg, about 200 mg or about 250 mg, and, for example, daily or twice daily, or lower or higher amounts.

In some embodiments, the dose range for adult humans is generally from 0.005 mg to 10 g/day. Binding agents and compositions thereof may be provided in Unit dosage form, e.g., in a unit which is effective at such dosage or as a multiple of the same, for instance, units containing 5 mg to 500 mg, usually around 10 mg to 200 mg. A dosage unit can include from, for example, 1 to 30 mg, 1 to 40 mg, 1 to 100 mg, 1 to 300 mg, 1 to 500 mg, 2 to 500 mg, 3 to 100 mg, 5 to 20 mg, 5 to 100 mg (e.g. 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, or 500 mg) of a binding agent or composition described herein.

In some embodiments, a GLP-1 agonist is administered to a subject in need thereof in a dose of about 0.005 mg to about 3.0 mg, e.g, a dose of about 0.005 mg, about 0.01 mg, about 0.05 mg, about 0.1 mg, about 0.25 mg, about 0.5 mg, about 1 mg, about 1.5 mg, about 2 mg, about 2.5 mg, or about 3.0 mg. In exemplary embodiments, the GLP-1 agonist is administered to a subject in need thereof in a dose of about 0.005 mg to about 3.0 mg. In other embodiments of the combination therapies, a GLP-1 agonist is administered to a subject in need thereof in a dose of about 1 to about 20 mg weekly, e.g., a dose of about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, or about 20 mg weekly. In other embodiments, the GLP-1 agonist is administered to a subject in need thereof in a dose of about 0.25 mg to about 2.4 mg weekly, e.g., a dose of about 0.25 mg, about 0.5 mg, about 1 mg, about 1.7 mg, about 2 mg, or about 2.4 mg weekly. In other embodiments, the GLP-1 agonist is administered to a subject in need thereof in a dose of about 2.5 mg to about 15 mg weekly, e.g., a dose of about 2.5 mg. about 5 mg, about 7.5 mg, about 10 mg, about 12.5 mg, or about 15 mg weekly.

It should be understood that the effective amount of a binding agent and/or GLP1 agonist for treatment of a metabolic disorder varies depending upon the manner of administration, the age, body weight, and general health of the subject. Ultimately, the attending physician or veterinarian decides the appropriate amount and dosage regimen. It should be understood that the dosage or amount of a binding agent and/or GLP1 agonist depends on the individual case and is, as is customary, to be adapted to the individual circumstances to achieve an optimum effect. For example, dosing and administration regimens depend on the nature and the severity of the disease or condition to be treated, and also on the sex, age, weight and individual responsiveness of the human or animal to be treated, on the efficacy and duration of action of the compounds used, on whether the therapy is acute or chronic or prophylactic, and/or on whether other active compounds are administered in addition to the combinations described herein.

Dosage regimens can be adjusted to provide the optimum therapeutic response. For example, several divided doses may be administered daily, weekly, biweekly, or monthly, or the dose may be proportionally reduced as indicated by the exigencies of the therapeutic situation. In some embodiments, a combination described herein or composition thereof is administered at a therapeutically effective dosage sufficient to prevent or treat a metabolic disorder in a subject.

In some embodiments, a binding agent described herein is administered to a subject prior to the administration of a GLP-1 agonist. In some embodiments, the binding agent is administered 1, 2, 3, 4, 5, 6, 7, 8, 9, 10, 11, 12, 13, 14, 15, 16, 17, 18, 19, 20, or 21 days prior to the administration of the GLP-1 agonist. In some embodiments, a binding agent described herein is administered after the administration of a GLP-1 agonist. In some embodiments, the binding agent is administered 1, 2, 3, 4, 5, 6, 7, 8, 9, 10, 11, 12, 13, 14, 15, 16, 17, 18, 19, 20, or 21 days after the administration of the GLP-1 agonist. In some embodiments, the binding agent and GLP-1 agonist are administered via the same route. In some embodiments, the binding agent and GLP-1 agonist are administered via different routes. For example, in some embodiments, the binding agent is administered intravenously and the GLP-1 agonist is administered subcutaneously. In some embodiments, the binding agent is administered subcutaneously and the GLP-1 agonist is administered intravenously.

In some embodiments, a binding agent and GLP-1 agonist are administered at the same time (co-administration). In some such embodiments, the binding agent and the GLP-1 agonist can be administered in separate compositions, optionally by separate routes of administration. In some such embodiments, the binding agent and the GLP-1 agonist are administered in the same composition. In some embodiments, the binding agent and the GLP-1 agonist are administered in the same composition at least twice. For example, the binding agent and the GLP-1 agonist are administered in the same composition on a weekly basis for at least 1, 2, 3, 4, 5, 6, 7, 8, 9, 10 or more weeks.

In some embodiments, the methods provided herein comprise co-administration of a combination described herein (e.g., a binding agent and a GLP-1 agonist) followed by one or more administrations of a GLP-1 agonist. For example, in some embodiments, the methods provided herein comprise co-administration of a combination described herein (e.g., a binding agent and a GLP-1 agonist) followed by weekly administration of a GLP1 agonist for at least 1, 2, 3, 4, 5, 6, 7, 8, 9, 10 or more weeks.

In some embodiments, the methods provided herein comprise co-administration of a combination described herein (e.g., a binding agent and a GLP-1 agonist) followed by one or more administrations of a binding agent. For example, in some embodiments, the methods provided herein comprise co-administration of a combination described herein (e.g., a binding agent and a GLP-1 agonist) followed by weekly or monthly administration of a binding agent for at least 1, 2, 3, 4, 5, 6, 7, 8, 9, 10 or more months.

In some embodiments, in accordance with the methods of the present disclosure, one or more symptom of development or progression of a metabolic disorder is reduced by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% in a subject. For example, in some embodiments, the methods of the present disclosure result in an increase in muscle mass compared to administration of either a binding agent or GLP-1 agonist alone. In some embodiments, the methods of the present disclosure result in a decrease of white fat and/or an increase in brown fat in a subject compared to administration of either a binding agent or GLP-1 agonist alone. In some embodiments, the methods of the present disclosure result in an increase in lean muscle mass in a subject compared to administration of either a binding agent or GLP-1 agonist alone. In some embodiments, the methods of the present disclosure result in a reduced decrease in lean muscle mass in a subject compared to administration of either a binding agent or GLP-1 agonist alone. In some embodiments, the methods of the present disclosure result in a decrease in body fat mass (BFM) in a subject compared to administration of either a binding agent or GLP-1 agonist alone. In some embodiments, the methods of the present disclosure result in a decrease in body fat mass (BFM) and an increase in lean muscle mass in a subject compared to administration of either a binding agent or GLP-1 agonist alone. In some embodiments, the methods of the present disclosure result in a decrease in body fat mass (BFM) and a reduced decrease in lean muscle mass in a subject compared to administration of either a binding agent or GLP-1 agonist alone.

### Kits

In accordance with the present disclosure, the combinations or compositions thereof described herein may be assembled into kits or pharmaceutical systems for use in treating or preventing a metabolic disorder. Kits or pharmaceutical systems may comprise a container (e.g., packaging, a box, a carton, a vial, etc.), having in close confinement therein one or more container, such as vials, tubes, ampoules, bottles, and the like, that contains the binding agent and/or GLP-1 agonist or pharmaceutical composition. Additional kit components may include acids, bases, buffering agents, inorganic salts, solvents, antioxidants, preservatives, or metal chelators. The additional kit components may be present as pure compositions, or as aqueous or organic solutions that incorporate one or more additional kit components. Any or all of the kit components optionally further comprise buffers. Kits may also include tools for administration, such as needles, syringes, and the like. The kit may be used according to the methods described herein and may include instructions for use in such methods. Kits may also include instructions for administration and use of the combinations or pharmaceutical compositions.

In some embodiments, a kit comprises one or more recipients (such as vials, ampoules, containers, syringes, bottles, bags) of any appropriate shape, size and material (preferably waterproof, e.g., plastic or glass) containing the combination or composition thereof of the present disclosure in an appropriate dosage for administration (as discussed above). The kit may additionally contain directions for use (e.g., in the form of a leaflet or instruction manual), means for administering the combinations of the present disclosure such as a syringe, pump, infuser or the like, means for reconstituting the combinations of the disclosure and/or means for diluting the combinations of the disclosure.

The disclosure also provides kits for a single-dose administration unit. The kit of the disclosure may also contain a first recipient comprising a dried/lyophilized combination or composition described herein and a second recipient comprising an aqueous formulation. In certain embodiments, kits containing single-chambered and multi-chambered pre-filled syringes (e.g., liquid syringes and lyosyringes) are provided.

### EXAMPLES

The present invention will be more readily understood by referring to the following examples, which are provided to illustrate the invention and are not to be construed as limiting the scope thereof in any manner.

Unless defined otherwise or the context clearly dictates otherwise, all technical and scientific terms used herein have the same meaning as commonly understood by one of ordinary skill in the art to which this invention belongs. It should be understood that any methods and materials similar or equivalent to those described herein can be used in the practice or testing of the invention.

### Example 1. Design and Characterization of ActRIIB-ECD Fusion Proteins that Neutralize TGFβ Superfamily Ligands

Characterization of the neutralization activities of the benchmark ActRIIB ECD-Fc fusion construct. Exemplary wild type human ActRIIB-ECD-Fc (also referred to as “ACVR2B-Fc”; referred to herein as P75 (SEQ ID NO: 171)) was produced, characterized, and used as a benchmark protein. P75 is comprised of the human wild type ActRIIB-ECD (also referred to as the ACVR2B ECD; SEQ ID NO: 2), linked to the human IgG1 Fc domain by a triple glycine linker. The sequence of P75 is shown in Table 6.

The inhibition potency (IC50) of the benchmark agent P75 (wild type ActRIIB) for activin A, activin B, GDF-8, and GDF-11 was determined using the TGFP-reporter HEK293 cell-based assay described below. P75 efficiently neutralized all four ligands tested (activin A, activin B, GDF-8, and GDF-11) in a dose-dependent manner. IC50 values for P75 on activin A, activin B, GDF-8, and GDF-11 were 2.3, 1.4, 0.67, and 0.76 nM, respectively. The IC50 values are reported in Table 7.

The inhibition potency (IC50) of the benchmark agent P75 for BMP-9 and BMP-10 was determined using the BMP-reporter HepG2 cell-based assay described below. P75 neutralized BMP-9 and BMP-10 in a dose-dependent manner, with an average IC50 value of 2.7 and 2.0 nM, respectively. The IC50 values are reported in Table 8.

### Example 2. Design and Characterization of ActRIIB-ECD Polypeptide Constructs that Neutralize Activin a, Activin B, GDF-8, and GDF-11, but not BMP-9 and/or BMP-10 Signaling

A series of TGFβ superfamily ligand binding and neutralization agents were tested. The sequences of representative ActRIIB-ECD polypeptide constructs are listed in Table 6.

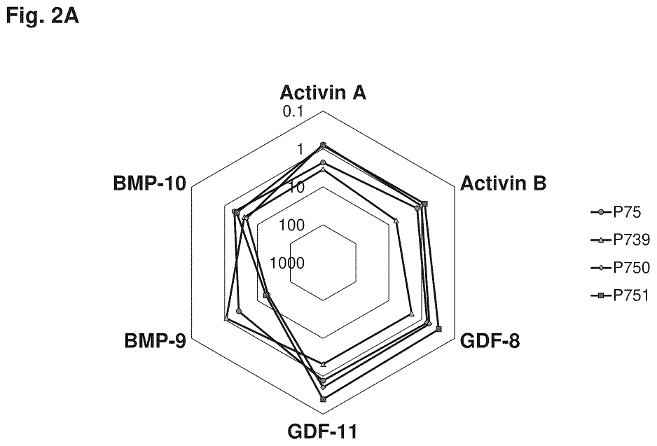

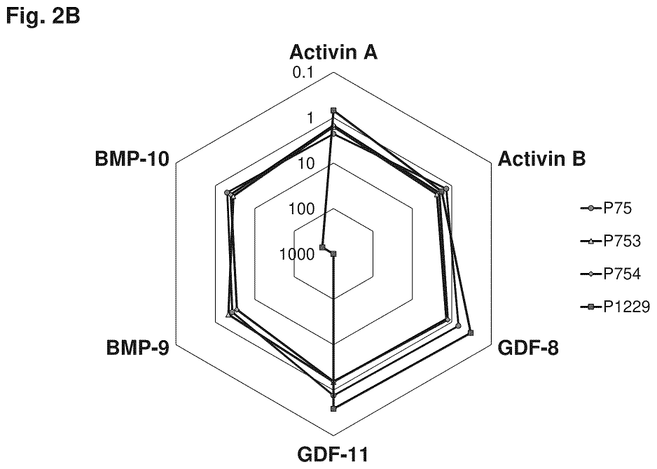

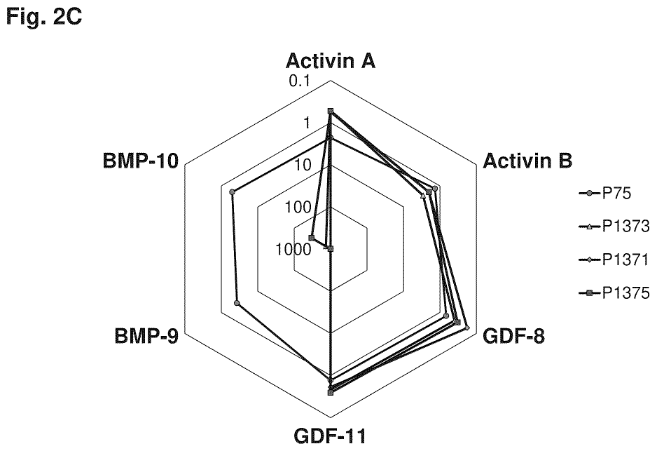

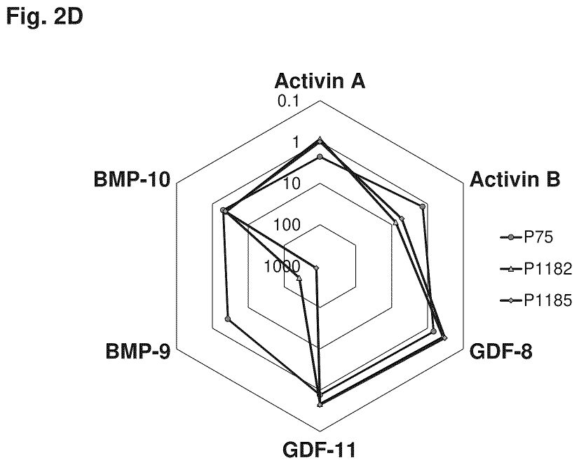

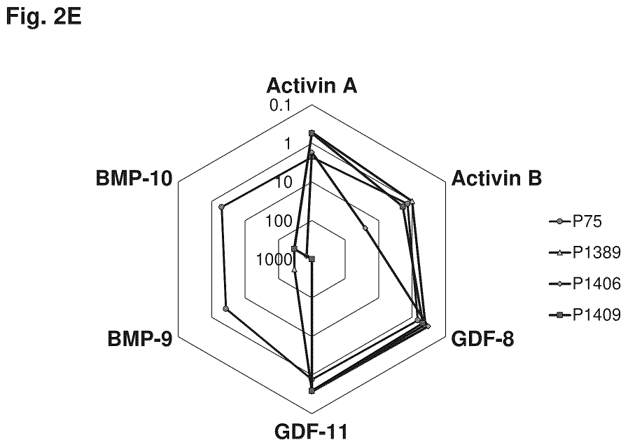

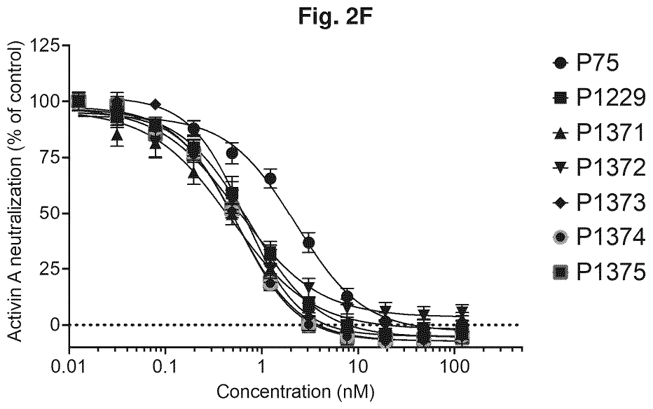

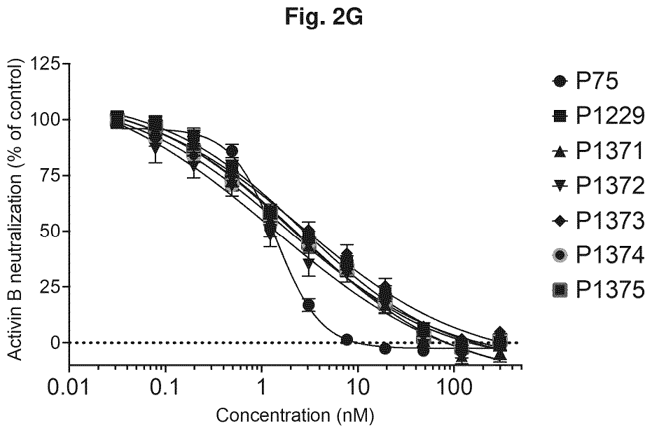

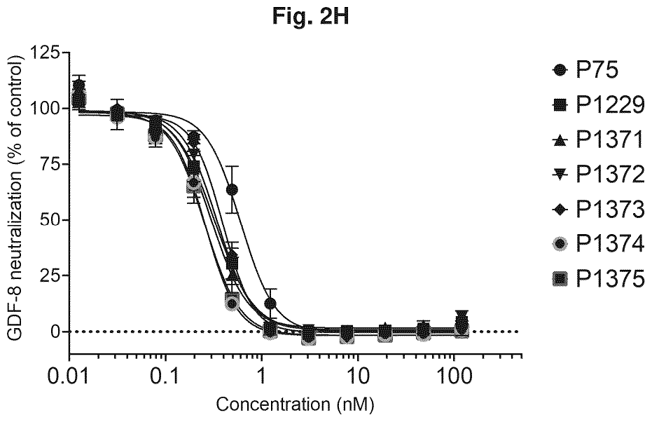

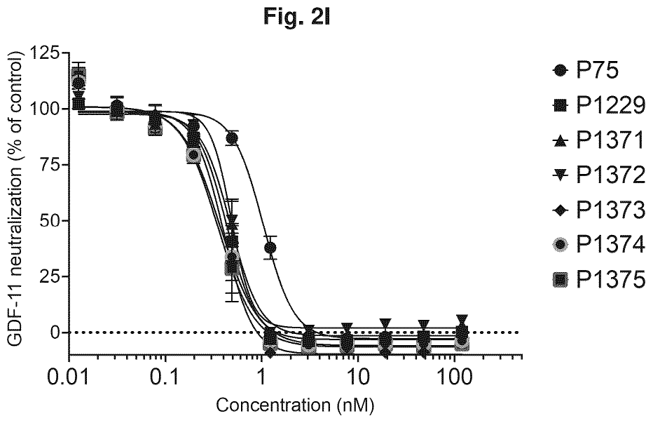

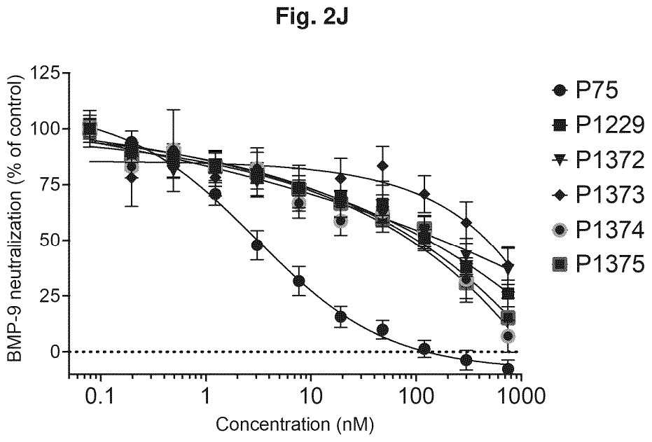

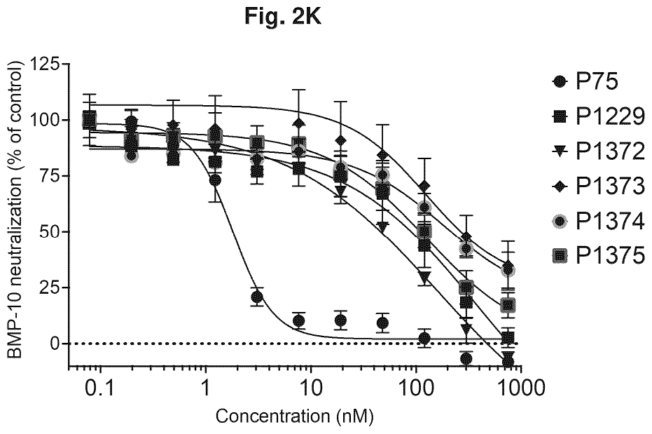

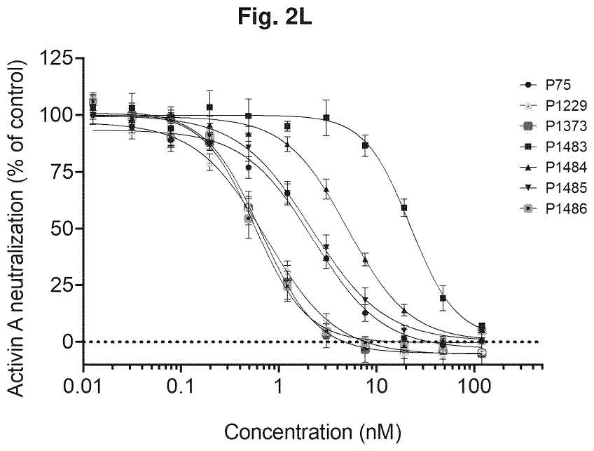

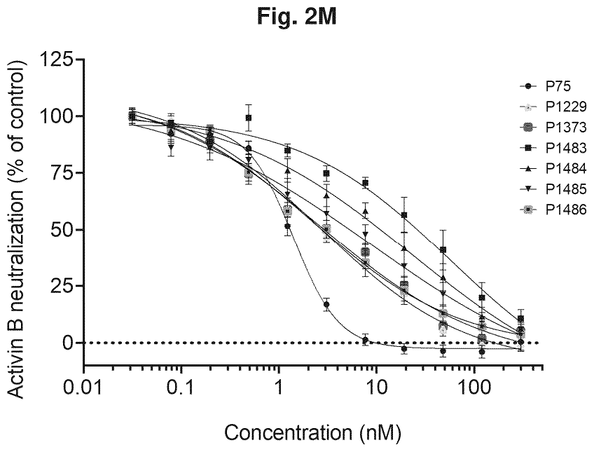

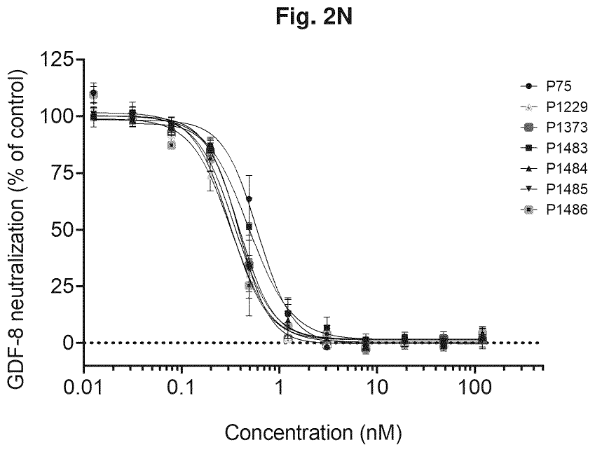

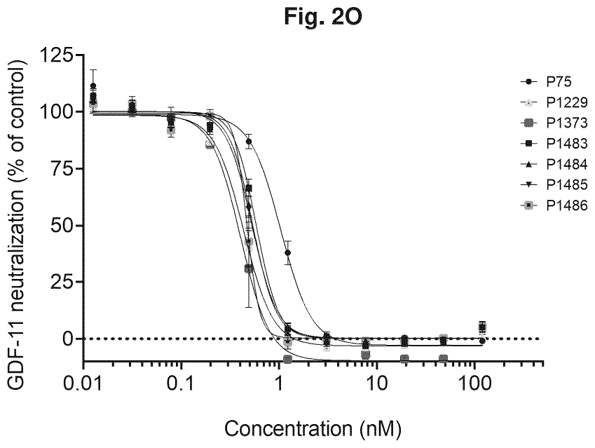

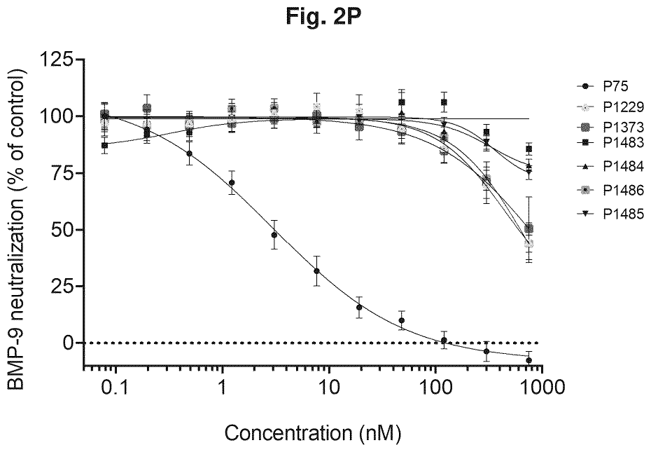

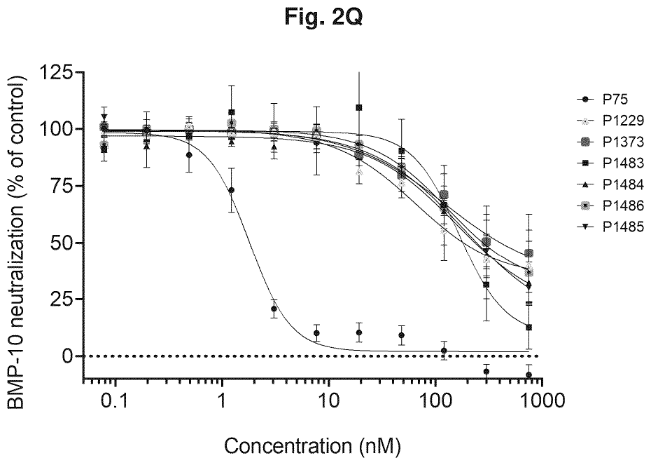

In order to assess the potency of these agents on TGFβ superfamily ligands known to be important in driving disease conditions, ActRIIB-ECD polypeptide variants were examined in the TGFβ-reporter HEK293 cells. Representative results for exemplary ActRIIB-ECD variants are shown in FIGS. 2A-2I. Among these examples, P750, P751, P1229, and P1373 displayed increased potency on activin A compared to P75, with IC50 values of 0.76, 0.82, 0.70, and 0.53 nM, respectively (FIGS. 2A-2C, 2F, and Table 7). P750 and P751 also showed increased potency relative to P75 on activin B, with IC50 values of 1.0 and 0.80 nM, respectively, relative to P75 (FIG. 2A and Table 7). In contrast, P1229 and P1373 showed comparable potency on activin B relative to P75, with IC50 values of 1.8 and 2.9 nM (FIGS. 2B-2C, 2G and Table 7). All four agents also demonstrated increased potency on GDF-8 compared to P75, with IC50 values of 0.58, 0.30,0.31, and 0.46 nM, respectively; and on GDF-11, with IC50 values of 0.51, 0.25, 0.36, and 0.50 nM, respectively (FIGS. 2A-C, 2H-2I, and Table 7). Other agents, notably P739, P753, and P754, displayed decreased potency relative to P75 on several ligands: activin B, with IC50 values of 6.0, 2.4, and 2.1 nM, respectively; GDF-8, with IC50 values of 2.0, 1.4, and 1.3 nM, respectively; and GDF-11, with IC50 values of 2.1, 1.5, and 1.6 nM, respectively (FIGS. 2A-2B and Table 7). Both P753 and P754 showed some increase in activin A potency, with IC50 values of 1.6 and 1.5 nM, respectively (FIG. 2B and Table 7). In contrast, P739 was less potent on activin A relative to P75, with an IC50 value of 3.5 nM (FIG. 2A and Table 7). These results indicate that it is possible to generate agents with distinct and unexpected neutralization profiles on activins and GDFs through mutation of the ActRIIB ECD.

Additional exemplary agents with mutations in the ActRIIB ectodomain were further investigated: P1182, P1185, P1389, P1406, and P1409. All agents, excluding P1406, demonstrated increased potency compared to P75 on activin A, GDF-8, and GDF-11 (FIGS. 2D-2E and Table 7). Activin A: IC50 values of 0.85, 1.0, 0.52, and 0.55 nM for P1182, P1185, P1389, and P1409, respectively; GDF-8: IC50 values of 0.37, 0.33, 0.40, and 0.48 nM for P1182, P1185, P1389, and P1407, respectively; GDF-11: IC50 values of 0.46, 0.44, 0.39, and 0.39 nM for P1182, P1185, P1389, and P1407, respectively (Table 7). The agent P1406 gained potency on GDF-8 (IC50: 0.33 nM) and GDF-11 (IC50: 0.37 nM), but not on activin A (IC50: 1.7 nM) relative to P75 (Table 7). All agents with two mutations, except P1389, demonstrated decreased activin B potency relative to P75, with IC50 values of 7.7, 5.2, >20, and 1.9 nM for P1182, P1185, P1406, and P1409, respectively (Table 7). P1389 was the only agent with two mutations to show a potency gain on activin B relative to P75, with an IC50 value of 1.0 nM (FIG. 2E and Table 7).

The impact of modifications in the Fc sequence on activin A, activin B, GDF-8, and GDF-11 potency was investigated. For all ligands, P1371 and P1372 were comparable to P1229, and P1374 and P1375 were comparable to P1373 (FIGS. 2B-2C, 2F-2I, and Table 8).

Finally, the impact of the length of the linker between the ectodomain and the Fc domain was investigated. Exemplary agents P1483, P1484, P1485, P1229, P1373, and P1486 contain linker length sequences of 3, 6, 10, 14, 19, and 36 amino acids, respectively. Potencies on activin A (FIG. 2L) and activin B (FIG. 2M) were linker dependent, i.e. activin A: IC50 values were 22, 5.1, 2.2, 0.70, 0.53, and 0.58 nM for P1483, P1484, P1485, P1229, P1373, and P1486, respectively; activin B: IC50 values were 57, 23, 8.8, 1.8, 2.9, and 2.2 nM for P1483, P1484, P1485, P1229, P1373, and P1486 (Table 7). These data show that linker lengths of 3, 6, 10 and 14 amino acids resulted in a stepwise increase in potency on activin A and activin B. Beyond that length, there were no additional apparent gains in potency. This phenomenon was not observed with GDF-8 and GDF-11 (FIGS. 2N-2O), where IC50 values were comparable between P1483, P1484, P1485, P1229, P1373, and P1486 (Table 7).

Characterization of the BMP-9 and BMP-10 neutralization activity of exemplary agents. The exemplary agents that were assessed for activin A, activin B, GDF-8, and GDF-11 neutralization were also tested in the BMP-reporter assays. The results obtained in these assays for the entire group of binding agents are presented in FIGS. 2A-2E, 2J-2K. Among these examples, P750, P751, P1229, and P1373 demonstrated significantly less neutralization of BMP-9 relative to P75, with IC50 values of 21, 18, >1,000, and >1,000 nM, respectively (FIGS. 2A-2C, 2J and Table 8). Moreover, these agents also demonstrated decreased potency on BMP-10, with IC50 values of 4.7, 2.4, 491, and 619 nM, respectively (FIGS. 2A-2C, 2K, and Table 8). Two exemplary agents, P739 and P753, exhibited increased inhibition potency on BMP-9, with IC50 values of 1.1 and 2.1 nM, respectively, while having reduced inhibition potency on BMP-10, with IC50 values of 4.0 and 2.9 nM, respectively (FIGS. 2A-B and Table 4). Finally, one exemplary agent, P754, had negligible change in both BMP-9 and BMP-10 potency relative to P75, with IC50 values of 3.5 and 2.5 nM, respectively (FIG. 2B and Table 4). Agents P1182 and P1185 demonstrated decreased BMP-9 potency relative to P75 (IC50 values of 265 and 791 nM, respectively), with no changes in BMP-10 potency (IC50 values of 2.4 and 2.3 nM, respectively; FIG. 2D and Table 8). As for P1389, P1406 and P1409, both demonstrated minimal inhibition of either BMP-9 or BMP-10 (FIG. 2E and Table 8).

As was done with the other ligands, the impact of modifications in the Fc sequence on BMP-9 and BMP-10 potency was investigated. For both ligands, P1372 was comparable to P1229, and P1375 was comparable to P1373 (FIGS. 2B-2C, 2J-2K and Table 8).

The impact of the length of the linker between the ectodomain and the Fc domain on BMP-9 and BMP-10 potency was also investigated. Exemplary agents P1483, P1484, and P1485, exhibited decreased apparent BMP-9 potency relative to P1229, P1373, and P1486 (FIG. 2P), though the IC50 differences could not be determined accurately (Table 8). All agents were still >100-fold less potent than P75 (ActRIIB-Fc wild-type). This was not observed with BMP-10, for which the potency was comparable between all six exemplary agents (FIG. 2Q and Table 8).

In summary, the results show that certain point mutations in the ActRIIB-ECD led to significant increases in inhibition potency for certain TGFβ superfamily ligands, particularly those shown to be involved in metabolic diseases, namely activin A, activin B, GDF-8, and GDF-11. Of note, the single point mutation in P750 and P751, and the double point mutations in P1182 and P1185, improved potency on activin A, activin B (except for P1182 and P1185), GDF-8, and GDF-11 relative to the wild-type ActRIIB-ECD (P75) by ~2-4-fold (Table 7), while decreasing the inhibition potency on BMP-9 by ~5-300-fold (Table 8); BMP-9 is considered a homeostatic ligand that should not be neutralized. For P750, P751, P1182, and P1185, inhibition potency on BMP-10 was unchanged or modestly reduced (Table 4). In contrast, the point mutation in P739 reduced neutralization of activin A, activin B, GDF-8, GDF-11, and BMP-10 by ~2-3-fold, while improving potency on BMP-9 by ~2-fold (Tables 7 and 8). Other point mutations (P753 and P754) reduced potency on most TGFβ superfamily ligands (e.g., activin B, GDF-8, GDF-11, BMP-9, and BMP-10) by ~2-fold, relative to P75, while specifically improving potency on activin A modestly, by ~1.5-fold (Tables 7 and 8). Furthermore, the single point mutation in P1229 and P1373, and double point mutations in P1409 improved potency on activin A, GDF-8, and GDF-11 relative to P75 by ~4-fold (Table 7), while simultaneously decreasing potency on both BMP-9 and BMP-10 by ~200-300-fold (Table 8). Unexpectedly, the single point mutation in P1229 and P1373 induced differential gains in potency on certain ligands, such as activin A and activin B, when the length of the linker was varied between the ectodomain and the Fc region. This was not the case for other ligands (for example, GDF-8 and -11). Taken together, the combination of the specific point mutation with an optimal linker length is required to provide the ideal ligand specificity profile for neutralization of disease-driving cytokines. Lastly, one exemplary test agent, P1406, demonstrated comparable activin A potency, increased GDF-8 and GDF-11 potency (~2-fold), and decreased activin B, BMP-9, and BMP-10 potency (~20-, ~300-, and ~350-fold, respectively) relative to P75. These data indicate that different point mutations (alone or in combination) had dramatically different and unexpected effects on the neutralization profile for TGFβ superfamily ligands.

TABLE 7
IC₅₀ values for ectodomain constructs for activin
A, activin B, GDF-8, and GDF-11 neutralization.

| Ectodomain | Activin A | Activin B | GDF-8 | GDF-11 |
|---|---|---|---|---|
| construct | IC₅₀ (nM) | IC₅₀ (nM) | IC₅₀ (nM) | IC₅₀ (nM) |
| P75 | 2.3 | 1.4 | 0.67 | 0.75 |
| P739 | 3.5 | 6.0 | 2.0 | 2.1 |
| P750 | 0.76 | 1.0 | 0.58 | 0.51 |
| P751 | 0.82 | 0.80 | 0.30 | 0.25 |
| P753 | 1.6 | 2.4 | 1.4 | 1.5 |
| P754 | 1.5 | 2.1 | 1.3 | 1.6 |
| P1229 | 0.70 | 1.8 | 0.33 | 0.39 |
| P1373 | 0.53 | 2.9 | 0.40 | 0.46 |
| P1371 | 0.56 | 2.7 | 0.23 | 0.60 |
| P1372 | 0.56 | 2.0 | 0.18 | 0.53 |
| P1374 | 0.53 | 1.9 | 0.37 | 0.40 |
| P1375 | 0.53 | 2.0 | 0.33 | 0.39 |
| P1182 | 0.85 | 7.7 | 0.37 | 0.46 |
| P1185 | 1.0 | 5.2 | 0.33 | 0.44 |
| P1389 | 0.52 | 1.0 | 0.40 | 0.39 |
| P1406 | 1.7 | >20 | 0.33 | 0.37 |
| P1409 | 0.55 | 1.9 | 0.48 | 0.39 |
| P1483 | 22 | 57 | 0.49 | 0.58 |
| P1484 | 5.1 | 23 | 0.39 | 0.54 |
| P1485 | 2.2 | 8.8 | 0.35 | 0.53 |
| P1486 | 0.58 | 2.2 | 0.32 | 0.47 |

TABLE 8
IC₅₀ values for ectodomain constructs
for BMP-9 and BMP-10 neutralization.

| | BMP-9 | BMP-10 |
|---|---|---|
| Ectodomain construct | IC₅₀ (nM) | IC₅₀ (nM) |
| P75 | 3.0 | 2.0 |
| P739 | 1.1 | 4.0 |
| P750 | 21 | 4.7 |
| P751 | 18 | 2.4 |
| P753 | 2.1 | 2.9 |
| P754 | 3.5 | 2.5 |
| P1229 | >1,000 | >100 |
| P1373 | >1,000 | >100 |
| P1371 | NT | NT |
| P1372 | >1,000 | >100 |
| P1374 | NT | NT |
| P1375 | >1,000 | >100 |
| P1182 | 265 | 2.4 |
| P1185 | 791 | 2.3 |
| P1389 | >100 | >100 |
| P1406 | >1,000 | >100 |
| P1409 | >1,000 | >100 |
| P1483 | >1,000 | >100 |
| P1484 | >1,000 | >100 |
| P1485 | >100 | >100 |
| P1486 | >100 | >100 |

NT: not tested

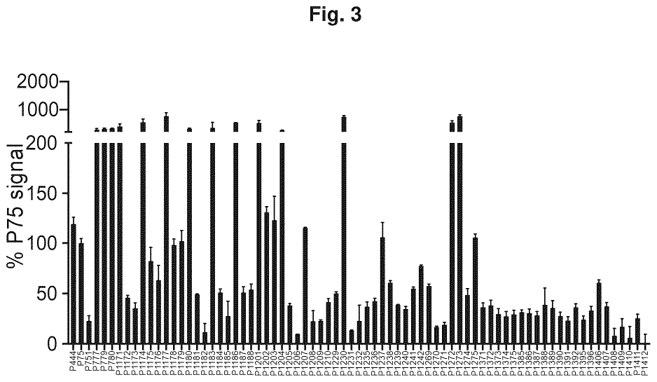

Additional exemplary agents were tested in binding assays to identify those with the desired ligand specificity profile, i.e., superior binding to activin A compared to the benchmark agent P75 (wild type ActRIIB-ECD-Fc), and decreased binding to BMP-9 and BMP-10 compared to P75. For activin A binding, our results showed that there was a broad spectrum of activin A binding potencies depending on the ActRIIB receptor modification. Several of the agents showed increased binding to activin A compared to P75 (i.e., decreased signal in the assay), whereas many did not bind to activin A as strongly as P75 (i.e., increased signal in the assay; see FIG. 3). For example, proteins P751, P1182, P1206, P1229, P1231, P1270, P1271, P1373, P1389, P1409, P1410, P1412, and others, had potent activin A binding. For P751, P1229, P1373, P1389, and P1409, this increased activin A binding relative to P75 is in agreement with its increased neutralization potency on activin A (FIG. 2 and Table 3). On the other hand, P777, P1174, P1177, P1230, P1272, P1273, and others, had lower binding to activin A relative to P75. Moreover, some exemplary agents showed comparable binding to activin A such as P75, e.g., P1175, P1178, P1179, P1237, or P1275. Of note, P444 (wild-type ActRIIA-ECD-Fc) had a comparable activin A binding profile to P75 in this assay.

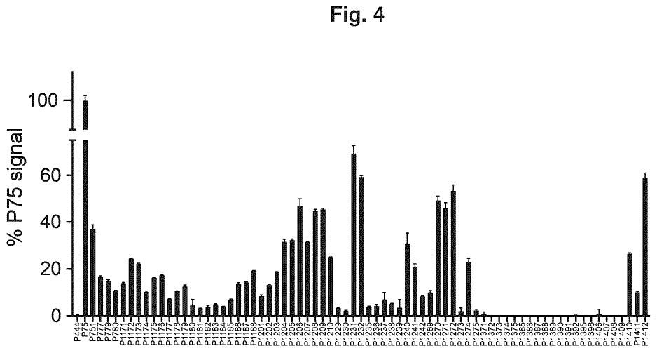

Our results also showed varying levels of BMP-9 binding depending on the ActRIIB-ECD modification. Of note, in the BMP-9 assay, P444 had near undetectable binding to BMP-9. All exemplary agents tested displayed decreased binding to BMP-9 as compared to P75 (i.e., decreased signal; see FIG. 4). Some proteins displayed near-undetectable binding to BMP-9 (like P444), such as P1273, P1372, P1373, P1374, P1375, P1389, P1406, P1409, and others (FIG. 4). Other examples of proteins with very low BMP-9 binding were P1181, P1229, P1239, and P1275. In contrast, some exemplary proteins demonstrated intermediate binding between P444 and P75, such as P1206, P1231, P1232, P1272, P1412, and others. For P751, P1229, P1373, P1389, P1406, and P1409, this decreased BMP-9 binding relative to P75 is in agreement with its decreased neutralization potency on BMP-9 (Table 4).

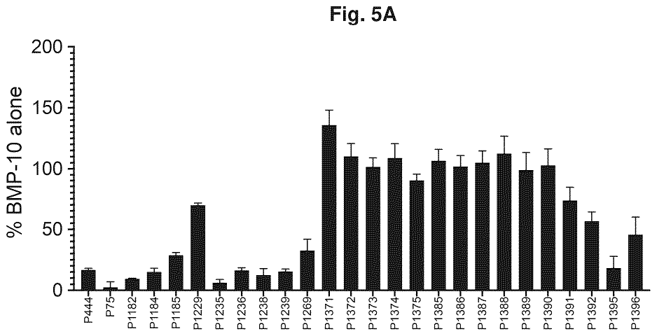

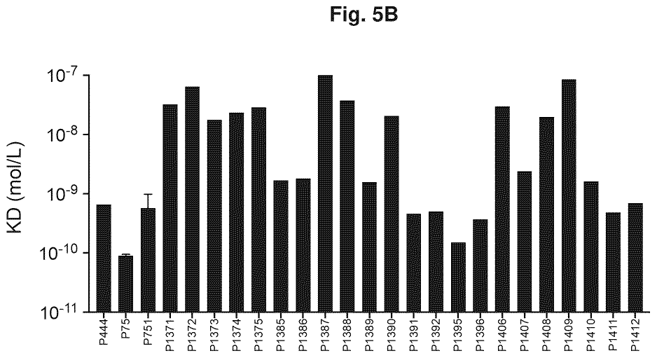

For BMP-10 neutralization, selected agents were analyzed for neutralization potency in the BMP-reporter cell-based assay (FIG. 5A) and/or for binding by bio-layer interferometry (BLI; FIG. 5B). At the concentration tested, P444 demonstrated less neutralization (i.e., greater signal) than P75 (FIG. 5A). In agreement with this, it exhibited a higher K_{D} (lower affinity) than P75 (FIG. 5B). In the cell-based assay, all agents tested demonstrated lower potency on BMP-10 than P75 (FIG. 5A). In agreement with this, by BLI, all agents exhibited higher K_{D}s than P75 (FIG. 5B). Results for exemplary agents P1371-P1392 and P1395-P1396 were well correlated between the two assays (FIG. 5A vs FIG. 5B). Other exemplary agents P1406-P1412 demonstrated comparable or greater K_{D} values relative to P444 (FIG. 5B).

Taken together, the results presented above show that certain exemplary agents have a desired ligand specificity profile, i.e., superior binding activity to activin A and significantly reduced binding and/or neutralization activity on BMP-9 and BMP-10. Such agents, including P750, P751, P1184, P1185, P1229, P1236, P1371, P1372, P1373, P1374, P1375, P1389, P1406, and P1409, are predicted to be useful in conditions where it is beneficial to inhibit the activity of TGFβ superfamily ligands that drive disease while not interfering with the activity of ligands associated with homeostasis.

### Example 3. Effects of Exemplary Agents In Vivo on Body Weight Gain

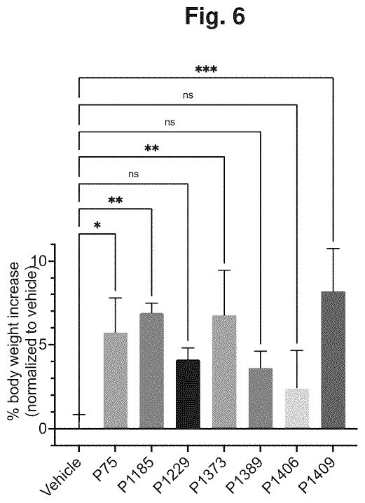

To investigate the target engagement of exemplary ActRIIB-ECD polypeptide constructs in vivo, mice (n=6/group) were injected with given agents (25 mg/kg, subcutaneous injection), and sacrificed after 4 days to assess body weight gain. Most agents tested (P75, P1185, P1373, and P1409) induced a statistically significant body weight gain over the course of 4 days, with P1409 exhibiting the greatest average weight gain (FIG. 6). Three agents, P1229 (p-value of 0.07), P1389, and P1406 induced an apparent body weight gain that did not reach statistical significance (FIG. 6).

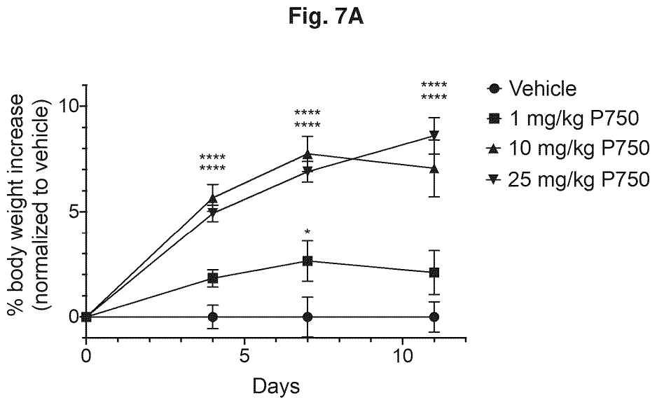

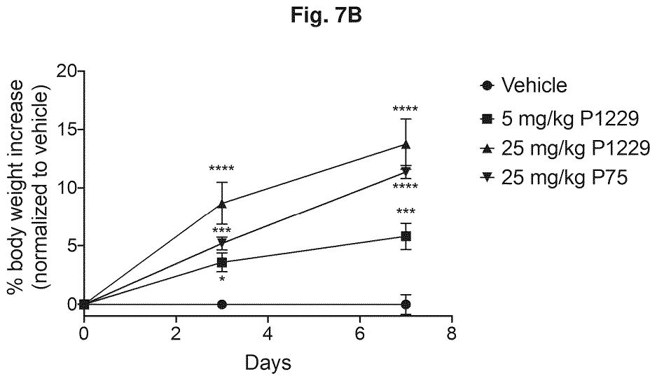

Additional in vivo evaluation of exemplary agents, P750 and P1229, was conducted and compared to P75. P750 (1, 10, and 25 mg/kg), P1229 (5 and 25 mg/kg), and P75 (25 mg/kg) were injected subcutaneously twice weekly in wild-type male mice (n=6/group), over the course of 11 days (P750; FIG. 7A) or 7 days (P1229 and P75; FIG. 7B). P750 (10 and 25 mg/kg), P1229 (5 and 25 mg/kg), and P75 (25 mg/kg) induced a significant body weight gain as early as Day 3 (between 5 and 10%), up until the end of the study (FIGS. 7A-7B). P750 at 1 mg/kg demonstrated a modest increase in body weight, which only reached statistical significance on Day 7 (FIG. 7A).

These data exemplify that the desired ligand specificity profile established in vitro translated to a significant body weight gain in vivo and show that most exemplary agents tested in these experiments demonstrated successful target engagement in vivo.

### Example 5: Exemplary Agent Effects on Body Composition and Metabolism in Lean Mice

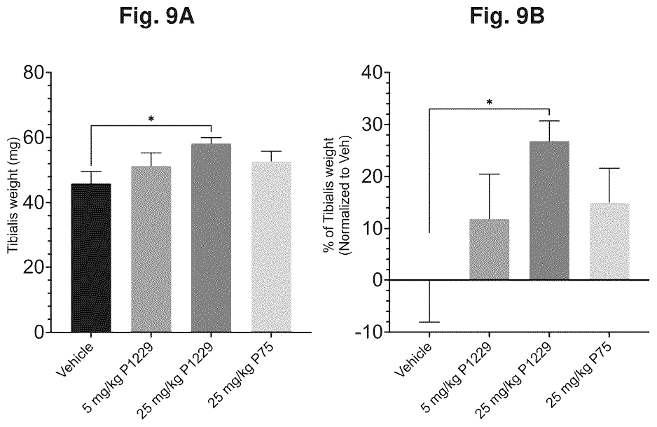

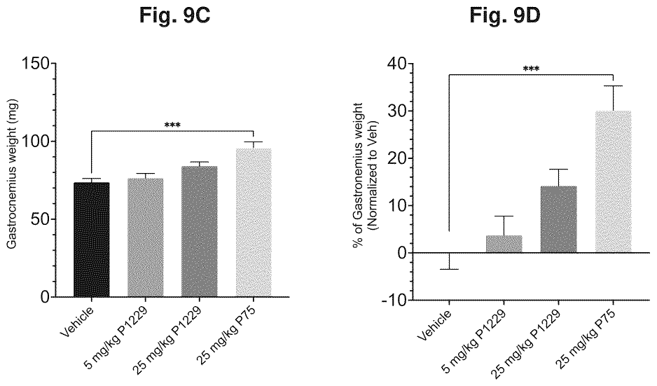

The effects of P1229 on body composition and metabolism in lean mice were further assessed. After seven days (twice weekly injections), P1229 and P75 at 25 mg/kg significantly increased tibialis anterior (FIGS. 9A-B) and gastrocnemius weight (FIGS. 9C-D), respectively.

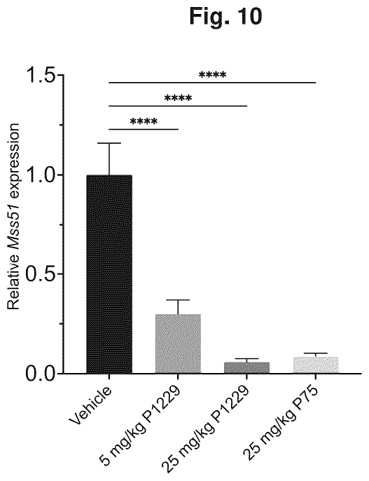

Additional assays were performed to assess the relative expression level of Mss51, a skeletal muscle-specific gene. Mss51 is a key target of myostatin signaling, with a role in fatty acid oxidation, glycolysis, and oxidative phosphorylation. Treatment with P1229 led to a dose-dependent decrease in Mss51 expression in the tibialis muscle relative to vehicle (FIG. 10). The effects of P1229 were comparable to those of P75 (FIG. 10).

### Example 6: Exemplary Agent Effects on Body Composition and Metabolism in Obese Mice

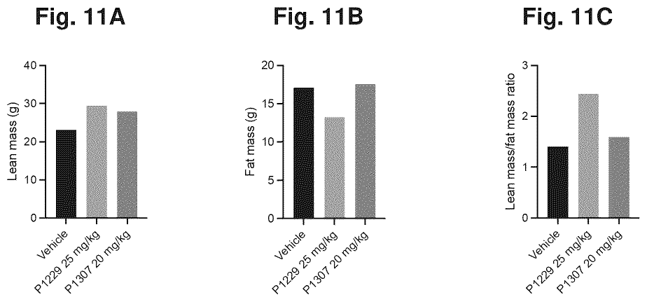

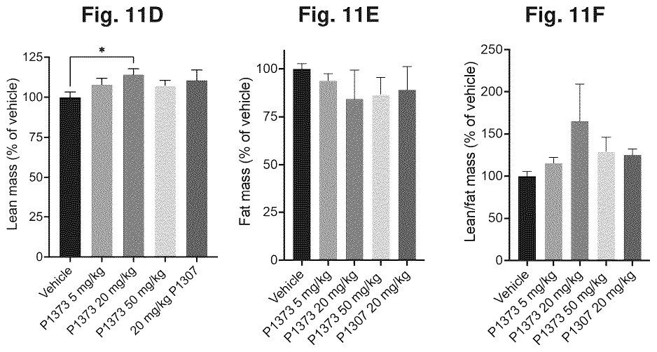

Experiments were performed to assess the effects of P1229 on body composition and metabolism in diet-induced obese (DIO) mice. DIO mice were purchased from Jackson Laboratories (#380050). Briefly, mice were fed a high-fat diet (HFD, 60% kcal from fat) from 6 weeks of age until 22 weeks of age (total of 16 weeks on the HFD). After a two-week acclimatization period, mice (n=8/group) were injected subcutaneously with vehicle, P75 (25 mg/kg twice weekly), P1229 (25 mg/kg twice weekly), P1373 (5, 20, or 50 mg/kg twice weekly), or 20 mg/kg of CDD866 (P1307), a murine version of bimagrumab (an antibody targeting ActRIIB and ActRIIA; once weekly, subcutaneously, as previously described in Morvan et al. (2017) PNAS 114(47): 12448-12453). After 3 weeks of treatments, some animals were sacrificed and scanned by EchoMRI (SkyScan1176) (FIGS. 11A-F), while others were sacrificed for tissue collection and RNA analysis. Animals were maintained on the HFD throughout the acclimatization and treatment periods. The murine version of bimagrumab used for these experiments comprises the heavy chain amino acid sequence of SEQ ID NO: 376 and the light chain sequence of SEQ ID NO: 377.

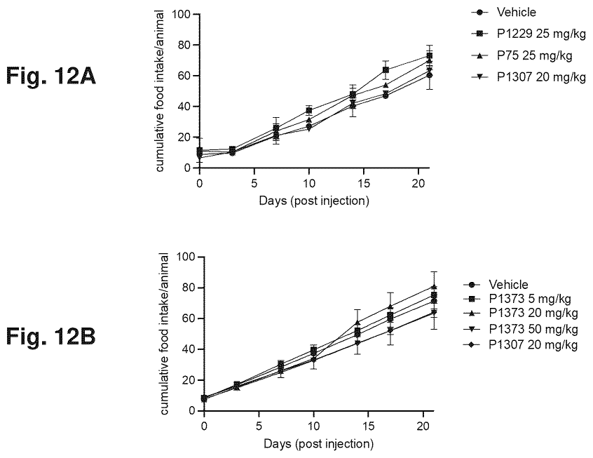

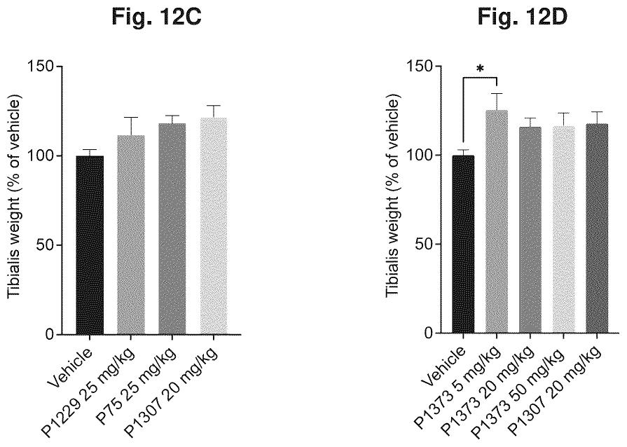

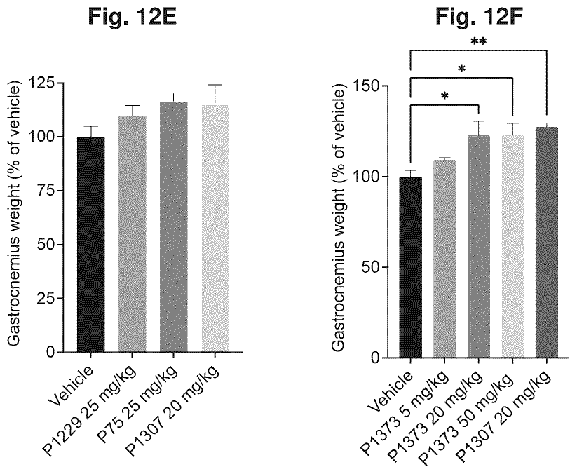

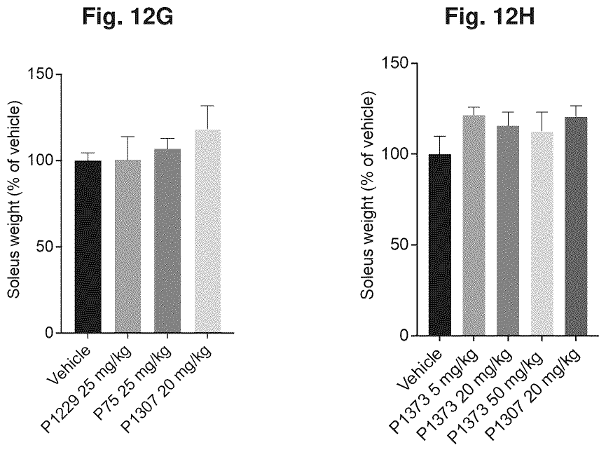

Importantly, treatment with P1229 or P1373 resulted in fat loss with concomitant lean mass gain. Treatment with bimagrumab, P1307 (CDD866), or P75 FIGS. 11A-F) also promoted lean mass gain but less fat loss, with the result that the ratio of lean mass to fat mass was higher in the P1229- and P1373-treated animals. Fat loss with P1229 and P1373 was not caused by decreased food consumption (FIGS. 12A-B). As for individual skeletal muscles, all treated groups showed significant increases or trends towards increased muscle weight (FIGS. 12C-H).

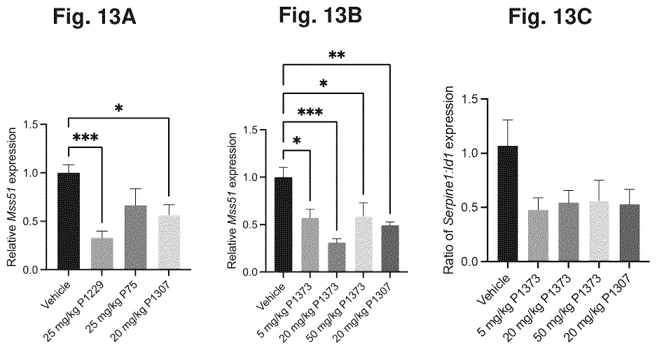

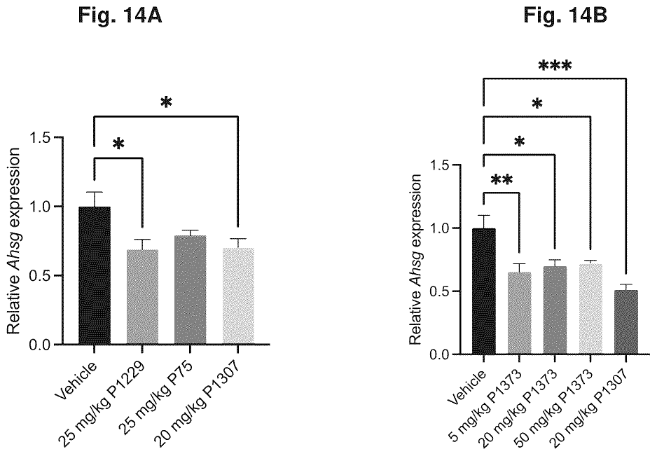

Furthermore, treatment with P1229, P1373 and P1307 (but not P75), improved muscle metabolism as demonstrated by reduced Mss51 gene expression in gastrocnemius muscle (FIGS. 13A-B). Additionally, P1373 led to a lower Serpine1:Id1 expression ratio in soleus muscle (FIG. 13C); this ratio is indicative of reduced activin/GDF signaling (Serpine1) and greater BMP signaling (Id1).

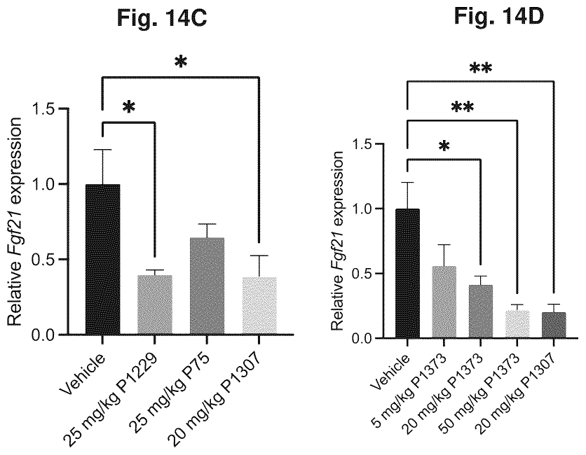

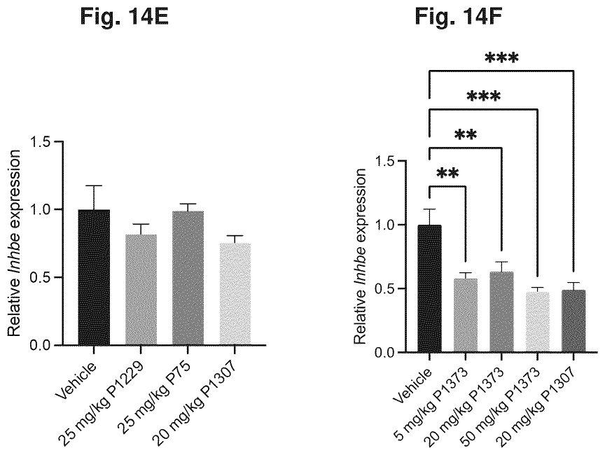

Next, hepatic parameters were assessed in DIO animals treated with P75, P1229, P1373, or P1307. Exemplary agents P1229, P1373, and P1307, but not P75, reduced Ahsg expression (which encodes fetuin-A, a marker of obesity and decreased insulin sensitivity, See Mathews et al (2002) 10.2337/diabetes.51.8.2450) (FIGS. 14A-B), as well as Fgf21 expression (a marker of increased triglycerides, adiposity, and insulin resistance, See Hale et al, Endocrinology, Volume 153, Issue 1, 1 Jan. 2012, Pages 69-80) (FIGS. 14C-D). Finally, P1373 and P1307 (in one of the two studies) significantly decreased Inhbe expression (FIGS. 14E-F), which encodes activin E, a TGF-β superfamily member linked to obesity (See Akbari et al. Nat Commun 13, 4844 (2022) and Deaton et al. Nat Commun 13, 4319 (2022)).

Although bimagrumab decreased fat mass while preserving lean body mass in a phase 2 clinical trial (Heymsfield et al (2021) JAMA Netw Open), its mechanism of action may limit its muscle anabolic potential relative to that of a ligand trap such as ramatercept (ActRIIB-Fc) or the exemplary agents disclosed herein. Without wishing to be bound by theory, the relatively low affinity of ActRIIB-based traps for classic BMPs, (e.g., BMP-2/4), which promote muscle anabolic effects, suggest that an ActRIIB-based trap may act primarily on activin A/GDF-8/GDF-11 without neutralizing positive BMP signaling to muscle. In contrast, the ability of BMPs to engage the cell surface activin type 2 receptors, which are the targets of bimagrumab, may render BMP signaling to muscle at least partially susceptible to inhibition by bimagrumab (see Lee et al. (2023) The Journals of Gerontology). Thus, the therapeutic strategy based on ActRIIB-Fc ligand traps, such as employed by the exemplary agents disclosed herein, may present advantages over direct targeting of the activin type 2 receptors (as done by bimagrumab).

### Example 7: Exemplary Agents Affect FSH Production in Obese Mice

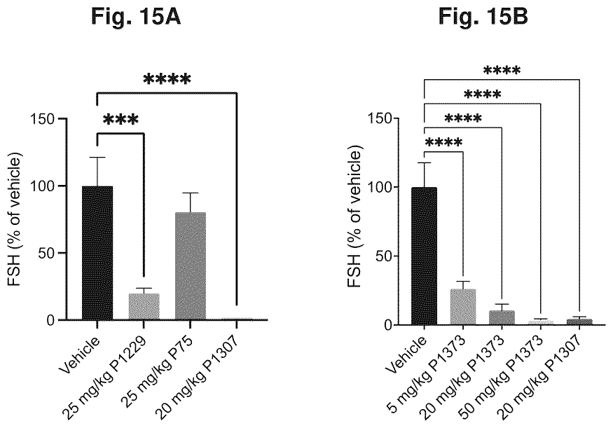

Follicle-stimulating hormone (FSH) is a known biomarker of in vivo target engagement for activin receptor-based traps (See Attie et al. (2013). Muscle Nerve, 47: 416-423). To further validate in vivo target engagement for exemplary agents, FSH production was evaluated in DIO mice, as described above. Agents P1229, P1373, and P1307, but not P75, demonstrated significant decreases in FSH production in DIO mice following 3 weeks of treatment (FIGS. 15A-B). Notably, P1373 demonstrated progressive dose-dependent target engagement between 5 and 50 mg/kg (FIG. 15B).

### Example 8. Effects of Exemplary Agents on Body Composition in Combination with Incretin Mimetics

Experiments were performed in vivo to investigate the potential for additive or synergistic effects between exemplary ActRIIB-ECD polypeptide constructs and incretin mimetics. Male mice were fed a high fat diet (Research Diet, D12492) until −28 weeks of age, at which point they were subcutaneously injected with vehicle, P1307 (CDD866, a murine bimagrumab analogue, 20 mg/kg weekly), P1229 (10 or 40 mg/kg twice weekly), semaglutide (40 μg/kg daily), or tirzepatide (45 μg/kg daily). Two groups of mice also received simultaneous, separate injections of semaglutide (40 μg/kg daily)+P1229 (40 mg/kg twice weekly), or tirzepatide (45 μg/kg daily)+P1229 (40 mg/kg twice weekly). One group of mice were treated with P1229 (40 mg/kg twice weekly) while having restricted access to food, based on the food consumption of the tirzepatide-treated group (45 μg/kg daily).

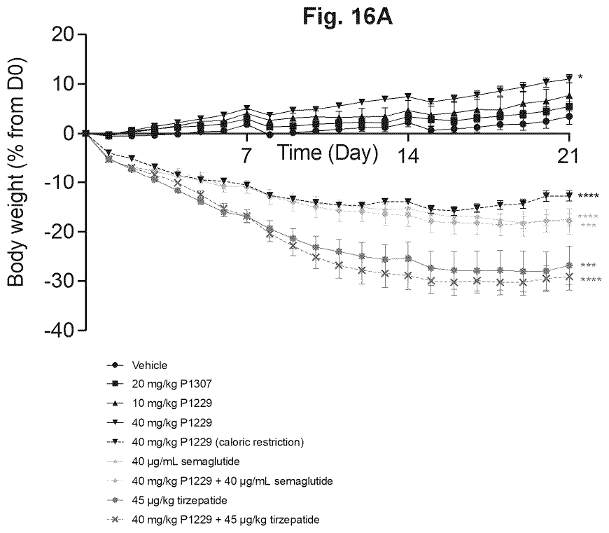

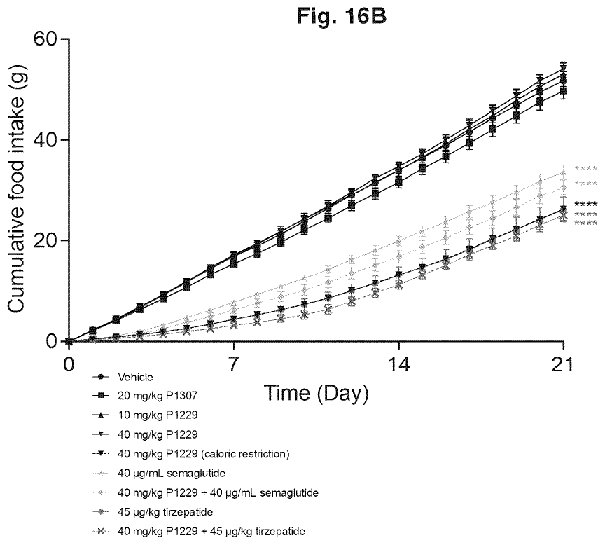

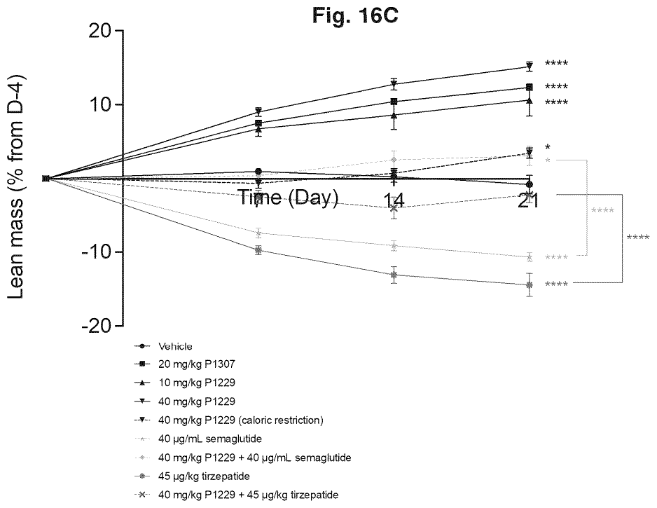

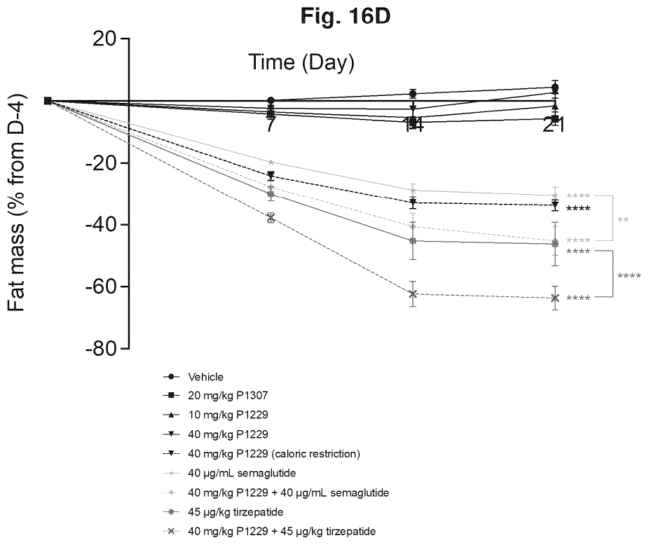

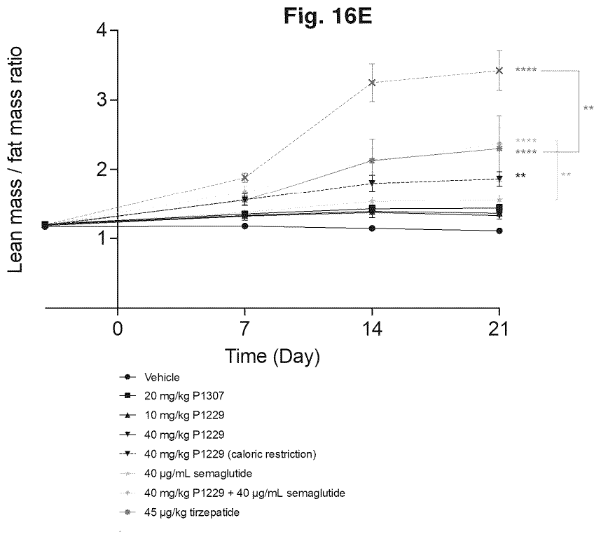

Mice injected with P1307 (20 mg/kg) or P1229 (10 mg/kg) showed no significant changes in body weight over the course of the study (FIG. 16A and Table 9); mice injected with 40 mg/kg P1229 showed a significant increase in body weight over the course of the study (FIG. 16A and Table 9). All mice injected with tirzepatide or semaglutide progressively lost body weight, with tirzepatide-treated animals losing more weight than semaglutide-treated animals (FIG. 16A and Table 9). The addition of P1229 had no significant impact on those trends. Interestingly, the calorie-restricted animals treated with P1229 closely matched the body weight loss observed with semaglutide alone (FIG. 16A and Table 9). As expected, food consumption was significantly decreased in all incretin-treated animals, with tirzepatide demonstrating a greater decrease than semaglutide. P1307 and P1229 did not significantly affect food intake (FIG. 16B).

Despite the clear reduction in body weights promoted by incretin mimetics, these compounds induce the loss of not only fat mass, but also lean mass. Indeed, based on body composition NMR data, it was observed that animals treated with semaglutide or tirzepatide alone lost −10.6% and −14.5% lean mass, and −30.3% and −46.2% fat mass, respectively (FIGS. 16C-D and Table 9). In contrast, both P1307 and P1229 alone demonstrated robust gains in lean mass (between +10% and +15%) and modest or no losses in fat mass (between −5.7 and +2.7%) (FIGS. 16C-D and Table 9).

The combination of semaglutide or tirzepatide with P1229 yielded additive effects on lean mass. Specifically, when adding the lean mass changes induced by each agent alone, a value of +4.5% is obtained for the combination of semaglutide and P1229; the observed value was very close at +3.1% (Table 9). Similarly, when adding the lean mass changes induced by tirzepatide and P1229 alone, a value of +0.6% is obtained; the observed value for the combination was close at −2.3% (Table 9).

In contrast to lean mass effects, surprisingly, the combination of semaglutide or tirzepatide with P1229 yielded synergistic effects on fat mass loss. Animals treated with semaglutide and P1229 lost −45.2% fat mass, and animals treated with tirzepatide and P1229 lost −63.6% fat mass (FIG. 16D and Table 9). If the effects had been additive between semaglutide and P1229, or between tirzepatide and P1229, one would have predicted less fat mass loss at −27.6% and −43.5%, respectively.

Finally, with regards to the ratio of lean mass over fat mass, P1307 and P1229 (10 mg/kg) induced a modest, non-statistically significant increase (FIG. 16E and Table 9). However, P1229 (40 mg/kg) with caloric restriction induced a significant increase in this ratio (FIG. 16E and Table 9). Tirzepatide alone, but not semaglutide alone, also induced a significant increase in lean mass over fat mass ratio (FIG. 16E and Table 9). Importantly, the addition of P1229 to either semaglutide or tirzepatide induced a statistically significant increase in the lean mass over fat mass ratio relative to vehicle, or relative to the incretin alone (FIG. 16E and Table 9).

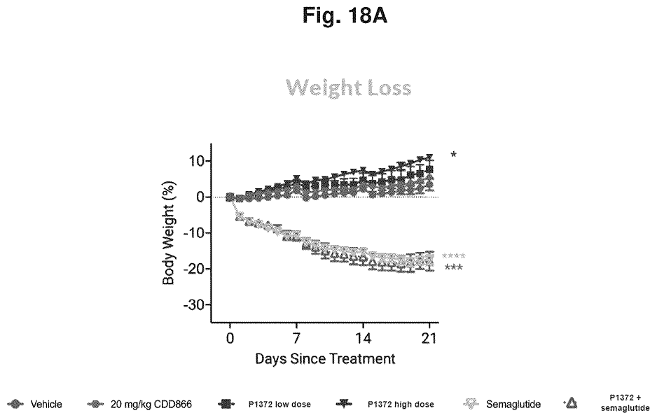

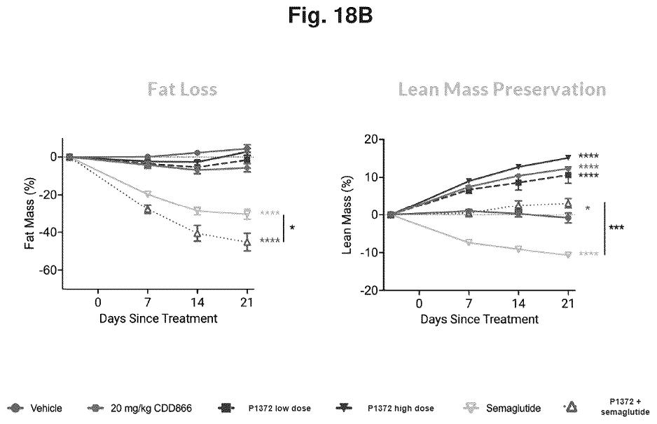

Similar results are further demonstrated with a coformulation of P1372 and semaglutide. See FIG. 18. As shown in FIG. 18A, animals treated with semaglutide or the coformulation of P1372 and semaglutide demonstrated significant weight loss 3 weeks after treatment. FIG. 18B shows that the coformulation of P1372 and semaglutide significantly increased fat mass loss in comparison to treatment with semaglutide alone. Furthermore, treatment with the coformulation of P1372 and semaglutide prevented loss of lean muscle mass. In contrast, treatment with semaglutide alone resulted in a significant loss of lean muscle mass.

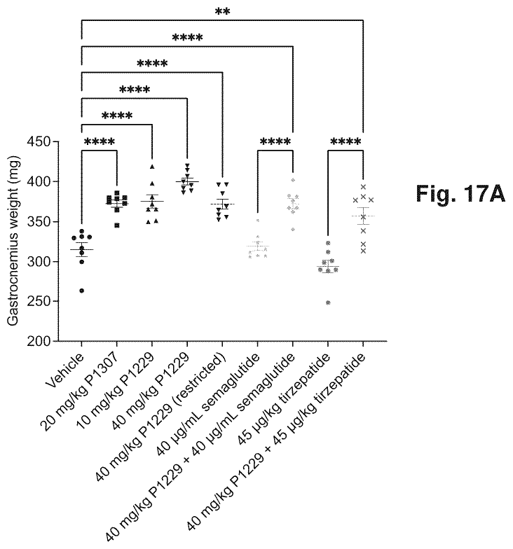

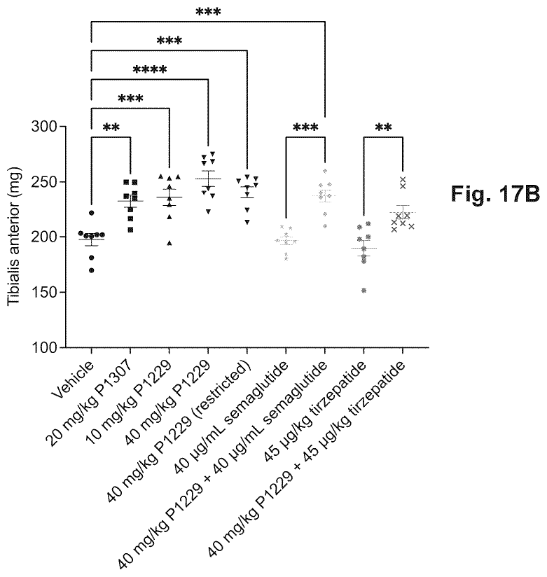

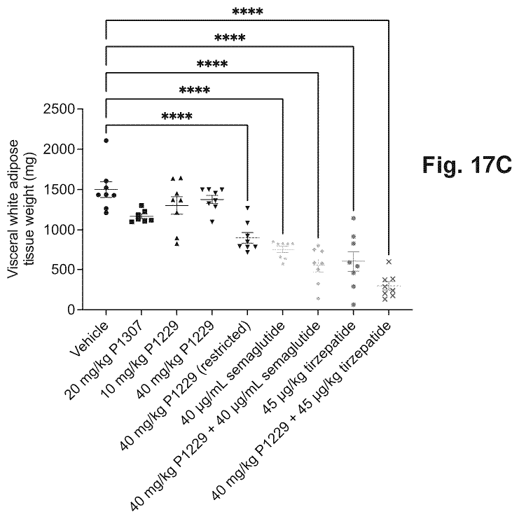

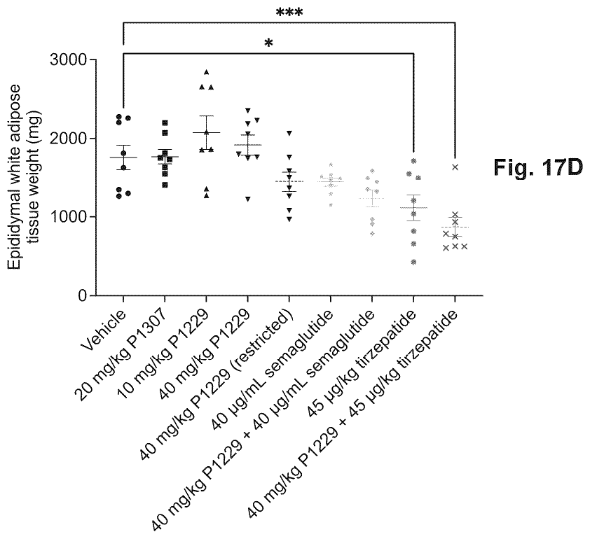

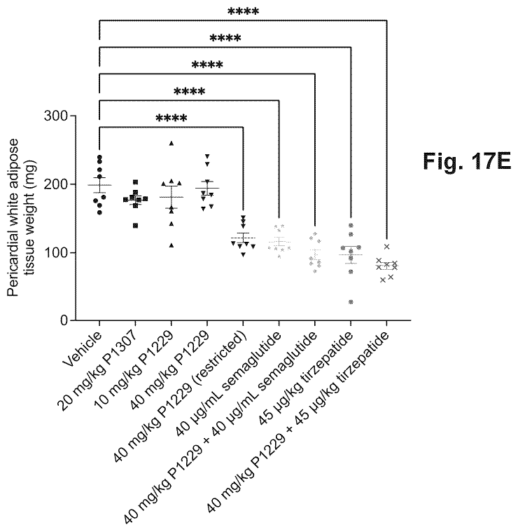

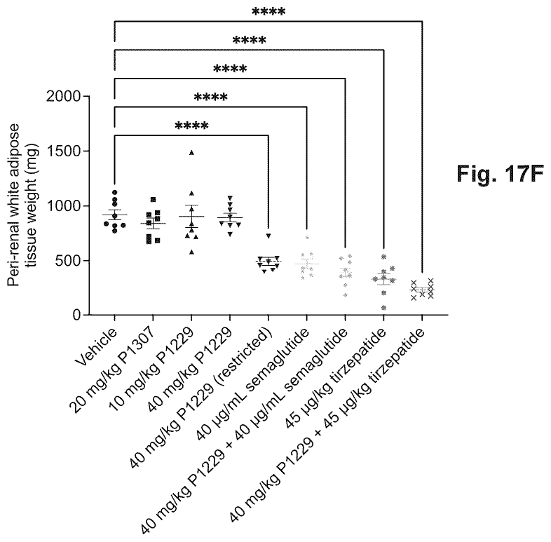

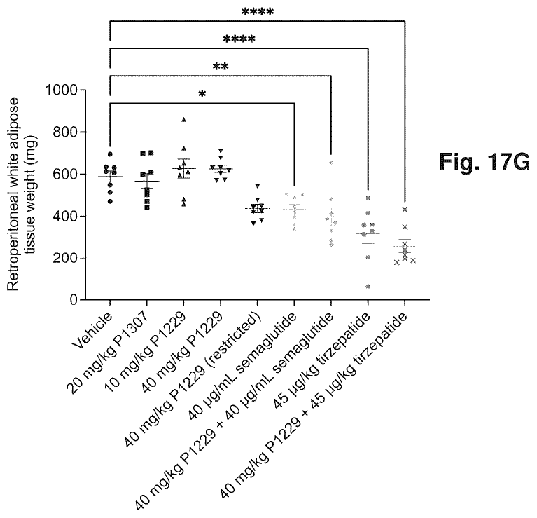

Organ weights obtained at the end of the study corroborated the observations made based on body composition NMR data. Gastrocnemius and tibialis anterior weights were significantly increased by P1307 and P1229, with or without caloric restriction (FIGS. 17A and 17B). Semaglutide and tirzepatide alone had no significant effect on muscle weights, but the addition of P1229 significantly increased muscle weights relative to the incretin mimetics alone (FIGS. 17A-B). Visceral, epididymal, pericardial, peri-renal, and retroperitoneal white adipose tissue weights were not significantly affected by P1307 or P1229 (FIGS. 17C-17G). P1229 with caloric restriction significantly decreased visceral, pericardial, and peri-renal (but not epididymal and retroperitoneal) white adipose tissue weights (FIGS. 17C-17G). Semaglutide and tirzepatide alone significantly decreased all white adipose tissue weights, and the addition of P1229 enhanced these effects, though this was not statistically significant (FIGS. 17C-17G).

Overall, these data indicate that incretin mimetics and ectodomain-based traps that neutralize activins and GDFs act synergistically on body composition, particularly on fat mass loss.

TABLE 9
Average body weights, and lean mass and fat mass
gains or losses after 21 days of treatment.

| | Body weight | Lean mass | Fat mass | |
|---|---|---|---|---|
| | (% change | (% change | (% change | Lean |
| | from | from | from | mass/fat |
| Group | baseline) | baseline) | baseline) | mass ratio |
| Vehicle | +3.6% | −0.8% | +4.4% | 1.1 |
| P1307 | +5.4% | +12.3% | −5.7% | 1.4 |
| P1229 (10 | +7.7% | +10.6% | −1.6% | 1.4 |
| mg/kg) | | | | |
| P1229 (40 | +11.1% | +15.1% | +2.7% | 1.3 |
| mg/kg) | | | | |
| P1229 (40 | −12.8% | +3.5% | −33.7% | 1.9 |
| mg/kg) + calorie | | | | |
| restriction | | | | |
| Semaglutide | −17.5% | −10.6% | −30.3% | 1.6 |
| Semaglutide + | −17.9% | +3.1% | −45.2% | 2.4 |
| P1229 (40 | | | | |
| mg/kg) | | | | |
| Tirzepatide | −26.8% | −14.5% | −46.2% | 2.3 |
| Tirzepatide + | −29.1% | −2.3% | −63.6% | 3.4 |
| P1229 (40 | | | | |
| mg/kg) | | | | |

### Example 9. Suitability of Co-Formulating Exemplary Agents with an Incretin Mimetic

The successful co-formulation of two biologically active molecules such as an acylated peptide and/or a protein is dependent, in part, on their respective isoelectric points (pI) and their resulting charge states in the formulation. The pI is defined as the pH at which the molecule has no net charge. At this pH hydrophobic interactions are promoted, possibly leading to aggregation. In contrast, at pH values sufficiently below or above the pI, the surface of the peptide or protein becomes charged (negatively above the pI, positively below the pI). This net charge increases the repulsive forces between molecules, which in turn decreases the risk of aggregation and precipitation. In the context of a co-formulation, both components should be above or below their respective isoelectric points; otherwise, the opposite charges on the molecules along with any exposed hydrophobic patches may facilitate aggregation and ultimately lead to precipitation of one or both molecules.

The pIs of different incretin mimetics and biologics are listed in Table 10. In the case of semaglutide, its predicted pI is 5.5. An ectodomain-based trap, like P1229, P1371, P1372, P1373, P1374, or P1375, has a predicted pI of 5.3-5.4. This fortuitously enables the co-formulation of an incretin mimetic (like semaglutide) with an ectodomain based trap at physiological pH (7.4) since the charge requirements of both molecules to remain in solution are satisfied.

Bimagrumab, an antibody targeting the activin receptors ActRIIA and ActRIIB, is currently under investigation in obese patients, with and without concomitant semaglutide injections (NCT05616013). However, given that the predicted pI of bimagrumab is 8.7, a co-formulation with an incretin at physiological pH would likely not be possible since semaglutide would have a net negative charge, while bimagrumab would have a net positive charge. This may be why these two agents are currently administered as separate injections. In fact, most antibodies have a pI at or above physiological pH (see examples in Table 10) thereby facilitating their solubility and stability at physiological or slightly acidic pH. Volagidemab is an antibody against the glucagon receptor, and is under investigation in type I (NCT04779645) and type II diabetes (NCT05093517). Other commonly used antibodies, such as adalimumab, pembrolizumab, ustekinumab, dupilumab, and nivolumab are also listed in Table 10.

Therefore, ectodomain-based traps have the unique advantage of being suitable for co-formulation with incretins, which is not feasible with most antibodies.

TABLE 10
Predicted isoelectric points of different incretin
mimetics, ectodomain-based traps, and antibodies

| Protein | Type | Predicted isoelectric point |
|---|---|---|
| Semaglutide | Peptide-based | 5.5 |
| Tirzepatide | incretin mimetic | 4.6 |
| Exenatide | | 4.7 |
| Retatrutide | | 4.3 |
| Mazdutide | | 5.0 |
| P1229 | Ectodomain-based | 5.4 |
| P1371 | trap | 5.4 |
| P1372 | | 5.3 |
| P1373 | | 5.4 |
| P1374 | | 5.4 |
| P1375 | | 5.3 |
| Bimagrumab | Antibody | 8.7 |
| Volagidemab | | 8.2 |
| Adalimumab | | 8.3 |
| Pembrolizumab | | 7.5 |
| Ustekinumab | | 8.7 |
| Dupilumab | | 6.8 |
| Nivolumab | | 7.8 |

To confirm the co-formulatability of ectodomain-based traps and incretins, lyophilized semaglutide was resuspended in solution (pH 7.5) with or without P1372, at a molar ratio of 1 (semaglutide) to ~1.3 (P1372). P1372 was formulated at 40 mg/mL and semaglutide at 1.6 mg/mL, in a solution of 10 mM NaPO₄, 100 mM glycine, and 75 mM sucrose (pH 7.5). These resuspensions were compared to P1372 alone and semaglutide alone. In each case, the following parameters were assessed: a) monomer content (size exclusion chromatography), b) activin A and GDF-8 neutralization (cell-based assays [CBAs]), c) activin A binding (ELISA), d) GLP-1 receptor binding (ELISA). The parameters from a stability study are summarized in Table 11 (DO, the day of the resuspension), Table 12 (7 days post-reconstitution, stored at 2-8° C.), Table 13 (7 days post-reconstitution, stored at room temperature), Table 14 (28 days post-reconstitution, stored at 2-8° C.), Table 15 (28 days post-reconstitution, stored at room temperature). Based on assay precision, potency fold differences between 0.5 and 2 (i.e., 2-fold below or above the reference standard) were deemed to be within assay error for the activin A (ELISA and CBA) and GDF-8 (CBA) assays. Similarly, potency fold differences between 0.25 and 4 (i.e., 4-fold below or above the reference) were deemed to be within assay error for the GLP-1R (ELISA) assay.

As summarized in Tables 11-15, co-formulating semaglutide with P1372 did not affect the integrity of either component on day 0 (Table 11), on day 7 after storage at 2-8° C. (Table 12) or room temperature (Table 13), or on day 28 after storage at 2-8° C. (Table 14) or room temperature (Table 15).

Together, these data, along with the predicted favourable pI values, suggest a unique advantage of ectodomain-based traps over traditional antibodies, where the former (but not the latter) can be co-formulated with incretin mimetics, and administered in patients as a single injection.

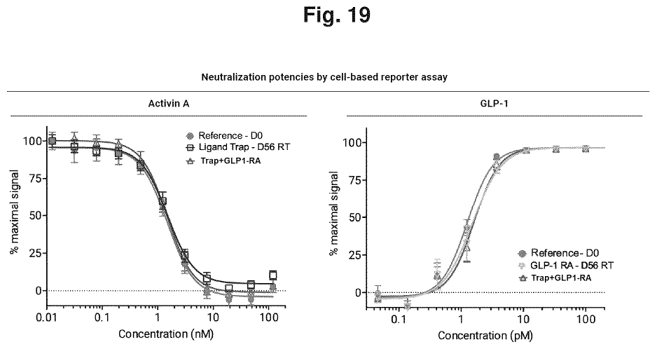

As demonstrated in FIG. 19, ActRIIB ligand trap P1372 can be coformulated with semaglutide without a loss in activity of either compound. Similar activity results were obtained with tirzepatide and retatrutide).

TABLE 11
Monomer content and potency fold-changes at D 0

| | Monomer content | | CBA potency | | ELISA potency | |
|---|---|---|---|---|---|---|
| | P1372 | semaglutide | Activin A | GDF-8 | Activin A | GLP-1R |
| P1372 | 99.3% | N/A | 1 (ref) | 1 (ref) | 1 (ref) | ND |
| Semaglutide alone | N/A | 99.8% | ND | ND | ND | 1 (ref) |
| Semaglutide + P1372 | 99.3% | 99.9% | 0.66 | 1.4 | 0.98 | 0.78 |

TABLE 12
Monomer content and potency fold-changes at D 7 (after storage at 2-8° C.)

| | Monomer content | | CBA potency | | ELISA potency | |
|---|---|---|---|---|---|---|
| | P1372 | semaglutide | Activin A | GDF-8 | Activin A | GLP-1R |
| P1372 | 99.3% | N/A | 1 (ref) | 1 (ref) | 1 (ref) | ND |
| Semaglutide alone | N/A | 99.9% | ND | ND | ND | 1 (ref) |
| Semaglutide + P1372 | 99.3% | 99.9% | 1.3 | 1.1 | 0.85 | 2.1 |

TABLE 13
Monomer content and potency fold-changes at
D 7 (after storage at room temperature)

| | Monomer content | | CBA potency | | ELISA potency | |
|---|---|---|---|---|---|---|
| | P1372 | semaglutide | Activin A | GDF-8 | Activin A | GLP-1R |
| P1372 | 99.2% | N/A | 1 (ref) | 1 (ref) | 1 (ref) | ND |
| Semaglutide alone | N/A | 99.9% | ND | ND | ND | 1 (ref) |
| Semaglutide + P1372 | 99.2% | 99.9% | 0.97 | 0.93 | 1.4 | 3.2 |

TABLE 14
Monomer content and potency fold-changes at D 28 (after storage at 2-8° C.)

| | Monomer content | | CBA Potency | | ELISA Potency | |
|---|---|---|---|---|---|---|
| | P1372 | semaglutide | Activin A | GDF-8 | Activin A | GLP-1R |
| P1372 | 99.2% | N/A | 1 (ref) | 1 (ref) | 1 (ref) | ND |
| Semaglutide alone | N/A | 100% | ND | ND | ND | 1 (ref) |
| Semaglutide + P1372 | 99.5% | 99.5% | 1.1 | 0.87 | 1.3 | 0.82 |

TABLE 15
Monomer content and potency fold-changes at
D 28 (after storage at room temperature)

| | Monomer content | | CBA potency | | ELISA potency | |
|---|---|---|---|---|---|---|
| | P1372 | Semaglutide | Activin A | GDF-8 | Activin A | GLP-1R |
| P1372 | 98.8% | N/A | 1 (ref) | 1 (ref) | 1 (ref) | ND |
| Semaglutide alone | N/A | 100% | ND | ND | ND | 1 (ref) |
| Semaglutide + P1372 | 99.1% | 96.3% | 1.0 | 0.93 | 0.79 | 1.1 |

To assess whether the exemplary agents can be co-formulated with other incretin mimetics, semaglutide, tirzepatide, or retatrutide was resuspended with or without P1372, at a molar ratio of 1 (semaglutide) to 1.3 (P1372), 4 (tirzepatide) to 1 (P1372), and 3.2 (retatrutide) to 1 (P1372). P1372 was co-formulated at 60 mg/mL with semaglutide (2.4 mg/mL), tirzepatide (15 mg/mL), or retatrutide (12 mg/mL) in a solution of 20 mM NaPO₄, 100 mM glycine, 75 mM sucrose, and 150 mM histidine (pH 7.5). These resuspensions were compared to P1372 alone and each incretin mimetic alone. In each case, the following parameters were assessed: a) monomer content (size exclusion chromatography), b) activin A and GDF-8 neutralization (cell-based assays [CBAs]), and c) GLP-1 receptor activation (CBA). The parameters from an 8-week stability study are summarized in Table 16 (semaglutide), Table 17 (tirzepatide), and Table 18 (retatrutide).

As summarized in Tables 16-18, co-formulating semaglutide, tirzepatide, or retatrutide with P1372 did not affect the integrity of any component over 8 weeks at room temperature or 2-8° C. Together, these data indicate that the exemplary agents may be co-formulated with any incretin mimetic.

TABLE 16
Monomer content and absolute potency values for semaglutide formulations

| | | | Monomer | | CBA Potency | | |
|---|---|---|---|---|---|---|---|
| | | | Content (%) | | Activin | GDF-8 | GLP-1R |
| Day | Temp | Agent | P1372) | semaglutide | A (nM) | (nM) | (pM) |
| D 0 | | P1372 alone | 99.6 | N/A | 0.76 | 0.22 | N/A |
| | | P1372 + semaglutide | 99.6 | 100 | 0.72 | 0.19 | 1.4 |
| | | Semaglutide alone | N/A | 100 | N/A | N/A | 2.5 |
| D 7 | 2-8° C. | P1372 alone | 99.6 | N/A | 1.0 | 0.33 | N/A |
| | | P1372 + semaglutide | 99.6 | 100 | 0.77 | 0.25 | 1.7 |
| | | Semaglutide alone | N/A | 100 | N/A | N/A | 3.2 |
| | RT | P1372 alone | 99.4 | N/A | 0.95 | 0.32 | N/A |
| | | P1372 + semaglutide | 99.5 | 100 | 0.84 | 0.31 | 2.3 |
| | | Semaglutide alone | N/A | 100 | N/A | N/A | 2.1 |
| D 14 | 2-8° C. | P1372 alone | 99.5 | N/A | 1.7 | 0.43 | N/A |
| | | P1372 + semaglutide | 99.4 | 100 | 0.96 | 0.22 | 2.7 |
| | | Semaglutide alone | N/A | 100 | N/A | N/A | 2.9 |
| | RT | P1372 alone | 99.3 | N/A | 1.8 | 0.41 | N/A |
| | | P1372 + semaglutide | 99.2 | 100 | 1.6 | 0.39 | 2.3 |
| | | Semaglutide alone | N/A | 100 | N/A | N/A | 3.0 |
| D 28 | 2-8° C. | P1372 alone | 99.5 | N/A | 1.3 | 0.32 | N/A |
| | | P1372 + semaglutide | 99.5 | 100 | 1.0 | 0.3 | 1.7 |
| | | Semaglutide alone | N/A | 99.7 | N/A | N/A | 2.1 |
| | RT | P1372 alone | 98.9 | N/A | 1.2 | 0.4 | N/A |
| | | P1372 + semaglutide | 99.1 | 98.5 | 1.1 | 0.34 | 1.6 |
| | | Semaglutide alone | N/A | 96.5 | N/A | N/A | 2.0 |
| D 56 | 2-8° C. | P1372 alone | 99.3 | N/A | 1.5 | 0.33 | N/A |
| | | P1372 + semaglutide | 99.4 | 100 | 1.1 | 0.24 | 1.3 |
| | | Semaglutide alone | N/A | 100 | N/A | N/A | 1.6 |
| | RT | P1372 alone | 98.2 | N/A | 1.7 | 0.45 | N/A |
| | | P1372 + semaglutide | 98.2 | 97.4 | 1.3 | 0.35 | 1.4 |
| | | Semaglutide alone | N/A | 98.3 | N/A | N/A | 2.1 |

TABLE 17
Monomer content and absolute potency values for tirzepatide formulations

| | | | Monomer | | CBA Potency | | | |
|---|---|---|---|---|---|---|---|---|
| | | | content (%) | | Activin | GDF-8 | GLP-1R | GIPR |
| Day | Temp | Agent | P1372 | tirzepatide | A (nM) | (nM) | (pM) | (pM) |
| D 0 | | P1372 alone | 99.6 | N/A | 0.76 | 0.22 | N/A | N/A |
| | | P1372 + tirzepatide | 99.6 | 100 | 0.56 | 0.16 | 9.3 | 1.5 |
| | | Tirzepatide alone | N/A | 100 | N/A | N/A | 11 | 1.6 |
| D 7 | 2-8° C. | P1372 alone | 99.6 | N/A | 1.0 | 0.33 | N/A | N/A |
| | | P1372 + tirzepatide | 99.6 | 100 | 0.78 | 0.24 | 13 | 1.7 |
| | | Tirzepatide alone | N/A | 100 | N/A | N/A | 11 | 2.8 |
| | RT | P1372 alone | 99.4 | N/A | 0.95 | 0.32 | N/A | N/A |
| | | P1372 + tirzepatide | 99.5 | 100 | 0.74 | 0.24 | 12 | 2.1 |
| | | Tirzepatide alone | N/A | 100 | N/A | N/A | 13 | 2.8 |
| D 14 | 2-8° C. | P1372 alone | 99.5 | N/A | 1.7 | 0.43 | N/A | N/A |
| | | P1372 + tirzepatide | 99.5 | 100 | 1.3 | 0.31 | 18 | 1.5 |
| | | Tirzepatide alone | N/A | 100 | N/A | N/A | 17 | 2.2 |
| | RT | P1372 alone | 99.3 | N/A | 1.8 | 0.41 | N/A | N/A |
| | | P1372 + tirzepatide | 99.2 | 100 | 1.5 | 0.36 | 13 | 1.8 |
| | | Tirzepatide alone | N/A | 100 | N/A | N/A | 23 | 1.7 |
| D 28 | 2-8° C. | P1372 alone | 99.5 | N/A | 1.3 | 0.32 | N/A | N/A |
| | | P1372 + tirzepatide | 99.5 | 100 | 0.99 | 0.26 | 14 | 2.0 |
| | | Tirzepatide alone | N/A | 100 | N/A | N/A | 14 | 2.2 |
| | RT | P1372 alone | 98.9 | N/A | 1.2 | 0.4 | N/A | N/A |
| | | P1372 + tirzepatide | 99 | 100 | 1.1 | 0.28 | 13 | 2.8 |
| | | Tirzepatide alone | N/A | 100 | N/A | N/A | 12 | 2.1 |
| D 56 | 2-8° C. | P1372 alone | 99.3 | N/A | 1.5 | 0.33 | N/A | N/A |
| | | P1372 + tirzepatide | 99.4 | 100 | 1.2 | 0.30 | 7.4 | 1.6 |
| | | Tirzepatide alone | N/A | 100 | N/A | N/A | 7.0 | 1.5 |
| | RT | P1372 alone | 98.2 | N/A | 1.7 | 0.45 | N/A | N/A |
| | | P1372 + tirzepatide | 98.1 | 100 | 1.3 | 0.34 | 7.7 | 1.7 |
| | | Tirzepatide alone | N/A | 100 | N/A | N/A | 6.7 | 1.5 |

TABLE 18
Monomer content and absolute potency values for retatrutide formulations

| | | | Monomer | | CBA potency | | | |
|---|---|---|---|---|---|---|---|---|
| | | | Content (%) | | Activin | GDF-8 | GLP-1R | GIPR |
| Day | Temp | Agent | P1372 | Retatrutide | A (nM) | (nM) | (pM) | (pM) |
| D 0 | | P1372 alone | 99.6 | N/A | 0.76 | 0.22 | N/A | N/A |
| | | P1372 + retatrutide | 99.6 | 100 | 0.58 | 0.15 | 3.0 | 0.94 |
| | | Retatrutide alone | N/A | 100 | N/A | N/A | 3.0 | 0.74 |
| D 7 | 2-8° C. | P1372 alone | 99.6 | N/A | 1.0 | 0.33 | N/A | N/A |
| | | P1372 + retatrutide | 99.6 | 100 | 0.83 | 0.21 | 2.0 | 0.98 |
| | | Retatrutide alone | N/A | 100 | N/A | N/A | 1.8 | 1.0 |
| | RT | P1372 alone | 99.4 | N/A | 0.95 | 0.32 | N/A | N/A |
| | | P1372 + retatrutide | 99.6 | 100 | 0.86 | 0.24 | 1.8 | 0.98 |
| | | Retatrutide alone | N/A | 100 | N/A | N/A | 2.5 | 0.87 |
| D 14 | 2-8° C. | P1372 alone | 99.5 | N/A | 1.7 | 0.43 | N/A | N/A |
| | | P1372 + retatrutide | 99.6 | 100 | 1.3 | 0.31 | 4.1 | 1.1 |
| | | Retatrutide alone | N/A | 100 | N/A | N/A | 3.1 | 1.0 |
| | RT | P1372 alone | 99.3 | N/A | 1.8 | 0.41 | N/A | N/A |
| | | P1372 + retatrutide | 99.4 | 100 | 1.5 | 0.38 | 3.3 | 0.76 |
| | | Retatrutide alone | N/A | 100 | N/A | N/A | 5.6 | 1.2 |
| D 28 | 2-8° C. | P1372 alone | 99.5 | N/A | 1.3 | 0.32 | N/A | N/A |
| | | P1372 + retatrutide | 99.5 | 100 | 0.87 | 0.20 | 2.9 | 1.0 |
| | | Retatrutide alone | N/A | 100 | N/A | N/A | 2.7 | 1.1 |
| | RT | P1372 alone | 98.9 | N/A | 1.2 | 0.4 | N/A | N/A |
| | | P1372 + retatrutide | 99.2 | 100 | 0.96 | 0.31 | 2.1 | 0.84 |
| | | Retatrutide alone | N/A | 100 | N/A | N/A | 2.7 | 1.1 |
| D 56 | 2-8° C. | P1372 alone | 99.3 | N/A | 1.5 | 0.33 | N/A | N/A |
| | | P1372 + retatrutide | 99.4 | 100 | 1.1 | 0.31 | 2.3 | 0.85 |
| | | Retatrutide alone | N/A | 100 | N/A | N/A | 2.1 | 0.84 |
| | RT | P1372 alone | 98.2 | N/A | 1.7 | 0.45 | N/A | N/A |
| | | P1372 + retatrutide | 98.4 | 100 | 1.3 | 0.35 | 2.3 | 0.75 |
| | | Retatrutide alone | N/A | 100 | N/A | N/A | 1.9 | 0.82 |

Finally, to further generalize the potential for co-formulation of incretin mimetics and ectodomain-based traps, other combinations of traps, incretin mimetics, and/or buffer formulations were tested.

First, P1372 (60 mg/mL) was co-formulated with semaglutide (2.4 mg/mL) in a solution of 20 mM NaPO₄, 100 mM Glycine, and 75 mM Histidine (pH 7.5). Results are shown in Table 19. Following 8 weeks of storage at room temperature or 2-8° C., neither component showed compromised integrity, indicating that the components can be co-formulated in different formulation buffers without impacting potency and integrity.

TABLE 19
Monomer content and absolute potency values for exemplary semaglutide formulations

| | | | Monomer | | CBA Potency | | |
|---|---|---|---|---|---|---|---|
| | | | Content (%) | | Activin | GDF-8 | GLP-1R |
| Day | Temp | Agent | P1372 | semaglutide | A (nM) | (nM) | (pM) |
| D 0 | | P1372 alone | 99.5% | N/A | 1.1 nM | 0.29 nM | N/A |
| | | P1372 + semaglutide | 99.4% | 100% | 1.3 nM | 0.34 nM | 1.6 pM |
| | | Semaglutide alone | N/A | 100% | N/A | N/A | 1.8 pM |
| D 56 | 2-8° C. | P1372 alone | 98.7% | N/A | 1.8 nM | 0.46 nM | N/A |
| | | P1372 + semaglutide | 99.1% | 100% | 1.4 nM | 0.47 nM | 1.2 pM |
| | | Semaglutide alone | N/A | 100% | N/A | N/A | 1.3 pM |
| | RT | P1372 alone | 97.6% | N/A | 1.5 nM | 0.51 nM | N/A |
| | | P1372 + semaglutide | 97.9% | 94.3% | 1.5 nM | 0.43 nM | 1.6 pM |
| | | Semaglutide alone | N/A | 95.3% | N/A | N/A | 1.5 pM |

Second, semaglutide was resuspended with or without a different exemplary agent, P1229. P1229 was formulated at 60 mg/mL and semaglutide at 2.4 mg/mL in a solution of 20 mM NaPO₄, 100 mM glycine and 75 mM histidine (pH 7.5). Results are shown in Table 20. Following 8 weeks of storage at room temperature or 2-8° C., neither component showed compromised potency and integrity.

TABLE 20
Monomer content and absolute potency values for semaglutide formulations

| | | | | | Monomer | Monomer | | | |
|---|---|---|---|---|---|---|---|---|---|
| | | | Monomer | Monomer | content | content | Activin A | GDF-8 | GLP-1R |
| | | | content | content | (P1229, | (semaglutide, | potency | potency | potency |
| | | | (P1229, | (semaglutide, | %) Reverse | %) Reverse | (CBA, | (CBA, | (CBA, |
| | | Agent | %) SEC | %) SEC | phase | phase | nM) | nM) | pM) |
| D 0 | | P1229 alone | 94.4% | N/A | 96.7 | N/A | 1.4 nM | 0.63 nM | N/A |
| | | P1229 + semaglutide | 94.6% | 100% | 96.1 | 99.8 | 1.4 nM | 0.56 nM | 6.0 pM |
| | | Semaglutide alone | N/A | 100% | N/A | 99.8 | N/A | N/A | 5.5 pM |
| D 14 | 2-8 degrees | P1229 alone | 95.7% | N/A | 97.1 | N/A | 1.2 nM | 0.51 nM | N/A |
| | | P1229 + semaglutide | 95.8% | 100% | 93.0 | 99.7 | 1.1 nM | 0.40 nM | 5.2 pM |
| | | Semaglutide alone | N/A | 100% | N/A | 99.8 | N/A | N/A | 4.7 pM |
| | Room | P1229 alone | 96.4% | N/A | 95.7 | N/A | 1.4 nM | 0.56 nM | N/A |
| | temperature | P1229 + semaglutide | 96.4% | 100% | 96.8 | 99.3 | 1.1 nM | 0.54 nM | 5.9 pM |
| | | Semaglutide alone | N/A | 100% | N/A | 99.6 | N/A | N/A | 5.9 pM |
| D 28 | 2-8 degrees | P1229 alone | 96.2% | N/A | 99.6 | N/A | 1.4 nM | 0.56 nM | N/A |
| | | P1229 + semaglutide | 96.2% | 100% | 96.0 | 99.8 | 1.2 nM | 0.41 nM | 5.0 pM |
| | | Semaglutide alone | N/A | 100% | N/A | 99.8 | N/A | N/A | 4.8 pM |
| | Room | P1229 alone | 96.3% | N/A | 94.4 | N/A | 1.4 nM | 0.56 nM | N/A |
| | temperature | P1229 + semaglutide | 96.2% | 100% | 97.1 | 98.7 | 1.4 nM | 0.59 nM | 6.2 pM |
| | | Semaglutide alone | N/A | 100% | N/A | 99.2 | N/A | N/A | 5.8 pM |
| D 56 | 2-8 degrees | P1229 alone | 96.3% | N/A | 97.1 | N/A | 1.3 nM | 0.49 nM | N/A |
| | | P1229 + semaglutide | 96.4% | 100% | 97.4 | 99.6 | 1.0 nM | 0.47 nM | 5.4 pM |
| | | Semaglutide alone | N/A | 100% | N/A | 99.8 | N/A | N/A | 4.7 pM |
| | Room | P1229 alone | 96% | N/A | 94.6 | N/A | 1.4 nM | 0.62 nM | N/A |
| | temperature | P1229 + semaglutide | 95.9% | 100% | 96.0 | 96.8 | 1.2 nM | 0.55 nM | 5.6 pM |
| | | Semaglutide alone | N/A | 100% | N/A | 98.6 | N/A | N/A | 6.2 pM |

Lastly, P1372 (40 mg/mL) was co-formulated with tirzepatide (10 mg/mL) in a different buffer comprised of 10 mM NaPO₄, 100 mM Glycine, and 75 mM sucrose (pH 7.5). Results are shown in Table 21. No component showed compromised integrity and potency over a stability period of 8 weeks.

TABLE 21
Monomer content and absolute potency values for tirzepatide formulations

| | | | Monomer | | CBA Potency | | | |
|---|---|---|---|---|---|---|---|---|
| | | | Content (%) | | Activin | GDF-8 | GLP-1R | GIPR |
| Day | Temp | Agent | P1372 | tirzepatide | A (nM) | (nM) | (pM) | (pM) |
| D 0 | | P1372 alone | 98.9% | N/A | 0.59 nM | 0.27 nM | N/A | N/A |
| | | P1372 + tirzepatide | 99% | 100% | 0.48 nM | 0.23 nM | 5.3 pM | 1.3 pM |
| | | Tirzepatide alone | N/A | 100% | N/A | N/A | 4.3 pM | 1.5 pM |
| D 7 | 2-8° C. | P1372 alone | 98.9% | N/A | 0.81 nM | 0.22 nM | N/A | N/A |
| | | P1372 + tirzepatide | 99% | 100% | 0.75 nM | 0.20 nM | 3.2 pM | 1.0 pM |
| | | Tirzepatide alone | N/A | 100% | N/A | N/A | 4.6 pM | 1.1 pM |
| | RT | P1372 alone | 98.8% | N/A | 0.78 nM | 0.24 nM | N/A | N/A |
| | | P1372 + tirzepatide | 99.1% | 100% | 0.62 nM | 0.21 nM | 3.3 pM | 0.99 pM |
| | | Tirzepatide alone | N/A | 100% | N/A | N/A | 3.7 pM | 1.2 pM |
| D 14 | 2-8° C. | P1372 alone | 98.8% | N/A | 0.63 nM | 0.35 nM | N/A | N/A |
| | | P1372 + tirzepatide | 98.8% | 100% | 0.50 nM | 0.27 nM | 2.9 pM | 0.98 pM |
| | | Tirzepatide alone | N/A | 100% | N/A | N/A | 2.4 pM | 0.84 pM |
| | RT | P1372 alone | 97.9% | N/A | 0.74 nM | 0.36 nM | N/A | N/A |
| | | P1372 + tirzepatide | 98.4% | 100% | 0.52 nM | 0.27 nM | 2.8 pM | 1.0 pM |
| | | Tirzepatide alone | N/A | 100% | N/A | N/A | 3.3 PM | 0.82 pM |
| D 28 | 2-8° C. | P1372 alone | 98.9% | N/A | 0.99 nM | 0.45 nM | N/A | N/A |
| | | P1372 + tirzepatide | 98.9% | 100% | 0.74 nM | 0.36 nM | 5.5 pM | 1.3 pM |
| | | Tirzepatide alone | N/A | 100% | N/A | N/A | 5.6 pM | 1.6 pM |
| | RT | P1372 alone | 98.6% | N/A | 0.9 nM | 0.44 nM | N/A | N/A |
| | | P1372 + tirzepatide | 98.7% | 100% | 1.0 nM | 0.42 nM | 5.4 pM | 1.5 pM |
| | | Tirzepatide alone | N/A | 100% | N/A | N/A | 5.0 pM | 1.4 pM |
| D 56 | 2-8° C. | P1372 alone | 98.8% | N/A | 1.4 nM | 0.34 nM | N/A | N/A |
| | | P1372 + tirzepatide | 98.9% | 99.9% | 1.3 nM | 0.30 nM | 9.3 pM | 1.9 pM |
| | | Tirzepatide alone | N/A | 99.9% | N/A | N/A | 8.3 pM | 1.9 pM |
| | RT | P1372 alone | 98.4% | N/A | 1.1 nM | 0.36 nM | N/A | N/A |
| | | P1372 + tirzepatide | 98.4% | 99.9% | 1.2 nM | 0.25 nM | 10 pM | 1.7 pM |
| | | Tirzepatide alone | N/A | 99.9% | N/A | N/A | 8.4 pM | 2.0 pM |

### Example 10. Co-Formulation of an Exemplary Agent with Semaglutide does not Impact the Pharmacokinetic Profile of Either Component

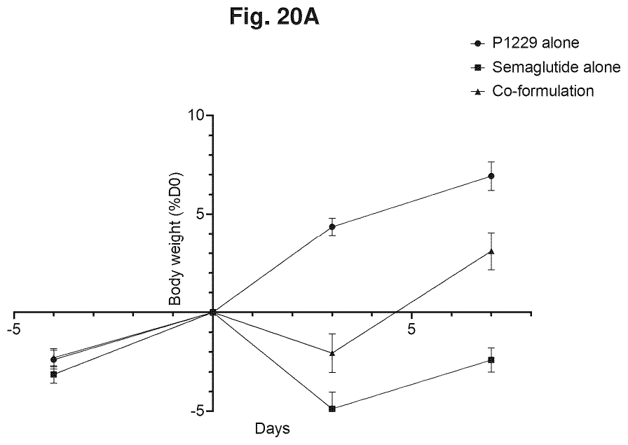

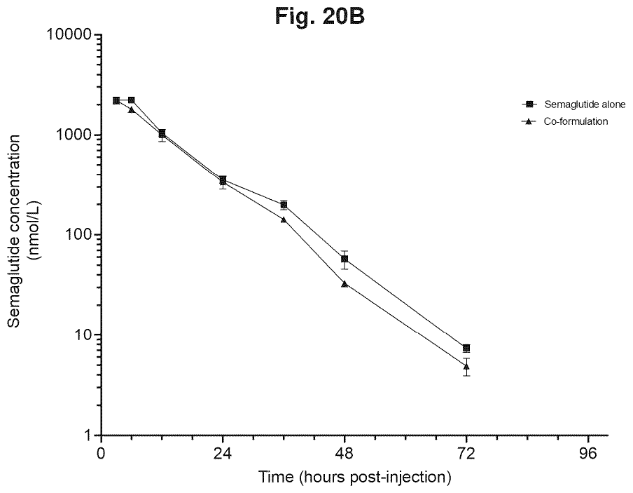

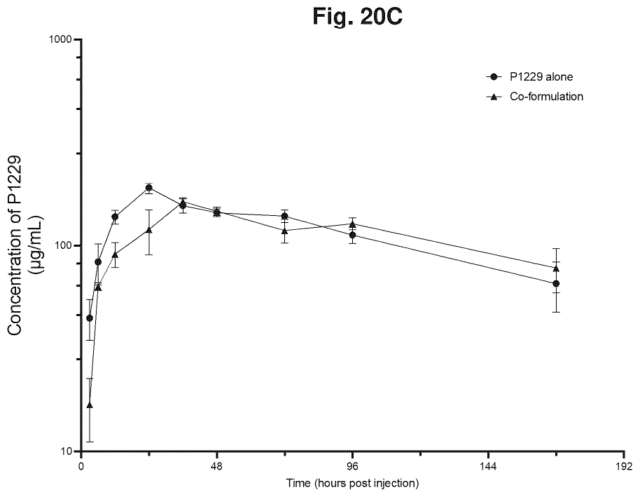

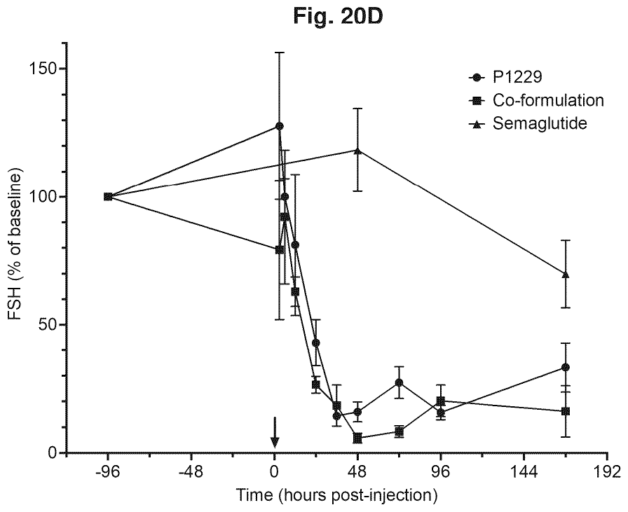

Wild-type CD-1 male mice were subcutaneously injected with 50 mg/kg P1229, 2 mg/kg semaglutide, or a co-formulation of the two agents (50 mg/kg P1229 and 2 mg/kg semaglutide). As expected, animals injected with P1229 showed increased body weight, while those injected with semaglutide showed decreased body weight (FIG. 20A). However, animals injected with the co-formulation showed an intermediate effect on body weight (FIG. 20A). The intermediate response in the group receiving the co-formulation indicated that both components were efficacious in vivo.

In addition, blood samples were collected over a period of 7 days, and the pharmacokinetic (PK) profiles of P1229 and semaglutide were assessed. Semaglutide, alone or in combination with P1229, reached a [C]max of ~2,000 nmol/L between 3 and 6 hours, which is in line with published data available at accessdata.fda.gov/drugsatfda_docs/nda/2017/209637Orig1s000PharmR.pdf, which is incorporated herein by reference in its entirety for purposes. Semaglutide, which has a half-life of ~7 h in mice, was detectable in circulation until 72 h. The PK profiles of semaglutide when administered alone or in the presence of P1229 were comparable indicating that the PK behaviour of the incretin mimetic was not affected when injected as a co-formulation (FIG. 20B). P1229 also showed a comparable PK profile between animals injected with P1229 alone or the co-formulation (FIG. 20C).

These data suggest that a co-formulation of an ectodomain-based trap and an incretin mimetic has PK parameters that are comparable to each individual component.

Finally, circulating FSH levels were assessed throughout the study (FIG. 20D). Both P1229 alone and the co-formulation alone decreased FSH levels by >85%, while semaglutide did not show a significant effect (FIG. 20D). This confirms that the expected pharmacodynamic effect of P1229 was not altered in the context of the co-formulation.

In summary, the results demonstrate that the exemplary agents can be co-formulated with incretin mimetics successfully. Importantly, the co-formulation can tolerate different conditions that may have a range of agent concentrations and different buffer compositions, which had no impact on the quality attributes of ectodomain-based traps and incretins, including their stability, integrity, potency, PK, and efficacy.

### Experimental Procedures for Examples

Production, Purification and Characterization of ActRIIB-ECD fusion proteins that neutralize TGFβ superfamily ligands. All constructs included a secretion signal sequence MDWTWRILFLVAAATGTHA (SEQ ID NO: 1) at the N-terminus when expressed. The complementary cDNAs coding for constructs were prepared synthetically (Genscript, Piscataway, NJ). The cDNAs were cloned into the EcoRI (5′ end) and BamH1 (3′ end) of the pTT5 mammalian expression plasmid vector (Durocher et al., 2002). The signal peptide is cleaved off in the cells during expression and was not included in the purified fusion proteins.

Fusion proteins were expressed by transient transfection of Chinese Hamster Ovary cells (ExpiCHO™, Thermo Fisher, Waltham, MA) at 37° C., 8% CO₂. Transfection conditions were as follows: DNA was transfected according to the manufacturer's instructions (100% fusion protein expression plasmid, 0.5 g/ml, following the recommended volumes for transfection at various scales from Gibco ExpiCHO™ Expression System user guide). At 18-22 hours post-transfection, feed was added (ExpiCHO™ feed) and ExpiFectamine CHO Enhancer, and the culture was transferred to a 32° C., 5% CO₂ incubator. This incubation was conducted to promote the production and secretion of the fusion protein. The culture was maintained for 6 days post-transfection (dpt) after which the cultures were harvested. In some instances, fusion proteins were expressed in Expi293™ (Thermo Fisher, Waltham, MA) cells following procedures similar to those described above. The harvest supernatant was filtered (0.22 μm) and purified by affinity chromatography using an Akta pure 25L (GE). Briefly, the supernatant was loaded onto an MabSelect PrismA protein A column, the column was washed with eight column volumes of PBS and the protein was eluted with five column volumes of 0.1 M sodium citrate, pH 3.2. Fractions containing the purified protein were pooled and buffer exchanged into formulation buffer (20 mM L-Histidine, 100 mM NaCl, pH 7.0) using a HiPrep 26/10 desalting column (GE). The protein was concentrated with a 30 kDa Amicon filter, followed by sterile filtration with a 0.22 um filter prior to further downstream applications.

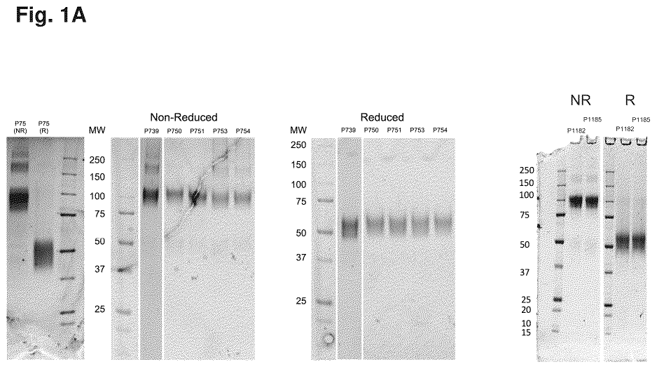

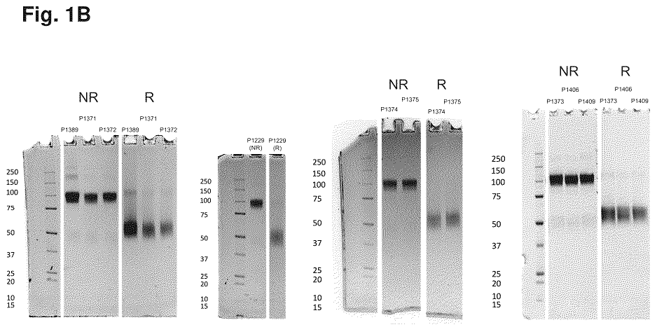

Polyacrylamide gel electrophoresis (SDS-PAGE) analysis of the fusion proteins was carried out under both reducing and non-reducing conditions. Proteins (P) were electrophoresed on a 4-12% Bis-Tris acrylamide gel (NuPAGE™ 4-12% Bis-Tris Protein Gels, Cat #NPO321BOX, Life Technologies). To visualize the proteins, the gels were stained using Coomassie Blue (see FIG. 1).

Given that protein aggregation can be indicative of reduced conformational stability and decreased activity, Size-Exclusion Chromatography-High Performance Liquid Chromatography (SEC-HPLC) was used to determine the purity of each ActRIIB-ECD construct. This method measured the percentage of intact monomeric species as well as the presence of aggregates and/or degradation products.

In some instances, the method used to assess the purity of an agent was based on a reversed-phase high-performance liquid chromatography (RP-HPLC) setup with a C18 column. The mobile phases consisted of acetonitrile with 0.1% formic acid (organic phase) and water with 0.1% formic acid (aqueous phase). A gradient elution was employed, where the proportion of the acetonitrile phase was gradually increased over the course of the run. Formic acid anions form an ion pair with positively charged peptides, increasing their hydrophobicity and therefore their retention time. Detection was performed using a UV detector, at 280 nm.

All proteins expressed efficiently in CHO and/or 293 cells and purification efficiency of each using Protein A affinity chromatography was similar. Although many of the properties were very similar between variants, the potential for aggregation (or high molecular weight (HMW) content), as measured by SEC-HPLC, revealed some distinctions, which could be indicative of improper folding. For the benchmark protein, wild type ActRIIB-ECD-Fc (P75), the purity (i.e., percent monomer content) was 84% monomer and 16% high HMW content. Some of the exemplary agents had aggregate levels comparable to those of P75, namely P739 and P753 with 86 and 84% monomer content, respectively, and corresponding HMW content of 14 and 16%. Strikingly, other agents produced under the same conditions displayed much higher monomer content compared to P75 as exemplified by P750, P751, P754, P1182, P1185, P1229, P1371, P1372, P1373, P1374, P1375, P1389, P1406, and P1409 that showed 96, 98, 93, 97, 96, 97, 99, 99, 98, 99, 99, 97, 97, and 98% monomer content, respectively, and correspondingly lower HMW at 4, 2, 7, 2, 3, 3, 1, 1, 2, 0, 0, 3, 3, and 2% HMW.

These results demonstrate that point mutations in the ActRIIB ectodomain had unpredictable and unexpected consequences on the manufacturability profile of the variant. In particular, P750, P751 and P754 showed a superior monomer content compared to the Fc fusion containing the wild type receptor ectodomain (P75).

Potency testing by neutralization on cell-based assays. Characterization of the activin A, activin B, GDF-8, and GDF-11 inhibition potency (IC₅₀) of the fusion proteins was performed using a cell-based reporter assay. TGFβ-reporter HEK293 cells were obtained from InvivoGen (San Diego, CA, cat. no. hkb-tgfb) and were maintained following the manufacturer's instructions. Briefly, cells were grown in high-glucose DMEM, containing 10% heat-inactivated FBS, 30 μg/mL blasticidin, 250 μg/mL hygromycin B, and 100 μg/mL zeocin (hereafter, “growth medium”). Once the cells reached 70-80% confluence, they were detached in growth medium by gently tapping on the flask. For all assays, cells were seeded in 96-well plates at a density of 20,000 cells per well in growth medium. Cells were left to attach for 10 minutes at room temperature (RT) prior to being transferred to a 37° C. incubator (5% CO₂) overnight. The next day, treatments were prepared by combining the appropriate concentration of agent with activin A, activin B, GDF-8, or GDF-11 for 30 min at RT (shaking at 150 rpm). Treatments were conducted in high-glucose DMEM (serum-free and with no selection agents), and the final concentration of cytokine (ligand) was 0.3 nM. Growth medium was removed from the culture plates, and cells were treated with the appropriate mixes of fusion protein and cytokine. Treatment duration was for 18-22 h at 37° C. (5% CO₂). All treatments were performed in technical duplicates and at least one or two independent experiments were conducted for each cytokine. At the end of the treatment period, supernatants were transferred to a new plate, and centrifuged at 300 RPM for 3 min. Forty microlitres of supernatant from each condition was transferred to a new plate, into which 180 μL of Quanti-Blue reagent (InvivoGen, cat. no. rep-qbs) was added. Quanti-Blue reagent was prepared following the manufacturer's instructions. Samples were incubated for 45 min at 37° C. At the end of the incubation, absorbance was read at 650 nm. The data were plotted in GraphPad Prism, which was used to calculate IC₅₀ values as a non-linear regression.

Characterization of the BMP-9 and BMP-10 inhibition potency (IC₅₀) of the fusion proteins was performed using a second cell-based reporter assay. Human hepatic HepG2 cells were transfected with a SEAP-encoding plasmid, in which SEAP expression is under the control of the murine Id1 promoter. A stable pool was generated from this transfection. Cells were grown in EMEM, containing 10% heat-inactivated FBS and 200 μg/mL zeocin (hereafter, “HepG2 growth medium”). Once cells reached 70% confluence, they were washed with PBS and detached using 0.25% trypsin. For all assays, cells were seeded in 96-well plates at a confluence of 100,000 cells per well in HepG2 growth medium. Cells were left to attach for 10 minutes at RT prior to being transferred to a 37° C. incubator (5% CO₂) overnight. The next day, treatments were prepared in EMEM (2.5% heat-inactivated FBS and with no selection agents) by combining the appropriate concentration of agent with BMP-9 or BMP-10 for 30 min at RT (shaking at 150 rpm). Treatments were conducted in EMEM (2.5% heat-inactivated FBS and with no selection agents), and the final concentration of BMP-9 and BMP-10 was 2 ng/mL and 40 ng/mL, respectively. HepG2 growth medium was removed from the culture plates, and cells were treated with the appropriate mixes of fusion protein and cytokine. Treatment duration was for 20-24 h at 37° C. (5% CO₂). All treatments were performed in technical duplicates and at least one or two independent experiments were conducted for each cytokine. At the end of the treatment period, supernatants were transferred to a new plate, and centrifuged at 300 RPM for 3 min. Forty microlitres of supernatant from each condition was transferred to a new plate, onto which 180 μL of Quanti-Blue reagent was added. Quanti-Blue reagent was prepared following the manufacturer's instructions. Samples were incubated for 5-6 h with Quanti-Blue reagent at 37° C. At the end of the incubation, absorbance was read at 650 nm. The data were plotted in GraphPad Prism, which was used to calculate IC₅₀ values as a non-linear regression.

Characterization of the activity of semaglutide, tirzepatide, or retatrutide on the GLP-1 receptor (GLP-1R) was assessed using CHO-K1 cells stably overexpressing GLP-1R. These reporter cells were purchased from Genscript (Piscataway, NJ, cat #M00451) and were maintained following the manufacturer's instructions. Briefly, cells were grown in Ham's F-12K (Kaighn's modified) medium, containing 10% heat-inactivated FBS, 200 μg/mL zeocin, and 100 my/mL hygromycin B (hereafter, “growth medium”). Once the cells reached 70-80% confluence, they were detached using trypsin. For all assays, cells were seeded in clear-bottom, opaque 96-well plates at a density of 10,000 cells per well in growth medium. Cells were left to attach for 10 minutes at room temperature prior to being transferred to a 37° C. incubator (5% CO₂) overnight. The next day, cells were treated with incretin mimetics to assess intracellular cAMP accumulation, using the cAMP-Glo™ Max Assay from Promega (Madison, WI, cat #V1682) following the manufacturer's instructions. Briefly, cells were starved in Ham's F-12K (Kaighn's modified) medium (no additional supplements) for 2 hours at 37° C. incubator (5% CO₂). Then, treatments were prepared in Ham's F-12K (Kaighn's modified) medium containing 30 mM MgCl₂, 500 μM IBMX (isobutyl-1-methylxanthine), and 100 μM Ro 20-1724 ([4-(3-butoxy-4-methoxy-benzyl) imidazolidone) (hereafter, “induction medium”). Medium was removed from the culture plates, and cells were treated with the appropriate mixes of incretin mimetics. Treatment duration was for 30 min at 37° C. (5% CO₂). All treatments were performed in technical duplicates and at least two independent experiments were conducted for each incretin mimetic. Cells were lyzed for 20 minutes at room temperature (150 rpm) using the kit's buffer. Following the kit's instructions, luciferin was then added for 10 minutes at room temperature (in the dark), following which luminescence was assessed. The data were plotted in GraphPad Prism, which was used to calculate IC₅₀ values as a non-linear regression.

Characterization of the activity of tirzepatide or retatrutide on the GIP receptor (GIPR) was assessed using CHO-K1 cells stably overexpressing GIPR. These reporter cells were purchased from Genscript (Piscataway, NJ, cat #M00486). The same methodology described above was applied for these cells.

Potency testing by ELISA. To determine potency on activin A, a 96-well plate was coated overnight at 4° C. with a solution of activin A (R&D 338-AC, 0.5 μg/mL in PBS, 100 μL/well). The next day, the plate was washed three times with buffer (Biolegend #421601). Blocking buffer (1% BSA, 0.05% tween-20 in PBS, 300 μL/well) was added to each well and the plate was incubated for 1 hour at RT. Starting at 2.5 μg/mL, test agents were diluted in assay diluent (1% BSA, 0.05% tween-20 in PBS, 100 μL/well) and loaded on the plate for 1 h at RT. The plate was washed three times and a solution of anti-human Fc antibody conjugated to HRP (Jackson ImmunoResearch Laboratories #109-035-098, 0.01 ug/mL diluted in assay diluent, 100 μL/well) was added to each well and incubated for 1 hour at RT. After washing three times, binding was revealed using standard colorimetric method.

To determine potency on GLP-1R, a 96-well plate was coated overnight at 4° C. with a solution of GLP-1 (R&D 5374, 0.25 μg/mL in carbonate-bicarbonate buffer, 100 μL/well). The next day, the plate was washed three times with buffer (Biolegend #421601). Blocking buffer (0.5% ovalbumin, in PBS, 300 μL/well) was added to each well and the plate was incubated for 1 hour at RT. Test agents were diluted in assay diluent (0.05% tween-20 in PBS, 100 μL/well), in the presence or absence of His-tagged GLP-1R (R&D 10956-GL, final concentration of 50 ng/mL) and loaded on the plate for 1 h at RT. The plate was washed three times and a solution of anti-His antibody conjugated to HRP (R&D MAB050H, 1:3000 diluted in assay diluent, 100 μL/well) was added to each well and incubated for 1 hour at RT. After washing three times, binding was revealed using standard colorimetric method.

Small-scale testing of ActRIIB-ECD fusion proteins. Additional investigation of a series of exemplary agents was performed using proteins expressed in CHO cells. Briefly, DNA encoding the ActRIIB-ECD-Fc fusion constructs was transfected in cells seeded in 12-well plates in a final volume of 1 ml. After 7 days, the supernatant was harvested, clarified, and the amount of expressed protein in each well was quantified using standard methods. The potency of each agent for inhibition of activin A, BMP-9 and BMP-10 was determined in three separate assays.

To determine potency on activin A, a 96-well plate was coated overnight at 4° C. with a solution of anti-activin A antibody (R&D #MAB3381, 0.25 μg/mL in a carbonate-bicarbonate buffer, 100 μL/well). The next day, the plate was washed three times with buffer (Biolegend #421601). Blocking buffer (5% BSA in PBS, 200 μL/well) was added to each well and the plate was incubated for 1 hour at RT. Expressed proteins in the supernatants described above were diluted to 2 μg/mL in assay diluent (0.5% BSA, 0.05% tween-20 in PBS), mixed 1:1 with recombinant human activin A (R&D #338-AC, 60 ng/mL, diluted in assay diluent). Complexes were immediately added to the 96-well plate in technical duplicates and incubated for 1 h at RT (concentration of expressed protein and activin A on the plate were 1 μg/mL and 30 ng/mL, respectively). The plate was washed three times and a solution of anti-activin A antibody conjugated to biotin (R&D #BAM3381, 1 μg/mL diluted in assay diluent, 100 μL/well) was added to each well and incubated for 1 hour at RT. After washing three times, binding was revealed using streptavidin-HRP using a standard colorimetric method. In this assay, a loss of signal indicates binding of the exemplary agent to activin A, which prevents activin A binding to the anti-activin A antibody on the plate. Results are expressed as a % of the signal obtained with wild-type ActRIIB-Fc (P75).

To determine potency on BMP-9, a 96-well plate was coated overnight at 4° C. with a solution of anti-human IgG antibody (SouthemBiotech #2049-01, 1/6000 in a carbonate-bicarbonate buffer, 100 μL/well). The next day, the plate was washed three times. Blocking buffer (5% BSA in PBS, 200 μL/well) was added to each well and the plate was incubated for 1 hour at RT. Expressed proteins in the supernatants were diluted 1 in 2 in assay diluent (0.5% BSA, 0.05% tween-20 in PBS) and 100 μl was added to the 96-well plate in technical duplicates. After a 1.5-hour incubation at RT, the plate was washed three times. BMP-9 (R&D #3209-BP, 10 ng/mL diluted in assay diluent, 100 μL/well) was added to each well and incubated for 1.5 h at RT. The plate was washed three times, and a solution of anti-BMP-9 antibody conjugated to biotin (R&D #BAF3209, 0.4 ug/mL diluted in assay diluent, 100 μL/well) was added to each well and incubated for 1 hour at RT. After washing three times, binding was revealed using streptavidin-HRP using a standard colorimetric method. In this assay, a loss of signal indicates less binding of the exemplary agent to BMP-9. Results are expressed as a % of the signal obtained with wild-type ActRIIB-Fc (P75).

To determine potency on BMP-10, the same BMP-reporter cell line was used as described above. A final concentration of 2.5 nM of each expressed protein was tested in the presence of 40 ng/mL of BMP-10 (R&D #2926-BP). In this assay, a loss of signal indicates neutralization of BMP-10 by the exemplary agent. Results are expressed as a % of the signal obtained with the cytokine alone. A separate assay was used to estimate BMP-10 binding, using bio-layer interferometry (BLI) on a Gator® Plus instrument (Gator Bio, Palo Alto, USA). An anti-Human IgG Fc Gen II probe (#160024, Gator Bio) was loaded with expressed proteins in CHO supernatants, following which BMP-10 binding kinetics (single concentration, 12.5 nM) were assessed.

Evaluation of pharmacokinetic profiles. Levels of semaglutide in circulation were assessed using the GLP-1R cell-based assay described above, with some modifications. First, cell density was 25,000 cells/well, and stimulation time lasted 15 minutes. Minimal required dilution for all serum samples was 1 in 1000, and all samples, standards, and quality controls (QCs) were diluted in induction medium containing 0.1% CD-1 mouse serum.

To determine the concentration of P1229 in mouse serum samples, a 96-well plate was coated 1 h at RT with a solution of anti-ectodomain antibody (1 μg/mL in carbonate buffer, 100 μL/well). The plate was then washed three times with buffer (Biolegend #421601). Blocking buffer (1% BSA, 0.05% tween-20 in TBS, 300 μL/well) was added to each well and the plate was incubated for 1 hour at RT with shaking at 300 rpm. The plate was washed three times and Standards, QCs and sample were diluted in assay diluent (1% BSA, 0.05% tween-20 in TBS, 100 μL/well) and loaded in duplicate on the plate for 1 h30 at RT with shaking at 300 rpm. The plate was washed three times and a solution of anti-human Fc antibody conjugated to HRP (Southern Biotech #2049-05, diluted 1 in 20,000 in assay diluent, 100 μL/well) was added to each well and incubated for 1 h at RT with shaking at 300 rpm. After washing three times, binding was revealed using a standard colorimetric method.

Although this invention is described in detail with reference to embodiments thereof, these embodiments are offered to illustrate but not to limit the invention. It is possible to make other embodiments that employ the principles of the invention and that fall within its spirit and scope as defined by the claims appended hereto.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

1. A combination comprising
(a) a TGFβ superfamily ligand binding agent comprising a first polypeptide and a second polypeptide, wherein the first and second polypeptides each comprise:
i. a polypeptide comprising an amino acid sequence that is at least 90% identical to an Activin receptor type IIB (ActRIIB) ectodomain (ECD) variant, the ActRIIB ECD variant comprising an amino acid substitution at the position corresponding to position 33 of SEQ ID NO: 2;
ii. an Fc domain monomer; and
iii. a peptide linker joining the polypeptide to the Fc domain monomer; and
(b) a Glucagon-like peptide 1 (GLP-1) agonist.

2. The combination of claim 1, wherein the ActRIIB ECD variant demonstrates reduced inhibition of BMP-9 and/or BMP-10 compared to a wild-type ActRIIB ectodomain.

3. The combination of claim 1 or claim 2, wherein the amino acid substitution is selected from L33F, L33Q, L33Y, L33W, L33H, L33R, L33E, L33K, and L33M.

4. The combination of any one of claims 1-3, wherein the ActRIIB ECD variant comprises the amino acid substitution L33F.

5. The combination of claim 4, wherein the ActRIIB ECD variant
(a) comprises an amino acid sequence that is at least 90%, at least 95%, at least 96%, at least 97%, at least 98%, or at least 99% identical to SEQ ID NO: 10; or
(b) comprises or consists of the amino acid sequence of SEQ ID NO: 10.

6. The combination of any one of claims 1-3, wherein the ActRIIB ECD variant comprises the amino acid substitution L33Q.

7. The combination of claim 6, wherein the ActRIIB ECD variant
(a) comprises an amino acid sequence that is at least 90%, at least 95%, at least 96%, at least 97%, at least 98%, or at least 99% identical to SEQ ID NO: 11; or
(b) comprises or consists of the amino acid sequence of SEQ ID NO: 11.

8. The combination of any one of claims 1-3, wherein the ActRIIB ECD variant comprises the amino acid substitution L33Y.

9. The combination of claim 8, wherein the ActRIIB ECD variant
(a) comprises an amino acid sequence that is at least 90%, at least 95%, at least 96%, at least 97%, at least 98%, or at least 99% identical to SEQ ID NO: 12; or
(b) comprises or consists of the amino acid sequence of SEQ ID NO: 12.

10. The combination of any one of claims 1-3, wherein the ActRIIB ECD variant comprises the amino acid substitution L33W.

11. The combination of claim 10, wherein the ActRIIB ECD variant
(a) comprises an amino acid sequence that is at least 90%, at least 95%, at least 96%, at least 97%, at least 98%, or at least 99% identical to SEQ ID NO: 13; or
(b) comprises or consists of the amino acid sequence of SEQ ID NO: 13.

12. The combination of any one of claims 1-3, wherein the ActRIIB ECD variant comprises the amino acid substitution L33H.

13. The combination of claim 12, wherein the ActRIIB ECD variant
(a) comprises an amino acid sequence that is at least 90%, at least 95%, at least 96%, at least 97%, at least 98%, or at least 99% identical to SEQ ID NO: 14; or
(b) comprises or consists of the amino acid sequence of SEQ ID NO: 14.

14. The combination of any one of claims 1-3, wherein the ActRIIB ECD variant comprises the amino acid substitution L33R.

15. The combination of claim 14, wherein the ActRIIB ECD variant
(a) comprises an amino acid sequence that is at least 90%, at least 95%, at least 96%, at least 97%, at least 98%, or at least 99% identical to SEQ ID NO: 15; or
(b) comprises or consists of the amino acid sequence of SEQ ID NO: 15.

16. The combination of any one of claims 1-3, wherein the ActRIIB ECD variant comprises the amino acid substitution L33E.

17. The combination of claim 16, wherein the ActRIIB ECD variant
(a) comprises an amino acid sequence that is at least 90%, at least 95%, at least 96%, at least 97%, at least 98%, or at least 99% identical to SEQ ID NO: 16; or
(b) comprises or consists of the amino acid sequence of SEQ ID NO: 16.

18. The combination of any one of claims 1-3, wherein the ActRIIB ECD variant comprises the amino acid substitution L33K.

19. The combination of claim 18, wherein the ActRIIB ECD variant
(a) comprises an amino acid sequence that is at least 90%, at least 95%, at least 96%, at least 97%, at least 98%, or at least 99% identical to SEQ ID NO: 17; or
(b) comprises or consists of the amino acid sequence of SEQ ID NO: 17.

20. The combination of any one of claims 1-3, wherein the ActRIIB ECD variant comprises the amino acid substitution L33M.

21. The combination of claim 20, wherein the ActRIIB ECD variant
(a) comprises an amino acid sequence that is at least 90%, at least 95%, at least 96%, at least 97%, at least 98%, or at least 99% identical to SEQ ID NO: 18; or
(b) comprises or consists of the amino acid sequence of SEQ ID NO: 18.

22. The combination of claim 21, wherein the ActRIIB ECD variant further comprises an amino acid substitution at position 27 of SEQ ID NO: 2.

23. The combination of claim 21 or 22, wherein the ActRIIB ECD variant further comprises an amino acid substitution at position 69 of SEQ ID NO: 2.

24. The combination of any one of claims 22-23, wherein the ActRIIB ECD variant further comprises one or more amino acid substitutions which is G27D, G27E, T69E, T69Q, or T69H.

25. The combination of any one of claims 1-24, wherein the ActRIIB ECD variant further comprises one or more additional amino acids at the N or C terminus.

26. The combination of claim 25, wherein the ActRIIB ECD variant further comprises the following amino acids at the N terminus: GRGEA (SEQ ID NO: 63) and/or the following amino acids at the C-terminus: APT.

27. The combination of any one of claims 1-26, wherein the first and the second polypeptides comprise the following structure, from N- to C-terminus: ActRIIB-ECD-peptide linker-Fc domain monomer.

28. The combination of any one of claims 1-27, wherein the Fc domain monomer is an IgG1, IgG2, IgG3 or IgG4 isotype.

29. The combination of any one of claims 1-28, wherein the Fc domain monomer is a human Fc domain monomer or a murine Fc domain monomer.

30. The combination of any one of claims 1-29, wherein the Fc domain monomer is engineered to reduce aggregation or to modulate stability of a dimer of the polypeptide.

31. The combination of claim 30, wherein the Fc domain monomer comprises the amino acid substitutions of M252Y, S254T, and T256E (YTE).

32. The combination of claim 30, wherein the Fc domain monomer comprises the M252Y amino acid substitution.

33. The combination of any one of claims 28-32, wherein the Fc domain monomer includes a D at position 356 and an L at position 358 (DL).

34. The combination of any one of claims 28-32, wherein the Fc domain monomer includes an E at position 356 and an M at position 358 (EM).

35. The combination of any one of claims 28-34, wherein the Fc domain monomer further comprises a Lysine residue (K) at the C terminus.

36. The combination of any one of claims 1-35, wherein the Fc domain monomer
(a) comprises an amino acid sequence that is at least 85%, at least 90%, at least 95%, at least 96%, at least 97%, at least 98%, or at least 99% identical to any one of SEQ ID NOs: 252-292; or
(b) comprises or consists of an amino acid sequence selected from any one of SEQ ID NOs: 252-292.

37. The combination of any one of claims 28-36, wherein the Fc domain monomer is an IgG1 isotype

38. The combination of claim 37, wherein the Fc domain monomer comprises or consists of the amino acid sequence set forth in SEQ ID NO: 253, SEQ ID NO: 255, or SEQ ID NO: 256.

39. The combination of any one of claims 1-38, wherein the Fc domain monomer in the first polypeptide forms a dimer with the Fc domain monomer in the second polypeptide.

40. The combination of any one of claims 1-39, wherein the peptide linker is Glycine-rich.

41. The combination of any one of claims 1-40, wherein the peptide linker is between 10 and 40 amino acids long.

42. The combination of claim 41, wherein the linker is at least 10 amino acids long, at least 14 amino acids long, at least 19 amino acids long, or at least 39 amino acids long.

43. The combination of claim 42, wherein the linker is 10 amino acids long, 14 amino acids long, 19 amino acids long, or 39 amino acids long.

44. The combination of any one of claims 40-43, wherein the peptide linker comprises the amino acid sequence set forth in any one of SEQ ID NOs: 89, 94, or 98.

45. The combination of any one of claims 1-44, wherein the ActRIIB-ECD
(a) comprises an amino acid sequence that is at least 95%, 96%, 97%, 98%, or 99% identical to any one of SEQ ID NOs:4-62; or
(b) comprises or consists of an amino acid sequence selected from any one of SEQ ID NOs: 4-62.

46. The combination of any one of claims 1-45, wherein the ActRIIB-ECD comprises or consists of the amino acid sequence of SEQ ID NO: 10-18, or a sequence at least 95%, at least 96%, at least 97%, at least 98%, or at least 99% identical thereto.

47. The combination of any one of claims 1-46, wherein the ActRIIB-ECD comprises or consists of the amino acid sequence of SEQ ID NO: 13 or a sequence at least 95%, at least 96%, at least 97%, at least 98%, or at least 99% identical thereto.

48. The combination of any one of claims 1-47, wherein the first and/or second polypeptide:
(a) comprise an amino acid sequence that is at least 95%, 96%, 97%, 98%, or 99% identical to an amino acid sequence selected from SEQ ID NOs: 174-251; or
(b) comprise or consist of an amino acid sequence selected from SEQ ID NOs: 174-251.

49. The combination of any one of claims 1-48, wherein the first and/or second polypeptide:
(a) comprises an amino acid sequence that is at least 95%, 96%, 97%, 98%, or 99% identical to an amino acid sequence selected from SEQ ID NOs: 175, 176, 180, 204-214, and 230-234; or
(b) comprises or consists of an amino acid sequence selected from SEQ ID NOs: 175, 176, 180, 204-214, and 230-234.

50. The combination of any one of claims 1-48, wherein the first and/or second polypeptide:
(a) comprises an amino acid sequence that is at least 95%, 96%, 97%, 98%, or 99% identical to an amino acid sequence selected from SEQ ID NOs: 211 and 230-234; or
(b) comprises or consists of an amino acid sequence selected from SEQ ID NOs: 211 and 230-234.

51. The combination of any one of claims 1-48, wherein the first and/or second polypeptide:
(a) comprises or consists of the amino acid sequence of SEQ ID NO: 231, or an amino acid sequence that is at least 95%, 96%, 97%, 98%, or 99% identical thereto; or
(b) comprises or consists of the amino acid sequence of SEQ ID NO: 231.

52. The combination of any one of claims 1-48, wherein the first and/or second polypeptide (a) comprises or consists of the amino acid sequence of SEQ ID NO: 234, or an amino acid sequence that is at least 95%, 96%, 97%, 98%, or 99% identical thereto; or
(b) comprises or consists of the amino acid sequence of SEQ ID NO: 234.

53. The combination of any one of claims 1-52, wherein the first and/or second polypeptide further comprise an albumin-binding domain, a fibronectin domain, or a human serum albumin domain fused to the N- or C-terminus of the ActRIIB-ECD via a linker.

54. The combination of any one of claims 1-53, wherein the first and/or second polypeptide further comprises a signal peptide of SEQ ID NO: 1 at the N-terminus of the ActRIIB-ECD.

55. The combination of claim 54, wherein the signal peptide is cleaved from the mature protein.

56. The combination of any one of claims 1-55, wherein the binding agent is conjugated with a targeting agent, a therapeutic moiety, a detectable moiety, or a diagnostic moiety.

57. The combination of claim 56, wherein the targeting agent, the therapeutic moiety, the detectable moiety, or the diagnostic moiety comprises an antibody or antigen binding fragment thereof, a binding agent having affinity for another member of the TGFβ superfamily or for another therapeutic target, a radiotherapy agent, an imaging agent, a fluorescent moiety, a cytotoxic agent, an anti-mitotic drug, a nanoparticle-based carrier, a polymer-conjugated drug, a nanocarrier, an imaging agent, a stabilizing agent, a drug, a nanocarrier, or a dendrimer.

58. The combination of any one of claims 1-57, wherein the first and second polypeptides form a dimer linked by at least one disulfide bond between the Fc domain monomer of the first polypeptide and the Fc domain monomer of the second polypeptide.

59. The combination of any one of claims 1-58, wherein the binding agent:
(a) demonstrates similar or increased binding to human activin A, activin B, GDF-8, and/or GDF-11 and demonstrates reduced binding to human BMP-9 and/or BMP-10 compared to a polypeptide comprising a WT ActRIIB-ECD;
(b) does not substantially bind to human BMP-9;
(c) demonstrates reduced binding to human BMP-10 compared to a polypeptide comprising a WT ActRIIB-ECD;
(d) inhibits signaling of one or more of human activin A, activin B, GDF-8, and GDF-11; and/or
(e) does not substantially inhibit human BMP-9 and/or BMP-10 signaling.

60. The combination of any one of claims 1-59, wherein the GLP-1 agonist is an antibody, small molecule, peptide, or aptamer.

61. The combination of any one of claims 1-60, wherein the GLP-1 agonist is selected from the group consisting of exenatide, exenatide extended-release, efpeglenatide, dulaglutide, liraglutide, lixisenatide, lotiglipron, semaglutide, tirzepatide, cotadutide, noiiglutide, oxyntomodulin, orforglipron, retatrutide, albiglutide, beinaglutide and PEG-loxenatide, pemvidutide, and danuglipron.

62. The combination of any one of claims 1-59, wherein the GLP-1 agonist is selected from semaglutide and tirzepatide.

63. The combination of any one of claims 1-59, wherein the GLP-1 agonist is a dual GLP-1 agonist and GIP agonist, a dual GLP-1 agonist and GCG agonist, or a triagonist of GIP/GLP-1/glucagon receptors.

64. The combination of any one of claims 1-63, wherein the binding agent and GLP-1 agonist are formulated in separate pharmaceutical compositions.

65. The combination of any one of claims 1-63, wherein the binding agent and GLP-1 agonist are formulated in the same pharmaceutical composition.

66. The combination of any one of claims 64-65, wherein the pharmaceutical composition is formulated for administration by injection or infusion.

67. The combination of claim 66, wherein the composition is formulated for intravenous, subcutaneous, intraperitoneal, or intramuscular administration.

68. A kit comprising the combination of any one of claims 1-67, and optionally, directions for use.

69. A method of treating or preventing a metabolic disease or condition in a subject in need thereof, the method comprising administering the combination of any one of claims 1-67 to the subject.

70. The method of claim 69, wherein the subject is a human.

71. The method of claim 69 or 70, wherein the metabolic disorder is selected from the group consisting of obesity, diabetes, metabolic syndrome, anti-psychotic drug-associated obesity, glucocorticoid-induced obesity, hypothalamic obesity associated with craniopharyngioma, Prader-Willi syndrome, and a monogenetic disorder associated with obesity such as Bardet-Biedl syndrome.

72. The method of claim 69 or 70, wherein the metabolic disorder is selected from obesity, Type 2 diabetes, and pre-diabetes.

73. The method of claim 71, wherein the metabolic disorder is obesity.

74. The method of any one of claims 69-73, wherein the method does not: cause a vascular complication in a subject; increase vascular permeability or leakage in a subject; increase red blood cell mass; increase hemoglobin; cause thrombocytopenia; cause a hematological complication in a subject; and/or does not cause a reduction in lean muscle mass in the subject.

75. The method of any one of claims 69-74, wherein the treatment increases lean mass in the subject.

76. The method of any one of claims 69-75, wherein the treatment reduces fat mass in the subject.
